(19) United States
(12) Patent Application Publication
(10) Pub. No.: US 2024/0287147 A1
(43) Pub. Date: Aug. 29, 2024

(54) PD-L1 BINDING PEPTIDES AND PEPTIDE COMPLEXES AND METHODS OF USE THEREOF

(71) Applicants: Fred Hutchinson Cancer Center, Seattle, WA (US); Blaze Bioscience, Inc., Seattle, WA (US)

(72) Inventors: Zachary CROOK, Seattle, WA (US); James OLSON, Seattle, WA (US); Natalie Winblade NAIRN, Seattle, WA (US); Colin CORRENTI, Seattle, WA (US)

(21) Appl. No.: 18/037,926

(22) PCT Filed: Nov. 29, 2021
(86) PCT No.: PCT/US2021/061039

§ 371 Date: May 19, 2023

Related U.S. Application Data
(60) Provisional application No. 63/119,195, filed on Nov. 30, 2020.
(60) Provisional application No. 63/273,103, filed on Oct. 28, 2021.

(51) Int. Cl. C07K 14/435

(52) U.S. Cl. CPC C07K 14/43522

(57) ABSTRACT

Described herein are cystine-dense peptides capable of binding to PD-L1. Such peptides may function as PD-L1 inhibitors by binding to the PD-1-binding interface of PD-L1 and inhibiting interactions between PD-L1 and PD-1. Methods of using PD-L1-binding peptides to treat cancers, autoimmune diseases, or other conditions are also described herein. Such methods include delivering active agents complexed with a PD-L1-binding peptide to a PD-L1 positive cell or using a bispecific immune cell engager to recruit immune cells to PD-L1 positive cells. Also described herein are chimeric antigen receptors comprising PD-L1-binding peptides.

### CROSS-REFERENCE

The present application claims the benefit of U.S. Provisional Application No. 63/119,195, entitled “COMPOSITIONS AND METHODS FOR SELECTIVE DEPLETION OF TARGET MOLECULES,” filed on Nov. 30, 2020; and U.S. Provisional Application No. 63/273,103, entitled “PD-L1 BINDING PEPTIDES AND PEPTIDE COMPLEXES AND METHODS OF USE THEREOF,” filed on Oct. 28, 2021, each of which applications are herein incorporated by reference in their entirety for all purposes.

### SEQUENCE LISTING

The instant application contains a Sequence Listing which has been submitted electronically in ASCII format and is hereby incorporated by reference in its entirety. Said ASCII copy, created on Nov. 23, 2021, is named 108406-709288_SL.txt and is 627,485 bytes in size.

### BACKGROUND

Programmed death-ligand 1 (PD-L1), a transmembrane protein that binds programmed cell death protein 1 (PD-1), is thought to play a role in immune suppression and inactivation of T cells. Cancer cells that express PD-L1 can evade the host immune response by binding to PD-1 receptors on host T cells and inactivate the T cells, preventing destruction of the cancer cell by the host immune system. Anti-PD-L1 antibodies may block binding of PD-L1 to PD-1. However, there is a need for additional PD-L1-binding agents that may be used to target PD-L1.

### SUMMARY

In various aspects, the present disclosure provides PD-L1-binding peptide comprising a first PD-L1-binding motif comprising a sequence of: (a) X¹X²X³X⁴X⁵X⁶CX⁷X⁸X⁹C (SEQ ID NO: 361), wherein X¹ is D, E, H, K, N, Q, S, T, L, V, F, Y, or P; X² is G, E, Q, or F; X³ is D or K; X⁴ is G, V, or P; X⁵ is G, H, R, V, F, W, or P; X⁶ is A, D, or K; X⁷ is E, H, Q, L, or F; X⁸ is D, E, R, S, T, M, L, or F; and X⁹ is G, A, D, E, H, K, R, M, L, or P; or (b) X¹FX²VFX²CLX³X³C (SEQ ID NO: 363), wherein X¹ is K or P; X² is independently D or K; and X³ is independently any non-cysteine amino acid.

In some aspects, the PD-L1-binding peptide comprises at least six cysteine residues. In some aspects, the at least six cysteine residues are located at amino acid positions n, n+4, n+14, n+28, n+38, and n+42, wherein n corresponds to a position of a first cysteine residue of the at least six cysteine residues. In some aspects, amino acid position n corresponds to amino acid position 4, such that the at least six cysteine amino acid residues are located at amino acid positions 4, 8, 18, 32, 42, and 46.

In some aspects, the PD-L1-binding peptide further comprises at least three disulfide bonds connecting the at least six cysteine residues. In some aspects, the at least three disulfide bonds connect: a first cysteine residue of the at least six cysteine residues to a sixth cysteine residue of the at least six cysteine residues, a second cysteine residue of the at least six cysteine residues to a fifth cysteine residue of the at least six cysteine residues, a third cysteine residue of the at least six cysteine residues to a forth cysteine residue of the at least six cysteine residues. In some aspects, the first cysteine residue is at amino acid position n, the second cysteine residue is at amino acid position n+4, the third cysteine residue is at amino acid position n+14, the fourth cysteine residue is at amino acid position n+28, the fifth cysteine residue is at amino acid position n+38, and the sixth cysteine residue is at amino acid position n+42. In some aspects, the first cysteine residue is at amino acid position 4, the second cysteine residue is at amino acid position 8, the third cysteine residue is at amino acid position 18, the fourth cysteine residue is at amino acid position 32, the fifth cysteine residue is at amino acid position 42, and the sixth cysteine residue is at amino acid position 46.

In some aspects, the PD-L1-binding peptide further comprises a first alpha helix comprising residues n to n+20, where n corresponds to an amino acid position of a first cysteine residue. In some aspects, the PD-L1-binding peptide further comprises a second alpha helix comprising residues n+34 to n+44, where n corresponds to an amino acid position of a first cysteine residue. In some aspects, the second alpha helix comprises residues n+29 to n+44.

In some aspects, the N-terminal amino acid residue of the first PD-L1-binding motif is located at amino acid residue position n+32, where n corresponds to an amino acid position of a first cysteine residue. In some aspects, the C-terminal amino acid residue of the first PD-L1-binding motif is located at amino acid position n+42, where n corresponds to an amino acid position of a first cysteine residue. In some aspects, the first PD-L1-binding motif comprises a sequence of KFDVFKCLDHC (SEQ ID NO: 365).

In some aspects, the PD-L1-binding peptide further comprises a second PD-L1-binding motif comprising a sequence of: (a) CX¹X²X³CX⁴X⁵X⁶X⁷X⁸X⁹X¹⁰X¹¹X¹²C (SEQ ID NO: 360), wherein X¹ is K, R, or V; X² is E, Q, S, M, L, or V; X³ is D, E, H, K, R, N, Q, S, or Y; X⁴ is D, M, or V; X⁵ is A, K, R, Q, S, or T; X⁶ is A, D, E, H, Q, S, T, M, I, L, V, or W; X⁷ is A, E, R, Q, S, T, W, or P; X⁸ is A, E, K, R, N, Q, T, M, I, L, V, or W; X⁹ is G, A, E, K, N, T, or Y; X¹⁰ is G, A, D, E, H, K, R, N, Q, S, T, M, I, L, V, W, Y, or P; X¹¹ is D, K, R, N, L, or V; and X¹² is G, A, D, T, L, W, or P; or (b) CKVX¹CVX¹X¹X¹X¹X²X³KX¹C (SEQ ID NO: 362), wherein X¹ is independently any non-cysteine amino acid; X² is M, I, L, or V; and X³ is Y, A, H, K, R, N, Q, S, or T. In some aspects, the N-terminal amino acid residue of the second PD-L1-binding motif is located at amino acid residue position n, where n corresponds to an amino acid position of a first cysteine residue. In some aspects, the C-terminal amino acid residue of the first PD-L1-binding motif is located at amino acid position n+14, where n corresponds to an amino acid position of a first cysteine residue. In some aspects, the second PD-L1-binding motif comprises a sequence of CKVHCVKEWMAGKAC (SEQ ID NO: 364).

In some aspects, the PD-L1-binding peptide comprises a sequence of SEQ ID NO: 358 or SEQ ID NO: 359. In some aspects, the PD-L1-binding peptide comprises a sequence having at least 80%, at least 85%, at least 90%, at least 95%, at least 97%, or 100% sequence identity to SEQ ID NO: 1. In some aspects, the PD-L1-binding peptide comprises a sequence having at least 80%, at least 85%, at least 90%, at least 95%, at least 97%, or 100% sequence identity to SEQ ID NO: 2. In some aspects, the PD-L1-binding peptide comprises a sequence having at least 80%, at least 85%, at least 90%, at least 95%, at least 97%, or 100% sequence identity to SEQ ID NO: 3. In some aspects, the PD-L1-binding peptide comprises a sequence having at least 80%, at least 85%, at least 90%, at least 95%, at least 97%, or 100% sequence identity to SEQ ID NO: 4.

In various aspects, the present disclosure provides a PD-L1-binding peptide comprising at least six cysteine residues are located at amino acid positions n, n+4, n+14, n+28, n+38, and n+42, wherein n corresponds to a position of a first cysteine residue of the at least six cysteine residues, and at least 80%, at least 85%, at least 90%, at least 95%, at least 97%, or 100% sequence identity to SEQ ID NO: 57 or SEQ ID NO: 59.

In various aspects, the present disclosure provides a PD-L1-binding peptide comprising at least eight cysteine residues are located at amino acid positions n, n+11, n+17, n+21, n+31, n+38, n+40, or n+44, wherein n corresponds to a position of a first cysteine residue of the at least six cysteine residues, and at least 80%, at least 85%, at least 90%, at least 95%, at least 97%, or 100% sequence identity to SEQ ID NO: 58.

In various aspects, the present disclosure provides a PD-L1-binding peptide comprising a sequence having at least 80%, at least 85%, at least 90%, at least 95%, at least 97%, or 100% sequence identity to any one of SEQ ID NO: 1-SEQ ID NO: 118, SEQ ID NO: 435, SEQ ID NO: 436, SEQ ID NO: 437, or SEQ ID NO: 554-SEQ ID NO: 567.

In some aspects, the PD-L1-binding peptide comprises a sequence of any one of SEQ ID NO: 1-SEQ ID NO: 118, SEQ ID NO: 435, SEQ ID NO: 436, SEQ ID NO: 437, or SEQ ID NO: 554-SEQ ID NO: 567.

In some aspects, the PD-L1-binding peptide is capable of binding to PD-L1 with an equilibrium dissociation constant (K_{D}) of not greater than 100 nM, not greater than 50 nM, not greater than 1 nM, not greater than 500 pM, not greater than 300 pM, not greater than 250 pM, or not greater than 200 pM. In some aspects, the PD-L1-binding peptide is capable of binding to PD-L1 with an equilibrium dissociation constant (K_{D}) of not greater than 1 nM. In some aspects, the PD-L1-binding peptide is capable of binding to a human PD-L1 and a cynomolgus PD-L1 with an equilibrium dissociation constant (K_{D}) that differs by no more than 1.5-fold, no more than 2-fold, no more than 5-fold, or no more than 10-fold.

In some aspects, the PD-L1-binding peptide comprises at least 43, at least 44, at least 45, at least 46, at least 47, at least 48, or at least 49 amino acid residues.

The PD-L1-binding peptide of any one of claims 1-29, wherein the PD-L1-binding peptide comprises from 43 to 51 amino acid residues. In some aspects, the PD-L1-binding peptide comprises not more than 50 amino acid residues. In some aspects, the PD-L1-binding peptide comprises from 43 to 49 amino acid residues.

In some aspects, the PD-L1-binding peptide further comprises a half-life modifying agent. In some aspects, the half-life modifying agent is selected from the group consisting of a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, albumin, and a molecule that binds to albumin. In some aspects, the half-life modifying agent is an albumin-binding peptide. In some aspects, the half-life modifying agent is an Fc domain. In some aspects, the half-life modifying agent is a polyethylene glycol. In some aspects, the half-life modifying agent is a fatty acid.

In various aspects, the present disclosure provides a peptide complex comprising a PD-L1-binding peptide complexed with an active agent, wherein the PD-L1-binding peptide comprises: at least six cysteine residues located at amino acid positions n, n+4, n+14, n+28, n+38, and n+42, where n corresponds to an amino acid position of a first cysteine residue of the at least six cysteine residues; at least three disulfide bonds connecting the first cysteine residue to a sixth cysteine residue of the at least six cysteine residues, a second cysteine residue of the at least six cysteine residues to a fifth cysteine residue of the at least six cysteine residues, a third cysteine residue of the at least six cysteine residues to a forth cysteine residue of the at least six cysteine residues.

In some aspects, amino acid position n corresponds to amino acid position 4, such that the at least six cysteine amino acid residues are located at amino acid positions 4, 8, 18, 32, 42, and 46. In some aspects, the PD-L1-binding peptide further comprises a first alpha helix comprising residues n to n+20, where n corresponds to an amino acid position of a first cysteine residue. In some aspects, the PD-L1-binding peptide further comprises a second alpha helix comprising residues n+34 to n+44, where n corresponds to an amino acid position of a first cysteine residue. In some aspects, the second alpha helix comprises residues n+29 to n+44.

In some aspects, the PD-L1-binding peptide comprises a first PD-L1-binding motif comprising a sequence of: (a) X¹X²X³X⁴X⁵X⁶CX⁷X⁸X⁹C (SEQ ID NO: 361), wherein X¹ is D, E, H, K, N, Q, S, T, L, V, F, Y, or P; X² is G, E, Q, or F; X³ is D or K; X⁴ is G, V, or P; X⁵ is G, H, R, V, F, W, or P; X⁶ is A, D, or K; X⁷ is E, H, Q, L, or F; X⁸ is D, E, R, S, T, M, L, or F; and X⁹ is G, A, D, E, H, K, R, M, L, or P; or (b) X¹FX²VFX²CLX³X³C (SEQ ID NO: 363), wherein X¹ is K or P; X² is independently D or K; and X³ is independently any non-cysteine amino acid. In some aspects, the N-terminal amino acid residue of the first PD-L1-binding motif is located at amino acid residue position n+32. In some aspects, the C-terminal amino acid residue of the first PD-L1-binding motif is located at amino acid position n+42. In some aspects, the first PD-L1-binding motif comprises a sequence of KFDVFKCLDHC (SEQ ID NO: 365).

In some aspects, the PD-L1-binding peptide further comprises a second PD-L1-binding motif comprising a sequence of: (a) CX¹X²X³CX⁴X⁵X⁶X⁷X⁸X⁹X¹⁰X¹¹X¹²C (SEQ ID NO: 360), wherein X¹ is K, R, or V; X² is E, Q, S, M, L, or V; X³ is D, E, H, K, R, N, Q, S, or Y; X⁴ is D, M, or V; X⁵ is A, K, R, Q, S, or T; X⁶ is A, D, E, H, Q, S, T, M, I, L, V, or W; X⁷ is A, E, R, Q, S, T, W, or P; X⁸ is A, E, K, R, N, Q, T, M, I, L, V, or W; X⁹ is G, A, E, K, N, T, or Y; X¹⁰ is G, A, D, E, H, K, R, N, Q, S, T, M, I, L, V, W, Y, or P; X¹¹ is D, K, R, N, L, or V; and X¹² is G, A, D, T, L, W, or P; or (b) CKVX¹CVX¹X¹X¹X¹X²X³KX¹C (SEQ ID NO: 362), wherein X¹ is independently any non-cysteine amino acid; X² is M, I, L, or V; and X³ is Y, A, H, K, R, N, Q, S, or T. In some aspects, the N-terminal amino acid residue of the second PD-L1-binding motif is located at amino acid residue position n. In some aspects, the C-terminal amino acid residue of the first PD-L1-binding motif is located at amino acid position n+14. In some aspects, the second PD-L1-binding motif comprises a sequence of CKVHCVKEWMAGKAC (SEQ ID NO: 364). In some aspects, amino acid position n corresponds to amino acid position 4 of the PD-L1-binding peptide, such that the at least six cysteine amino acid residues are located at amino acid positions 4, 8, 18, 32, 42, and 46 of the PD-L1-binding peptide.

In some aspects, the PD-L1-binding peptide is capable of binding to PD-L1 with an equilibrium dissociation constant (K_{D}) of not greater than 100 nM, not greater than 50 nM, not greater than 30 nM, not greater than 20 nM, not greater than 1 nM, not greater than 500 pM, not greater than 300 pM, not greater than 250 pM, or not greater than 200 pM. In some aspects, the PD-L1-binding peptide comprises at least 43, at least 44, at least 45, at least 46, at least 47, at least 48, or at least 49 amino acid residues. In some aspects, the PD-L1-binding peptide comprises from 43 to 51 amino acid residues. In some aspects, the PD-L1-binding peptide comprises from 43 to 49 amino acid residues.

In various aspects, the present disclosure provides a peptide complex comprising any PD-L1-binding peptide described herein complexed with an active agent.

In some aspects, the active agent comprises an immune cell targeting agent. In some aspects, the immune cell targeting agent is an immune cell targeting peptide. In some aspects, the immune cell targeting agent comprises a single chain variable fragment (scFv), a cysteine-dense peptide, an avimer, a kunitz domain, an affibody, an adnectin, a nanofittin, a fynomer, a 8-hairpin, a stapled peptide, a bicyclic peptide, an antibody, an antibody fragment, a protein, a peptide, a peptide fragment, a binding domain, a small molecule, or a nanobody capable of binding to an immune cell. In some aspects, the immune cell targeting agent is capable of binding a T cell, a B cell, a macrophage, a natural killer cell, a fibroblast, a regulatory T cell, a regulatory immune cell, a neural stem cell, or a mesenchymal stem cell. In some aspects, the immune cell targeting agent is capable of binding a T cell. In some aspects, the immune cell targeting agent is capable of binding a regulatory T cell. In some aspects, the immune cell targeting agent is capable of binding CD3, 4-1BB, CD28, CD137, CD89, CD16, CD25, CD13, CD29, CD44, CD71, CD73, CD90, CD105, CD166, CD27, GITR, TIGIT, LAG3, TCR, CD40L, OX40, PD-1, CTLA-4, or STRO-1. In some aspects, the immune cell targeting agent is capable of binding CD3. In some aspects, the immune cell targeting agent is capable of binding CD25. In some aspects, the immune cell targeting agent is capable of binding 4-1BB. In some aspects, the immune cell targeting agent is capable of binding CD28. In some aspects, the immune cell targeting agent comprises a sequence having at least 90% sequence identity to any one of SEQ ID NO: 122 or SEQ ID NO: 442-SEQ ID NO: 491.

In some aspects, the immune cell targeting agent is fused to a first heterodimerization domain and the PD-L1-binding peptide is fused to a second heterodimerization domain. In some aspects, the first heterodimerization domain complexes with the second heterodimerization domain to form a heterodimer. In some aspects, the first heterodimerization domain, the second dimerization domain, or both comprises a Fc domain. In some aspects, the first heterodimerization domain, the second dimerization domain, or both comprises a sequence of any one of SEQ ID NO: 124-SEQ ID NO: 153. In some aspects, the first heterodimerization domain comprises a sequence of any one of SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, or SEQ ID NO: 152 and the second heterodimerization domain comprises a sequence of any one of SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, or SEQ ID NO: 153. In some aspects, the first heterodimerization domain comprises Chain 1 of a heterodimerization pair provided in TABLE 3. In some aspects, the second heterodimerization domain comprises Chain 2 of a heterodimerization pair provided in TABLE 3. In some aspects, the second heterodimerization domain comprises a sequence of any one of SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, or SEQ ID NO: 152 and the first heterodimerization domain comprises a sequence of any one of SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, or SEQ ID NO: 153. In some aspects, the first heterodimerization domain comprises Chain 2 of a heterodimerization pair provided in TABLE 3. In some aspects, the second heterodimerization domain comprises Chain 1 of a heterodimerization pair provided in TABLE 3. In some aspects, the peptide complex comprises a sequence having at least 90% sequence identity to SEQ ID NO: 119 or SEQ ID NO: 120. In some aspects, the peptide complex comprises a sequence having at least 90% sequence identity to SEQ ID NO: 123.

In some aspects, the immune cell targeting agent and the PD-L1-binding peptide are fused to a homodimerization domain. In some aspects, the immune cell targeting agent and the PD-L1-binding peptide form a single polypeptide chain. In some aspects, the peptide complex comprises a sequence having at least 90% sequence identity to any one of SEQ ID NO: 121 or SEQ ID NO: 438-SEQ ID NO: 441.

In some aspects, the immune cell targeting agent is linked to the PD-L1-binding peptide via a linker. In some aspects, the linker comprises a peptide linker. In some aspects, linker comprises a small molecule linker. In some aspects, the linker comprises an Fc domain. In some aspects, the peptide complex further comprises an albumin-binding domain, a polyethylene glycol, or both.

In some aspects, the active agent comprises a transmembrane domain, an intracytoplasmic domain, or a combination thereof. In some aspects, the active agent comprises a chimeric antigen receptor. In some aspects, the peptide complex further comprises a T cell.

In some aspects, the active agent comprises a therapeutic agent, a detectable agent, or a combination thereof. In some aspects, the detectable agent comprises a fluorophore, a near-infrared dye, a contrast agent, a nanoparticle, a metal-containing nanoparticle, a metal chelate, an X-ray contrast agent, a PET agent, a radionuclide, or a radionuclide chelator. In some aspects, the therapeutic agent comprises an anti-cancer agent, a chemotherapeutic agent, a radiotherapy agent, an anti-inflammatory agent, a proinflammatory cytokine, an oligonucleotide, an immuno-oncology agent, or a combination thereof. In some aspects, the active agent comprises a radioisotope. In some aspects, the radioisotope comprises an alpha emitter, a beta emitter, a positron emitter, a gamma emitter, a metal, actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, yttrium, actinium-225, lead-212, ¹¹C or ¹⁴C, ¹³N, ¹⁸F, ⁶⁷Ga, ⁶⁸Ga, ⁶⁴Cu, ⁶⁷Cu, ⁸⁹Zr, ¹⁷⁷Lu, indium-111, technetium-99m, yttrium-90, iodine-131, iodine-123, or astatine-211.

In some aspects, the oligonucleotide comprises a DNA, an RNA, an antisense oligonucleotide, an aptamer, an miRNA, an siRNA, an alternative splicing modulator, a mRNA-binding sequence, an miRNA-binding sequence, an siRNA-binding sequence, an RNaseH1-binding oligonucleotide, a RISC-binding oligonucleotide, a polyadenylation modulator, or a combination thereof. In some aspects, the oligonucleotide comprises a sequence of any one of SEQ ID NO: 366-SEQ ID NO: 396, SEQ ID NO: 492-SEQ ID NO: 545, or SEQ ID NO: 552. In some aspects, the oligonucleotide binds a target sequence comprising any one of SEQ ID NO: 397-SEQ ID NO: 430 or SEQ ID NO: 546-SEQ ID NO: 549. In some aspects, the peptide complex remains intact after incubation in human serum. In some aspects, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% remains intact after incubation in human serum. In some aspects, the PD-L1-binding peptide retains an equilibrium dissociation constant (K_{D}) for PD-L1 of no more than 10 nM, 5 nM, 1 nM, 800 pM, 600 pM, 500 pM, 400 pM, 300 pM, 250 pM, or 200 pM when complexed with the oligonucleotide. In some aspects, the PD-L1-binding peptide has a lower affinity for PD-L1 at pH 5.5, 6.0, or 6.5 than at pH 7.4.

In some aspects, the anti-inflammatory agent comprises an anti-inflammatory cytokine, a steroid, a glucocorticoid, a corticosteroid, a cytokine inhibitor, a RORgamma inhibitor, a JAK inhibitor, a tyroskine kinase inhibitor, or a nonsteroidal anti-inflammatory drug. In some aspects, the anti-cancer agent comprises an antineoplastic agent, a cytotoxic agent, a tyrosine kinase inhibitor, an mTOR inhibitor, a retinoid, a microtubule polymerization inhibitor, a pyrrolobenzodiazepine dimer, or an anti-cancer antibody. In some aspects, the proinflammatory cytokine comprises a TNFα, an IL-2, an IL-6, an IL-12, an IL-15, an IL-21, or an IFNγ. In some aspects, the therapeutic agent comprises an oncolytic viral vector.

In some aspects, the peptide complex further comprises a half-life modifying agent. In some aspects, the half-life modifying agent is selected from the group consisting of a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, and a molecule that binds to albumin. In some aspects, the molecule that binds to albumin is a serum albumin-binding peptide.

In some aspects, the peptide complex further comprises a cell-penetrating peptide. In some aspects, the cell-penetrating peptide comprises a sequence of any one of SEQ ID NO: 249-SEQ ID NO: 341.

In various aspects, the present disclosure provides a pharmaceutical composition comprising any PD-L1-binding peptide described herein, or any peptide complex described herein, and a pharmaceutically acceptable carrier.

In various aspects, the present disclosure provides a method of inhibiting PD-L1 in a subject, the method comprising: administering to the subject a composition comprising a PD-L1-binding peptide, the PD-L1-binding peptide comprising at least six cysteine residues, and at least three disulfide bonds connecting the at least six cysteine residues; binding the PD-L1-binding peptide to PD-L1 on a PD-L1 positive cell; and inhibiting the PD-L1.

In some aspects, the at least six cysteine residues are located at amino acid positions n, n+4, n+14, n+28, n+38, and n+42, where n corresponds to an amino acid position of a first cysteine residue of the at least six cysteine residues. In some aspects, amino acid position n corresponds to amino acid position 4, such that the at least six cysteine amino acid residues are located at amino acid positions 4, 8, 18, 32, 42, and 46.

In various aspects, the present disclosure provides a method of inhibiting PD-L1 in a subject, the method comprising: administering to the subject a composition comprising any PD-L1-binding peptide described herein; binding the PD-L1-binding peptide to PD-L1 on a PD-L1 positive cell; and inhibiting the PD-L1.

In some aspects, inhibiting PD-L1 comprises inhibiting binding of PD-1 to PD-L1. In some aspects, the method further comprises reducing immunosuppression, reducing T cell exhaustion, restoring immune function, or a combination thereof. In some aspects, the method further comprises treating a condition in the subject. In some aspects, the condition is cancer, and wherein the PD-L1 positive cell is a cancer cell. In some aspects, treating the cancer comprises enhancing an immune response against the cancer cell.

In various aspects, the present disclosure provides a method of delivering an active agent to a PD-L1 positive cell of a subject, the method comprising: administering to the subject a peptide complex comprising a PD-L1-binding peptide complexed with an active agent, the PD-L1-binding peptide comprising at least six cysteine residues, and at least three disulfide bonds connecting the at least six cysteine residues; binding the PD-L1-binding peptide to a PD-L1 positive cell; and delivering the active agent to the PD-L1 positive cell.

In some aspects, the at least six cysteine residues are located at amino acid positions n, n+4, n+14, n+28, n+38, and n+42, where n corresponds to an amino acid position of a first cysteine residue of the at least six cysteine residues. In some aspects, amino acid position n corresponds to amino acid position 4, such that the at least six cysteine amino acid residues are located at amino acid positions 4, 8, 18, 32, 42, and 46.

In various aspects, the present disclosure provides a method of delivering an active agent to a PD-L1 positive cell of a subject, the method comprising: administering to the subject a peptide complex comprising any PD-L1-binding peptide described herein complexed with an active agent, or the peptide complex of any one of claims 39-107; binding the PD-L1-binding peptide to a PD-L1 positive cell; and delivering the active agent to the PD-L1 positive cell.

In some aspects, the active agent comprises an anti-cancer agent, a chemotherapeutic agent, a radiotherapy agent, or a proinflammatory cytokine. In some aspects, the active agent comprises an oligonucleotide. In some aspects, the peptide complex remains intact after incubation in human serum. In some aspects, the PD-L1-binding peptide binds to PD-L1 with equilibrium dissociation constant (K_{D}) of no more than 10 nM, 5 nM, 1 nM, 800 pM, 600 pM, 500 pM, 400 pM, 300 pM, 250 pM, or 200 pM when complexed with the oligonucleotide.

In some aspects, the method further comprises binding the oligonucleotide to a target sequence upon delivery to the PD-L1 positive cell. In some aspects, the method further comprises modulating alternative splicing of the target sequence, dictating the location of a polyadenylation site of the target sequence, inhibiting translation of the target sequence, inhibiting binding of the target sequence to a secondary target sequence, recruiting RISC to the target sequence, recruiting RNaseH1 to the target sequence, inducing cleavage of the target sequence, or regulating the target sequence upon binding of the oligonucleotide to the target sequence. In some aspects, the active agent comprises an anti-inflammatory cytokine, a steroid, a glucocorticoid, a corticosteroid, or a nonsteroidal anti-inflammatory drug. In some aspects, the active agent comprises an immune cell targeting agent.

In some aspects, the method further comprises binding the immune cell targeting agent to an immune cell and recruiting the immune cell to the PD-L1 positive cell. In some aspects, recruiting the immune cell to the PD-L1-positive cell comprises forming an immunological synapse. In some aspects, the immunological synapse has a width of from 3 nm to 25 nm, from 5 nm to 20 nm, or from 10 nm to 15 nm. In some aspects, the immunological synapse has a width of no greater than 3 nm, no greater than 5 nm, no greater than 8 nm, no greater than 10 nm, no greater than 13 nm, no greater than 15 nm, no greater than 18 nm, no greater than 20 nm, no greater than 23 nm, no greater than 25 nm, no greater than 30 nm, no greater than 35 nm, no greater than 40 nm, no greater than 45 nm, or no greater than 50 nm.

In some aspects, the immune cell comprises a T cell, a B cell, a macrophage, a natural killer cell, a fibroblast, a regulatory T cell, a regulatory immune cell, a neural stem cell, or a mesenchymal stem cell. In some aspects, the immune cell targeting agent binds CD3, 4-1BB, CD28, CD137, CD89, CD16, CD25, CD13, CD29, CD44, CD71, CD73, CD90, CD105, CD166, CD27, GITR, TIGIT, LAG3, TCR, CD40L, OX40, PD-1, CTLA-4, or STRO-1. In some aspects, the immune cell targeting agent binds CD3. In some aspects, the immune cell targeting agent binds CD25. In some aspects, the immune cell targeting agent binds 4-1BB. In some aspects, the immune cell targeting agent binds CD28.

In some aspects, the method further comprises killing the PD-L1 positive cell upon delivery of the immune cell to the PD-L1 positive cell. In some aspects, the method further comprises suppressing the PD-L1 positive cell upon delivery of the immune cell to the PD-L1 positive cell. In some aspects, the immune cell targeting agent comprises a single chain variable fragment (scFv), a cysteine-dense peptide, an avimer, a kunitz domain, an affibody, an adnectin, a nanofittin, a fynomer, a ß-hairpin, a stapled peptide, a bicyclic peptide, an antibody, an antibody fragment, a protein, a peptide, a peptide fragment, a binding domain, a small molecule, or a nanobody.

In some aspects, the immune cell targeting agent is fused to a first heterodimerization domain and the PD-L1-binding peptide is fused to a second heterodimerization domain. In some aspects, the first heterodimerization domain complexes with the second heterodimerization domain to form a heterodimer. In some aspects, the first heterodimerization domain, the second dimerization domain, or both comprises a Fc domain.

In some aspects, the immune cell targeting agent is linked to the PD-L1-binding peptide via a linker. In some aspects, the immune cell targeting agent is linked to the PD-L1-binding peptide via an Fc domain. In some aspects, the immune cell targeting agent and the PD-L1-binding peptide form a single polypeptide chain

In some aspects, the peptide complex comprises a chimeric antigen receptor. In some aspects, the active agent comprises a transmembrane domain, an intracytoplasmic domain, or a combination thereof. In some aspects, the peptide complex further comprises a T cell. In some aspects, the method further comprises delivering the T cell to the PD-L1 positive cell. In some aspects, the method further comprises killing the PD-L1 positive cell.

In some aspects, the method further comprises treating a condition in the subject. In some aspects, the condition is cancer. In some aspects, the PD-L1 positive cell is a cancer cell. In some aspects, the cancer comprises melanoma, skin cancer, non-small cell lung cancer, small cell lung cancer, renal cancer, esophageal cancer, oral cancer, hepatocellular cancer, ovarian cancer, cervical cancer, colorectal cancer, colon cancer, rectal cancer, head and neck cancer, lymphoma, bladder cancer, liver cancer, gastric cancer, stomach cancer, breast cancer, triple negative breast cancer, pancreatic cancer, prostate cancer, Merkel cell carcinoma, mesothelioma, brain cancer, or a PD-L1-expressing cancer. In some aspects, the brain cancer comprises glioblastoma, astrocytoma, meningioma, primary brain cancer, metastatic brain cancer, a PDL1-expressing cancer, or a metastatic brain cancer.

In some aspects, the condition is hyperglycemia, type 1 diabetes, or type 2 diabetes. In some aspects, the PD-L1 positive cell comprises a pancreatic beta cell. In some aspects, the immune cell is a regulatory T cell, and wherein recruitment of the regulatory T cell to the pancreatic beta cell protects the pancreatic beta cell, and prevents, mitigates effect of, reduces symptoms of, slows onset of the hyperglycemia, the type 1 diabetes, or the type 2 diabetes in the subject, thereby treating the hyperglycemia, the type 1 diabetes, or the type 2 diabetes.

In some aspects, the condition is an autoimmune or inflammatory disorder. In some aspects, the PD-L1 positive cell comprises a pancreatic beta cell. In some aspects, the immune cell comprises a regulatory T cell or a mesenchymal stem cell. In some aspects, the immune cell targeting agent binds CD25, CD13, CD29, CD44, CD71, CD73, CD90, CD105, CD166, or STRO-1. In some aspects, upon recruitment to PD-L1 positive cell, the immune cell inhibits an autoimmune or inflammatory response, thereby treating the autoimmune or inflammatory disorder. In some aspects, the autoimmune or inflammatory disorder comprises rheumatoid arthritis, atherosclerosis, ischemia-reperfusion injury, colitis, psoriasis, lupus, inflammatory bowel disease, Crohn's disease, ulcerative colitis, multiple sclerosis, type 1 diabetes, type 2 diabetes, or neuroinflammation.

In some aspects, the PD-L1-binding peptide binds to PD-L1 with an equilibrium dissociation constant (K_{D}) of not greater than 100 nM, not greater than 50 nM, not greater than 1 nM, not greater than 500 pM, not greater than 300 pM, not greater than 250 pM, or not greater than 200 pM.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1D illustrate mammalian display and library screening methods for selection of cystine dense peptide (CDP) scaffolds for PD-L1-binding peptides.

FIG. 1A schematically illustrates a general mammalian surface display screening method using lentiviral vectors to screen for target-binding properties or to assess peptide quality (right, “Vector SDPR”). Mammalian cells, such as HEK 293F cells, express and display GFP-tagged cystine dense peptides (CDPs) from a library of CDPs. In a first assay (left, “Vector SDGF”), CDPs are screened for binding to a fluorescently labeled target protein (e.g., a biotinylated protein labeled with a fluorescent streptavidin). Cells are sorted based on co-stain fluorescence to select for CDPs that bind to the target protein. In a second assay, tagged CDPs from the CDP library are assessed for quality. Tagged CPDs (e.g., CDPs with a 6×His tag (SEQ ID NO: 248)) that are intact are labeled with a co-stain (e.g., a fluorescently labeled anti-6×His antibody). Cells are sorted based on co-stain fluorescence to select for CDPs that are expressed and displayed in-tact.

FIG. 1B shows a histogram distribution of the surface folding scores for CDPs from a taxonomically diverse CDP library. The top scoring CDPs (N=953), corresponding to cystine scaffolds predicted to have high surface expression, protease resistance, or both, were selected for further analysis.

FIG. 1C schematically illustrates a system used to identify members for a new CDP library. The approximately 100,000 curated CDP sequences were searched for CDPs with sequence homology or cysteine pattern homology to the top scoring CDPs identified in FIG. 1B. The resulting second-generation optimized library (“Gen 2 Library”) contained 8,893 CDPs.

FIG. 1D shows a flow-based comparison of CDP stability between a first-generation diversified CDP library and the second-generation optimized CDP library identified in FIG. 1C and containing peptides with homology to predicted high stability CDP scaffolds. Cells SDPR-cloned with 6×His-tagged (SEQ ID NO: 248) CDPs from either the diversified (left) or optimized (right) library were treated with 5 μg/mL trypsin (thin solid line), 20 μg/mL trypsin (bold solid line), or PBS (dashed line, “No Trypsin”) for 5 minutes. Cells were subsequently treated with DTT for 5 minutes and stained with a fluorescent anti-6×His antibody. Histogram distributions of CDPs were normalized to the mode. The optimized library showed improved surface expression and protease resistance over the diversified library.

FIG. 2A-FIG. 2C illustrate selection of various PD-L1-binding peptides that align with three-dimensional structure, computational modeling and phylogeny resulting in high confidence for selection of PD-L1-binding peptides with PD-L1 binding activity.

FIG. 2A schematically illustrates a structural modeling pipeline used to predict three-dimensional structure and disulfide pairing of members of an optimized CDP library. I-TASSER version 5.1 modeling software was used to create a structural model from a CDP sequence. The likeliest disulfide pairings were determined by minimizing the average pairwise distances between bonded cysteine sulfur atoms in the structural model. Rosetta ForceDislufides program relaxed the disulfide-bonded structures with an all-atom refinement and repacking algorithm to minimize steric clashes.

FIG. 2B shows representative alignments of crystal structures and computational models, obtained as described in FIG. 2A, for twenty CDPs from an optimized CDP library. The crystal structures of these CDPs were not included in the structural database used for computational modeling. Crystal structures available from the RCSB Protein Data Bank are referenced by PDB ID number. Root-mean-square deviations (RMSDs) of the alignment between the crystal structure and the modeled structure are provided below the PDB ID number or the SEQ ID NO. For crystal structures with multiple polypeptide chains in the asymmetric unit, the RMSD represents the average alignment between the modeled structure and each polypeptide in the asymmetric unit.

FIG. 2C shows a graphical comparison of the root-mean-square deviation (RMSD) of the twenty CDP alignments shown in FIG. 2B. The computational model aligned to an asymmetric unit (AU) of the crystal structure with an average RMSD of 0.924 Å (left circles). Different asymmetric units of the crystals aligned to each other with an average RMSD of 0.333 Å (right circles), providing a quantitative assessment of the accuracy of the models used in FIG. 2A and FIG. 2B.

FIG. 3 shows a structural model of center of mass clusters (mesh cages denoted with arrows) of CDP library peptides docked to programmed death-ligand 1 (PD-L1). PD-1 bound to PD-L1 is shown as a ribbon structure based on its crystal structure (PDB ID NO: 4ZQK; Zak et al., Structure 23, 2341-2348 (2015)). These center of mass clusters are examples of where PD-L1 binding peptides may bind to PD-L1. A center of mass cluster located near the PD-1 binding site is denoted by an unfilled arrow. Any of these PD-L1 binding peptides may modulate the activity of PD-1 on its receptor PD-L1, including through steric or direct effects, with those binding on or near the PD-1 binding site potentially directly inhibiting PD-1 binding at the PD-L1 active site.

FIG. 4A and FIG. 4B illustrate the selection of various PD-L1-binding peptides that align with crystal structure, computational modeling, and phylogeny, resulting in high confidence for PD-L1-binding cystine dense peptides with PD-L1 binding activity. PD-L1-binding cystine dense peptides were tested as shown.

FIG. 4A shows a plot of Rosetta energy of dock scores for various docked PD-L1-binding cystine dense peptides plotted against the solvent accessible surface area (SASA) of the CDP scaffolds. Each point, representing a peptide docked to the PD-L1 structure, is color-coded based on the dominant structural element (e.g., coil, α-helix, or ß-sheet) in the scaffold. Gray points correspond to coil-rich CDPs, darker points and solid triangles correspond to helix-rich CDPs, and light points and open triangles correspond to sheet-rich CDPs. Triangles denote CDPs identified as binding hits, as determined by staining with fluorescently labeled PD-L1 and flow sorting, enriching for peptides that bind well to PD-L1. Four triangles are shown, with two nearly overlapping. Light gray shading, corresponding to PD-L1-binding peptide clusters with a dock cluster score of less than approximately 26 at the PD-1-binding interface, represents scaffolds from the optimized library that were used to generate a docking-enriched Met/Tyr scanning (DEMYS) library. Dark gray shading, corresponding to peptide clusters with a dock cluster score of about a median 50% of scores representing scaffolds not predicted to bind well with PD-L1, which in fact did not bind well as determined by staining with fluorescently labeled PD-L1 and flow sorting. These results indicate that the PD-L1-binding peptides selected and enriched for binding have an increased potential for binding PD-L1 and PD-L1-binding peptide utility as described herein.

FIG. 4B illustrates a phylogenetic tree showing the taxonomical diversity of high-scoring PD-L1-binding CDPs predicted to dock with PD-L1 near the PD-1-binding interface, having center of mass cluster for PD-L1 binding peptides located near the PD-1 binding site, and having a high utility to bind PD-L1 and to disrupt PD-1 interaction.

FIG. 5A-FIG. 5F illustrate enrichment and binding of PD-L1-binding peptides to PD-L1 for PD-L1-binding peptides expressed in mammalian display systems.

FIG. 5A illustrates a workflow (top) for seeding novel protein:protein interactions using docking-enriched methionine (M)-tyrosine (Y) scanning (DEMYS). This shows an example of how DEMYS enrichment further diversifies the optimized library and change residues from hydrophilic to hydrophobic residues, in this case methionine (M) and tyrosine (Y), in the reference sequence. A sample scaffold to which DEMYS was applied is also shown (bottom). The sample scaffold is color coded by hydrophobicity such that lightest shading indicates carbon atoms not contacting a polar atom, intermediate shading indicates acidic atoms, darker shading indicates basic atoms, and the remaining atoms are shown in white. The reference sequence scaffold, which would not be expected to bind well to PD-L1 but shows how the method of diversifying the library, is shown in its parental form (SEQ ID NO: 356), along with three examples of Met or Tyr mutations corresponding to D18M (SEQ ID NO: 5), R26Y (SEQ ID NO: 6), and R36M (SEQ ID NO: 7).

FIG. 5B shows results of an optimized CDP library fluorescence-based mammalian display screen. Results are plotted as PD-L1 bound versus CDP expression. Cells displaying CDPs that bound to PD-L1 are indicated by a box.

FIG. 5C shows results of the optimized library further diversified using a docking-enriched methionine-tyrosine scanning (DEMYS) CDP library fluorescence-based mammalian display screen. Results are plotted as PD-L1 bound versus CDP expression. Cells displaying CDPs that bound to PD-L1 are indicated by a box.

FIG. 5D illustrates structural model of a CDP scaffold parent of SEQ ID NO: 4, identified in the optimized CDP library mammalian display screen shown in FIG. 5B.

FIG. 5E shows a flow plot, plotted as CDP expression versus co-stain fluorescence, for a transiently transfected SDGF-cloned CDP, corresponding to SEQ ID NO: 4, identified in the PD-L1 mammalian display screen shown in FIG. 5B. Dark gray lines correspond to staining in the presence of PD-L1. Light gray lines correspond to staining in the absence of PD-L1.

FIG. 5F shows flow plots, plotted as CDP expression versus co-stain fluorescence, for four transiently transfected SDGF-cloned CDPs, corresponding to SEQ ID NO: 4 (top left), SEQ ID NO: 57 (top right), SEQ ID NO: 58 (bottom left), and SEQ ID NO: 59 (bottom right), identified in the PD-L1 DEMYS library screen shown in FIG. 5C. Dark gray lines correspond to staining in the presence of PD-L1. Light gray lines correspond to staining in the absence of PD-L1. These data show that SEQ ID NO: 4, SEQ ID NO: 57, SEQ ID NO: 58, and SEQ ID NO: 59 bind to PD-L1 and that the PD-L1 DEMYS library yielded more molecules that bind PD-L1 than the optimized library yielded.

FIG. 6A-FIG. 6C illustrate structural and competitive binding aspects of PD-L1-binding peptides binding to PD-L1. This data shows that SEQ ID NO: 4 is competitive with PD-1 and that it binds human and cynomolgus monkey PD-L1.

FIG. 6A shows a structural model of the binding interface between the top 200 PD-L1-binding peptides (mesh), identified in the optimized CDP library screen shown in FIG. 5B, docked to PD-L1. The surface rendition is color-coded for cynomolgus monkey (left) and mouse (right) homology. PD-1 bound to PD-L1 is shown as a ribbon structure.

FIG. 6B graphically illustrates the results of a competition assay to assess the ability of a PD-L1-binding CDP, corresponding to SEQ ID NO: 4, to disrupt PD-1 interactions with PD-L1. HEK 293F cells surface-expressing SEQ ID NO: 4 via SDGF were stained with fluorescently labeled PD-L1. Staining was compared in the presence of 50 nM, 150 nM, 500 nM, 1.5 μM, 5 μM, or 15 μM of a PD-1 competitor Fc fusion protein (striped bars) or in the presence of 50 nM, 150 nM, 500 nM, 1.5 μM, 5 μM, or 15 μM of a control Fc fusion protein (dotted bars).

FIG. 6C shows a flow staining assay of cells surface-expressing SEQ ID NO: 4 binding to either human (darkest gray lines), cynomolgus monkey (lightest gray lines, “Cyno”), or mouse (intermediate gray lines) PD-L1.

FIG. 7A-FIG. 7C illustrate identification and enrichment of high affinity binders of the PD-L1-binding peptides using site saturated mutagenesis and flow sorting for binding to PD-L1.

FIG. 7A shows a first round of flow sorting of site-saturation mutagenesis (SSM) variants of SEQ ID NO: 4. Cells surface displaying CDP variants of SEQ ID NO: 4 were stained with fluorescent PD-L1, and flow sorted based on binding to PD-L1.

FIG. 7B shows a second round of flow sorting of site-saturation mutagenesis (SSM) variants of SEQ ID NO: 4. Cells surface displaying CDP variants of SEQ ID NO: 4 were stained with fluorescent PD-L1, and flow sorted based on binding to PD-L1. The top 7% of PD-L1 binders were further analyzed in FIG. 8.

FIG. 7C shows flow sorting of site-saturation mutagenesis (SSM) variants of SEQ ID NO: 4 stained with co-stain only in the absence of PD-L1.

FIG. 8 shows an exemplary affinity maturation heat map of SEQ ID NO: 4 variants to identify high performance residues and point mutations within PD-L1-binding peptides. The heat map was used to identify PD-L1-binding peptides, including SEQ ID NO: 3, with high affinity for PD-L1. Shading corresponds to the relative enrichment of each amino acid point mutation (vertical axis) relative to SEQ ID NO: 4 (horizontal axis). Lighter shaded squares indicate more (positive) enrichment, and darker shaded squares indicate depletion. That is, lighter shaded squares indicate sequences whose relative abundance increased after two rounds of selection for PD-L1 binding, indicating improved binding relative to that of SEQ ID NO: 4 and darker shaded squares indicate sequences whose relative abundance decreased after two rounds of selection for PD-L1 binding, indicating disruption of binding relative to that of SEQ ID NO: 4. A binary color map of this data indicating residues with positive enrichment is provided in FIG. 21 and quantification of enrichment data is provided in TABLE 12. The heat map represents log₂-transformed enrichment of each variant after two rounds of sorting and regrowth, normalized to both initial variant abundance and performance of SEQ ID NO: 4 itself. Positive enrichment scores represent increased relative abundance and therefore improved binding. Sequences of the parental scaffold (SEQ ID NO: 353), a primary hit (SEQ ID NO: 4), and an affinity matured variant (SEQ ID NO: 3) are provided below the heat map. Mutations are color coded based on benefit or likely passenger status. Asterisk indicates novel N-linked glycosites. “Beneficial in SEQ ID NO: 3” indicates those point mutations that were combined and included in SEQ ID NO: 3 to increase affinity of SEQ ID NO: 3 for PD-L1 relative to SEQ ID NO: 4. “Omitted in SEQ ID NO: 3” indicates a point mutation that appeared beneficial in isolation but, when combined with other beneficial point mutations, became disruptive to binding. This point mutation was omitted in SEQ ID NO: 3. “Disruptive reversions” indicate point mutations that, when reverted to the parent amino acid, reduced binding, indicating that those point mutations in SEQ ID NO: 4 were beneficial to PD-L1 binding. “Neutral reversions” indicate reversions to the parental amino acid present in SEQ ID NO: 353 that do not impact binding to PD-L1, showing that these mutations were acquired during library generation but were not instrumental to SEQ ID NO: 4 binding to PD-L1. Such neutral reversions are also referred to as passenger mutations.

FIG. 9 shows a comparison of PD-L1 binding for six PD-L1-binding cystine dense peptides containing amino acid substitutions enriched in the affinity maturation assay shown in FIG. 8. Cystine dense peptides contained either all six of the amino acid substitutions E11W, A13M, Y15G, I22N, Y36K, and W40F (SEQ ID NO: 8, “All 6”) relative to SEQ ID NO: 4, or five of the six substitutions (SEQ ID NO: 9-SEQ ID NO: 14, corresponding to reversion mutants W11E, M13A, G15Y, N22I, K36Y, and F40W, respectively). Staining was performed as either one step (solid bars) or two steps (striped bars). Reversion mutants W11E (SEQ ID NO: 9), G15Y (SEQ ID NO: 11), and K36Y (SEQ ID NO: 13) showed increased affinity for PD-L1 relative to SEQ ID NO: 8, indicating that the corresponding reversions were beneficial to PD-L1-binding relative to that of SEQ ID NO: 8 (“All 6”).

FIG. 10A-FIG. 10D illustrate production, purity, purification, stability, and PD-L1-binding affinity of PD-L1-binding cystine dense peptides.

FIG. 10A shows reversed phase high-performance liquid chromatography (RP-HPLC) chromatograms (top) and SDS-PAGE gels (bottom) of three recombinant PD-L1-binding cystine dense peptides (SEQ ID NO: 4, SEQ ID NO: 3, and SEQ ID NO: 1). RP-HPLC and SDS-PAGE assays were performed under non-reducing (NR) conditions or in the presence of 10 mM DTT. The arrow in the RP-HPLC chromatogram indicates a minor species possibly representing glycosylated SEQ ID NO: 3. This data illustrates successful production, purity, purification and disulfide bond formation of SEQ ID NO: 4, SEQ ID NO: 3, and SEQ ID NO: 1. The RP-HPLC data also illustrates that the matured SEQ ID NO: 3 and SEQ ID NO: 1 undergo less unfolding upon exposure to DTT than the original hit SEQ ID NO: 4.

FIG. 10B shows liquid chromatography-mass spectrometry (LC-MS) data for three recombinant PD-L1-binding cystine dense peptides (SEQ ID NO: 4, SEQ ID NO: 3, and SEQ ID NO: 1). This data validates the identity of the foregoing PD-L1-binding peptides based on the predicted molar mass of the CDPs and that that cystine disulfide bonds indicative of a CDP structure were formed, as the DTT-treated (reducing conditions) peptides showed an increase of approximately 6 Da in mass.

FIG. 10C shows surface plasmon resonance (SPR) plots for three recombinant PD-L1-binding cystine dense peptides (SEQ ID NO: 4, SEQ ID NO: 3, and SEQ ID NO: 1). SEQ ID NO: 4 bound PD-L1 with an equilibrium dissociation constant (K_{D}) of 39.6±0.3 nM, SEQ ID NO: 3 bound PD-L1 with K_{D}=160±1 pM (kₐ=1.25±0.01×10⁸ M⁻¹ s, k_{d}=2.00±0.01×10⁻² s⁻¹), and SEQ ID NO: 1 bound PD-L1 with K_{D}=202±2 pM (kₐ=9.73±0.06×10⁷ M⁻¹ s, k_{d}=1.96±0.01×10⁻² s⁻¹). This data illustrates that SEQ ID NO: 4, SEQ ID NO: 3, and SEQ ID NO: 1 bind PD-L1 with high affinity. These data also show that the matured variant binders, SEQ ID NO: 1 and SEQ ID NO: 3, bind PD-L1 with a higher affinity than SEQ ID NO: 4 from which they were derived.

FIG. 10D shows an SPR plot of SEQ ID NO: 1 competing with PD-1 for binding to PD-L1. PD-1 (SEQ ID NO: 349) binding to PD-L1 was compared in the absence of SEQ ID NO: 1 or in the presence of 0.6 μM or 3 μM SEQ ID NO: 1. This data illustrates that SEQ ID NO: 1 is able to compete with PD-1 for binding to PD-L1.

FIG. 11 schematically illustrates two exemplary bispecific immune cell engager (BiICE) complexes capable of engaging T-cells by bispecifically binding to PD-L1 and to CD3. The CDP-based BiICE (“CS-BiICE”) is formed from a first fusion protein (SEQ ID NO: 342) containing a PD-L1-binding CDP (SEQ ID NO: 2) with an N-terminal signal peptide (SEQ ID NO: 247; “SP”) and FLAG tag (DYKDEGGS; SEQ ID NO: 246) fused to an Fc “hole” sequence via a linker and a second fusion protein (SEQ ID NO: 347) containing an anti-CD3 single-chain fragment variable (scFv) fused to an Fc “knob” sequence with an N-terminal signal peptide and C-terminal 6×His tag (SEQ ID NO: 248). The “hole” sequence heterodimerizes with the “knob” sequence. The scFv-based BiICE (“SS-BiICE”) is formed from a first fusion protein (SEQ ID NO: 346) containing an anti-PD-L1 scFc with an N-terminal signal peptide and FLAG tag fused to an Fc “hole” sequence and a second fusion protein (SEQ ID NO: 347) containing an anti-CD3 single-chain fragment variable (scFv) fused to an Fc “knob” sequence with an N-terminal signal peptide and C-terminal 6×His tag (SEQ ID NO: 248). Sequence organization of the CS-BiICE and SS-BiICE components are shown in the top panel. Structural organization of the heterodimerized CS-BiICE and SS-BiICE are shown in the bottom panel.

FIG. 12 shows the results of a fluorescence-based binding assay to assess the cross-reactivity of various PD-L1 binding moieties with different PD-L1 orthologs. Cells expressing surface-tethered PD-L1 binding moieties, including a PD-L1-binding cystine dense peptide (SEQ ID NO: 3), a single-chain fragment variable (scFv) derived from an anti-PD-L1 antibody (SEQ ID NO: 345), or an scFv derived from the anti-PD-L1 drug atezolizumab (SEQ ID NO: 348), were stained with either human, cynomolgus monkey (“Cyno”), or mouse his-tagged PD-L1 orthologs and co-stained with anti-His-iFluor647 antibody. Anti-His-iFluor647 antibody alone (“Costain only”) was used as a comparison. Co-stain fluorescence of cells is shown on the horizontal axis. This data shows that SEQ ID NO: 3 binds human and cynomolgus monkey PD-L1 but not mouse PD-L1, SEQ ID NO: 345 binds human PD-L1 but not cynomolgus monkey or mouse PD-L1, and that SEQ ID NO: 348 binds human, cynomolgus monkey, and mouse PD-L1 to various extents.

FIG. 13A-FIG. 13D illustrate production, purity, purification, and binding to PD-L1 of BiICE complexes containing either PD-L1-binding cystine dense peptide or a PD-L1-binding scFv.

FIG. 13A shows an SDS-PAGE gel of a purified CS-BiICE molecule (SEQ ID NO: 342 heterodimerized with SEQ ID NO: 347). Separate bands corresponding to heterodimer (H) and anti-CD3-scFv-Fc monomer (M) were seen under non-reducing (NR) conditions. Separate bands corresponding to CDP-Fc and scFv-Fc species were seen under reducing conditions (DTT). This data indicates successful production, purity, purification, heterodimer formation, and disulfide bonding of a CDP-based BiICE.

FIG. 13B shows SPR measurements of CS-BiICE binding to PD-L1. The CS-BiICE bound to PD-L1 with a K_{D} of 11.2 nM (kₐ=5.42×10⁵ M⁻¹ s, k_{d}=6.07±0.01×10⁻³ s⁻¹). This data indicates that the CDP-based BiICE binds PD-L1 with high affinity.

FIG. 13C show an SDS-PAGE gel of a purified SS-BiICE molecule (SEQ ID NO: 346 heterodimerized with SEQ ID NO: 347). Separate bands corresponding to heterodimer (H) and anti-CD3-scFv-Fc monomer (M) were seen under non-reducing (NR) conditions. A single band corresponding to scFv-Fc species was seen under reducing conditions (DTT). This data indicates successful production, purity, dimer formation, and purification of an scFv-based BiICE.

FIG. 13D shows SPR measurements of SS-BiICE binding to PD-L1. The SS-BiICE bound to PD-L1 with a K_{D} of 65 nM. This data indicates that the scFv BiICE binds to PD-L1 with lower affinity than the CDP-based BiICE shown in FIG. 13B.

FIG. 14A-FIG. 14E illustrate validation of PD-L1-binding BiICE complexes by binding to T-cells and inducing tumor cell death.

FIG. 14A shows flow staining of human primary T cells from patient derived peripheral blood mononuclear cells (PMBCs) using CS-BiICE and SS-BiICE molecules. Cells co-stained with a fluorescent anti-6×His antibody. Co-stain alone was included as a control. Both CS-BiICE and SS-BiICE showed increased staining over co-stain alone, indicating T cell binding. This data indicates that PD-L1/CD3-binding CS-BiICE and SS-BiICE molecules were able to bind to T-cells via CD3.

FIG. 14B shows the results of a human T-cell killing assay in a PC3 cancer cell line with increasing concentrations of CS-BiICE or SS-BiICE. CS-BiICE induced T-cell killing with a half maximal effective concentration (EC50) of 28 pM in PD-L1-expressing (“WT”) PC3 cells. SS-BiICE induced T-cell killing with an EC50 of 97 pM in PD-L1-expressing (“WT”) PC3 cells. The T-cell killing assay was also performed in PD-L1 knock out (KO) PC3 cells. T-cell killing was lower in PD-L1 KO cells than in WT cells for both CS-BiICE and SS-BiICE. This data indicates that CS-BiICE and SS-BiICE molecules were able to recruit T-cells to cancer cells and induce cancer cell death in PC3 cells in a PD-L1-dependent manner. This data also indicates that CS-BiICE was more potent at inducing cancer cell death than SS-BiICE.

FIG. 14C shows the results of a human T-cell killing assay in an MDA-MB-231 cancer cell line with increasing concentrations of CS-BiICE or SS-BiICE. CS-BiICE induced T-cell killing with an EC50 of 142 pM. SS-BiICE induced T-cell killing with an EC50 of 333 pM. This data indicates that CS-BiICE and SS-BiICE molecules were able to recruit T-cells to cancer cells and induce cancer cell death in MDA-MB-231 cells. This data also indicates that CS-BiICE was more potent at inducing cancer cell death than SS-BiICE.

FIG. 14D shows the results of a human T-cell killing assay in a PBT-05 cancer cell line with increasing concentrations of CS-BiICE or SS-BiICE. CS-BiICE induced T-cell killing with an EC50 of 2.4 pM. SS-BiICE induced T-cell killing with an EC50 of 7.7 pM. This data indicates that CS-BiICE and SS-BiICE molecules were able to recruit T-cells to cancer cells and induce cancer cell death in PBT-05 cells. This data also indicates that CS-BiICE was more potent at inducing cancer cell death than SS-BiICE.

FIG. 14E schematically illustrates a representative BiICE facilitating contact between an T cell and a cancer cell, enabling the T-Cell to kill the cancer cell. The immunological synapse is narrow enough to enable signal exchange between the targeted cell, engaged by the PD-L1-binding peptide, and the targeted immune cell, engaged by the immune cell binding moiety, such as an anti-CD3 moiety, resulting in an immune response against the cancer cell. In this example SEQ ID NO: 2 is used. It is understood that any PD-L1-binding peptide (e.g., any one of SEQ ID NO: 1-SEQ ID NO: 118) may be used with an immune cell binding moiety (e.g., any one of SEQ ID NO: 122 or SEQ ID NO: 422-SEQ ID NO: 491) to make a BiICE resulting in an immune response against the targeted cancer.

FIG. 15A and FIG. 15B illustrate additional purification of SS-BiICE and CS-BiICE complexes and induction of tumor cell death via T-cell recruitment.

FIG. 15A shows SDS-PAGE gels of IMAC purified and extra-purified (IMAC and FLAG) BiICE preparations. Purified SS-BiICE preparations are shown on the left, and purified CS-BiICE preparations are shown on the right. This data indicates that additional FLAG purification removed an impurity, anti-CD3-scFv-Fc monomer, present in SS-BiICE and CS-BiICE preparations following IMAC purification.

FIG. 15B shows the results of a human T-cell killing assay in a PC3 cancer cell line with varying concentrations of standard purified (IMAC) or extra-purified (IMAG+FLAG) CS-BiICE or SS-BiICE. Extra-purified BiICE preparations underwent FLAG purification in addition to IMAC purification. T-cell killing activity was retained following the additional FLAG purification step. This data indicates that T-cell recruitment and tumor cell killing properties of SS-BiICE and CS-BiICE complexes were retained following removal of impurities by an additional purification step, and that the tumor cell killing was due to the function of the heterodimeric BiICE molecules not due to the impurity.

FIG. 16A-FIG. 16H illustrate the ability of PD-L1-binding CDP-based BiICE complexes to shrink tumors and increase cancer survival rates in vivo.

FIG. 16A shows an experimental timeline for an in vivo tumor shrinking assay. Nude mice were implanted with PC3 or MDA-MB-231 tumors. Day zero was set as the day on which tumors reached 100-200 mm² in size. Mice were received four treatments of either SS-BiICE or CS-BiICE on days 1, 4, 8, and 11 and two treatments of activated human T-cells on days 2 and 7. Tumor size and mortality were tracked up to Day 95 and compared with control groups receiving vehicle only or T cells only.

FIG. 16B shows the survival probabilities of the first cohort of mice implanted with PC3 tumors and treated as described in FIG. 16A. This data indicates that CS-BiICE complexes significantly increased probability of survival in vivo for mice with implanted PC3 tumors compared to SS-BiICE complexes or to T cells or vehicle alone. ns: not statistically significant. *: P<0.05. **: P<0.01. ***: P<0.001. Unlabeled: P<0.0001. Kaplan-Meier curve P values were determined by log-rank (Mantel-Cox) test.

FIG. 16C shows tumor sizes of mice implanted PC3 tumors and treated with vehicle only (no T cells, no BiICE, upper left), T cells only (no BiICE, upper right), T cells and SS-BiICE (lower left), or T cells and CS-BiICE (lower right). This data indicates that, while both SS-BiICE and CS-BiICE complexes demonstrated the ability to shrink tumors in vivo, the tumors returned in mice treated with SS-BiICE but not in mice treated with CS-BiICE.

FIG. 16D shows the survival probabilities of the second cohort of mice implanted with PC3 tumors and treated as described in FIG. 16A, with the exception that two arms were treated with clinical anti-PD-L1 antibody durvalumab in either 1 nmol or 0.1 nmol amounts. An additional arm of CS-BiICE treatment at 0.1 nmol was also included alongside a repeat of 1 nmol CS-BiICE. This data shows that CS-BiICE complexes, at 1 nmol or 0.1 nmol dose, increased probability of survival in vivo for mice with implanted PC3 tumors compared to durvalumab or to vehicle alone. P<0.0001 for 1 nmol and 0.1 nmol CS-BiICE versus vehicle and both durvalumab groups. ns: not statistically significant. Kaplan-Meier curve P values were determined by log-rank (Mantel-Cox) test.

FIG. 16E shows tumor sizes of mice implanted PC3 tumors and treated with vehicle only (no T cells, no BiICE, upper left), T cells and 1 nmol durvalumab (upper center, “Durva (1)”), T cells and 0.1 nmol durvalumab (upper right, “Durva (0.1)”), T cells and 1 nmol CS-BiICE (lower left, “CS-BiICE (1)”), or T cells and 0.1 nmol CS-BiICE (lower center, “CS-BiICE (0.1)”). This data indicates that the tumors shrank and did not return in the mice treated with 1 nmol or 0.1 nmol CS-BiICE.

FIG. 16F shows the survival probabilities of mice implanted with MDA-MB-231 tumors and treated as described in FIG. 16A. This data shows that CS-BiICE complexes increased probability of survival in vivo for mice with implanted MDA-MB-231 tumors compared to T cells or vehicle alone. P<0.0001 for CS-BiITE versus vehicle or T cells only. ns: not statistically significant. Kaplan-Meier curve P values were determined by log-rank (Mantel-Cox) test.

FIG. 16G shows days for tumor volume to triple in size in mice implanted MDA-MB-231 tumors and treated as described in FIG. 16A. This data shows that treatment with CS-BiICE complexes slowed MDA-MB-231 tumor growth in vivo compared to treatment with SS-BiICE complexes or with T cells or vehicle alone. ns: not statistically significant. *: P<0.05. **: P<0.01. ***: P<0.001. Unlabeled: P<0.0001. Kaplan-Meier curve P values were determined by log-rank (Mantel-Cox) test.

FIG. 16H shows tumor sizes of mice implanted MDA-MB-231 tumors and treated with vehicle only (no T cells, no BiICE, upper left), T cells only (no BiICE, upper right), T cells and SS-BiICE (lower left), or T cells and CS-BiICE (lower right). This data shows that treatment with CS-BiICE complexes slowed MDA-MB-231 tumor growth in vivo compared to treatment with SS-BiICE complexes or with T cells or vehicle alone.

FIG. 17 shows the weights of mice implanted with PC3 tumors and treated as described in FIG. 16A over the course of the experiment. This data shows that mice treated with CS-BiICE maintained a healthy weight throughout the study. ns: not statistically significant. Kaplan-Meier curve P values were determined by log-rank (Mantel-Cox) test.

FIG. 18 shows full SDS-PAGE gels of recombinant proteins described herein. Either PBS or 10 mM DTT were included in the sample buffer prior to boiling and loading. All Fc-containing proteins, including knob-and-hole bispecific molecules SS-BiICE and CS-BiICE, contained the IgG1 hinge region between the binder of interest (PD-1, control target, CDP, or scFv) and the Fc domain, which included two cysteines that create a pair of interdomain disulfide bonds between the paired hinge regions. This further stabilized the dimer between the Fc domains in an SDS-stable fashion. Anti-CD3 scFv Fc Knob protein expressed by itself (bottom right) was not a disulfide-stabilized dimer as the knob mutations do not permit stable dimerization and resulting disulfide formation. “*” and “#” denote SDS-PAGE mobility of non-BiICE contaminants in SS-BiICE and CS-BiICE that correspond to monomeric anti-CD3 scFv Fc Knob.

FIG. 19 illustrates an example of a histidine substitution scan to introduce pH-dependent binding affinity into a target-binding peptide. A histidine substitution scan of a PD-L1-binding CDP (SEQ ID NO: 1) is shown. The peptide sequence is provided above and to the side, and each black box represents a first and second site in which His could be substituted. Those falling along the diagonal from the top-left to the bottom-right represent single His substitutions.

FIG. 20A shows a co-crystal structure of a high-affinity PD-L1-binding CDP (SEQ ID NO: 1, cartoon) binding to or docked with PD-L1 (surface).

FIG. 20B shows relative binding enrichment, shown as absolute value of average SSM enrichment, of PD-L1-binding CDP variants containing amino acid substitutions in resolved (R) residues or unresolved (UR) residues, as seen in the co-crystal structure of FIG. 20A. Substitutions at resolved residues had a greater impact, either positive or negative, on binding than substitutions at unresolved residues (**: P=0.0055), showing that resolved played a greater role in interactions with PD-L1 than unresolved residues.

FIG. 20C shows an overlay of PD-1 (mesh) with SEQ ID NO: 1 (cartoon) at the binding interface with PD-L1 (surface). The PD-1 binding site overlaps with SEQ ID NO: 1, showing that SEQ ID NO: 1 would be expected to compete with PD-1 for binding to PD-L1.

FIG. 20D shows a zoomed in view of the SEQ ID NO: 1 PD-L1 co-crystal structure of FIG. 20A from two different angles. Residues of SEQ ID NO: 1 that interact with PD-L1, including K5, V9, W12, M13, K16, V39, F40, L43, and D44, are shown as sticks. Residues of PD-L1 that interact with SEQ ID NO: 1, including Y56, Q66, R113, M115, A121, and Y123, are also labeled.

FIG. 20E shows isolated side chains of select residues in SEQ ID NO: 1 (gray) at the PD-L1-binding interface relative the parent CDP (SEQ ID NO: 353, black, minimally clashing rotamers). Labeled residues of SEQ ID NO: 1, including M13, V39, F40, and L43, correspond to substitutions relative to SEQ ID NO: 353 that improved binding to PD-L1.

FIG. 20F shows a zoomed in view of the binding interface between SEQ ID NO: 1 (cartoon) and PD-L1 (surface). The PD-L1 surface is color-coded for human (Hs) versus murine (Mm) homology, wherein white corresponds to identical residues, darker shading corresponds to similar residues, and lighter shading corresponds to dissimilar residues. These differences in the binding interface between human and murine PD-L1 are consistent with the lack of murine PD-L1 cross-reactivity seen with SEQ ID NO: 1. Moreover, the data presented in FIG. 6B, FIG. 6C, and FIG. 8 is consistent with the crystal structure.

FIG. 20G shows a co-crystal structure of SEQ ID NO: 1 and PD-L1 in which SEQ ID NO: 1 is illustrated as a wire diagram with side chains of interest shown with thick sticks (top). PD-1 binding to PD-L1 is shown at bottom for comparison. Overlap of the SEQ ID NO: 1 and PD-1 binding interfaces on PD-L1 is consistent with the observed competition data shown in FIG. 6B, FIG. 6C, and FIG. 8.

FIG. 21 shows a binary recolor of the enrichment score map shown in FIG. 8. Amino acid substitutions relative to SEQ ID NO: 4 determined to have a neutral or beneficial effect are colored in gray. Original residues of the SEQ ID NO: 4 sequence are colored in black. Amino acids shaded in either gray or black are considered to facilitate binding to PD-L1.

FIG. 22 illustrates oligonucleotide mechanisms of action. Oligonucleotides, which are targeted to a specific sequence for its regulation, complexed with a PD-L1-binding CDP enter into cells through PD-L1-binding and natural endocytosis of PD-L1, resulting in oligonucleotide compartmentalization into endosomes. Oligonucleotides are released from the endocytic compartments into the cytoplasm where they can freely move between the nucleus and the cytoplasm. Upon entry into the nucleus, oligonucleotides can (1) modulate alternative splicing of a targeted sequence, (2) dictate the location of the polyadenylation (polyA) site of a targeted sequence, and (3) recruit RNaseH1 to induce cleavage of a targeted sequence. Oligonucleotides in the cytoplasm can be designed to (4) directly bind to microRNA (miRNA) or messenger (mRNA) sequences. siRNAs, which are targeted to a specific sequence for its regulation, may alternatively be used to (5) bind and regulate a targeted sequence in the cytosol, engaging an RNA-induced silencing complex (RISC) which is a multiprotein complex that incorporates one strand of a small interfering RNA (siRNA) or micro RNA (miRNA), using the siRNA or miRNA as a template for recognizing complementary mRNA of the targeted sequence. When it finds a complementary strand, the RISC complex cleaves the targeted sequence. An aptamer, targeted to a specific sequence for its regulation, may alternatively be used to (6) bind and regulate a target molecule. An aptamer directly binds and inhibits its intracellular or extracellular target.

FIG. 23 illustrates examples of structures of various peptide oligonucleotide complexes (e.g., a CDP-oligonucleotide complexes in which the peptide portion comprises a CDP) containing alternative and nonconventional bases, as represented in single-stranded, double-stranded, and hairpin structures. Examples of oligonucleotides include an aptamer, a gapmer, an anti-miR, an siRNA, a splice blocker ASO, and a U1 adapter. The CDP portion of the CDP-oligonucleotide complex can be used to guide the oligonucleotide sequence to a specific tissue, target, or cell, or to cause endocytosis of the oligonucleotide sequence by a cell. The legend is as follows: grey circle with black border=2′-H (DNA); white circle with black border=2′-OH (RNA); circle with horizontal stripes and black border=2′-O-ME; circle with vertical stripes=2′-O-MOE; black circle with grey border=2′-F; spotted circle with grey border=LNA; hatched circle with grey border=morpholino (unique phosphorodiamidate linkages not shown); grey angle=PO linkage; black angle=PS linkage.

FIG. 24A-FIG. 24E illustrate incorporation of the shown groups on RNA or DNA.

FIG. 24A illustrates structures of oligonucleotides containing a 5′-thiol (thiohexyl; C6) modification (left), and a 3′-thiol (C3) modification (right).

FIG. 24B illustrates an MMT-hexylaminolinker phosphoramidite.

FIG. 24C illustrates a TFA-pentylaminolinker phosphoramidite.

FIG. 24D illustrates RNA residues incorporating amine or thiol residues.

FIG. 24E illustrates oligonucleotides with aminohexyl modifications at the 5′ (left) and 3′ ends (right).

FIG. 25 illustrates generation of a cleavable disulfide linkage between a peptide (e.g., a PD-L1-binding peptide of SEQ ID NO: 1) and a cyclic dinucleotide.

FIG. 26 shows flow sorting data illustrating enrichment of peptides with pH-dependent binding to PD-L1. This data shows that pH-dependent binding peptides can be generated through flow sorting. A histidine-doped library based on a PD-L1-binding peptide (SEQ ID NO: 1), prepared as described in FIG. 19, was screened for peptides that exhibited stronger PD-L1 binding at neutral pH (7.4) and weaker binding at acidic pH (5.5). The input library was initially screened for high PD-L1 binding at pH 7.4. The second and third rounds of screening (“Sort 1” and “Sort 2,” respectively) were performed at pH 5.5 to mimic endosomal pH, enriching for poor PD-L1 binding at this pH. The final round of screening (“Sort 3”) was performed at pH 7.4. Differential binding at pH 7.4 and pH 5.5 was observed following screening (“Sort 4”). The areas encompassed by the 5-sided polygon in each graph denotes the population that was selected during sorting. Darker topographical density maps indicate staining with PD-L1 under pH 7.4 conditions and lighter topographical density maps indicate staining with PD-L1 under pH 5.5 conditions.

FIG. 27 shows binding data at pH 7.4 (left bars) and at pH 5.5 (right bars) for pH-dependent PD-L1-binding peptide variants identified in FIG. 26. Variants of SEQ ID NO: 1 with E2H, M13H, and K16H substitutions, individually and in combination, were screened for pH-dependent binding to PD-L1. Peptide variants containing substitutions at E2H (SEQ ID NO: 234), M13H (SEQ ID NO: 235), K16H (SEQ ID NO: 236), E2H and M13H (SEQ ID NO: 237), E2H and K16H (SEQ ID NO: 233), M13H and K16H (SEQ ID NO: 238), or E2H, M13H, and K16H (SEQ ID NO: 239) exhibited varying degrees of pH-dependent binding to PD-L1. “UTF” indicates untransfected cells (negative control). The parent peptide (SEQ ID NO: 187) exhibited some degree of pH-dependent binding to PD-L1. Some variants of SEQ ID NO: 187 exhibited more pH-dependence in PD-L1 binding than the parent, while some variants of SEQ ID NO: 187 exhibited less pH-dependence in PD-L1 binding than the parent. The peptide of SEQ ID NO: 234 was shown to have a high difference in binding at pH 7.4 versus pH 5.5, demonstrating higher binding at pH 7.4 than at pH 5.5. The peptide of SEQ ID NO: 233 (black arrow) is shown to have a particularly high difference in binding at pH 7.4 versus pH 5.5, also demonstrating higher binding at pH 7.4 than at pH 5.5. This data illustrates the generation of peptides that bind PD-L1 at higher levels at pH 7.4 and at lower levels at pH 5.5.

### DETAILED DESCRIPTION

Programmed death-ligand 1 (PD-L1) has proven to be a valuable therapeutic target for small molecule therapeutics and anti-PD-L1 antibodies, which function as immune checkpoint inhibitors and may be used as anti-cancer therapies. However, both small molecule therapeutics, typically less than 1000 Da in size, and antibody therapeutics, frequently larger than 140 kDa in size, have significant limitations in their efficacy and utility. Small molecule therapeutics often lack selectivity for their intended target, leading to serious off-target effects and a limited therapeutic window (i.e., the range of drug doses that may be therapeutically effective without toxicity). Additionally, the small target-binding interface may be prone to mutational selection in which a single amino acid change in the target at the binding interface may drastically alter binding of the small molecule to the target. At the opposite end of the spectrum, the large size of antibodies may prevent them from accessing central nervous system targets or penetrating throughout solid tumors. Furthermore, antibodies may require substantial engineering, such as humanization, to make them suitable for pharmaceutical use. Moreover, PD-L1 targeting offers an opportunity for cancer targeting via bispecific immune cell engagers. Bispecific immune cell engagers (BiICEs) can have moieties that bind both a cancer cell (e.g., via PD-L1) and an immune cell, thus holding an immune cell in direct proximity to the cancer cell to direct cell killing. However, the specific geometry of the immune synapse (e.g., the distance between the cancer cell and the immune cell) can be important for potent cell killing. Antibodies and antibody fragments, such as scFvs or nanobodies with molecular weights around 15-27 kDa, may not be able to create an immune synapse close enough to drive optimal cell killing.

Described herein are small, typically less than about 10 kDa in size and often less than 6 kDa in size, drug-like proteins engineered to bind to therapeutic targets, including PD-L1, and exert a therapeutic effect. These small proteins, or “miniproteins,” may themselves provide a therapeutic effect, for example by binding and inhibiting a target protein, or by delivering an additional active agent (e.g., a detectable agent, a small molecule or protein drug, an immune cell engaging moiety, or an additional active agent) to a target region. For example, due to the overexpression of PD-L1 by some cancer cells, the PD-L1-binding peptides described herein can be used to target therapeutic moieties to cancer cells and tissues, promote cell killing of cancer cells, or both. The present disclosure provides PD-L1-binding peptides, also referred to herein as miniproteins, that binding to PD-L1. These proteins may be cystine-dense peptides (CDPs), or cystine-dense miniproteins, which are stabilized by disulfide bridges formed between cysteine amino acid residues. Cystine-dense peptides may have the additional benefit of being thermostable, protease-resistant, of low immunogenicity, smaller size, and tissue penetrating. Also described herein are methods of using PD-L1-binding peptides (also referred to as PD-L1-binding CDPs) as therapeutic or diagnostic agents.

The present disclosure utilizes a peptide design approach based on the 3D protein structure to select peptides or proteins capable of binding PD-L1 and designed to bind at a specific interface of the protein (e.g., the PD-L1 protein) based on the 3D structure of the protein. For example, a peptide may be designed to bind at the PD-1-binding interface of PD-L1.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

The terms “peptide,” “polypeptide,” “miniprotein,” “protein,” “hitchin,” “cystine-dense peptide,” “knotted peptides,” or “CDP” can be used interchangeably herein to refer to a polymer of amino acid residues. In various embodiments, “peptides,” “polypeptides,” and “proteins” can be chains of amino acids whose alpha carbons are linked through peptide bonds. The terminal amino acid at one end of the chain (e.g., amino terminal, or N-terminal) therefore can have a free amino group, while the terminal amino acid at the other end of the chain (e.g., carboxy terminal, or C-terminal) can have a free carboxyl group. As used herein, the term “amino terminus” (e.g., abbreviated N-terminus) can refer to the free α-amino group on an amino acid at the amino terminal of a peptide or to the α-amino group (e.g., imino group when participating in a peptide bond) of an amino acid at any other location within the peptide. Similarly, the term “carboxy terminus” can refer to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether or thioether as opposed to an amide bond.

As used herein, the term “peptide construct” or “peptide complex” can refer to a molecule comprising one or more peptides of the present disclosure that can be conjugated to, linked to, or fused to one or more peptides or cargo molecules. In some cases, cargo molecules are active agents. The term “active agent” can refer to any molecule, e.g., any molecule that is capable of eliciting a biological effect and/or a physical effect (e.g., emission of radiation) which can allow the localization, detection, or visualization of the respective peptide construct. In various embodiments, the term “active agent” refers to a therapeutic and/or diagnostic agent. A peptide construct of the present disclosure can comprise a PD-L1-binding peptide that is linked to one or more active agents via one or more linker moieties (e.g., cleavable or stable linker) as described herein. As used herein, the term “peptide complex” can also refer to one or more peptides of the present disclosure that are fused, linked, conjugated, or otherwise connected to form a complex. In some cases, the one or more peptides can comprise a PD-L1-binding peptide, an additional peptide active agent, a peptide that binds immune cells (e.g., T cells), a half-life modifying peptide, a peptide that modifies pharmacodynamics and/or pharmacokinetic properties, or combinations thereof.

The terms “nucleotide,” “oligonucleotide,” “polynucleotide,” “polynucleic acid,” or “nucleic acid” may refer to any molecule comprising nucleic acids, such as short single- or double-stranded DNA or RNA molecules. A nucleotide may comprise deoxyribonucleotides, ribonucleotides, modified deoxyribonucleotides or ribonucleotides, derivatives of deoxyribonucleotides or ribonucleotides, synthetic nucleotides, other nucleotides comprising various nucleobases or various sugars, or combinations thereof. As used herein, “nucleotide,” “oligonucleotide,” “polynucleotide,” “polynucleic acid,” or “nucleic acid” include any single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), oligonucleotide complementary to natural antisense transcripts (NATs) sequences, siRNA, snRNA, aptamer, gapmer, anti-miR, splice blocker ASO, or U1 Adapter. Within the peptide oligonucleotide complexes described herein, “nucleotide,” “oligonucleotide,” “polynucleotide,” “polynucleic acid,” or “nucleic acid” may be intended for modulating gene or protein expression, or for modulating intermolecular pr intramolecular interactions, and may each be considered a target-binding agent capable of binding a target molecule. The target may be a protein, nucleic acid, or other non-nucleic acid molecule. When the target is a nucleic acid, the sequence of a target molecule may be derived from an RNA (e.g., an mRNA or a pre-mRNA) or an open reading frame (ORF) of a gene or protein coding sequence. The sequence of a target molecule may be found in or derived from the coding region or the non-coding region of a gene, or it may be found in or derived from the mature mRNA (e.g., an mRNA which has been spliced, polyadenylated, capped, and exported to the cytosol for translation) or the immature pre-mRNA. The target binding agent may be the complement to such target molecule sequence (e.g., an open reading frame, non-coding sequence, or RNA).

As used herein, the term “complement” or “reverse complement” may refer to a nucleotide sequence that is fully or partially reverse complementary to a target or reference sequence. The term “complementary” may be used interchangeably with “reverse complementary” or “antisense” to describe nucleotide sequences that form base-pairing interactions (e.g., A/T, A/U, or C/G interactions) with a target or reference nucleotide sequence.

As used herein, the term “antisense oligonucleotide” includes small, noncoding, and diffusible molecules, containing about 15-35 nucleotides that form a reverse complement of a nucleic acid target sequence (e.g., a transcript or an mRNA molecule). In some embodiments, the antisense molecule may be fully reverse complementary to the target sequence. In some embodiments, the antisense molecule may comprise one or more base mismatches relative to the target sequence. As used herein, “antisense” may refer to nucleotides of varying chemistries, whether natural (RNA and/or DNA) or synthetic (e.g. 2′ pentose sugar modifications, 2′F, 2′OMe, LNA, PNA, and/or morpholino) with natural or synthetic linkages (e.g. phosphodiester, phosphorothioate, phosphorodiamidate, or thiophosphorodiamidate), as the context requires, and can comprise oligonucleotides, ribonucleotides, ribonucleosides, deoxyribonucleotides, deoxyribonucleosides, may be single stranded or double stranded in whole or in part or in any combination, and any of the forgoing in a modified form and in any combination to form a polynucleic acid. Similarly, thiophosphorodiamidate linkages may be used. Such polynucleic acid can further contain modified bases (e.g. synthetic purines or pyrimidines whose chemistries differ from that of adenine, cytosine, guanine, thymine, or uracil) or contain other atypical elements or chemistries. In various embodiments, antisense RNA containing 19-23 nucleotides (nt), or 15-35 nt, that complement target RNA. Antisense RNAs are about 5 to 30 nt in length, 10 to 25 nt in length, 15 to 25 nt in length, 19 to 23 nt in length, or at least 10 nt in length, at least 15 nt in length, at least 20 nt in length, at least 25 nt in length, or at least 30 nt in length, at least 50 nt in length, at least 100 nucleotides in length. Non-limiting examples of antisense oligonucleotides (ASOs) include aptamers, gapmers, anti-miRs, siRNAs, miRNAs, snRNAs, splice blocker ASOs, and U1 adapters.

As used herein, the term “interfering RNA” or “inhibitory RNA” is used interchangeably and includes RNA molecules that are involved in sequence-specific suppression of gene expression by forming a double-stranded RNA. As used herein, “interfering RNA” or “inhibitory RNA” can comprise ribonucleotides, ribonucleosides, deoxyribonucleotides, deoxyribonucleosides, may be single stranded or double stranded in whole or in part or in any combination, and any of the forgoing in a modified form and in any combination to form a polynucleic acid. Such polynucleic acid can further contain modified bases or contain other atypical elements or chemistries. Common forms of “interfering RNA” or “inhibitory RNA” include small inhibitory RNA (siRNA or RNAi), and dsRNA, ssRNA, hairpin RNA and other known structures. In various embodiments, inhibitory RNAs are about 5 to 30 nt in length, 10 to 25 nt in length, 15 to 25 nt in length, 19 to 23 nt in length, or at least 10 nt in length, at least 15 nt in length, at least 20 nt in length, at least 25 nt in length, or at least 30 nt in length, at least 50 nt in length, at least 100 nucleotides in length.

As used herein, the term “nuclear RNA” includes any RNA molecules that are present in the nucleus of a cell. As used herein, “nuclear RNA” can comprise small nuclear RNA (snRNA), spliceosomal RNA, and other known structures.

As used herein, the term “U1 adaptor” includes bifunctional oligonucleotides with a target domain complementary to a site in the vicinity of the target gene's polyadenylation (polyA) site and a U1 domain that binds to the U1 small nuclear RNA component of the U1 small nuclear ribonucleoprotein (U1 snRNP). U1 Adaptors can be used as synthetic oligonucleotides to recruit endogenous U1 snRNP to a target sequence or site. As used herein, U1 adapters can comprise any nucleotide sequence complementary to the ssRNA component of the U1 small nuclear ribonucleoprotein (U1 snRNP). In various embodiments, U1 adapters are about 5 to 30 nt in length, 10 to 25 nt in length, 15 to 25 nt in length, 19 to 23 nt in length, or at least 10 nt in length, at least 15 nt in length, at least 20 nt in length, at least 25 nt in length, or at least 30 nt in length, at least 50 nt in length, at least 100 nucleotides in length. nucleotides in length and complementary to any sequence along the U1 domain or U1 small nuclear ribonucleoprotein (U1 snRNP) splicing factor.

As used herein, the terms “comprising” and “having” can be used interchangeably. For example, the terms “a peptide comprising an amino acid sequence of SEQ ID NO: 1” and “a peptide having an amino acid sequence of SEQ ID NO: 1” can be used interchangeably.

As used herein, and unless otherwise stated, the term “PD-L1” or “programmed death-ligand 1” is a class of protein used herein and can refer to a PD-L1 from any species (e.g., human or murine PD-L1 or any human or non-human animal PD-L1). In some cases, and as used herein, the term “PD-L1” or “programmed death-ligand 1” refers to human PD-L1 and can include PD-L1 or any combination or fragment (e.g., ectodomain) thereof. In some cases, PD-L1 may also be referred to as “CD274,” “B7-H,” “B7H1,” “PDCD1L1,” “PDCD1LG1”, or “PDL1.”

The term “engineered,” when applied to a polynucleotide, denotes that the polynucleotide has been removed from its natural genetic milieu and is thus free of other extraneous or unwanted coding sequences, and is in a form suitable for use within genetically engineered protein production systems. Such engineered molecules are those that are separated from their natural environment and include cDNA and genomic clones (i.e., a prokaryotic or eukaryotic cell with a vector containing a fragment of DNA from a different organism). Engineered DNA molecules of the present invention are free of other genes with which they are ordinarily associated but can include naturally occurring or non-naturally occurring 5′ and 3′ untranslated regions such as enhancers, promoters, and terminators.

An “engineered” polypeptide or protein is a polypeptide or protein that is found in a condition other than its native environment, such as apart from blood and animal tissue. In a preferred form, the engineered polypeptide is substantially free of other polypeptides, particularly other polypeptides of animal origin. It is preferred to provide the polypeptides in a highly purified form, e.g., greater than 90% pure, greater than 95% pure, more preferably greater than 98% pure or greater than 99% pure. When used in this context, the term “engineered” does not exclude the presence of the same polypeptide in alternative physical forms, such as dimers, heterodimers and multimers, heteromultimers, or alternatively glycosylated, carboxylated, modified, or derivatized forms.

An “engineered” peptide or protein is a polypeptide that is distinct from a naturally occurring polypeptide structure, sequence, or composition. Engineered peptides include non-naturally occurring, artificial, isolated, synthetic, designed, modified, or recombinantly expressed peptides. Provided herein are engineered PD-L1-binding peptides, variants, or fragments thereof. These engineered PD-L1-binding peptides can be further linked to an active agent or a half-life extending moiety or can be further linked to an active agent or detectable agent, or any combination of the foregoing.

Polypeptides of the disclosure include polypeptides that have been modified in any way, for example, to: (1) reduce susceptibility to proteolysis, (2) reduce susceptibility to reduction, (3) alter binding affinity for forming protein complexes, (4) alter binding affinities, (5) alter binding affinity at certain pH values, and (6) confer or modify other physicochemical or functional properties. For example, single or multiple amino acid substitutions (e.g., conservative amino acid substitutions) are made in the naturally occurring sequence (e.g., in the portion of the polypeptide outside the domain(s) forming intermolecular contacts). A “conservative amino acid substitution” can refer to the substitution in a polypeptide of an amino acid with a functionally similar amino acid. The following six groups each contain amino acids that can be conservative substitutions for one another: i) Alanine (A), Serine (S), and Threonine (T); ii) Aspartic acid (D) and Glutamic acid (E); iii) Asparagine (N) and Glutamine (Q); iv) Arginine (R) and Lysine (K); v) Isoleucine (I), Leucine (L), Methionine (M), and Valine (V); vi) Phenylalanine (F), Tyrosine (Y), and Tryptophan (W). In some embodiments, a conserved amino acid substitution can comprise a non-natural amino acid. For example, substitution of an amino acid for a non-natural derivative of the same amino acid can be a conserved substitution.

The terms “polypeptide fragment” and “truncated polypeptide” as used herein can refer to a polypeptide that has an amino-terminal and/or carboxy-terminal deletion as compared to a corresponding full-length peptide or protein. In various embodiments, fragments are at least 5, at least 10, at least 15, at least 20, at least 25, at least 30, at least 35, at least 40, at least 45, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 45, at most 40, at most 35, at most 30, at most 25, at most 20, at most 15, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

As used herein, the terms “peptide” or “polypeptide” in conjunction with “variant” “mutant” or “enriched mutant” or “permuted enriched mutant” can refer to a peptide or polypeptide that can comprise an amino acid sequence wherein one or more amino acid residues are inserted into, deleted from and/or substituted into the amino acid sequence relative to another polypeptide sequence. In various embodiments, the number of amino acid residues to be inserted, deleted, or substituted is at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Variants of the present disclosure include peptide conjugates or fusion molecules (e.g., peptide constructs or peptide complexes).

A “derivative” of a peptide or polypeptide can be a peptide or polypeptide that can have been chemically modified, e.g., conjugation to another chemical moiety such as, for example, polyethylene glycol, albumin (e.g., human serum albumin), phosphorylation, and glycosylation.

The term “% sequence identity” can be used interchangeably herein with the term “% identity” and can refer to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99% or more up to 100% sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The terms “% sequence homology” or “percent sequence homology” or “percent sequence identity” can be used interchangeably herein with the terms “% homology,” “% sequence identity,” or “% identity” and can refer to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% or more up to 100% sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

A protein or polypeptide can be “substantially pure,” “substantially homogeneous”, or “substantially purified” when at least about 60% to 75% of a sample exhibits a single species of polypeptide. The polypeptide or protein can be monomeric or multimeric. A substantially pure polypeptide or protein can typically comprise about 50%, 60%, 70%, 80% or 90% W/W of a protein sample, more usually about 95%, and e.g., will be over 98% or 99% pure. Protein purity or homogeneity can be indicated by a number of means well known in the art, such as polyacrylamide gel electrophoresis of a protein sample, followed by visualizing a single polypeptide band upon staining the gel with a stain well known in the art. For certain purposes, higher resolution is provided by using high-pressure liquid chromatography (e.g., HPLC) or other high-resolution analytical techniques (e.g., LC-mass spectrometry).

As used herein, the term “pharmaceutical composition” can generally refer to a composition suitable for pharmaceutical use in a subject such as an animal (e.g., human or mouse). A pharmaceutical composition can comprise a pharmacologically effective amount of an active agent and a pharmaceutically acceptable carrier. The term “pharmacologically effective amount” can refer to that amount of an agent effective to produce the intended biological or pharmacological result.

As used herein, the term “pharmaceutically acceptable carrier” can refer to any of the standard pharmaceutical carriers, vehicles, buffers, and excipients, such as a phosphate buffered saline solution, or a buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” can be a salt that can be formulated into a compound for pharmaceutical use including, e.g., metal salts (sodium, potassium, magnesium, calcium, etc.) and salts of ammonia or organic amines.

As used herein, the terms “treat”, “treating” and “treatment” can refer to a method of alleviating or abrogating a biological disorder and/or at least one of its attendant symptoms. As used herein, to “alleviate” a disease, disorder, or condition, for example, means reducing the severity and/or occurrence frequency of the symptoms of the disease, disorder, or condition. Further, references herein to “treatment” can include references to curative, palliative, and prophylactic or diagnostic treatment.

Generally, a cell of the present disclosure can be a eukaryotic cell or a prokaryotic cell. A cell can be an epithelial cell, a cancer cell, or a cell of the immune system. A cell can be a microorganism, bacterial, yeast, fungal or algae cell. A cell can be an animal cell or a plant cell. An animal cell can include a cell from a marine invertebrate, fish, insects, amphibian, reptile, or mammal. A mammalian cell can be obtained from a primate, ape, equine, bovine, porcine, canine, feline, or rodent. A mammal can be a primate, ape, dog, cat, rabbit, ferret, or the like. A rodent can be a mouse, rat, hamster, gerbil, hamster, chinchilla, or guinea pig. A bird cell can be from a canary, parakeet, or parrots. A reptile cell can be from a turtles, lizard, or snake. A fish cell can be from a tropical fish. For example, the fish cell can be from a zebrafish (e.g., Danino rerio). A worm cell can be from a nematode (e.g., C. elegans). An amphibian cell can be from a frog. An arthropod cell can be from a tarantula or hermit crab.

A mammalian cell can also include cells obtained from a primate (e.g., a human or a non-human primate). A mammalian cell can include a blood cell, a stem cell, an epithelial cell, connective tissue cell, hormone secreting cell, a nerve cell, a skeletal muscle cell, or an immune system cell.

As used herein, the term “vector,” generally refers to a DNA molecule capable of replication in a host cell and/or to which another DNA segment can be operatively linked so as to bring about replication of the attached segment. A plasmid is an exemplary vector.

As used herein, the term “subject,” generally refers to a human or to another animal. A subject can be of any age, for example, a subject can be an infant, a toddler, a child, a pre-adolescent, an adolescent, an adult, or an elderly individual.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are in relation to the other endpoint, and independently of the other endpoint. The term “about” as used herein refers to a range that is 15% plus or minus from a stated numerical value within the context of the particular usage. For example, about 10 can include a range from 8.5 to 11.5.

### Peptides

The PD-L1-binding peptides described herein may comprise PD-L1-binding cystine-dense peptides (CDPs). A PD-L1-binding peptide may be engineered to bind to PD-L1 (e.g., human PD-L1). In some cases, a PD-L1-binding peptide may be engineered to bind at a specific interface of PD-L1 (e.g., at the PD-1 binding interface). The PD-L1-binding peptides and PD-L1-binding peptide complexes of the present disclosure can comprise one or more peptides. For example, a PD-L1-binding peptide complex may comprise a PD-L1-binding peptide and an additional peptide therapeutic agent. A PD-L1-binding peptide of the present disclosure may be engineered to retain binding to PD-L1 when complexed with an additional active agent. In some instances, an PD-L1-binding peptide may be engineered to contain one or more amino acid residues capable of modification (e.g., with a linker).

In some instances, a peptide as disclosed herein can contain only one lysine residue, or no lysine residues. In some instances, one or more or all of the lysine residues in the peptide are replaced with arginine residues. In some instances, one or more or all of the methionine residues in the peptide are replaced by leucine or isoleucine. One or more or all of the tryptophan residues in the peptide can be replaced by phenylalanine or tyrosine. In some instances, one or more or all of the asparagine residues in the peptide are replaced by glutamine. In some embodiments, one or more or all of the aspartic acid residues can be replaced by glutamic acid residues. In some instances, one or more or all of the lysine residues in the peptide are replaced by alanine or arginine. In some embodiments, the N-terminus of the peptide is blocked or protected, such as by an acetyl group or a tert-butyloxycarbonyl group. Alternatively or in combination, the C-terminus of the peptide can be blocked or protected, such as by an amide group or by the formation of an ester (e.g., a butyl or a benzyl ester). In some embodiments, the peptide is modified by methylation on free amines. For example, full methylation is accomplished through the use of reductive methylation with formaldehyde and sodium cyanoborohydride.

In some embodiments, an N-terminal dipeptide can be absent as shown in SEQ ID NO: 1-SEQ ID NO: 59, SEQ ID NO: 435, or SEQ ID NO: 554-SEQ ID NO: 560, or the dipeptide GS can be added as the first two N-terminal amino acids, as shown in SEQ ID NO: 60-SEQ ID NO: 118, SEQ ID NO: 436, or SEQ ID NO: 561-SEQ ID NO: 567, or can be substituted by any other one or two amino acids. In some embodiments, the dipeptide GS is used as a linker or used to couple to a linker to form a peptide conjugate or fusion molecules such as a peptide construct or peptide complex. In some embodiments, the linker comprises a GₓS_{y} (SEQ ID NO: 155) peptide, wherein x and y independently are any whole number, such as 1, 2, 3, 4, 5, 6, 7, 8, 9, 10, 12, 16, or 20 and the G and S residues are arranged in any order. In some embodiments, the peptide linker comprises (GS)x (SEQ ID NO: 156), wherein x can be any whole number, such as 1, 2, 3, 4, 5, 6, 7, 8, 9, and 10. In some embodiments, the peptide linker comprises GGSSG (SEQ ID NO: 157), GGGGG (SEQ ID NO: 158), GSGSGSGS (SEQ ID NO: 159), GSGG (SEQ ID NO: 160), GGGGS (SEQ ID NO: 161), GGGS (SEQ ID NO: 154), GGS (SEQ ID NO: 162), GGGSGGGSGGGS (SEQ ID NO: 163), or a variant or fragment thereof or any number of repeats and combinations thereof. Additionally, KKYKPYVPVTTN (SEQ ID NO: 166) from DkTx, and EPKSSDKTHT (SEQ ID NO: 167) from human IgG₃ can be used as a peptide linker or any number of repeats and combinations thereof. In some embodiments, the peptide linker comprises GGGSGGSGGGS (SEQ ID NO: 164) or a variant or fragment thereof or any number of repeats and combinations thereof. In some embodiments, a peptide linker comprises any of SEQ ID NO: 154-SEQ ID NO: 241 or SEQ ID NO: 433. Additional linkers that may be linked, fused, or conjugated to a PD-L1-binding peptide of the present disclosure are provided in TABLE 9. It is understood that any of the foregoing linkers or a variant or fragment thereof can be used with any number of repeats or any combinations thereof. It is also understood that other peptide linkers in the art or a variant or fragment thereof can be used with any number of repeats or any combinations thereof. The length of the linker can be tailored to maximize binding of the PD-L1-binding peptide complex to both PD-L1 and an additional target (e.g., a target on an immune cell) at the same time including accounting for steric access. In some embodiments, the linker between the PD-L1-binding and immune cell-binding peptides is at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36 at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 residues incrementally up to 100 residues long.

In some embodiments of the present disclosure, a peptide or peptide complex as described herein comprises an amino acid sequence set forth in any one of SEQ ID NO: 1-SEQ ID NO: 118, SEQ ID NO: 435, SEQ ID NO: 436, or SEQ ID NO: 554-SEQ ID NO: 567. A peptide as disclosed herein can be a fragment comprising a contiguous fragment of any one of SEQ ID NO: 1-SEQ ID NO: 118, SEQ ID NO: 435, SEQ ID NO: 436, or SEQ ID NO: 554-SEQ ID NO: 567 that is at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36 at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 residues long, wherein the peptide fragment is selected from any portion of the peptide. In some embodiments, the peptide sequence is flanked by additional amino acids. One or more additional amino acids, for example, confer a particular in vivo charge, isoelectric point, chemical conjugation site, stability, or physiologic property to a peptide.

In some instances, the CDPs described herein that are capable of targeting and binding to a PD-L1 comprise no more than 80 amino acids in length, or no more than 70, no more than 65, no more than 60, no more than 55, no more than 50, no more than 49, no more than 45, no more than 40, no more than 35, no more than 30, no more than 25, no more than 20, no more than 15, or no more than 10 amino acids in length. In some instances, a PD-L1-binding moiety (e.g., an scFv) described herein that is capable of targeting and binding to a PD-L1 comprises a length of from about 100 to about 400, from about 200 to about 300, or from about 240 to about 250 amino acids in length.

In other embodiments, peptides can be conjugated to, linked to, or fused to a carrier or a molecule with targeting or homing function for a cell of interest or a target cell (e.g., an immune cell). In other embodiments, peptides can be conjugated to, linked to, or fused to a molecule that extends half-life or modifies the pharmacodynamic and/or pharmacokinetic properties of the peptides, or any combination thereof, such as an Fc region or polyethylene glycol.

In some instances, a peptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 positively charged residues, such as Arg or Lys, or any combination thereof. In some instances, one or more lysine residues in the peptide are replaced with arginine residues. In some embodiments, peptides comprise one or more Arg patches. In some embodiments, 1 or more, 2 or more, 3 or more, 4 or more, 5 or more, 6 or more, 7 or more, 8 or more, 9 or more, or 10 or more Arg or Lys residues are solvent exposed on a peptide. In some instances, a peptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 histidine residues.

The peptides of the present disclosure can further comprise neutral amino acid residues. In some embodiments, the peptide has 35 or fewer neutral amino acid residues. In other embodiments, the peptide has 81 or fewer neutral amino acid residues, 70 or fewer neutral amino acid residues, 60 or fewer neutral amino acid residues, 50 or fewer neutral amino acid residues, 40 or fewer neutral amino acid residues, 36 or fewer neutral amino acid residues, 33 or fewer neutral amino acid residues, 30 or fewer neutral amino acid residues, 25 or fewer neutral amino acid residues, or 10 or fewer neutral amino acid residues.

The peptides of the present disclosure can further comprise negative amino acid residues. In some embodiments the peptide has 10 or fewer, 9 or fewer, 8 or fewer, 7 or fewer, 6 or fewer negative amino acid residues, 5 or fewer negative amino acid residues, 4 or fewer negative amino acid residues, 3 or fewer negative amino acid residues, 2 or fewer negative amino acid residues, or 1 or fewer negative amino acid residues. While negative amino acid residues can be selected from any negatively charged amino acid residues, in some embodiments, the negative amino acid residues are either E, or D, or a combination of both E and D.

In some embodiments of the present disclosure, a three-dimensional or tertiary structure of a peptide is primarily comprised of beta-sheets and/or alpha-helix structures. In some embodiments, designed or engineered PD-L1-binding peptides or peptide complexes of the present disclosure are small, compact peptides or polypeptides stabilized by intra-chain disulfide bonds (e.g., mediated by cysteines) to form cystine bonds. In some embodiments, engineered PD-L1-binding peptides have structures comprising helix-turn-helix motifs with at least one disulfide bridge between each of the alpha helices, thereby stabilizing the peptides. In some embodiments, the engineered PD-L1-binding peptides or peptide complexes comprise structures with an alpha helix, one or more beta sheets, one or more alpha helices or one or more intra-chain disulfide bonds. In some embodiments, the engineered PD-L1-binding peptides or peptide complexes contain no hydrophobic core.

### Cystine-Dense Peptides

In some embodiments, PD-L1-binding peptides or peptide complexes of the present disclosure comprise one or more cysteine (Cys) amino acid residues, or one or more disulfide bonds. In some embodiments, the PD-L1-binding peptides or peptide complexes are derived from cystine-dense peptides (CDPs), knotted peptides, or hitchins. In some embodiments, CDPs contain at least 3 intramolecular cystine bonds. As used herein, the term “peptide” is considered to be interchangeable with the terms “knotted peptide,” “cystine-dense peptide,” “CDP,” “knottin,” and “hitchin,” (See, for example, Correnti et al. Screening, large-scale production, and structure-based classification for cystine-dense peptides. Nat Struct Mol Biol. 2018 March; 25(3): 270-278).

The PD-L1-binding peptides of the present disclosure, or derivatives, fragments, or variants thereof, can be have an affinity and selectively for PD-L1, or a derivative or analog thereof. In some cases, the PD-L1-binding peptides of the present disclosure can be engineered using site-saturation mutagenesis (SSM) to exhibit improved PD-L1-binding properties or to alter the properties of the binding interface with PD-L1. In some cases, the peptides of the present disclosure are cystine-dense peptides (CDPs), related to knotted peptides or hitchin-derived peptides or knottin-derived peptides. The PD-L1-binding peptides can be cystine-dense peptides (CDPs). The PD-L1-binding CDPs can have a “pontoon” structure featuring a pair of alpha-helices separated by an unstructured loop and stabilized with disulfide bonds resembling the underside of a pontoon boat, with an example disulfide connectivity being 1-6, 2-5, 3-4 for a three-disulfide pontoon scaffold. The PD-L1-binding CDPs can also have a hitchin-like structure. Hitchins can be a subclass of CDPs wherein six cysteine residues form disulfide bonds according to the connectivity [1-4], 2-5, 3-6 indicating that the first cysteine residue forms a disulfide bond with the fourth residue, the second with the fifth, and the third cysteine residue with the sixth. The brackets in this nomenclature indicate cysteine residues form the knotting disulfide bond. (See e.g., Correnti et al. Screening, large-scale production, and structure-based classification for cystine-dense peptides. Nat Struct Mol Biol. 2018 March; 25(3): 270-278). Knottins can be a subclass of CDPs wherein six cysteine residues form disulfide bonds according to the connectivity 1-4, 2-5, [3-6]. Knottins are a class of peptides, usually ranging from about 20 to about 80 amino acids in length that are often folded into a compact structure. Knottins are typically assembled into a complex tertiary structure that is characterized by a number of intramolecular disulfide crosslinks and can contain beta strands and other secondary structures. The presence of the disulfide bonds gives CDPs, including knottins, pontoons, and hitchins, remarkable environmental stability, allowing them to withstand extremes of temperature and pH and to resist the proteolytic enzymes and reducing molecules of the blood stream. In some cases, the peptides described herein can be derived from knotted peptides. The amino acid sequences of peptides as disclosed herein can comprise a plurality of cysteine residues. In some cases, at least cysteine residues of the plurality of cysteine residues present within the amino acid sequence of a peptide participate in the formation of disulfide bonds. In some cases, all cysteine residues of the plurality of cysteine residues present within the amino acid sequence of a peptide participate in the formation of disulfide bonds. As described herein, the term “knotted peptide” can be used interchangeably with the terms “cystine-dense peptide”, “CDP”, or “peptide”.

Provide herein are methods of identification, maturation, characterization, and utilization of CDPs that bind PD-L1 and allow selection, optimization and characterization of PD-L1-binding CDPs that can be used alone or in peptide complexes, including for use as bioactive molecules at therapeutically relevant concentrations in a subject (e.g., a human or non-human animal). This disclosure demonstrates the utility of CDPs as a diverse scaffold family that can be screened for applicability to modern drug discovery strategies. CDPs comprise alternatives to existing biologics, primarily antibodies, which can bypass some of the liabilities of the immunoglobulin scaffold, including poor tissue permeability, immunogenicity, larger size, and long serum half-life that can become problematic if toxicities arise. Peptides of the present disclosure in the 20-80 amino acid range represent medically relevant therapeutics that are mid-sized, with many of the favorable binding specificity and affinity characteristics of antibodies but with improved stability, reduced immunogenicity, and simpler manufacturing methods. The intramolecular disulfide architecture of CDPs provides particularly high stability metrics, reducing fragmentation and immunogenicity, while their smaller size could improve tissue penetration or cell penetration and facilitate tunable serum half-life. Disclosed herein are peptides representing candidate peptides that can bind and inhibit PD-L1 or serve as vehicles for active agent delivery to PD-L1 positive cells.

In some embodiments, PD-L1-binding peptides can be engineered peptides. An engineered peptide can be a peptide that is non-naturally occurring, artificial, isolated, synthetic, designed, or recombinantly expressed. In some embodiments, the PD-L1-binding peptides of the present disclosure comprise one or more properties of CDPs, knotted peptides, or hitchins, such as stability, resistance to proteolysis, resistance to reducing conditions, and/or ability to cross the blood brain barrier.

CDPs can be advantageous for intra-tumoral delivery, intracellular delivery, or delivery to the CNS, as compared to other molecules such as antibodies due to smaller size, greater tissue or cell penetration, lack of Fc function, and quicker clearance from serum, and as compared to smaller peptides due to resistance to proteases (both for stability and for immunogenicity reduction). In some embodiments, the PD-L1-binding peptides of the present disclosure, or engineered PD-L1-binding complexes (e.g., comprising one or more PD-L1-binding peptides and one or additional active agents) can have properties that are superior to PD-L1-binding antibodies or target-binding antibodies (e.g., bispecific antibodies or chimeric antigen receptors). For example, the peptides and complexes described herein can provide superior, deeper, and/or faster tissue or cell penetration to cells and targeted tissues (e.g., brain parenchyma penetration, solid tumor penetration) and faster clearance from non-targeted tissues and serum. The PD-L1-binding peptides or PD-L1-binding peptide complexes of this disclosure can have lower molecular weights than PD-L1-binding antibodies. The lower molecular weight can confer advantageous properties on the PD-L1-binding peptides or PD-L1-binding peptide complexes of this disclosure as compared to PD-L1-binding antibodies. For example, the PD-L1-binding peptides or PD-L1-binding peptide complexes of this disclosure can penetrate a cell or tissue more readily than an anti-PD-L1 antibody or can have lower molar dose toxicity than an anti-PD-L1 antibody. In addition, the PD-L1 binding peptides or PD-L1-binding peptide complexes of this disclosure can form an immune synapse between an immune cell and a cancer cell that is of a better geometry to induce cancer cell killing. The PD-L1-binding peptides or PD-L1-binding peptide complexes of this disclosure can be advantageous for lacking the Fc function of an antibody. The PD-L1-binding peptides or PD-L1-binding peptide complexes of this disclosure can be advantageous for allowing higher concentrations, on a molar basis, of formulations. The PD-L1-binding peptides or PD-L1-binding peptide complexes of this disclosure can have a higher affinity or faster on-rate to PD-L1 than antibody or antibody fragments. The PD-L1-binding peptides or PD-L1-binding peptide complexes of this disclosure can also be targeted to cancer cells in the CNS or brain via blood brain barrier (BBB) penetrating moieties, such as BBB-penetrating CDPs, to better access CNS tumors which are otherwise inaccessible to antibodies.

CDPs (e.g., knotted peptides or hitchins) are a class of peptides, usually ranging from about 11 to about 81 amino acids in length that are often folded into a compact structure. Knotted peptides are typically assembled into a complex tertiary structure that is characterized by a number of intramolecular disulfide crosslinks and can contain beta strands, alpha helices, and other secondary structures. The presence of the disulfide bonds gives knotted peptides remarkable environmental stability, allowing them to withstand extremes of temperature and pH and to resist the proteolytic enzymes of the blood stream. The presence of a disulfide knot can provide resistance to reduction by reducing agents. The rigidity of knotted peptides also allows them to bind to targets without paying the “entropic penalty” that a floppy peptide accrues upon binding a target. For example, binding is adversely affected by the loss of entropy that occurs when a peptide binds a target to form a complex. Therefore, “entropic penalty” is the adverse effect on binding, and the greater the entropic loss that occurs upon this binding, the greater the “entropic penalty.” Furthermore, unbound molecules that are flexible lose more entropy when forming a complex than molecules that are rigidly structured, because of the loss of flexibility when bound up in a complex. However, rigidity in the unbound molecule also generally increases specificity by limiting the number of complexes that molecule can form. The peptides can bind targets with affinity comparable to or higher than that of an antibody, or with nanomolar or picomolar affinity. A wider examination of the sequence structure and sequence identity or homology of knotted peptides reveals that they have arisen by convergent evolution in all kinds of animals and plants. In animals, they are often found in venoms, for example, the venoms of spiders and scorpions and have been implicated in the modulation of ion channels. The knotted proteins of plants can inhibit the proteolytic enzymes of animals or have antimicrobial activity, suggesting that knotted peptides can function in molecular defense systems found in plants.

A peptide of the present disclosure (e.g., PD-L1-binding peptide) can comprise a cysteine amino acid residue. In some embodiments, the peptide has at least 1, at least 2, at least 3, at least 4, at least 5, at least 6, at least 7, at least 8, at least 9, or at least 10 cysteine amino acid residues. In some embodiments, the peptide has at least 6 cysteine amino acid residues. In some embodiments, the peptide has at least 8 cysteine amino acid residues. In other embodiments, the peptide has at least 10 cysteine amino acid residues, at least 12 cysteine amino acid residues, at least 14 cysteine amino acid residues or at least 16 cysteine amino acid residues. In some embodiments, a peptide of the present disclosure has an even number of cysteine residues. In some embodiments, all cysteines in a peptide of the present disclosure are engaged within cystine disulfide bonds.

A knotted peptide can comprise disulfide bridges. A knotted peptide can be a peptide wherein 5% or more of the residues are cysteines forming intramolecular disulfide bonds. A disulfide-linked peptide can be a drug scaffold. In some embodiments, the disulfide bridges form a knot. A disulfide bridge can be formed between cysteine residues, for example, between cysteines 1 and 4, 2 and 5, or, 3 and 6. In some embodiments, one disulfide bridge passes through a loop formed by the other two disulfide bridges, for example, to form the knot. In other embodiments, the disulfide bridges can be formed between any two cysteine residues.

Some peptides of the present disclosure can comprise at least one amino acid residue in an L configuration. A peptide can comprise at least one amino acid residue in D configuration. In some embodiments, a peptide is 15-75 amino acid residues long. In other embodiments, a peptide is 11-55 amino acid residues long. In still other embodiments, a peptide is 11-65 amino acid residues long. In further embodiments, a peptide is at least 20 amino acid residues long.

Some CDPs can be derived or isolated from a class of proteins known to be present or associated with toxins or venoms. In some cases, the peptide can be derived from toxins or venoms associated with scorpions or spiders. The peptide can be derived from venoms and toxins of spiders and scorpions of various genus and species. For example, the peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Haplopelma huwenum, Haplopelma hainanum, Haplopelma schmidti, Agelenopsis aperta, Haydronyche versuta, Selenocosmia huwena, Heteropoda venatoria, Grammostola rosea, Ornithoctonus huwena, Hadronyche versuta, Atrax robustus, Angelenopsis aperta, Psalmopoeus cambridgei, Hadronyche infensa, Paracoelotes luctosus, and Chilobrachys jingzhaoor another suitable genus or species of scorpion or spider. In some cases, a peptide can be derived from a Buthus martensii Karsh (scorpion) toxin. In some cases, a CDP may be derived from subunit 6 of a cytochrome BC1 oxidoreductase or from an antimicrobial defensin.

In some embodiments, a peptide of the present disclosure (e.g., a PD-L1-binding peptide) can comprise a sequence having cysteine residues at one or more of corresponding positions 4, 8, 18, 32, 42, and 46, for example with reference to SEQ ID NO: 1. For example, in certain embodiments, a peptide can comprise a sequence having a cysteine residue at corresponding position 4. In certain embodiments, a peptide can comprise a sequence having a cysteine residue at corresponding position 8. In certain embodiments, a peptide can comprise a sequence having a cysteine residue at corresponding position 18. In certain embodiments, a peptide can comprise a sequence having a cysteine residue at corresponding position 32. In certain embodiments, a peptide can comprise a sequence having a cysteine residue at corresponding position 42. In certain embodiments, a peptide can comprise a sequence having a cysteine residue at corresponding position 46. In some embodiments, a peptide comprises cysteines at corresponding positions n, n+4±2, n+14±2, n+28±2, n+38±2, or n+42±2, or any combination thereof, where n corresponds to an amino acid position of a first cysteine residue (e.g., position 4 of SEQ ID NO: 1). In some embodiments, a peptide comprises cysteines at corresponding positions n, n+4, n+14, n+28, n+38, or n+42, or any combination thereof, where n corresponds to an amino acid position of a first cysteine residue (e.g., position 4 of SEQ ID NO: 1). For example, a peptide of the present disclosure can comprise a sequence having cysteines positioned such that a second cysteine residue is positioned 4 amino acid residues toward the peptide C-terminus from a first cysteine residue, a third cysteine residue is positioned 14 amino acid residues toward the peptide C-terminus from the first cysteine residue, a fourth cysteine residue is positioned 28 amino acid residues toward the peptide C-terminus from the first cysteine residue, a fifth cysteine residue is positioned 38 amino acid residues toward the peptide C-terminus from the first cysteine residue, a sixth cysteine residue is positioned 42 amino acid residues toward the peptide C-terminus from the first cysteine residue, or combinations thereof. In some embodiments, a peptide of the present disclosure can comprise a sequence having cysteines spaced such that there are 3 amino acid residues between a first cysteine and a second cysteine, 9 amino acid residues between a second cysteine and a third cysteine, 13 amino acid residues between a third cysteine and a fourth cysteine, 9 amino acid residues between a fourth cysteine and a fifth cysteine, 3 amino acid residues between a fifth cysteine and a sixth cysteine, or combinations thereof.

In some embodiments, a peptide of the present disclosure (e.g., a PD-L1-binding peptide) can comprise a sequence having cysteine residues at one or more of corresponding positions 4, 15, 21, 25, 35, 42, 44, 48, for example with reference to SEQ ID NO: 58. For example, in certain embodiments, a peptide can comprise a sequence having a cysteine residue at corresponding position 4. In certain embodiments, a peptide can comprise a sequence having a cysteine residue at corresponding position 15. In certain embodiments, a peptide can comprise a sequence having a cysteine residue at corresponding position 21. In certain embodiments, a peptide can comprise a sequence having a cysteine residue at corresponding position 25. In certain embodiments, a peptide can comprise a sequence having a cysteine residue at corresponding position 35. In certain embodiments, a peptide can comprise a sequence having a cysteine residue at corresponding position 42. In certain embodiments, a peptide can comprise a sequence having a cysteine residue at corresponding position 44. In certain embodiments, a peptide can comprise a sequence having a cysteine residue at corresponding position 48. In some embodiments, a peptide comprises cysteines at corresponding positions n, n+11±2, n+17±2, n+21±2, n+31±2, n+38±2, n+40±2, or n+44±2 or any combination thereof, where n corresponds to an amino acid position of a first cysteine residue (e.g., position 4 of SEQ ID NO: 58). In some embodiments, a peptide comprises cysteines at corresponding positions n, n+11, n+17, n+21, n+31, n+38, n+40, or n+44, or any combination thereof, where n corresponds to an amino acid position of a first cysteine residue (e.g., position 4 of SEQ ID NO: 58). For example, a peptide of the present disclosure can comprise a sequence having cysteines positioned such that a second cysteine residue is positioned 11 amino acid residues toward the peptide C-terminus from a first cysteine residue, a third cysteine residue is positioned 17 amino acid residues toward the peptide C-terminus from the first cysteine residue, a fourth cysteine residue is positioned 21 amino acid residues toward the peptide C-terminus from the first cysteine residue, a fifth cysteine residue is positioned 31 amino acid residues toward the peptide C-terminus from the first cysteine residue, a sixth cysteine residue is positioned 38 amino acid residues toward the peptide C-terminus from the first cysteine residue, a seventh cysteine residue is positioned 40 amino acid residues toward the peptide C-terminus from the first cysteine residue, an eighth cysteine residue is positioned 44 amino acid residues toward the peptide C-terminus from the first cysteine residue, or combinations thereof. In some embodiments, a peptide of the present disclosure can comprise a sequence having cysteines spaced such that there are 10 amino acid residues between a first cysteine and a second cysteine, 5 amino acid residues between a second cysteine and a third cysteine, 3 amino acid residues between a third cysteine and a fourth cysteine, 9 amino acid residues between a fourth cysteine and a fifth cysteine, 6 amino acid residues between a fifth cysteine and a sixth cysteine, 1 amino acid residue between a sixth cysteine and a seventh cysteine, 3 amino acid residues between a seventh cysteine and an eighth cysteine, or combinations thereof.

In some embodiments, peptides of the present disclosure (e.g., PD-L1-binding peptides) comprise at least one cysteine residue. In some embodiments, peptides of the present disclosure comprise at least two cysteine residues. In some embodiments, peptides of the present disclosure comprise at least three cysteine residues. In some embodiments, peptides of the present disclosure comprise at least four cysteine residues. In some embodiments, peptides of the present disclosure comprise at least five cysteine residues. In some embodiments, peptides of the present disclosure comprise at least six cysteine residues. In some embodiments, peptides of the present disclosure comprise at least eight cysteine residues. In some embodiments, peptides of the present disclosure comprise at least ten cysteine residues. In some embodiments, a peptide of the present disclosure comprises six cysteine residues. In some embodiments, a peptide of the present disclosure comprises seven cysteine residues. In some embodiments, a peptide of the present disclosure comprises eight cysteine residues. In some embodiments, a peptide of the present disclosure comprises nine cysteine residues.

In some embodiments, the first cysteine residue in the sequence can be disulfide bonded with the 4th cysteine residue in the sequence, the 2nd cysteine residue in the sequence can be disulfide bonded to the 5th cysteine residue in the sequence, and the 3rd cysteine residue in the sequence can be disulfide bonded to the 6th cysteine residue in the sequence. Optionally, a peptide can comprise one disulfide bridge that passes through a ring formed by two other disulfide bridges, also known as a “two-and-through” structure system. In some embodiments, the peptides disclosed herein can have one or more cysteines mutated to serine. In some embodiments, the first cysteine residue in the sequence (e.g., at position 4 of SEQ ID NO: 1) can be disulfide bonded with the 6th cysteine residue in the sequence (e.g., at position 46 of SEQ ID NO: 1), the 2nd cysteine residue in the sequence (e.g., at position 8 of SEQ ID NO: 1) can be disulfide bonded to the 5th cysteine residue in the sequence (e.g., at position 42 of SEQ ID NO: 1), and the 3rd cysteine residue in the sequence (e.g., at position 18 of SEQ ID NO: 1) can be disulfide bonded to the 4th cysteine residue in the sequence (e.g., at position 32 of SEQ ID NO: 1). In some embodiments, this disulfide bond structure may be present in a peptide of any one of SEQ ID NO: 1-SEQ ID NO: 4, SEQ ID NO: 8-SEQ ID NO: 57, SEQ ID NO: 59-SEQ ID NO: 63, SEQ ID NO: 67-SEQ ID NO: 116, SEQ ID NO: 118, SEQ ID NO: 435, SEQ ID NO: 436, or SEQ ID NO: 554-SEQ ID NO: 567.

In some embodiments, the first cysteine residue in the sequence (e.g., at position 4 of SEQ ID NO: 58) can be disulfide bonded with the 8th cysteine residue in the sequence (e.g., at position 48 of SEQ ID NO: 58), the 2nd cysteine residue in the sequence (e.g., at position 15 of SEQ ID NO: 58) can be disulfide bonded to the 5th cysteine residue in the sequence (e.g., at position 35 of SEQ ID NO: 58), the 3rd cysteine residue in the sequence (e.g., at position 21 of SEQ ID NO: 58) can be disulfide bonded to the 6th cysteine residue in the sequence (e.g., at position 42 of SEQ ID NO: 58), and the 4th cysteine residue in the sequence (e.g., at position 25 of SEQ ID NO: 58) can be disulfide bonded to the 7th cysteine residue in the sequence (e.g., at position 44 of SEQ ID NO: 58).

In some embodiments, a peptide of the present disclosure (e.g., a PD-L1-binding peptide) comprises an amino acid sequence having cysteine residues at one or more positions, for example with reference to SEQ ID NO: 1. In some embodiments, the one or more cysteine residues are located at any of the corresponding amino acid positions 4, 8, 18, 32, 42, 46, or any combination thereof. In some embodiments, the one or more cysteine residues are located at any of the corresponding amino acid positions 4, 15, 21, 25, 35, 42, 44, 48, or any combination thereof. In some aspects of the present disclosure, the one or more cysteine (C) residues participate in disulfide bonds with various pairing patterns (e.g., C¹⁰-C²⁰). In some embodiments, the peptides as described herein comprise at least one, at least two, at least three, or at least four disulfide bonds. In some embodiments, peptides as described herein comprise three disulfide bonds with the corresponding pairing patterns C⁴-C⁴⁶, C⁸-C⁴², and C¹⁸-C³². In some embodiments, peptides as described herein comprise four disulfide bonds with the corresponding pairing patterns C⁴-C⁴⁸, C¹⁵-C³⁵, C²¹-C⁴², and C²⁵-C⁴⁴. In some embodiments, peptides as described herein comprise three disulfide bonds with the corresponding pairing patterns C¹⁵-C³⁵, C²¹-C⁴², and C²⁵-C⁴⁴.

In some instances, one or more or all of the methionine residues in the peptide are replaced by leucine or isoleucine. In some instances, one or more or all of the tryptophan residues in the peptide are replaced by phenylalanine or tyrosine. In some instances, one or more or all of the asparagine residues in the peptide are replaced by glutamine. In some embodiments, the N-terminus of the peptide is blocked, such as by an acetyl group. Alternatively or in combination, in some instances, the C-terminus of the peptide is blocked, such as by an amide group. In some embodiments, the peptide is modified by methylation on free amines. For example, full methylation can be accomplished through the use of reductive methylation with formaldehyde and sodium cyanoborohydride.

### PD-L1-Binding Peptides

Disclosed herein are peptide sequences, such as those listed in TABLE 1, capable of binding to PD-L1, or any combination or fragment (e.g., ectodomain) thereof. A peptide capable of binding PD-L1 can be referred to herein as a PD-L1-binding peptide. In some embodiments, peptides disclosed herein can penetrate, cross, or enter target cells or can be modified to penetrate, cross, or enter target cells (e.g., PD-L1 positive cells). In some embodiments, peptides disclosed herein can penetrate or cross, or can be modified to penetrate or cross, a blood brain barrier (BBB). In some cases, a PD-L1-binding peptide may be part of a PD-L1-binding peptide complex comprising a PD-L1-binding peptide conjugated to, linked to, or fused to an additional active agent (e.g., a therapeutic agent, a detectable agent, or an immune cell targeting agent) such as a small molecule or a peptide that has an affinity for an additional target protein (e.g., an immune cell surface protein). In some cases, a peptide complex of the present disclosure exerts a biological effect that is mediated by the PD-L1-binding peptide, the additional active agent, or a combination thereof.

In some embodiments, PD-L1-binding peptides of the present disclosure, including peptides with amino acid sequences set forth in SEQ ID NO: 1-SEQ ID NO: 118, SEQ ID NO: 435, SEQ ID NO: 436, or SEQ ID NO: 554-SEQ ID NO: 567, and any derivatives or variant thereof, prevent or decrease the binding of endogenous PD-L1 binders (e.g., PD-1) to PD-L1. The PD-L1-binding peptides of the present disclosure (e.g., any one of SEQ ID NO: 1-SEQ ID NO: 118, SEQ ID NO: 435, SEQ ID NO: 436, or SEQ ID NO: 554-SEQ ID NO: 567) may compete with PD-1 for binding to PD-L1. A PD-L1-binding peptide may displace PD-1 from PD-L1. In some embodiments, a PD-L1-binding peptide may inhibit PD-1 binding to PD-L1 or displace PD-1 from PD-L1 in a subject (e.g., a human subject) with a half maximum inhibitory concentration (IC₅₀) of from about 1 pM to about 10 μM, from about 10 pM to about 10 μM, from about 100 pM to about 10 μM, from about 300 pM to about 10 μM, from about 500 pM to about 10 μM, from about 1 pM to about 1 pM, from about 10 pM to about 1 μM, from about 100 pM to about 1 μM, from about 300 pM to about 1 μM, from about 500 pM to about 1 μM, from about 1 pM to about 100 nM, from about 10 pM to about 100 nM, from about 100 pM to about 100 nM, from about 300 pM to about 100 nM, from about 500 pM to about 100 nM, from about 1 pM to about 10 nM, from about 10 pM to about 10 nM, from about 100 pM to about 10 nM, from about 300 pM to about 10 nM, from about 500 pM to about 10 nM, from about 1 pM to about 1 nM, from about 10 pM to about 1 nM, from about 100 pM to about 1 nM, from about 1 pM to about 500 pM, from about 10 pM to about 500 pM, or from about 100 pM to about 500 pM. In some embodiments, a PD-L1-binding peptide may inhibit PD-1 binding to PD-L1 or displace PD-1 from PD-L1 on a cell (e.g., a human cell) under physiological conditions with a half maximum inhibitory concentration (IC₅₀) of from about 1 pM to about 1 pM, from about 10 pM to about 1 μM, from about 100 pM to about 1 μM, from about 300 pM to about 1 pM, from about 500 pM to about 1 μM, from about 1 pM to about 100 nM, from about 10 pM to about 100 nM, from about 100 pM to about 100 nM, from about 300 pM to about 100 nM, from about 500 pM to about 100 nM, from about 1 pM to about 10 nM, from about 10 pM to about 10 nM, from about 100 pM to about 10 nM, from about 300 pM to about 10 nM, from about 500 pM to about 10 nM, from about 1 pM to about 1 nM, from about 10 pM to about 1 nM, from about 100 pM to about 1 nM, from about 1 pM to about 500 pM, from about 10 pM to about 500 pM, or from about 100 pM to about 500 pM.

In some embodiments, peptides of the present disclosure comprise derivatives and variants with at least 40% homology, at least 50% homology, at least 60% homology, at least 70% homology, at least 75% homology, at least 80% homology, at least 85% homology, at least 90% homology, at least 91% homology, at least 92% homology, at least 93% homology, at least 94% homology, at least 95% homology, at least 96% homology, at least 97% homology, at least 98% homology, or at least 99% homology or 100% homology to amino acid sequences set forth in SEQ ID NO: 1-SEQ ID NO: 118, SEQ ID NO: 435, SEQ ID NO: 436, or SEQ ID NO: 554-SEQ ID NO: 567. For example, a PD-L1-binding peptide may comprise a sequence having at least 40% homology, at least 50% homology, at least 60% homology, at least 70% homology, at least 75% homology, at least 80% homology, at least 85% homology, at least 90% homology, at least 91% homology, at least 92% homology, at least 93% homology, at least 94% homology, at least 95% homology, at least 96% homology, at least 97% homology, at least 98% homology, or at least 99% homology or 100% homology to an amino acid sequence set forth in SEQ ID NO: 1-SEQ ID NO: 118, SEQ ID NO: 435, SEQ ID NO: 436, or SEQ ID NO: 554-SEQ ID NO: 567.

In some embodiments, PD-L1-binding peptides bind to PD-L1 with equal, similar, or greater affinity (e.g., lower equilibrium dissociation constant, K_{D}) as compared to endogenous molecules (e.g., PD-1), or any other endogenous PD-L1 ligands) or other exogenous molecules (e.g., PD-L1-binding antibodies or antibody fragments). In some embodiments, the peptide or peptide complex can have a K_{D} for PD-L1 binding of no greater than 50 μM, no greater than 5 μM, no greater than 500 nM, no greater than 100 nM, no greater than 40 nM, no greater than 30 nM, no greater than 20 nM, no greater than 15 nM, no greater than 10 nM, no greater than 5 nM, no greater than 2 nM, no greater than 1 nM, no greater than 0.9 nM, no greater than 0.8 nM, no greater than 0.7 nM, no greater than 0.6 nM, no greater than 0.5 nM, no greater than 0.4 nM, no greater than 0.3 nM, no greater than 0.2 nM, or no greater than 0.1 nM. In some embodiments, PD-L1-binding peptides that exhibit an improved PD-L1 binding show improved recruitment to PD-L1 positive cells, improved inhibition of PD-L1 or of PD-1 binding, improved active agent delivery, improved immune cell recruitment, improved cell killing, improved tumor regression, or combinations thereof. In some embodiments, the K_{A}, K_{D}, kₒₙ, k_{off} values, or combinations thereof of a PD-L1-binding peptide can be modulated and optimized (e.g., via amino acid substitutions) to provide a preferred ratio of PD-L1-binding affinity, rate of binding to PD-L1, rate of release from PD-L1, or combinations thereof. In some embodiments, the PD-L1-binding peptide binds at a site of low homology between human and murine PD-L1, reducing cross-reactivity of the PD-L1-binding peptides for human and murine PD-L1. Binding kinetics of CDPs differ from those of antibodies, so bispecific molecules containing a PD-L1-binding CDP and an antibody could have different behaviors than antibody-based molecules. The PD-L1-binding CDPs described herein may have a faster on-rate to PD-L1 than that of an antibody-based molecule.

In some embodiments, peptides disclosed herein or variants thereof bind to PD-L1 at residues found in the binding interface (e.g., the binding domain or the binding pocket) of PD-L1 with other exogenous or endogenous ligands (e.g., PD-1, PD-1 derivatives, or PD-1-like peptides or proteins). In some embodiments, a peptide disclosed herein or a variant thereof, which binds to PD-L1, comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to a sequence that binds residues of PD-L1, which makeup the binding pocket. In some embodiments, a peptide disclosed herein or a variant thereof, which binds to PD-L1, comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to an endogenous or exogenous polypeptide known to bind PD-L1, for example, endogenous PD-1 or any one of the peptides listed in TABLE 1. In other embodiments, a peptide described herein binds to a protein of interest, which comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to PD-L1, a fragment, homolog, or a variant thereof.

In other embodiments, a nucleic acid, vector, plasmid, or donor DNA comprises a sequence that encodes a peptide, peptide construct, a peptide complex, or variant or functional fragment thereof, as described in the present disclosure. In further embodiments, certain parts or fragments of PD-L1-binding motifs (e.g., conserved binding motifs) can be grafted onto a peptide or peptide complex with a sequence of any one of SEQ ID NO: 1-SEQ ID NO: 118, SEQ ID NO: 435, SEQ ID NO: 436, or SEQ ID NO: 554-SEQ ID NO: 567.

In some embodiments, a peptide can be selected for further testing or use based upon its ability to bind to the certain amino acid residue or motif of amino acid residues. The certain amino acid residue or motif of amino acid residues in PD-L1 can be identified an amino acid residue or sequence of amino acid residues that are involved in the binding of PD-L1 to PD-1. A certain amino acid residue or motif of amino acid residues can be identified from a crystal structure of the PD-L1:PD-1 complex. In some embodiments, peptides (e.g., CDPs) demonstrate the resistance to heat, protease (e.g., pepsin, trypsin, or other), and reduction.

The peptides and peptide complexes (e.g., peptide conjugates or fusion peptides) comprising one or more of the amino acid sequences set forth in SEQ TD NO: 1-SEQ ID NO: 118, SEQ ID NO: 435, SEQ ID NO: 436, or SEQ ID NO: 554-SEQ ID NO: 567 can bind to a protein of interest. In some embodiments, the protein of interest is a PD-L1. In some embodiments, the peptides and peptide complexes (e.g., peptide conjugates or fusion peptides) that bind to a PD-L1 comprise at least one of the amino acid sequences set forth in SEQ TD NO: 1-SEQ ID NO: 118, SEQ ID NO: 435, SEQ ID NO: 436, or SEQ ID NO: 554-SEQ ID NO: 567. TABLE 1 lists exemplary peptide sequences according to the methods and compositions of the present disclosure.

TABLE 1
Exemplary PD-L1-Binding Peptides

| SEQ ID NO: | Sequence |
|---|---|
| SEQ ID NO: 1 | EEDCKVHCVKEWMAGKACAERQKSYTIGRAHCSGQKFDVFKCLD |
| | HCAAP |
| SEQ ID NO: 2 | EEDCKVHCVKEWMAGKACAERDKSYTIGRAHCSGQKFDVFKCLD |
| | HCAAP |
| SEQ ID NO: 3 | EEDCKVHCVKEWMAGKACAERNKSYTIGRAHCSGQKFDVFKCLD |
| | HCAAP |
| SEQ ID NO: 4 | EEDCKVHCVKEWAAYKACAERIKSYTIGRAHCSGQYFDVWKCLD |
| | HCAAP |
| SEQ ID NO: 5 | ARTCESQSHRFKGPCVSMTNCASVCRTERFSGGHCRGFRRRCLCT |
| | KHC |
| SEQ ID NO: 6 | ARTCESQSHRFKGPCVSDTNCASVCYTERFSGGHCRGFRRRCLCTK |
| | HC |
| SEQ ID NO: 7 | ARTCESQSHRFKGPCVSDTNCASVCRTERFSGGHCMGFRRRCLCT |
| | KHC |
| SEQ ID NO: 8 | EEDCKVHCVKWWMAGKACAERNKSYTIGRAHCSGQKFDVFKCL |
| | DHCAAP |
| SEQ ID NO: 9 | EEDCKVHCVKEWMAGKACAERNKSYTIGRAHCSGQKFDVFKCLD |
| | HCAAP |
| SEQ ID NO: 10 | EEDCKVHCVKWWAAGKACAERNKSYTIGRAHCSGQKFDVFKCL |
| | DHCAAP |
| SEQ ID NO: 11 | EEDCKVHCVKWWMAYKACAERNKSYTIGRAHCSGQKFDVFKCL |
| | DHCAAP |
| SEQ ID NO: 12 | EEDCKVHCVKWWMAGKACAERIKSYTIGRAHCSGQKFDVFKCLD |
| | HCAAP |
| SEQ ID NO: 13 | EEDCKVHCVKWWMAGKACAERNKSYTIGRAHCSGQYFDVFKCL |
| | DHCAAP |
| SEQ ID NO: 14 | EEDCKVHCVKWWMAGKACAERNKSYTIGRAHCSGQKFDVWKCL |
| | DHCAAP |
| SEQ ID NO: 15 | EEDCKVHCVKWWAAYKACAERIKSYTIGRAHCSGQYFDVWKCL |
| | DHCAAP |
| SEQ ID NO: 16 | EEDCKVHCVKEWMAYKACAERIKSYTIGRAHCSGQYFDVWKCLD |
| | HCAAP |
| SEQ ID NO: 17 | EEDCKVHCVKEWAAGKACAERIKSYTIGRAHCSGQYFDVWKCLD |
| | HCAAP |
| SEQ ID NO: 18 | EEDCKVHCVKEWAAYKACAERNKSYTIGRAHCSGQYFDVWKCL |
| | DHCAAP |
| SEQ ID NO: 19 | EEDCKVHCVKEWAAYKACAERIKSYTIGRAHCSGQKFDVWKCLD |
| | HCAAP |
| SEQ ID NO: 20 | EEDCKVHCVKEWAAYKACAERIKSYTIGRAHCSGQYFDVFKCLD |
| | HCAAP |
| SEQ ID NO: 21 | EEDCKVHCVKEWMAGKACAERIKSYTIGRAHCSGQYFDVWKCLD |
| | HCAAP |
| SEQ ID NO: 22 | EEDCKVHCVKEWMAYKACAERNKSYTIGRAHCSGQYFDVWKCL |
| | DHCAAP |
| SEQ ID NO: 23 | EEDCKVHCVKEWMAYKACAERIKSYTIGRAHCSGQKFDVWKCLD |
| | HCAAP |
| SEQ ID NO: 24 | EEDCKVHCVKEWMAYKACAERIKSYTIGRAHCSGQYFDVFKCLD |
| | HCAAP |
| SEQ ID NO: 25 | EEDCKVHCVKEWAAGKACAERNKSYTIGRAHCSGQYFDVWKCL |
| | DHCAAP |
| SEQ ID NO: 26 | EEDCKVHCVKEWAAGKACAERIKSYTIGRAHCSGQKFDVWKCLD |
| | HCAAP |
| SEQ ID NO: 27 | EEDCKVHCVKEWAAGKACAERIKSYTIGRAHCSGQYFDVFKCLD |
| | HCAAP |
| SEQ ID NO: 28 | EEDCKVHCVKEWAAYKACAERNKSYTIGRAHCSGQKFDVWKCL |
| | DHCAAP |
| SEQ ID NO: 29 | EEDCKVHCVKEWAAYKACAERNKSYTIGRAHCSGQYFDVFKCLD |
| | HCAAP |
| SEQ ID NO: 30 | EEDCKVHCVKEWAAYKACAERIKSYTIGRAHCSGQKFDVFKCLD |
| | HCAAP |
| SEQ ID NO: 31 | EEDCKVHCVKEWMAGKACAERQKSDTTGQAHCSGQKFDVFKCL |
| | DHCAAP |
| SEQ ID NO: 32 | EEDCKVHCVKEWMAGKACAERNKSDTTGQAHCSGQKFDVFKCL |
| | DHCAAP |
| SEQ ID NO: 33 | EEDCKVHCVKEWAAYKACAERIKSDTTGQAHCSGQYFDVWKCL |
| | DHCAAP |
| SEQ ID NO: 34 | EEDCKVHCVKWWMAGKACAERNKSDTTGQAHCSGQKFDVFKCL |
| | DHCAAP |
| SEQ ID NO: 35 | EEDCKVHCVKEWMAGKACAERNKSDTTGQAHCSGQKFDVFKCL |
| | DHCAAP |
| SEQ ID NO: 36 | EEDCKVHCVKWWAAGKACAERNKSDTTGQAHCSGQKFDVFKCL |
| | DHCAAP |
| SEQ ID NO: 37 | EEDCKVHCVKWWMAYKACAERNKSDTTGQAHCSGQKFDVFKCL |
| | DHCAAP |
| SEQ ID NO: 38 | EEDCKVHCVKWWMAGKACAERIKSDTTGQAHCSGQKFDVFKCL |
| | DHCAAP |
| SEQ ID NO: 39 | EEDCKVHCVKWWMAGKACAERNKSDTTGQAHCSGQYFDVFKCL |
| | DHCAAP |
| SEQ ID NO: 40 | EEDCKVHCVKWWMAGKACAERNKSDTTGQAHCSGQKFDVWKC |
| | LDHCAAP |
| SEQ ID NO: 41 | EEDCKVHCVKWWAAYKACAERIKSDTTGQAHCSGQYFDVWKCL |
| | DHCAAP |
| SEQ ID NO: 42 | EEDCKVHCVKEWMAYKACAERIKSDTTGQAHCSGQYFDVWKCL |
| | DHCAAP |
| SEQ ID NO: 43 | EEDCKVHCVKEWAAGKACAERIKSDTTGQAHCSGQYFDVWKCL |
| | DHCAAP |
| SEQ ID NO: 44 | EEDCKVHCVKEWAAYKACAERNKSDTTGQAHCSGQYFDVWKCL |
| | DHCAAP |
| SEQ ID NO: 45 | EEDCKVHCVKEWAAYKACAERIKSDTTGQAHCSGQKFDVWKCL |
| | DHCAAP |
| SEQ ID NO: 46 | EEDCKVHCVKEWAAYKACAERIKSDTTGQAHCSGQYFDVFKCLD |
| | HCAAP |
| SEQ ID NO: 47 | EEDCKVHCVKEWMAGKACAERIKSDTTGQAHCSGQYFDVWKCL |
| | DHCAAP |
| SEQ ID NO: 48 | EEDCKVHCVKEWMAYKACAERNKSDTTGQAHCSGQYFDVWKCL |
| | DHCAAP |
| SEQ ID NO: 49 | EEDCKVHCVKEWMAYKACAERIKSDTTGQAHCSGQKFDVWKCL |
| | DHCAAP |
| SEQ ID NO: 50 | EEDCKVHCVKEWMAYKACAERIKSDTTGQAHCSGQYFDVFKCLD |
| | HCAAP |
| SEQ ID NO: 51 | EEDCKVHCVKEWAAGKACAERNKSDTTGQAHCSGQYFDVWKCL |
| | DHCAAP |
| SEQ ID NO: 52 | EEDCKVHCVKEWAAGKACAERIKSDTTGQAHCSGQKFDVWKCL |
| | DHCAAP |
| SEQ ID NO: 53 | EEDCKVHCVKEWAAGKACAERIKSDTTGQAHCSGQYFDVFKCLD |
| | HCAAP |
| SEQ ID NO: 54 | EEDCKVHCVKEWAAYKACAERNKSDTTGQAHCSGQKFDVWKCL |
| | DHCAAP |
| SEQ ID NO: 55 | EEDCKVHCVKEWAAYKACAERNKSDTTGQAHCSGQYFDVFKCL |
| | DHCAAP |
| SEQ ID NO: 56 | EEDCKVHCVKEWAAYKACAERIKSDTTGQAHCSGQKFDVFKCLD |
| | HCAAP |
| SEQ ID NO: 57 | EESCKPQCVKAWLEYQACAERVEKDESGEAHCTGQYFDLWGCVD |
| | KCVAP |
| SEQ ID NO: 58 | ARTCESQSHRFKGPCVSDTMCASVCRTERFSGGHCRGFRRRCLCS |
| | KHC |
| SEQ ID NO: 59 | EERCMPQCVKSLYEYEKCLKRVENDDTGHKHCTGHYFDYWSCID |
| | KCVAS |
| SEQ ID NO: 435 | EEDCRVHCVREWMAGRACAERDRSYTIGRAHCSGQRFDVFRCLD |
| | HCAAP |
| SEQ ID NO: 554 | EHDCKVHCVKEWMAGHACAERQKSYTIGRAHCSGQKFDVFKCLD |
| | HCAAP |
| SEQ ID NO: 555 | EHDCKVHCVKEWMAGKACAERQKSYTIGRAHCSGQKFDVFKCLD |
| | HCAAP |
| SEQ ID NO: 556 | EEDCKVHCVKEWHAGKACAERQKSYTIGRAHCSGQKFDVFKCLD |
| | HCAAP |
| SEQ ID NO: 557 | EEDCKVHCVKEWMAGHACAERQKSYTIGRAHCSGQKFDVFKCLD |
| | HCAAP |
| SEQ ID NO: 558 | EHDCKVHCVKEWHAGKACAERQKSYTIGRAHCSGQKFDVFKCLD |
| | HCAAP |
| SEQ ID NO: 559 | EEDCKVHCVKEWHAGHACAERQKSYTIGRAHCSGQKFDVFKCLD |
| | HCAAP |
| SEQ ID NO: 560 | EHDCKVHCVKEWHAGHACAERQKSYTIGRAHCSGQKFDVFKCLD |
| | HCAAP |
| SEQ ID NO: 60 | GSEEDCKVHCVKEWMAGKACAERQKSYTIGRAHCSGQKFDVFKC |
| | LDHCAAP |
| SEQ ID NO: 61 | GSEEDCKVHCVKEWMAGKACAERDKSYTIGRAHCSGQKFDVFKC |
| | LDHCAAP |
| SEQ ID NO: 62 | GSEEDCKVHCVKEWMAGKACAERNKSYTIGRAHCSGQKFDVFKC |
| | LDHCAAP |
| SEQ ID NO: 63 | GSEEDCKVHCVKEWAAYKACAERIKSYTIGRAHCSGQYFDVWKC |
| | LDHCAAP |
| SEQ ID NO: 64 | GSARTCESQSHRFKGPCVSMTNCASVCRTERFSGGHCRGFRRRCL |
| | CTKHC |
| SEQ ID NO: 65 | GSARTCESQSHRFKGPCVSDTNCASVCYTERFSGGHCRGFRRRCLC |
| | TKHC |
| SEQ ID NO: 66 | GSARTCESQSHRFKGPCVSDTNCASVCRTERFSGGHCMGFRRRCL |
| | CTKHC |
| SEQ ID NO: 67 | GSEEDCKVHCVKWWMAGKACAERNKSYTIGRAHCSGQKFDVFK |
| | CLDHCAAP |
| SEQ ID NO: 68 | GSEEDCKVHCVKEWMAGKACAERNKSYTIGRAHCSGQKFDVFKC |
| | LDHCAAP |
| SEQ ID NO: 69 | GSEEDCKVHCVKWWAAGKACAERNKSYTIGRAHCSGQKFDVFKC |
| | LDHCAAP |
| SEQ ID NO: 70 | GSEEDCKVHCVKWWMAYKACAERNKSYTIGRAHCSGQKFDVFK |
| | CLDHCAAP |
| SEQ ID NO: 71 | GSEEDCKVHCVKWWMAGKACAERIKSYTIGRAHCSGQKFDVFKC |
| | LDHCAAP |
| SEQ ID NO: 72 | GSEEDCKVHCVKWWMAGKACAERNKSYTIGRAHCSGQYFDVFK |
| | CLDHCAAP |
| SEQ ID NO: 73 | GSEEDCKVHCVKWWMAGKACAERNKSYTIGRAHCSGQKFDVWK |
| | CLDHCAAP |
| SEQ ID NO: 74 | GSEEDCKVHCVKWWAAYKACAERIKSYTIGRAHCSGQYFDVWKC |
| | LDHCAAP |
| SEQ ID NO: 75 | GSEEDCKVHCVKEWMAYKACAERIKSYTIGRAHCSGQYFDVWKC |
| | LDHCAAP |
| SEQ ID NO: 76 | GSEEDCKVHCVKEWAAGKACAERIKSYTIGRAHCSGQYFDVWKC |
| | LDHCAAP |
| SEQ ID NO: 77 | GSEEDCKVHCVKEWAAYKACAERNKSYTIGRAHCSGQYFDVWK |
| | CLDHCAAP |
| SEQ ID NO: 78 | GSEEDCKVHCVKEWAAYKACAERIKSYTIGRAHCSGQKFDVWKC |
| | LDHCAAP |
| SEQ ID NO: 79 | GSEEDCKVHCVKEWAAYKACAERIKSYTIGRAHCSGQYFDVFKCL |
| | DHCAAP |
| SEQ ID NO: 80 | GSEEDCKVHCVKEWMAGKACAERIKSYTIGRAHCSGQYFDVWKC |
| | LDHCAAP |
| SEQ ID NO: 81 | GSEEDCKVHCVKEWMAYKACAERNKSYTIGRAHCSGQYFDVWK |
| | CLDHCAAP |
| SEQ ID NO: 82 | GSEEDCKVHCVKEWMAYKACAERIKSYTIGRAHCSGQKFDVWKC |
| | LDHCAAP |
| SEQ ID NO: 83 | GSEEDCKVHCVKEWMAYKACAERIKSYTIGRAHCSGQYFDVFKC |
| | LDHCAAP |
| SEQ ID NO: 84 | GSEEDCKVHCVKEWAAGKACAERNKSYTIGRAHCSGQYFDVWK |
| | CLDHCAAP |
| SEQ ID NO: 85 | GSEEDCKVHCVKEWAAGKACAERIKSYTIGRAHCSGQKFDVWKC |
| | LDHCAAP |
| SEQ ID NO: 86 | GSEEDCKVHCVKEWAAGKACAERIKSYTIGRAHCSGQYFDVFKCL |
| | DHCAAP |
| SEQ ID NO: 87 | GSEEDCKVHCVKEWAAYKACAERNKSYTIGRAHCSGQKFDVWK |
| | CLDHCAAP |
| SEQ ID NO: 88 | GSEEDCKVHCVKEWAAYKACAERNKSYTIGRAHCSGQYFDVFKC |
| | LDHCAAP |
| SEQ ID NO: 89 | GSEEDCKVHCVKEWAAYKACAERIKSYTIGRAHCSGQKFDVFKCL |
| | DHCAAP |
| SEQ ID NO: 90 | GSEEDCKVHCVKEWMAGKACAERQKSDTTGQAHCSGQKFDVFK |
| | CLDHCAAP |
| SEQ ID NO: 91 | GSEEDCKVHCVKEWMAGKACAERNKSDTTGQAHCSGQKFDVFK |
| | CLDHCAAP |
| SEQ ID NO: 92 | GSEEDCKVHCVKEWAAYKACAERIKSDTTGQAHCSGQYFDVWKC |
| | LDHCAAP |
| SEQ ID NO: 93 | GSEEDCKVHCVKWWMAGKACAERNKSDTTGQAHCSGQKFDVFK |
| | CLDHCAAP |
| SEQ ID NO: 94 | GSEEDCKVHCVKEWMAGKACAERNKSDTTGQAHCSGQKFDVFK |
| | CLDHCAAP |
| SEQ ID NO: 95 | GSEEDCKVHCVKWWAAGKACAERNKSDTTGQAHCSGQKFDVFK |
| | CLDHCAAP |
| SEQ ID NO: 96 | GSEEDCKVHCVKWWMAYKACAERNKSDTTGQAHCSGQKFDVFK |
| | CLDHCAAP |
| SEQ ID NO: 97 | GSEEDCKVHCVKWWMAGKACAERIKSDTTGQAHCSGQKFDVFK |
| | CLDHCAAP |
| SEQ ID NO: 98 | GSEEDCKVHCVKWWMAGKACAERNKSDTTGQAHCSGQYFDVFK |
| | CLDHCAAP |
| SEQ ID NO: 99 | GSEEDCKVHCVKWWMAGKACAERNKSDTTGQAHCSGQKFDVW |
| | KCLDHCAAP |
| SEQ ID NO: 100 | GSEEDCKVHCVKWWAAYKACAERIKSDTTGQAHCSGQYFDVWK |
| | CLDHCAAP |
| SEQ ID NO: 101 | GSEEDCKVHCVKEWMAYKACAERIKSDTTGQAHCSGQYFDVWK |
| | CLDHCAAP |
| SEQ ID NO: 102 | GSEEDCKVHCVKEWAAGKACAERIKSDTTGQAHCSGQYFDVWKC |
| | LDHCAAP |
| SEQ ID NO: 103 | GSEEDCKVHCVKEWAAYKACAERNKSDTTGQAHCSGQYFDVWK |
| | CLDHCAAP |
| SEQ ID NO: 104 | GSEEDCKVHCVKEWAAYKACAERIKSDTTGQAHCSGQKFDVWKC |
| | LDHCAAP |
| SEQ ID NO: 105 | GSEEDCKVHCVKEWAAYKACAERIKSDTTGQAHCSGQYFDVFKC |
| | LDHCAAP |
| SEQ ID NO: 106 | GSEEDCKVHCVKEWMAGKACAERIKSDTTGQAHCSGQYFDVWK |
| | CLDHCAAP |
| SEQ ID NO: 107 | GSEEDCKVHCVKEWMAYKACAERNKSDTTGQAHCSGQYFDVWK |
| | CLDHCAAP |
| SEQ ID NO: 108 | GSEEDCKVHCVKEWMAYKACAERIKSDTTGQAHCSGQKFDVWK |
| | CLDHCAAP |
| SEQ ID NO: 109 | GSEEDCKVHCVKEWMAYKACAERIKSDTTGQAHCSGQYFDVFKC |
| | LDHCAAP |
| SEQ ID NO: 110 | GSEEDCKVHCVKEWAAGKACAERNKSDTTGQAHCSGQYFDVWK |
| | CLDHCAAP |
| SEQ ID NO: 111 | GSEEDCKVHCVKEWAAGKACAERIKSDTTGQAHCSGQKFDVWKC |
| | LDHCAAP |
| SEQ ID NO: 112 | GSEEDCKVHCVKEWAAGKACAERIKSDTTGQAHCSGQYFDVFKC |
| | LDHCAAP |
| SEQ ID NO: 113 | GSEEDCKVHCVKEWAAYKACAERNKSDTTGQAHCSGQKFDVWK |
| | CLDHCAAP |
| SEQ ID NO: 114 | GSEEDCKVHCVKEWAAYKACAERNKSDTTGQAHCSGQYFDVFKC |
| | LDHCAAP |
| SEQ ID NO: 115 | GSEEDCKVHCVKEWAAYKACAERIKSDTTGQAHCSGQKFDVFKC |
| | LDHCAAP |
| SEQ ID NO: 116 | GSEESCKPQCVKAWLEYQACAERVEKDESGEAHCTGQYFDLWGC |
| | VDKCVAP |
| SEQ ID NO: 117 | GSARTCESQSHRFKGPCVSDTMCASVCRTERFSGGHCRGFRRRCL |
| | CSKHC |
| SEQ ID NO: 118 | GSEERCMPQCVKSLYEYEKCLKRVENDDTGHKHCTGHYFDYWSC |
| | IDKCVAS |
| SEQ ID NO: 436 | GSEEDCRVHCVREWMAGRACAERDRSYTIGRAHCSGQRFDVFRC |
| | LDHCAAP |
| SEQ ID NO: 561 | GSEHDCKVHCVKEWMAGHACAERQKSYTIGRAHCSGQKFDVFKC |
| | LDHCAAP |
| SEQ ID NO: 562 | GSEHDCKVHCVKEWMAGKACAERQKSYTIGRAHCSGQKFDVFKC |
| | LDHCAAP |
| SEQ ID NO: 563 | GSEEDCKVHCVKEWHAGKACAERQKSYTIGRAHCSGQKFDVFKC |
| | LDHCAAP |
| SEQ ID NO: 564 | GSEEDCKVHCVKEWMAGHACAERQKSYTIGRAHCSGQKFDVFKC |
| | LDHCAAP |
| SEQ ID NO: 565 | GSEHDCKVHCVKEWHAGKACAERQKSYTIGRAHCSGQKFDVFKC |
| | LDHCAAP |
| SEQ ID NO: 566 | GSEEDCKVHCVKEWHAGHACAERQKSYTIGRAHCSGQKFDVFKC |
| | LDHCAAP |
| SEQ ID NO: 567 | GSEHDCKVHCVKEWHAGHACAERQKSYTIGRAHCSGQKFDVFKC |
| | LDHCAAP |

In some embodiments, a PD-L1-binding peptide disclosed herein comprises a sequence of X¹X²X³CX⁴X⁵X⁶CX⁷X⁸X⁹X¹⁰X¹¹X¹²X¹³X¹⁴X¹⁵CX¹⁶X¹⁷X¹⁸X¹⁹X²⁰X²¹X²²X²³X²⁴X²⁵X²⁶X²⁷X²⁸C X²⁹X³⁰X³¹X³²X³³X³⁴X³⁵X³⁶X³⁷CX³⁸X³⁹X⁴⁰ CX⁴¹X⁴²X⁴³ (SEQ ID NO: 358), wherein X¹ can independently be selected from E, M, V, or W; X² can independently be selected from G, E, L, or F; X³ can independently be selected from D, E, or S; X⁴ can independently be selected from K, R, or V; X⁵ can independently be selected from E, Q, S, M, L, or V; X⁶ can independently be selected from D, E, H, K, R, N, Q, S, or Y; X⁷ can independently be selected from D, M, or V; X⁸ can independently be selected from A, K, R, Q, S, or T; X⁹ can independently be selected from A, D, E, H, Q, S, T, M, I, L, V, or W; X¹⁰ can independently be selected from A, E, R, Q, S, T, W, or P; X¹¹ can independently be selected from A, E, K, R, N, Q, T, M, I, L, V, or W; X¹² can independently be selected from G, A, E, K, N, T, or Y; X¹³ can independently be selected from G, A, D, E, H, K, R, N, Q, S, T, M, I, L, V, W, Y, or P; X¹⁴ can independently be selected from D, K, R, N, L, or V; X¹⁵ can independently be selected from G, A, D, T, L, W, or P; X¹⁶ can independently be selected from G, A, E, H, K, N, S, F, or P; X¹⁷ can independently be selected from G, A, D, E, N, or P; X¹⁸ can independently be selected from G, D, H, K, R, N, Q, S, T, V, or Y; X¹⁹ can independently be selected from G, D, E, H, K, N, Q, S, T, M, I, F, W, Y, or P; X²⁰ can independently be selected from G, A, D, E, H, K, R, N, Q, S, Y, or P; X²¹ can independently be selected from G, A, D, H, N, Q, S, V, F, or P; X²² can independently be selected from A, D, H, N, Q, S, T, M, I, V, Y, or P; X²³ can independently be selected from G, A, D, K, R, T, W, or Y; X²⁴ can independently be selected from G, A, E, N, Q, T, I, V, or P; X²⁵ can independently be selected from G, D, N, Q, T, L, V, F, or P; X²⁶ can independently be selected from G, A, E, K, R, N, Q, S, T, I, Y, or P; X²⁷ can independently be selected from A, D, N, or I; X²⁸ can independently be selected from G, D, E, H, N, F, or W; X²⁹ can independently be selected from G, A, E, N, S, Y, or P; X³⁰ can independently be selected from G, M, or L; X³¹ can independently be selected from G, A, D, K, N, Q, or W; X³² can independently be selected from D, E, H, K, N, Q, S, T, L, V, F, Y, or P; X³³ can independently be selected from G, E, Q, or F; X³⁴ can independently be selected from D or K; X³⁵ can independently be selected from G, V, or P; X³⁶ can independently be selected from G, H, R, V, F, W, or P; X³⁷ can independently be selected from A, D, or K; X³⁸ can independently be selected from E, H, Q, L, or F; X³⁹ can independently be selected from D, E, R, S, T, M, L, or F; X⁴⁰ can independently be selected from G, A, D, E, H, K, R, M, L, or P; X⁴¹ can independently be selected from G, A, K, S, I, or L; X⁴² can independently be selected from G, A, D, E, R, Q, T, or F; and X⁴³ can independently be selected from A, H, N, Q, S, F, or P.

In some embodiments, a binding peptide disclosed herein comprises a sequence of EEDCKVX¹CVX¹X¹X¹X¹X²X³KX¹CX¹EX¹X⁴X¹X¹X¹X¹X¹X¹X¹AX¹CX¹GX¹X⁵FX⁶VFX⁶CLX¹X¹CX¹X¹X¹ (SEQ ID NO: 359), wherein X¹ can independently be selected from any non-cysteine amino acid; X² can independently be selected from M, I, L, or V; X³ can independently be selected from Y, A, H, K, R, N, Q, S, or T; X⁴ can independently be selected from D, E, N, Q, or P; X⁵ can independently be selected from K or P; and X⁶ can independently be selected from D or K.

A PD-L1-binding peptide may comprise a PD-L1-binding motif that forms part or all of a binding interface with PD-L1. One or more residues of a PD-L1-binding motif may interact with one or more residues of PD-L1 at the binding interface between the PD-L1-binding peptide and PD-L1. In some embodiments, multiple PD-L1-binding motifs may be present in a PD-L1-binding peptide. A PD-L1-binding motif may comprise a sequence of CX¹X²X³CX⁴X⁵X⁶X⁷X⁸X⁹X¹⁰X¹¹X¹²C (SEQ ID NO: 360), wherein X¹ can independently be selected from K, R, or V; X² can independently be selected from E, Q, S, M, L, or V; X³ can independently be selected from D, E, H, K, R, N, Q, S, or Y; X⁴ can independently be selected from D, M, or V; X⁵ can independently be selected from A, K, R, Q, S, or T; X⁶ can independently be selected from A, D, E, H, Q, S, T, M, I, L, V, or W; X⁷ can independently be selected from A, E, R, Q, S, T, W, or P; X⁸ can independently be selected from A, E, K, R, N, Q, T, M, I, L, V, or W; X⁹ can independently be selected from G, A, E, K, N, T, or Y; X¹⁰ can independently be selected from G, A, D, E, H, K, R, N, Q, S, T, M, I, L, V, W, Y, or P; X¹¹ can independently be selected from D, K, R, N, L, or V; and X¹² can independently be selected from G, A, D, T, L, W, or P. In some embodiments, a PD-L1-binding motif may comprise a sequence of CKVX¹CVX¹X¹X¹X¹X²X³KX¹C (SEQ ID NO: 362), wherein X¹ can independently be selected from any non-cysteine amino acid; X² can independently be selected from M, I, L, or V; and X³ can independently be selected from Y, A, H, K, R, N, Q, S, or T. In some embodiments, a PD-L1-binding motif may comprise a sequence of CKVHCVKEWMAGKAC (SEQ ID NO: 364). In some embodiments, a PD-L1-binding motif may comprise at least about 70%, at least about 75%, at least about 80%, at least about 85%, at least about 90%, at least about 95%, at least about 97%, or at least about 99% identity to SEQ ID NO: 364.

A PD-L1-binding motif may comprise a sequence of X¹X²X³X⁴X⁵X⁶CX⁷X⁸X⁹C (SEQ ID NO: 361), wherein X¹ can independently be selected from D, E, H, K, N, Q, S, T, L, V, F, Y, or P; X² can independently be selected from G, E, Q, or F; X³ can independently be selected from D or K; X⁴ can independently be selected from G, V, or P; X⁵ can independently be selected from G, H, R, V, F, W, or P; X⁶ can independently be selected from A, D, or K; X⁷ can independently be selected from E, H, Q, L, or F; X⁸ can independently be selected from D, E, R, S, T, M, L, or F; and X⁹ can independently be selected from G, A, D, E, H, K, R, M, L, or P. In some embodiments, a PD-L1-binding motif may comprise a sequence of X⁴FX²VFX²CLX³X³C (SEQ ID NO: 363), wherein X¹ can independently be selected from K or P; X² can independently be selected from D or K; and X³ can independently be selected from any non-cysteine amino acid. In some embodiments, a PD-L1-binding motif may comprise a sequence of KFDVFKCLDHC (SEQ ID NO: 365). In some embodiments, a PD-L1-binding motif may comprise at least about 70%, at least about 75%, at least about 80%, at least about 85%, at least about 90%, at least about 95%, at least about 97%, or at least about 99% identity to SEQ ID NO: 365.

A PD-L1-binding peptide of the present disclosure (e.g., any one of SEQ ID NO: 1-SEQ ID NO: 118, SEQ ID NO: 435, SEQ ID NO: 436, or SEQ ID NO: 554-SEQ ID NO: 567) may comprise one or more secondary structural elements. In some embodiments, a PD-L1-binding peptide may comprise an α-helix, a ß-sheet, a loop, or combinations thereof. A PD-L1-binding peptide (e.g., any one of SEQ ID NO: 1-SEQ ID NO: 56 or SEQ ID NO: 60-SEQ ID NO: 115) may comprise an α-helix comprising amino acid residues, n through n+20, where n corresponds to an amino acid position of a first cysteine residue. For example, a PD-L1-binding peptide of SEQ ID NO: 3 may comprise an α-helix comprising amino acid residues C4 through S24. A PD-L1-binding peptide (e.g., any one of SEQ ID NO: 1-SEQ ID NO: 56 or SEQ ID NO: 60-SEQ ID NO: 115) may comprise an α-helix comprising amino acid residues n+29 through n+44, where n corresponds to an amino acid position of a first cysteine residue. For example, a PD-L1-binding peptide of SEQ ID NO: 3 may comprise an α-helix comprising amino acid residues S33 through A48. A PD-L1-binding peptide (e.g., any one of SEQ ID NO: 1-SEQ ID NO: 56 or SEQ ID NO: 60-SEQ ID NO: 115) may comprise an α-helix comprising amino acid residues n+34 through n+44, where n corresponds to an amino acid position of a first cysteine residue. For example, a PD-L1-binding peptide of SEQ ID NO: 3 may comprise an α-helix comprising amino acid residues D38 through A48. In some embodiments, a PD-L1-binding peptide of the present disclosure can bind to PD-L1 by forming hydrophobic interactions with 154, Y56, R113, M115, or Y123 of PD-L1. For example, residues V9, W12, M13, V39, or F40 of SEQ ID NO: 1 may hydrophobic interactions with PD-L1. In some embodiments, a PD-L1-binding peptide of the present disclosure can form a salt bridge with Q66, A121, and Y123 of PD-L1. For example, residues K5, K16, L43, and D44 of SEQ ID NO: 1 may form salt bridges with PD-L1. In some embodiments, a PD-L1-binding peptide of the present disclosure can bind to PD-L1 in a similar manner to that of natural binding partner PD-1 which uses K78, 1126, L128, A132, 1134, and E136 to interact with the same sites on PD-L1 as K5, L43, V9, W12, F40, and D44, respectively, of SEQ ID NO: 1. In some embodiments, any one of SEQ ID NO: 358-SEQ ID NO: 365 may comprise a portion of a PD-L1-binding peptide that interacts with PD-L1 (e.g., to form hydrophobic interactions or salt bridges). For example, any one of SEQ ID NO: 358-SEQ ID NO: 365 may comprise K5, V9, W12, M13, K16, V39, F40, L43, D44, or combinations thereof, with respect to SEQ ID NO: 1.

In some embodiments, a PD-L1-binding peptide of the present disclosure can bind to PD-L1 with an affinity that is pH-independent. For example, a PD-L1-binding peptide can bind PD-L1 at an extracellular pH (about pH 7.4) with an affinity that is substantially the same the binding affinity at an endocytic pH (such as about pH 5.5 or about pH 6.5). In some embodiments, a PD-L1-binding peptide can bind PD-L1 at an extracellular pH (about pH 7.4) with an affinity that is lower than the binding affinity at an endocytic pH (such as about pH 5.5 or about pH 6.5). In some embodiments, a PD-L1-binding peptide can bind PD-L1 at an extracellular pH (about pH 7.4) with an affinity that is higher than the binding affinity at an endocytic pH (such as about pH 5.5 or about pH 6.5). In some embodiments, the binding affinity of a PD-L1-binding peptide for PD-L1 at extracellular pH (about pH 7.4) the binding affinity of a PD-L1-binding peptide for PD-L1 at endocytic pH (about pH 5.5) can differ by no more than about 1%, no more than about 2%, no more than about 3%, no more than about 4%, no more than about 5%, no more than about 6%, no more than about 7%, no more than about 8%, no more than about 9%, no more than about 10%, no more than about 12%, no more than about 15%, no more than about 17%, no more than about 20%, no more than about 25%, no more than about 30%, no more than about 35%, no more than about 40%, no more than about 45%, or no more than about 50%. In some embodiments, the affinity of the PD-L1-binding peptide for PD-L1 at pH 7.4 and at pH 5.5 can differ by no more than 5-fold, no more than 10-fold, no more than 15-fold, no more than 20-fold, no more than 25-fold, no more than 30-fold, no more than 40-fold, or no more than 50-fold. In some embodiments, a PD-L1-binding peptide (e.g., any one of SEQ ID NO: 1-SEQ ID NO: 118, SEQ ID NO: 435, SEQ ID NO: 436, or SEQ ID NO: 554-SEQ ID NO: 567) can be modified to remove one or more histidine amino acids in the PD-L1 binding interface, thereby reducing the pH-dependence of the binding affinity of the PD-L1-binding peptide for PD-L1. In some embodiments, a PD-L1-binding peptide (e.g., any one of SEQ ID NO: 1-SEQ ID NO: 118, SEQ ID NO: 435, SEQ ID NO: 436, or SEQ ID NO: 554-SEQ ID NO: 567) can lack histidine amino acids in the PD-L1 binding interface.

In some embodiments, a PD-L1-binding peptide can bind to PD-L1 with an equilibrium dissociation constant (K_{D}) of not greater than 50 μM, not greater than 5 μM, not greater than 500 nM, not greater than 100 nM, not greater than 40 nM, not greater than 30 nM, not greater than 20 nM, not greater than 10 nM, not greater than 5 nM, not greater than 2 nM, not greater than 1 nM, not greater than 0.5 nM, not greater than 0.4 nM, not greater than 0.3 nM, not greater than 0.25 nM, not greater than 0.2 nM, or not greater than 0.1 nM, for example at extracellular pH (about pH 7.4). In some embodiments, a PD-L1-binding peptide with pH-independent binding can bind to PD-L1 with a dissociation constant (K_{D}) of not greater than 50 μM, not greater than 5 μM, not greater than 500 nM, not greater than 100 nM, not greater than 40 nM, not greater than 30 nM, not greater than 20 nM, not greater than 10 nM, not greater than 5 nM, not greater than 2 nM, not greater than 1 nM, not greater than 0.5 nM, not greater than 0.2 nM, or not greater than 0.1 nM at endosomal pH (about pH 5.5). In some embodiments, the PD-L1-binding peptide can bind to PD-L1 with an affinity that is pH-dependent. For example, the PD-L1-binding molecule can bind to PD-L1 with higher affinity at extracellular pH (about pH 7.4) and with lower affinity at endosomal pH (about pH 5.5), thereby releasing the peptide or peptide complex from PD-L1 upon entry into and acidification of the endosomal compartment.

A PD-L1-binding peptide of the present disclosure may be cross-reactive with two or more species of PD-L1, or a PD-L1-binding peptide may be selective for one or more species of PD-L1. For example, a PD-L1-binding peptide may be cross-reactive for both human and cynomolgus PD-L1. A PD-L1-binding peptide may be cross-reactive for two species if it binds to both species with an equilibrium dissociation constant (K_{D}) that differs by no more than 1.5-fold, no more than 2-fold, no more than 5-fold, or no more than 10-fold. In some embodiments, a PD-L1-binding peptide may not be cross-reactive with one or more species of PD-L1. For example, a PD-L1-binding peptide may bind human PD-L1 with an equilibrium dissociation constant (K_{D}) of not greater than 100 nM, not greater than 50 nM, not greater than 1 nM, not greater than 500 pM, not greater than 300 pM, not greater than 250 pM, or not greater than 200 pM but may bind murine PD-L1 with an equilibrium dissociation constant (K_{D}) that is at least 10-fold, 50-fold, or 100-fold greater.

### Sequence Identity and Homology

Percent (%) sequence identity or homology is determined by conventional methods. (See e.g., Altschul et al. (1986), Bull. Math. Bio. 48:603 (1986), and Henikoff and Henikoff (1992), Proc. Natl. Acad. Sci. USA 89:10915). Briefly, two amino acid sequences can be aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Various methods and software programs can be used to determine the homology between two or more peptides, such as NCBI BLAST, Clustal W, MAFFT, Clustal Omega, AlignMe, Praline, or another suitable method or algorithm. Pairwise sequence alignment can be used to identify regions of similarity that can indicate functional, structural and/or evolutionary relationships between two biological sequences (e.g., amino acid or nucleic acid sequences). In addition, multiple sequence alignment (MSA) is the alignment of three or more biological sequences. From the output of MSA applications, homology can be inferred and the evolutionary relationship between the sequences assessed. As used herein, “sequence homology” and “sequence identity” and “percent (%) sequence identity” and “percent (%) sequence homology” are used interchangeably to mean the sequence relatedness or variation, as appropriate, to a reference polynucleotide or amino acid sequence.

Additionally, there are several established algorithms available to align two amino acid sequences. For example, the “FASTA” similarity search algorithm of Pearson and Lipman can be a suitable protein alignment method for examining the level of sequence identity or homology shared by an amino acid sequence of a peptide disclosed herein and the amino acid sequence of a peptide variant. The FASTA algorithm is described, for example, by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. For example, illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid sequences or molecules using a ratio as disclosed above. For nucleic acid sequence comparisons, the ktup value can range between one to six, preferably from three to six, most preferably three, with other parameters set as described herein.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that can be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments, determination of structure can typically be accompanied by evaluating activity of modified molecules.

### Peptide Active Agent Complexes

In some embodiments, PD-L1-binding CDPs, such as those described in TABLE 1, including engineered, non-naturally occurring CDPs and those found in nature, can be conjugated to, linked to, or fused to an additional active agent to selectively deliver the active agent to a PD-L1 positive cell. The cell can be a cancer cell, an immune cell, a pancreatic beta cell, or any combination thereof. The cell can be any cell that expresses PD-L1. An engineered peptide can be a peptide that is non-naturally occurring, artificial, synthetic, designed, or recombinantly expressed. In some embodiments, a PD-L1-binding peptide complex comprising a PD-L1-binding peptide (e.g., a PD-L1-binding bispecific immune cell engager or a PD-L1-binding chimeric antigen receptor), enables PD-L1-mediated delivery of an additional active agent (e.g., a therapeutic agent, a detectable agent, or an immune cell) to a target cell. The target cell (e.g., a PD-L1 positive target cell) may be associated with a disease or condition. In some embodiments, delivering the active agent to the target cell may treat (e.g., prevent, reduce, eliminate, diagnose, or alleviate symptoms of) the disease or condition. In some cases, the target cell is a cancer cell. Cancers can include melanoma, non-small cell lung cancer, small cell lung cancer, renal cancer, esophageal cancer, oral cancer, hepatocellular cancer, ovarian cancer, cervical cancer, colorectal cancer, lymphoma, bladder cancer, liver cancer, gastric cancer, breast cancer, pancreatic cancer, prostate cancer, Merkel cell carcinoma, mesothelioma, brain cancer, metastatic brain cancer, primary brain cancer, glioblastoma, or a PD-L1-overexpressing cancer. In some cases, PD-L1-binding peptides or peptide complexes and are capable of crossing the blood brain barrier to deliver PD-L1-binding peptides or other active agents to target cells in the central nervous system.

A PD-L1-binding peptide of the present disclosure may be linked, fused, conjugated, or otherwise complexed with an additional active agent to form a PD-L1-binding peptide complex. In some embodiments, the PD-L1-binding peptide and the additional active agent may be complexed via a linker (e.g., a peptide linker or a small molecule linker). The activity (e.g., binding, inhibitory, or activating activity) of both the PD-L1-binding peptide and the additional active agent may be retained upon complex formation. In some embodiments, an appropriate linker is selected such that activities are retained. An active agent may be any agent capable of performing a function. Such functions may include binding, inhibition, activation, inactivation, recruitment, signal generation, synthesis, destruction, or combinations thereof. In some embodiments, an active agent may be a therapeutic agent (e.g., a therapeutic small molecule or therapeutic peptide) or a detectable agent (e.g., a fluorophore or radioisotope).

The active agent may be complexed with the PD-L1 binding peptide such that it does not disrupt binding with PD-L1. In some embodiments, a peptide active agent complex may bind to PD-L1 with an equilibrium dissociation constant (K_{D}) of not greater than 100 nM, not greater than 50 nM, not greater than 30 nM, not greater than 20 nM, not greater than 1 nM, not greater than 500 pM, not greater than 300 pM, not greater than 250 pM, or not greater than 200 pM.

### Peptide Therapeutic Agent Complexes

A PD-L1-binding peptide of the present disclosure (e.g., any one of SEQ ID NO: 1-SEQ ID NO: 118, SEQ ID NO: 435, SEQ ID NO: 436, or SEQ ID NO: 554-SEQ ID NO: 567) may be complexed with a therapeutic agent to form a peptide therapeutic agent complex. The therapeutic agent of the peptide complex may perform a therapeutic function upon delivery to a cell (e.g., a PD-L1 positive cell). Therapeutic functions may include activation or inhibition of a target (e.g., a target enzyme), recruitment of an additional component to the cell, or killing the cell (e.g., a PD-L1 positive cancer cell). Examples of therapeutic agents that may be complexed with a PD-L1-binding peptide include anti-cancer agents, chemotherapeutic agents, radiotherapy agents, anti-inflammatory agents, proinflammatory cytokines, oligonucleotides, or combinations thereof. In some embodiments, an active agent (e.g., an Fc domain or globular protein) may function as a steric blocker. For example, an Fc domain linked to a PD-L1-binding peptide may enhance disruption of PD-1 binding to PD-L1 by the PD-L1-binding peptide by sterically blocking access to PD-L1. In some embodiments, a PD-L1-binding peptide may be complexed with an oncolytic viral vector to deliver the viral vector to a PD-L1 positive cell.

Chemotherapeutic or anti-cancer agents may function by killing or inhibiting proliferation of a target cancer cell (e.g., a PD-L1 positive cancer cell). Examples of chemotherapeutics or anti-cancer agents that may be complexed with a PD-L1-binding peptide of the present disclosure include antineoplastic agents, cytotoxic agents, tyrosine kinase inhibitors, mTOR inhibitors, retinoids, microtubule polymerization inhibitors, pyrrolobenzodiazepine dimers, or anti-cancer antibodies. Proinflammatory cytokines may function by stimulating an immune response against a target (e.g., a PD-L1 positive cancer cell). Examples of proinflammatory cytokines that may be complexed with a PD-L1-binding peptide of the present disclosure include TNFα, IL-2, IL-6, IL-12, IL-15, IL-21, or IFNγ. Anti-inflammatory agents may function by inhibiting an inflammatory response in or around the target (e.g., by inhibiting a cyclooxygenase enzyme or stimulating a glucocorticoid receptor). Examples of anti-inflammatory agents that may be complexed with a PD-L1-binding peptide of the present disclosure include anti-inflammatory cytokines, steroids, glucocorticoids, corticosteroids, cytokine inhibitors, RORgamma inhibitors, JAK inhibitors, tyroskine kinase inhibitors, or nonsteroidal anti-inflammatory drugs (NSAIDs).

In some embodiments, an active agent is an immunotherapeutic agent, an immuno-oncology agent, a CTLA-4 targeting agent, a PD-1 targeting agent, a PD-L1 targeting agent, an IL15 agent, a fused IL-15/IL-15Ra complex agent, an IFNgamma agent, an anti-CD3 agent, an ion channel modulator, an auristatin, MMAE, a maytansinoid, DM1, DM4, doxorubicin, a calicheamicin, a platinum compound, cisplatin, a taxane, paclitaxel, SN-38, a BACE inhibitor, a Bcl-xL inhibitor, WEHI-539, venetoclax, ABT-199, navitoclax, AT-101, obatoclax, a pyrrolobenzodiazepine or pyrrolobenzodiazepine dimer, a dolastatin, an immuno-oncology agent, an agent that targets an immune cell, (e.g., targets GITR, 4-1BB, CD27, TIGIT, LAG3, TCR, CD40L, OX40, PD-1, CTLA-4, CD28), or an agent that targets a tumor cell (e.g., targets GITRL, 4-1BBL, CD17, CD156/CD112/CD113, MHC11, CD40, OX40L, PD-L1/L2, CD80/86).

In some embodiments, PD-L1-binding peptides can direct the active agent (e.g., a target-binding nucleotide, small molecule, peptide, or protein active agent) into the cell. In further embodiments, PD-L1-binding peptides can direct the active agent into the nucleus. In some embodiments, the active agent has intrinsic tumor-homing properties, or the active agent can be engineering to have tumor-homing properties. In some embodiments, 1, 2, 3, 4, 5, 6, 7, 8, 9, or 10 active agents can be linked to a peptide or nucleotide. Multiple active agents (e.g., multiple target-binding nucleotides) can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015)). Examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an oligonucleotide, antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter an antibody, a single chain variable fragment (scFv), an antibody fragment, an aptamer, a cytokine, an interferon, a hormone, an enzyme, a growth factor, a checkpoint inhibitor, a PD-1 inhibitor, a PD-L1 inhibitor, a CD47 inhibitor, a CTLA4 inhibitor, a CD antigen, a chemokine, an ion channel inhibitor, an ion channel activator, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, an immuno-oncology agent, a complement fixing peptide or protein, a tumor necrosis factor inhibitor, a tumor necrosis factor activator, a tumor necrosis factor receptor family agonist, a tumor necrosis receptor antagonist, a Tim-3 inhibitor, a protease inhibitor, an amino sugar, a chemotherapeutic, a cytotoxic molecule, a toxin, a tyrosine kinase inhibitor, an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc region, or an active fragment or a modification thereof.

Only a small fraction of currently available drug molecules have applicability in CNS diseases due to their poor BBB penetration capabilities. About 98% of small molecule drugs do not or do only to a very limited degree cross the BBB. In addition, nearly 100% of macromolecular drug molecules (e.g., antibodies) do not exhibit significant BBB penetration capabilities. (See e.g., Mikitsh et al. Pathways for Small Molecule Delivery to the Central Nervous System Across the Blood-Brain Barrier, Perspect Medicin Chem. 2014; 6: 11-24). PD-L1-binding antibodies may not be able to cross the BBB at therapeutically sufficient levels. PD-L1 binding peptides of this disclosure may be able to cross the BBB by virtue of CDP properties, PD-L1-binding properties, or other properties. PD-L1 binding peptides of this disclosure may also be complexed with other agents, such as a transferrin-receptor (TfR) binding agent, to enable the complex to cross the BBB. PD-L1-expressing cells, such as cancer cells in the brain (e.g., from primary or metastatic cancers) may be beneficially targeted by administering PD-L1-binding molecules that can cross the BBB. Thus, the PD-L1 binding peptides of this disclosure may be administered for therapeutic utility in the CNS, such as to block PD-L1 in the brain, to deliver active agents to the brain (e.g., T-cell binders or oligonucleotides). The PD-L1 binding peptides of this disclosure may also be complexed with a TfR-binding peptide (e.g., SEQ ID NO: 350) in order to deliver the PD-L1 binding peptides of this disclosure across the BBB to the CNS. Brain tumors that can be treated or prevented using conjugates or fusion molecules comprising one or more PD-L1-binding peptides of the present disclosure can include glioblastoma, astrocytoma, glioma, medulloblastoma, ependymoma, choroid plexus carcinoma, midline glioma, metastatic cancer including but not limited to metastatic melanoma, breast cancer, and lung cancer, and diffuse intrinsic pontine glioma.

Active agents that may be used in combination with the PD-L1-binding peptides described herein include cytotoxic molecules. For example, cytotoxic molecules that can be used include auristatins, MMAE, MMAF, dolostatin, auristatin F, monomethylaurstatin D, DM1, DM4, maytansinoids, maytansine, calicheamicins, N-acetyl-γ-calicheamicin, pyrrolobenzodiazepines, PBD dimers, doxorubicin, vinca alkaloids (4-deacetylvinblastine), duocarmycins, cyclic octapeptide analogs of mushroom amatoxins, epothilones, and anthracylines, CC-1065, taxanes, paclitaxel, cabazitaxel, docetaxel, SN-38, irinotecan, vincristine, vinblastine, platinum compounds, cisplatin, methotrexate, BACE (beta-secretase 1) inhibitors such as verubecestat, chlorambucil, mitomycin C. Additional examples of active agents are described in McCombs, J. R., AAPS J, 17(2): 339-51 (2015), Ducry, L., Antibody Drug Conjugates (2013), and Singh, S. K., Pharm Res. 32(11): 3541-3571 (2015). Additional examples of therapeutic payloads which therapeutic efficacy can be significantly improved when used in combination with the compositions and methods of the present disclosure include Carmustine, Cisplatin, Cyclophosphamide, Etoposide, Irinotecan, Lomustine, Procarbazine, Temozolomide, Vincristine, and Bevacizumab. Additional examples of therapeutic payloads are compounds that have therapeutic benefit in neurodegenerative diseases such as BACE inhibitors or auto-immunity diseases.

### Peptide Detectable Agent and Peptide Radiotherapeutic Agent Complexes

A PD-L1-binding peptide (e.g., any one of SEQ ID NO: 1-SEQ ID NO: 118, SEQ ID NO: 435, SEQ ID NO: 436, or SEQ ID NO: 554-SEQ ID NO: 567) or peptide complex of the present disclosure may be complexed with a detectable agent or radiotherapeutic agent to form a peptide detectable agent complex or peptide radiotherapeutic agent complex. The peptide or peptide complex (e.g., a PD-L1-binding peptide complex) of the present disclosure may be conjugated, linked, or fused to a detectable agent or a radiotherapeutic agent. In some embodiments, a detectable agent or a therapeutic agent may be complexed with a PD-L1-binding peptide in combination with an additional active agent (e.g., a therapeutic agent, an oligonucleotide, or a therapeutic oligonucleotide). For example, a detectable agent may be conjugated to a PD-L1-binding peptide oligonucleotide complex. In some embodiments, the detectable agent or radiotherapeutic agent may be directly or indirectly linked to a PD-L1-binding peptide. In some embodiments, the detectable agent or radiotherapeutic agent may be directly or indirectly linked to an active agent of a PD-L1-binding peptide complex (e.g., an oligonucleotide of a PD-L1-binding peptide oligonucleotide complex). A peptide complex comprising a detectable agent may be referred to as a detectable agent peptide conjugate or a detectable agent peptide complex. A peptide (e.g., a PD-L1-binding peptide) can be conjugated to, linked to, or fused to an agent used in imaging, research, therapeutics, theranostics, pharmaceuticals, chemotherapy, chelation therapy, targeted drug delivery, and radiotherapy. In some embodiments, a peptide is conjugated to, linked to, or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging.

In some embodiments, 1, 2, 3, 4, 5, 6, 7, 8, 9, or 10 detectable agents can be linked to a peptide or nucleotide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that could be used as a conjugating molecule in the present disclosure include DyLight-680, DyLight-750, VivoTag-750, DyLight-800, IRDye-800, VivoTag-680, Cy5.5, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, Indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177.

In some embodiments, a peptide of the present disclosure (e.g., PD-L1-binding peptide) may further comprise or be complexed with a radioisotope, radiochelator, radiosensitizer, or photosensitizer. In some embodiments, the radioisotope, radiochelator, radiosensitizer, or photosensitizer may be incorporated into, or directly or indirectly linked to the PD-L1-binding peptide. In some embodiments, a radioisotope, radiochelator, radiosensitizer, or photosensitizer. In some embodiments, the radioisotope, radiochelator, radiosensitizer, or photosensitizer may be incorporated into or complexed with a PD-L1-binding peptide complex comprising an additional active agent (e.g., a therapeutic agent, an oligonucleotide, or a therapeutic oligonucleotide). For example, a radioisotope, radiochelator, radiosensitizer, or photosensitizer may be incorporated into, or directly or indirectly linked to an oligonucleotide of PD-L1-binding peptide oligonucleotide complex. The radioisotope, radiochelator, radiosensitizer, photosensitizer may function as a detectable agent, a therapeutic agent, or both. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. Additionally, the following radionuclides can be used for diagnosis and/or therapy: carbon (e.g., ¹¹C or ¹⁴C), nitrogen (e.g., ¹³N), fluorine (e.g., ¹⁸F), gallium (e.g., ⁶⁷Ga or ⁶⁸Ga), copper (e.g., ⁶⁴Cu or ⁶⁷Cu), zirconium (e.g., ⁸⁹Zr), lutetium (e.g., ¹⁷⁷Lu). In some embodiments, the radioisotope is indium-111, technetium-99m, yttrium-90, iodine-131, iodine-123, or astatine-211.

A PD-L1-binding peptide or an active agent of a PD-L1-binding peptide complex can be conjugated to, linked to, or fused to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers can include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach can allow for highly specific targeting of diseased cells (e.g., cancer cells) using both a therapeutic agent (e.g., drug) and electromagnetic energy (e.g., radiation or light) concurrently. In some embodiments, the peptide is conjugated to, linked to, fused with, or covalently or non-covalently linked to the agent, e.g., directly or via a linker. Exemplary linkers suitable for use with the embodiments herein are discussed in further detail below.

A PD-L1-binding peptide or an active agent of a PD-L1-binding peptide complex can be conjugated to, linked to, or fused to a radionuclide via chelator. In some embodiments, the radionuclide may be linked to the peptide of the peptide oligonucleotide complex or the nucleotide of the peptide oligonucleotide complex via the chelator. In some aspects of the present disclosure, the radionuclide is attached to a peptide oligonucleotide complex as described herein using a chelator. Exemplary chelator moieties can include 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid; 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontain-37-yl)thioureido)benzyl)-1,4,7-triazonane-2,5,8-triyl)triacetic acid; 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontain-37-yl)thioureido)benzyl)-1,4,7-triazonane-1,4-diyl)diacetic acid; 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontain-31-yl)thioureido)benzyl)-1,4,7-triazonane-2,5,8-triyl)triacetic acid; 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontain-43-yl)thioureido)benzyl)-1,4,7-triazonane-2,5,8-triyl)triacetic acid; 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid; 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid; 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid; 2,2′,2″-(2-(4-(1-(4-(6-methyl-1,2,4,5-tetrazin-3-yl)phenoxy)-3,6,9,12,15,18,21,24,27,30,33-undecaoxahexatriacontain-36-amido)benzyl)-1,4,7-triazonane-1,4,7-triyl)triacetic acid; 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid; 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid; 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

### Bispecific Immune Cell Engagers (BiICEs)

In some embodiments, an active agent of the present disclosure may be an additional binding moiety (e.g., an immune cell binding moiety). A PD-L1-binding peptide of the present disclosure (e.g., any one of SEQ ID NO: 1-SEQ ID NO: 118, SEQ ID NO: 435, SEQ ID NO: 436, or SEQ ID NO: 554-SEQ ID NO: 567) may be complexed with one or more additional binding moieties to form a bispecific or multi-specific molecule. A bispecific or multi-specific molecule may bind two or more target molecules (e.g., PD-L1 and one or more additional target molecules). An example of a bispecific molecule includes a bispecific immune cell engager (BiICE) comprising a PD-L1-binding peptide (e.g., any one of SEQ ID NO: 1-SEQ ID NO: 118, SEQ ID NO: 435, SEQ ID NO: 436, or SEQ ID NO: 554-SEQ ID NO: 567) and an immune cell targeting agent (e.g., an immune cell targeting antibody or antibody fragment, or an immune cell targeting CDP, peptide, or peptide fragment). The immune cell targeting agent may bind to a molecule on the surface of an immune cell. For example, the immune cell targeting agent may bind to CD3, 4-1BB, CD28, CD137, CD89, CD16, CD25, CD13, CD29, CD44, CD71, CD73, CD90, CD105, CD166, CD27, GITR, TIGIT, LAG3, TCR, CD40L, OX40, PD-1, CTLA-4, or STRO-1. Bispecific immune cell engagers may function by recruiting an immune cell to a PD-L1 positive cell (e.g., a PD-L1 positive cancer cell, an immune cell associated with an autoimmune response, or a pancreatic beta cell). In some embodiments, the immune cell binding agent may bind to a T cell, a B cell, a macrophage, a natural killer cell, a fibroblast, a regulatory T cell, a regulatory immune cell, a neural stem cell, or a mesenchymal stem cell and may recruit the T cell, B cell, macrophage, natural killer cell, fibroblast, regulatory T cell, regulatory immune cell, neural stem cell, or mesenchymal stem cell to the PD-L1 positive cell. For example, an immune cell targeting agent that binds CD3, 4-1BB, CD28, or CD137 may recruit T cells to the PD-L1 positive cell. In another example, an immune cell targeting agent that binds CD89 may recruit macrophages to the PD-L1 positive cell. In another example, an immune cell targeting agent that binds CD16 may recruit natural killer cells to the PD-L1 positive cell. In another example, an immune cell targeting agent that binds CD25 may recruit regulatory T cells to the PD-L1 positive cell. In another example, an immune cell targeting agent that binds CD13, CD29, CD44, CD71, CD73, CD90, CD105, CD166, CD27, GITR, TIGIT, LAG3, TCR, CD40L, OX40, PD-1, CTLA-4, or STRO-1 may recruit mesenchymal stem cells or other immune cells to the PD-L1 positive cell. An example of an immune cell targeting agent that binds CD3 may comprise a sequence of SEQ ID NO: 122 or SEQ ID NO: 442-SEQ ID NO: 471. An example of an immune cell targeting agent that binds CD28 may comprise a sequence of SEQ ID NO: 472-SEQ ID NO: 481. An example of an immune cell targeting agent that binds CD25 may comprise a sequence of SEQ ID NO: 482-SEQ ID NO: 491. Examples of immune cell targeting agents are provided in TABLE 2.

TABLE 2
Exemplary Immune Cell Targeting Agents

| SEQ ID | | |
|---|---|---|
| NO | Sequence | Target |
| SEQ ID | EVQLVESGGGLVQPGGSLKLSCAASGFTFNKYAMNWVRQAPGK | CD3 |
| NO: 122 | GLEWVARIRSKYNNYATYYADSVKDRFTISRDDSKNTAYLQMN | |
| | NLKTEDTAVYYCVRHGNFGNSYISYWAYWGQGTLVTVSSGGGG | |
| | SGGGGSGGGGSQTVVTQEPSLTVSPGGTVTLTCGSSTGAVTSGN | |
| | YPNWVQQKPGQAPRGLIGGTKFLAPGTPARFSGSLLGGKAALTL | |
| | SGVQPEDEAEYYCVLWYSNRWVFGGGTKLTVL | |
| SEQ ID | EVQLLESGGGLVQPGGSLRLSCAASGFTFSSFPMAWVRQAPGKG | CD3 |
| NO: 442 | LEWVSTISTSGGRTYYRDSVKGRFTISRDNSKNTLYLQMNSLRAE | |
| | DTAVYYCAKFRQYSGGFDYWGQGTLVTVSS | |
| SEQ ID | DIQLTQPNSVSTSLGSTVKLSCTLSSGNIENNYVHWYQLYEGRSP | CD3 |
| NO: 443 | TTMIYDDDKRPDGVPDRFSGSIDRSSNSAFLTIHNVAIEDEAIYFC | |
| | HSYVSSFNVFGGGTKLTVL | |
| SEQ ID | SFPMA | CD3 |
| NO: 444 | | |
| SEQ ID | TISTSGGRTYYRDSVKG | CD3 |
| NO: 445 | | |
| SEQ ID | FRQYSGGFDY | CD3 |
| NO: 446 | | |
| SEQ ID | TLSSGNIENNYVH | CD3 |
| NO: 447 | | |
| SEQ ID | DDDKRPD | CD3 |
| NO: 448 | | |
| SEQ ID | HSYVSSFNV | CD3 |
| NO: 449 | | |
| SEQ ID | EVQLLESGGGLVQPGGSLRLSCAASGFTFSSFPMAWVRQAPGKG | CD3 |
| NO: 450 | LEWVSTISTSGGRTYYRDSVKGRFTISRDNSKNTLYLQMNSLRAE | |
| | DTAVYYCAKFRQYSGGFDYWGQGTLVTVSSGGGGSGGGGSGG | |
| | GGSDIQLTQPNSVSTSLGSTVKLSCTLSSGNIENNYVHWYQLYEG | |
| | RSPTTMIYDDDKRPDGVPDRFSGSIDRSSNSAFLTIHNVAIEDEAIY | |
| | FCHSYVSSFNVFGGGTKLTVL | |
| SEQ ID | DIQLTQPNSVSTSLGSTVKLSCTLSSGNIENNYVHWYQLYEGRSP | CD3 |
| NO: 451 | TTMIYDDDKRPDGVPDRFSGSIDRSSNSAFLTIHNVAIEDEAIYFC | |
| | HSYVSSFNVFGGGTKLTVLGGGGSGGGGSGGGGSEVQLLESGGG | |
| | LVQPGGSLRLSCAASGFTFSSFPMAWVRQAPGKGLEWVSTISTSG | |
| | GRTYYRDSVKGRFTISRDNSKNTLYLQMNSLRAEDTAVYYCAKF | |
| | RQYSGGFDYWGQGTLVTVSS | |
| SEQ ID | QVQLVESGGGVVQPGRSLRLSCAASGFKFSGYGMHWVRQAPGK | CD3 |
| NO: 452 | GLEWVAVIWYDGSKKYYVDSVKGRFTISRDNSKNTLYLQMNSL | |
| | RAEDTAVYYCARQMGYWHFDLWGRGTLVTVSS | |
| SEQ ID | EIVLTQSPATLSLSPGERATLSCRASQSVSSYLAWYQQKPGQAPR | CD3 |
| NO: 453 | LLIYDASNRATGIPARFSGSGSGTDFTLTISSLEPEDFAVYYCQQRS | |
| | NWPPLTFGGGTKVEIK | |
| SEQ ID | GYGMH | CD3 |
| NO: 454 | | |
| SEQ ID | VIWYDGSKKYYVDSVKG | CD3 |
| NO: 455 | | |
| SEQ ID | QMGYWHFDL | CD3 |
| NO: 456 | | |
| SEQ ID | RASQSVSSYLA | CD3 |
| NO: 457 | | |
| SEQ ID | DASNRAT | CD3 |
| NO: 458 | | |
| SEQ ID | QQRSNWPPLT | CD3 |
| NO: 459 | | |
| SEQ ID | QVQLVESGGGVVQPGRSLRLSCAASGFKFSGYGMHWVRQAPGK | CD3 |
| NO: 460 | GLEWVAVIWYDGSKKYYVDSVKGRFTISRDNSKNTLYLQMNSL | |
| | RAEDTAVYYCARQMGYWHFDLWGRGTLVTVSSGGGGSGGGGS | |
| | GGGGSEIVLTQSPATLSLSPGERATLSCRASQSVSSYLAWYQQKP | |
| | GQAPRLLIYDASNRATGIPARFSGSGSGTDFTLTISSLEPEDFAVY | |
| | YCQQRSNWPPLTFGGGTKVEIK | |
| SEQ ID | EIVLTQSPATLSLSPGERATLSCRASQSVSSYLAWYQQKPGQAPR | CD3 |
| NO: 461 | LLIYDASNRATGIPARFSGSGSGTDFTLTISSLEPEDFAVYYCQQRS | |
| | NWPPLTFGGGTKVEIKGGGGSGGGGSGGGGSQVQLVESGGGVV | |
| | QPGRSLRLSCAASGFKFSGYGMHWVRQAPGKGLEWVAVIWYD | |
| | GSKKYYVDSVKGRFTISRDNSKNTLYLQMNSLRAEDTAVYYCA | |
| | RQMGYWHFDLWGRGTLVTVSS | |
| SEQ ID | QVQLVQSGGGVVQPGRSLRLSCKASGYTFTRYTMHWVRQAPGK | CD3 |
| NO: 462 | GLEWIGYINPSRGYTNYNQKVKDRFTISRDNSKNTAFLQMDSLRP | |
| | EDTGVYFCARYYDDHYCLDYWGQGTPVTVSS | |
| SEQ ID | DIQMTQSPSSLSASVGDRVTITCSASSSVSYMNWYQQTPGKAPKR | CD3 |
| NO: 463 | WIYDTSKLASGVPSRFSGSGSGTDYTFTISSLQPEDIATYYCQQWS | |
| | SNPFTFGQGTKLQI | |
| SEQ ID | RYTMH | CD3 |
| NO: 464 | | |
| SEQ ID | YINPSRGYTNYNQKVKD | CD3 |
| NO: 465 | | |
| SEQ ID | YYDDHYCLDY | CD3 |
| NO: 466 | | |
| SEQ ID | SASSSVSYMN | CD3 |
| NO: 467 | | |
| SEQ ID | DTSKLAS | CD3 |
| NO: 468 | | |
| SEQ ID | QQWSSNPFT | CD3 |
| NO: 469 | | |
| SEQ ID | QVQLVQSGGGVVQPGRSLRLSCKASGYTFTRYTMHWVRQAPGK | CD3 |
| NO: 470 | GLEWIGYINPSRGYTNYNQKVKDRFTISRDNSKNTAFLQMDSLRP | |
| | EDTGVYFCARYYDDHYCLDYWGQGTPVTVSSGGGGSGGGGSG | |
| | GGGSDIQMTQSPSSLSASVGDRVTITCSASSSVSYMNWYQQTPGK | |
| | APKRWIYDTSKLASGVPSRFSGSGSGTDYTFTISSLQPEDIATYYC | |
| | QQWSSNPFTFGQGTKLQI | |
| SEQ ID | DIQMTQSPSSLSASVGDRVTITCSASSSVSYMNWYQQTPGKAPKR | CD3 |
| NO: 471 | WIYDTSKLASGVPSRFSGSGSGTDYTFTISSLQPEDIATYYCQQWS | |
| | SNPFTFGQGTKLQIGGGGSGGGGSGGGGSQVQLVQSGGGVVQPG | |
| | RSLRLSCKASGYTFTRYTMHWVRQAPGKGLEWIGYINPSRGYTN | |
| | YNQKVKDRFTISRDNSKNTAFLQMDSLRPEDTGVYFCARYYDDH | |
| | YCLDYWGQGTPVTVSS | |
| SEQ ID | QVQLVQSGAEVKKPGASVKVSCKASGYTFTSYYIHWVRQAPGQ | CD28 |
| NO: 472 | GLEWIGCIYPGNVNTNYNEKFKDRATLTVDTSISTAYMELSRLRS | |
| | DDTAVYFCTRSHYGLDWNFDVWGQGTTVTVSS | |
| SEQ ID | DIQMTQSPSSLSASVGDRVTITCHASQNIYVWLNWYQQKPGKAP | CD28 |
| NO: 473 | KLLIYKASNLHTGVPSRFSGSGSGTDFTLTISSLQPEDFATYYCQQ | |
| | GQTYPYTFGGGTKVEIK | |
| SEQ ID | SYYIH | CD28 |
| NO: 474 | | |
| SEQ ID | CIYPGNVNTNYNEKFKD | CD28 |
| NO: 475 | | |
| SEQ ID | SHYGLDWNFDV | CD28 |
| NO: 476 | | |
| SEQ ID | HASQNIYVWLN | CD28 |
| NO: 477 | | |
| SEQ ID | KASNLHT | CD28 |
| NO: 478 | | |
| SEQ ID | QQGQTYPYT | CD28 |
| NO: 479 | | |
| SEQ ID | QVQLVQSGAEVKKPGASVKVSCKASGYTFTSYYIHWVRQAPGQ | CD28 |
| NO: 480 | GLEWIGCIYPGNVNTNYNEKFKDRATLTVDTSISTAYMELSRLRS | |
| | DDTAVYFCTRSHYGLDWNFDVWGQGTTVTVSSGGGGSGGGGS | |
| | GGGGSDIQMTQSPSSLSASVGDRVTITCHASQNIYVWLNWYQQK | |
| | PGKAPKLLIYKASNLHTGVPSRFSGSGSGTDFTLTISSLQPEDFAT | |
| | YYCQQGQTYPYTFGGGTKVEIK | |
| SEQ ID | DIQMTQSPSSLSASVGDRVTITCHASQNIYVWLNWYQQKPGKAP | CD28 |
| NO: 481 | KLLIYKASNLHTGVPSRFSGSGSGTDFTLTISSLQPEDFATYYCQQ | |
| | GQTYPYTFGGGTKVEIKGGGGSGGGGSGGGGSQVQLVQSGAEV | |
| | KKPGASVKVSCKASGYTFTSYYIHWVRQAPGQGLEWIGCIYPGN | |
| | VNTNYNEKFKDRATLTVDTSISTAYMELSRLRSDDTAVYFCTRS | |
| | HYGLDWNFDVWGQGTTVTVSS | |
| SEQ ID | QVQLVQSGAEVKKPGSSVKVSCKASGYTFTSYRMHWVRQAPGQ | CD25 |
| NO: 482 | GLEWIGYINPSTGYTEYNQKFKDKATITADESTNTAYMELSSLRS | |
| | EDTAVYYCARGGGVFDYWGQGTLVTVSS | |
| SEQ ID | DIQMTQSPSTLSASVGDRVTITCSASSSISYMHWYQQKPGKAPKL | CD25 |
| NO: 483 | LIYTTSNLASGVPARFSGSGSGTEFTLTISSLQPDDFATYYCHQRS | |
| | TYPLTFGQGTKVEVK | |
| SEQ ID | SYRMH | CD25 |
| NO: 484 | | |
| SEQ ID | YINPSTGYTEYNQKFKD | CD25 |
| NO: 485 | | |
| SEQ ID | GGGVFDY | CD25 |
| NO: 486 | | |
| SEQ ID | SASSSISYMH | CD25 |
| NO: 487 | | |
| SEQ ID | TTSNLAS | CD25 |
| NO: 488 | | |
| SEQ ID | HQRSTYPLT | CD25 |
| NO: 489 | | |
| SEQ ID | QVQLVQSGAEVKKPGSSVKVSCKASGYTFTSYRMHWVRQAPGQ | CD25 |
| NO: 490 | GLEWIGYINPSTGYTEYNQKFKDKATITADESTNTAYMELSSLRS | |
| | EDTAVYYCARGGGVFDYWGQGTLVTVSSGGGGSGGGGSGGGG | |
| | SDIQMTQSPSTLSASVGDRVTITCSASSSISYMHWYQQKPGKAPK | |
| | LLIYTTSNLASGVPARFSGSGSGTEFTLTISSLQPDDFATYYCHQR | |
| | STYPLTFGQGTKVEVK | |
| SEQ ID | DIQMTQSPSTLSASVGDRVTITCSASSSISYMHWYQQKPGKAPKL | CD25 |
| NO: 491 | LIYTTSNLASGVPARFSGSGSGTEFTLTISSLQPDDFATYYCHQRS | |
| | TYPLTFGQGTKVEVKGGGGSGGGGSGGGGSQVQLVQSGAEVKK | |
| | PGSSVKVSCKASGYTFTSYRMHWVRQAPGQGLEWIGYINPSTGY | |
| | TEYNQKFKDKATITADESTNTAYMELSSLRSEDTAVYYCARGGG | |
| | VFDYWGQGTLVTVSS | |

An immune cell targeting agent (e.g., a binding moiety that binds CD3, 4-1BB, CD28, CD137, CD89, CD16, CD25, CD13, CD29, CD44, CD71, CD73, CD90, CD105, CD166, CD27, GITR, TIGIT, LAG3, TCR, CD40L, OX40, PD-1, CTLA-4, or STRO-1) may be complexed with a PD-L1-binding peptide via a linker (e.g., a peptide linker). In some embodiments, the immune cell targeting agent and the PD-L1-binding peptide may form a single polypeptide chain. In some embodiments, the immune cell targeting agent and the PD-L1-binding peptide may be complexed by forming a heterodimer via a heterodimerization domain. The immune cell targeting agent may be linked or fused to a first heterodimerization domain and the PD-L1-binding peptide may be linked or fused to a second heterodimerization domain. The first heterodimerization domain may bind to the second heterodimerization domain to form a heterodimeric complex comprising the immune cell targeting agent and the PD-L1-binding peptide. For example, the PD-L1-binding peptide may be linked or fused to an Fc “knob” peptide (e.g., SEQ ID NO: 124) and the immune cell targeting agent may be linked or fused ton an Fc “hole” peptide (e.g., SEQ ID NO: 125). In another example, the PD-L1-binding peptide may be linked or fused to an Fc “hole” peptide (e.g., SEQ ID NO: 125) and the immune cell targeting agent may be linked or fused ton an Fc “knob” peptide (e.g., SEQ ID NO: 124). An example of a PD-L1-binding half of a heterodimeric T cell engager may comprise a sequence of SEQ ID NO: 119 or SEQ ID NO: 120. An example of immune cell targeting half of a heterodimeric T cell engager that binds CD3 may comprise a sequence of SEQ ID NO: 123. In some embodiments, a PD-L1-binding peptide (e.g., any one of SEQ ID NO: 1-SEQ ID NO: 118, SEQ ID NO: 435, SEQ ID NO: 436, or SEQ ID NO: 554-SEQ ID NO: 567) may form a heterodimer with an immune cell binding moiety via a heterodimerization domain provided in TABLE 3. For example, the PD-L1-binding peptide may be fused to chain 1 of an Fc pair (e.g., SEQ ID NO: 126) and the immune cell binding moiety may be fused to chain 2 of the Fc pair (e.g., SEQ ID NO: 127). In another example, the PD-L1-binding peptide may be fused to chain 2 of an Fc pair (e.g., SEQ ID NO: 129) and the immune cell binding moiety may be fused to chain 1 of the Fc pair (e.g., SEQ ID NO: 128). It is understood that the paired heterodimerization domains denoted by “Pairs” in TABLE 3 (e.g., chain 1 and 2 of a given Pair) that one of “Chains” the Pair may be fused to the immune cell binding moiety and the other “Chain” fused to PD-L1-binding peptide, or vice versa, to form the heterodimer.

TABLE 3
Exemplary Heterodimerization Domains

| Fc | SEQ ID | |
|---|---|---|
| Name | NO | Sequence |
| Fc | SEQ ID | EPKSSDKTHTCPPCPAPELLGGPSVFLFPPKPKDTLMISRTPEVTCV |
| knob | NO: 124 | VVDVSHEDPEVKFNWYVDGVEVHNAKTKPREEQYASTYRVVSV |
| Chain 1 | | LTVLHQDWLNGKEYKCKVSNKALGAPIEKTISKAKGQPREPQVY |
| Pair 0 | | TLPPSRDELTKNQVSLWCLVKGFYPSDIAVEWESNGQPENNYKT |
| | | TPPVLDSDGSFFLYSKLTVDKSRWQQGNVFSCSVMHEALHNHYT |
| | | QKSLSLSPGK |
| Fc hole | SEQ ID | EPKSSDKTHTCPPCPAPELLGGPSVFLFPPKPKDTLMISRTPEVTCV |
| Chain 2 | NO: 125 | VVDVSHEDPEVKFNWYVDGVEVHNAKTKPREEQYASTYRVVSV |
| Pair 0 | | LTVLHQDWLNGKEYKCKVSNKALGAPIEKTISKAKGQPREPQVY |
| | | TLPPSRDELTKNQVSLSCAVKGFYPSDIAVEWESNGQPENNYKTT |
| | | PPVLDSDGSFFLVSKLTVDKSRWQQGNVFSCSVMHEALHNHYTQ |
| | | KSLSLSPGK |
| Chain 1 | SEQ ID | EPKSCDKTHTCPPCPAPELLGGPSVFLFPPKPKDTLMISRTPEVTC |
| Pair 1 | NO: 126 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLWCLVKGFYPSDIAVEWESNGQPENNYK |
| | | TTPPVLDSDGSFFLYSKLTVDKSRWQQGNVFSCSVMHEALHNHY |
| | | TQKSLSLSPGK |
| Chain 2 | SEQ ID | EPKSCDKTHTCPPCPAPELLGGPSVFLFPPKPKDTLMISRTPEVTC |
| Pair 1 | NO: 127 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLSCAVKGFYPSDIAVEWESNGQPENNYKT |
| | | TPPVLDSDGSFFLVSKLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 2 | NO: 128 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPCRDELTKNQVSLWCLVKGFYPSDIAVEWESNGQPENNYK |
| | | TTPPVLDSDGSFFLYSKLTVDKSRWQQGNVFSCSVMHEALHNHY |
| | | TQKSLSLSPGK |
| Chain 2 | SEQ ID | EPKSCDKTHTCPPCPAPELLGGPSVFLFPPKPKDTLMISRTPEVTC |
| Pair 2 | NO: 129 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | CTLPPSRDELTKNQVSLSCAVKGFYPSDIAVEWESNGQPENNYKT |
| | | TPPVLDSDGSFFLVSKLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 3 | NO: 130 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLTCLVKGFYPSDIAVEWESNGQPENNYDT |
| | | TPPVLDSDGSFFLYSDLTVDKSRWQQGNVFSCSVMHEALHNHYT |
| | | QKSLSLSPGK |
| Chain 2 | SEQ ID | EPKSCDKTHTCPPCPAPELLGGPSVFLFPPKPKDTLMISRTPEVTC |
| Pair 3 | NO: 131 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRKELTKNQVSLTCLVKGFYPSDIAVEWESNGQPENNYKT |
| | | TPPVLKSDGSFFLYSKLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 4 | NO: 132 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVHLTCLVKGFYPSDIAVEWESNGQPENNYK |
| | | TTPPVLDSDGSFALYSKLTVDKSRWQQGNVFSCSVMHEALHNHY |
| | | TQKSLSLSPGK |
| Chain 2 | SEQ ID | EPKSCDKTHTCPPCPAPELLGGPSVFLFPPKPKDTLMISRTPEVTC |
| Pair 4 | NO: 133 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | TTLPPSRDELTKNQVSLTCLVKGFYPSDIAVEWESNGQPENNYKT |
| | | FPPVLDSDGSFFLYSKLTVDKSRWQQGNVFSCSVMHEALHNHYT |
| | | QKSLSLSPGK |
| Chain 1 | SEQ ID | EPKSCEKTHTCPECPAPELLGGPSVFLFPPKPKDTLMISRTPEVTC |
| Pair 5 | NO: 134 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLTCEVKGFYPSDIAVEWESNGQPENNYKT |
| | | TPPVLDSDGSFFLYSKLTVDKSRWQQGNVFSCSVMHEALHNHYT |
| | | QKSLSLSPGK |
| Chain 2 | SEQ ID | EPKSCRKTHTCPRCPAPELLGGPSVFLFPPKPKDTLMISRTPEVTC |
| Pair 5 | NO: 135 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSRLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 6 | NO: 136 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFLLYSKLTVDKSRWQQGNVFSCSVMHEALHNHYT |
| | | QKSLSLSPGK |
| Chain 2 | SEQ ID | EPKSCDKTHTCPPCPAPELLGGPSVFLFPPKPKDTLMISRTPEVTC |
| Pair 6 | NO: 137 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLTCLVKGFYPSDIAVEWESNGQPENNYKT |
| | | TPPVLDSDGSFFLYSRLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 7 | NO: 138 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YVLPPSRDELTKNQVSLLCLVKGFYPSDIAVEWESNGQPENNYLT |
| | | WPPVLDSDGSFFLYSKLTVDKSRWQQGNVFSCSVMHEALHNHY |
| | | TQKSLSLSPGK |
| Chain 2 | SEQ ID | EPKSCDKTHTCPPCPAPELLGGPSVFLFPPKPKDTLMISRTPEVTC |
| Pair 7 | NO: 139 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YVYPPSRDELTKNQVSLTCLVKGFYPSDIAVEWESNGQPENNYK |
| | | TTPPVLDSDGSFALVSKLTVDKSRWQQGNVFSCSVMHEALHNHY |
| | | TQKSLSLSPGK |
| Chain 1 | SEQ ID | EPKSCDKTHTCPPCPAPELLGGPSVFLFPPKPKDTLMISRTPEVTC |
| Pair 8 | NO: 140 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTENQVSLTCLVKGFYPSDIAVEWESNGQPENNYKT |
| | | TPPVLDSDGSFFLYSWLTVDKSRWQQGNVFSCSVMHEALHNHY |
| | | TQKSLSLSPGK |
| Chain 2 | SEQ ID | EPKSCDKTHTCPPCPAPELLGGPSVFLFPPKPKDTLMISRTPEVTC |
| Pair 8 | NO: 141 | VVVDVSHEDPEVKFNWYVDGVEVHNAKTKPREEQYNSTYRVVS |
| | | VLTVLHQDWLNGKEYKCKVSNKALPAPIEKTISKAKGQPREPRV |
| | | YTLPPSRDELTKNQVSLTCLVKGFYPSDIAVEWESNGQPENNYKT |
| | | TPPVLVSDGSFTLYSKLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 9 | NO: 142 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | CTLPPSRDELTENQVSLTCLVKGFYPSDIAVEWESNGQPENNYKT |
| | | TPPVLDSDGSFFLYSWLTVDKSRWQQGNVFSCSVMHEALHNHY |
| | | TQKSLSLSPGK |
| Chain 2 | SEQ ID | EPKSCDKTHTCPPCPAPELLGGPSVFLFPPKPKDTLMISRTPEVTC |
| Pair 9 | NO: 143 | VVVDVSHEDPEVKFNWYVDGVEVHNAKTKPREEQYNSTYRVVS |
| | | VLTVLHQDWLNGKEYKCKVSNKALPAPIEKTISKAKGQPREPRV |
| | | YTLPPCRDELTKNQVSLTCLVKGFYPSDIAVEWESNGQPENNYKT |
| | | TPPVLVSDGSFTLYSKLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 10 | NO: 144 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLTCLVEGFYPSDIAVEWESNGQPENNYKT |
| | | TPPVLDSDGSFFLYSWLTVDKSRWQQGNVFSCSVMHEALHNHY |
| | | TQKSLSLSPGK |
| Chain 2 | SEQ ID | EPKSCDKTHTCPPCPAPELLGGPSVFLFPPKPKDTLMISRTPEVTC |
| Pair 10 | NO: 145 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDNLTKNQVSLTCLVKGFYPSDIAVEWESNGQPENNYKT |
| | | TPPVLVSDGSFTLYSKLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 11 | NO: 146 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTDNQVSLTCLVKGFYPSDIAVEWESNGQPENNYKT |
| | | TPPVLMSDGSFFLASKLTVDKSRWQQGNVFSCSVMHEALHNHYT |
| | | QKSLSLSPGK |
| Chain 2 | SEQ ID | EPKSCDKTHTCPPCPAPELLGGPSVFLFPPKPKDTLMISRTPEVTC |
| Pair 11 | NO: 147 | VVVDVSHEDPEVKFNWYVDGVEVHNAKTKPREEQYNSTYRVVS |
| | | VLTVLHQDWLNGKEYKCKVSNKALPAPIEKTISKAKGQPRRPRV |
| | | YTLPPSRDELTKNQVSLVCLVKGFYPSDIAVEWESNGQPENNYKT |
| | | TPPVLDSDGSFFLYSVLTVDKSRWQQGNVFSCSVMHEALHNHYT |
| | | QKSLSLSPGK |
| Chain 1 | SEQ ID | EPKSCDKTHTCPPCPAPELLGGPSVFLFPPKPKDTLMISRTPEVTC |
| Pair 12 | NO: 148 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | STLPPSRDELTKNQVSLMCLVYGFYPSDIAVEWESNGQPENNYKT |
| | | TPPVLDSDGSFFLYSVLTVDKSRWQQGNVFSCSVMHEALHNHYT |
| | | QKSLSLSPGK |
| Chain 2 | SEQ ID | EPKSCDKTHTCPPCPAPELLGGPSVFLFPPKPKDTLMISRTPEVTC |
| Pair 12 | NO: 149 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRGDLTKNQVQLTCLVKGFYPSDIAVEWESNGQPENNYK |
| | | TTPPVLDSDGSFFLASKLTVDKSRWQQGNVFSCSVMHEALHNHY |
| | | TQKSLSLSPGK |
| Chain 1 | SEQ ID | EPKSCDKTHTCPPCPAPELLGGPSVFLFPPKPKDTLMISRTPEVTC |
| Pair 13 | NO: 150 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTDPPSRDELTKNQVSLTCEVKGFYPSDIAVEWESNGQPENNYKT |
| | | TPPVLDSDGSFFLYSKLTVDKSRWQQGNVFSCSVMHEALHNHYT |
| | | QKSLSLSPGK |
| Chain 2 | SEQ ID | EPKSCDKTHTCPPCPAPELLGGPSVFLFPPKPKDTLMISRTPEVTC |
| Pair 13 | NO: 151 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTKPPSRDELTKNQVSLKCLVKGFYPSDIAVEWESNGQPENNYK |
| | | TTPPVLDSDGSFFLYSKLTVDKSRWQQGNVFSCSVMHEALHNHY |
| | | TQKSLSLSPGK |
| Chain 1 | SEQ ID | EPKSCDKTHTCPPCPAPELLGGPSVFLFPPKPKDTLMISRTPEVTC |
| Pair 14 | NO: 152 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDELTKNQVSLTCDVSGFYPSDIAVEWESNGQPENNYKT |
| | | TPPVLDSDGSFFLYSKLTVDKSRWQQGNVFSCSVMHEALHNHYT |
| | | QKSLSLSPGK |
| Chain 2 | SEQ ID | EPKSCDKTHTCPPCPAPELLGGPSVFLFPPKPKDTLMISRTPEVTC |
| Pair 14 | NO: 153 | VVVDVSHEDPEVKFNWYVDGVEVHNAKTKPREEQYNSTYRVVS |
| | | VLTVLHQDWLNGKEYKCKVSNKALPAPIEKTISKAKGQPREPQV |
| | | YTLPPSRDQLTKNQVKLTCLVKGFYPSDIAVEWESNGQPENNYK |
| | | TTPPVLDSDGSFFLYSKLTVDKSRWQQGNVFSCSVMHEALHNHY |
| | | TQKSLSLSPGK |

In some cases, the PD-L1 binding peptide and the immune cell targeting agent may both be presented as dimers, such as by placing or positioning the PD-L1 binding peptide on the N- or C-terminus of a homodimeric Fc fusion and placing or positioning the immune cell targeting agent on the other end (the C- or N-terminus) of a homodimeric Fc fusion. An exemplary homodimeric Fc of SEQ ID NO: 1 and an anti-CD3 scFv (EVQLVESGGGLVQPGGSLKLSCAASGFTFNKYAMNWVRQAPGKGLEWVARIRSKYN NYATYYADSVKDRFTISRDDSKNTAYLQMNNLKTEDTAVYYCVRHGNFGNSYISYWA YWGQGTLVTVSSGGGGSGGGGSGGGGSQTVVTQEPSLTVSPGGTVTLTCGSSTGAVTS GNYPNWVQQKPGQAPRGLIGGTKFLAPGTPARFSGSLLGGKAALTLSGVQPEDEAEYY CVLWYSNRWVFGGGTKLTVL; SEQ TD NO: 122) is shown in SEQ TD NO: 121. Additional examples of Shomodimeric F fusions are provided in SEQ TD NO: 438-SEQ ID NO: 441. Examples of components that may be homodimerized or heterodimerized to form a bispecific immune cell engager are provided in TABLE 4. For example, a PD-L1-binding Fe hole component (e.g., SEQ ID NO: 119 or SEQ TD NO: 120) may heterodimerize with an anti-CD3 Fe knob component (e.g., SEQ TD NO: 123) to form a PD-L1/CD3-binding BiICE.

TABLE 4
Exemplary BiICE Components

| Name | SEQ ID NO | Sequence |
|---|---|---|
| PD-L1- | SEQ ID NO: | EEDCKVHCVKEWMAGKACAERDKSYTIGRAHCSGQKFDVFK |
| binder Fc | 119 | CLDHCAAPGGGGSGGGGSGGGGSEPKSSDKTHTCPPCPAPELL |
| hole | | GGPSVFLFPPKPKDTLMISRTPEVTCVVVDVSHEDPEVKFNWY |
| | | VDGVEVHNAKTKPREEQYASTYRVVSVLTVLHQDWLNGKEY |
| | | KCKVSNKALGAPIEKTISKAKGQPREPQVYTLPPSRDELTKNQ |
| | | VSLSCAVKGFYPSDIAVEWESNGQPENNYKTTPPVLDSDGSFF |
| | | LVSKLTVDKSRWQQGNVFSCSVMHEALHNHYTQKSLSLSPGK |
| PD-L1- | SEQ ID NO: | EEDCKVHCVKEWMAGKACAERNKSYTIGRAHCSGQKFDVFK |
| binder Fc | 120 | CLDHCAAPGGGGSGGGGSGGGGSEPKSSDKTHTCPPCPAPELL |
| hole | | GGPSVFLFPPKPKDTLMISRTPEVTCVVVDVSHEDPEVKFNWY |
| | | VDGVEVHNAKTKPREEQYASTYRVVSVLTVLHQDWLNGKEY |
| | | KCKVSNKALGAPIEKTISKAKGQPREPQVYTLPPSRDELTKNQ |
| | | VSLSCAVKGFYPSDIAVEWESNGQPENNYKTTPPVLDSDGSFF |
| | | LVSKLTVDKSRWQQGNVFSCSVMHEALHNHYTQKSLSLSPGK |
| Anti-CD3 Fc | SEQ ID NO: | EVQLVESGGGLVQPGGSLKLSCAASGFTFNKYAMNWVRQAP |
| PD-L1- | 121 | GKGLEWVARIRSKYNNYATYYADSVKDRFTISRDDSKNTAYL |
| binder | | QMNNLKTEDTAVYYCVRHGNFGNSYISYWAYWGQGTLVTVS |
| | | SGGGGSGGGGSGGGGSQTVVTQEPSLTVSPGGTVTLTCGSSTG |
| | | AVTSGNYPNWVQQKPGQAPRGLIGGTKFLAPGTPARFSGSLLG |
| | | GKAALTLSGVQPEDEAEYYCVLWYSNRWVFGGGTKLTVLGG |
| | | GGSEPKSSDKTHTCPPCPAPELLGGPSVFLFPPKPKDTLMISRTP |
| | | EVTCVVVDVSHEDPEVKFNWYVDGVEVHNAKTKPREEQYAS |
| | | TYRVVSVLTVLHQDWLNGKEYKCKVSNKALGAPIEKTISKAK |
| | | GQPREPQVYTLPPSRDELTKNQVSLTCLVKGFYPSDIAVEWES |
| | | NGQPENNYKTTPPVLDSDGSFFLYSKLTVDKSRWQQGNVFSCS |
| | | VMHEALHNHYTQKSLSLSPGKGGGGSGGGGSGGGGSEEDCK |
| | | VHCVKEWMAGKACAERQKSYTIGRAHCSGQKFDVFKCLDHC |
| | | AAP |
| Anti-CD3 Fc | SEQ ID NO: | EVQLVESGGGLVQPGGSLKLSCAASGFTFNKYAMNWVRQAP |
| knob | 123 | GKGLEWVARIRSKYNNYATYYADSVKDRFTISRDDSKNTAYL |
| | | QMNNLKTEDTAVYYCVRHGNFGNSYISYWAYWGQGTLVTVS |
| | | SGGGGSGGGGSGGGGSQTVVTQEPSLTVSPGGTVTLTCGSSTG |
| | | AVTSGNYPNWVQQKPGQAPRGLIGGTKFLAPGTPARFSGSLLG |
| | | GKAALTLSGVQPEDEAEYYCVLWYSNRWVFGGGTKLTVLGG |
| | | GGSEPKSSDKTHTCPPCPAPELLGGPSVFLFPPKPKDTLMISRTP |
| | | EVTCVVVDVSHEDPEVKFNWYVDGVEVHNAKTKPREEQYAS |
| | | TYRVVSVLTVLHQDWLNGKEYKCKVSNKALGAPIEKTISKAK |
| | | GQPREPQVYTLPPSRDELTKNQVSLWCLVKGFYPSDIAVEWES |
| | | NGQPENNYKTTPPVLDSDGSFFLYSKLTVDKSRWQQGNVFSCS |
| | | VMHEALHNHYTQKSLSLSPGK |
| PD-L1- | SEQ ID NO: | EEDCKVHCVKEWMAGKACAERDKSYTIGRAHCSGQKFDVFK |
| binder Fc | 437 | CLDHCAAPGGGGSGGGGSGGGGSEPKSSDKTHTCPPCPAPELL |
| | | GGPSVFLFPPKPKDTLMISRTPEVTCVVVDVSHEDPEVKFNWY |
| | | VDGVEVHNAKTKPREEQYASTYRVVSVLTVLHQDWLNGKEY |
| | | KCKVSNKALGAPIEKTISKAKGQPREPQVYTLPPSRDELTKNQ |
| | | VSLTCLVKGFYPSDIAVEWESNGQPENNYKTTPPVLDSDGSFFL |
| | | YSKLTVDKSRWQQGNVFSCSVMHEALHNHYTQKSLSLSPGK |
| PD-L1- | SEQ ID NO: | EEDCKVHCVKEWMAGKACAERDKSYTIGRAHCSGQKFDVFK |
| binder Anti- | 438 | CLDHCAAPGGGGSGGGGSGGGGSEVQLVESGGGLVQPGGSLK |
| CD3-scFc | | LSCAASGFTFNKYAMNWVRQAPGKGLEWVARIRSKYNNYAT |
| | | YYADSVKDRFTISRDDSKNTAYLQMNNLKTEDTAVYYCVRHG |
| | | NFGNSYISYWAYWGQGTLVTVSSGGGGSGGGGSGGGGSQTV |
| | | VTQEPSLTVSPGGTVTLTCGSSTGAVTSGNYPNWVQQKPGQAP |
| | | RGLIGGTKFLAPGTPARFSGSLLGGKAALTLSGVQPEDEAEYY |
| | | CVLWYSNRWVFGGGTKLTVL |
| PD-L1- | SEQ ID NO: | EEDCKVHCVKEWMAGKACAERDKSYTIGRAHCSGQKFDVFK |
| binder Anti- | 439 | CLDHCAAPEPKSSDKTHTEVQLVESGGGLVQPGGSLKLSCAAS |
| CD3-scFc | | GFTFNKYAMNWVRQAPGKGLEWVARIRSKYNNYATYYADSV |
| | | KDRFTISRDDSKNTAYLQMNNLKTEDTAVYYCVRHGNFGNSY |
| | | ISYWAYWGQGTLVTVSSGGGGSGGGGSGGGGSQTVVTQEPSL |
| | | TVSPGGTVTLTCGSSTGAVTSGNYPNWVQQKPGQAPRGLIGGT |
| | | KFLAPGTPARFSGSLLGGKAALTLSGVQPEDEAEYYCVLWYSN |
| | | RWVFGGGTKLTVL |
| PD-L1- | SEQ ID NO: | EEDCKVHCVKEWMAGKACAERDKSYTIGRAHCSGQKFDVFK |
| binder Anti- | 440 | CLDHCAAPGGGGSGGGGSGGGGSLKEAKEKAIEELKKAGITSD |
| CD3-scFc | | YYFDLINKAKTVEGVNALKDEILKAGGGGSGGGGSGGGGSEV |
| | | QLVESGGGLVQPGGSLKLSCAASGFTFNKYAMNWVRQAPGK |
| | | GLEWVARIRSKYNNYATYYADSVKDRFTISRDDSKNTAYLQM |
| | | NNLKTEDTAVYYCVRHGNFGNSYISYWAYWGQGTLVTVSSG |
| | | GGGSGGGGSGGGGSQTVVTQEPSLTVSPGGTVTLTCGSSTGAV |
| | | TSGNYPNWVQQKPGQAPRGLIGGTKFLAPGTPARFSGSLLGGK |
| | | AALTLSGVQPEDEAEYYCVLWYSNRWVFGGGTKLTVL |
| PD-L1- | SEQ ID NO: | EEDCKVHCVKEWMAGKACAERDKSYTIGRAHCSGQKFDVFK |
| binder Anti- | 441 | CLDHCAAPEPKSSDKTHTLKEAKEKAIEELKKAGITSDYYFDLI |
| CD3-scFc | | NKAKTVEGVNALKDEILKAEPKSSDKTHTEVQLVESGGGLVQ |
| | | PGGSLKLSCAASGFTFNKYAMNWVRQAPGKGLEWVARIRSKY |
| | | NNYATYYADSVKDRFTISRDDSKNTAYLQMNNLKTEDTAVYY |
| | | CVRHGNFGNSYISYWAYWGQGTLVTVSSGGGGSGGGGSGGG |
| | | GSQTVVTQEPSLTVSPGGTVTLTCGSSTGAVTSGNYPNWVQQ |
| | | KPGQAPRGLIGGTKFLAPGTPARFSGSLLGGKAALTLSGVQPE |
| | | DEAEYYCVLWYSNRWVFGGGTKLTVL |

In some embodiments, the immune cell targeting agent may be a single chain variable fragment (scFv), a cysteine-dense peptide, an avimer, a kunitz domain, an affibody, an adnectin, a nanofittin, a fynomer, a ß-hairpin, a stapled peptide, a bicyclic peptide, an antibody, an antibody fragment, a protein, a peptide, a peptide fragment, a binding domain, a small molecule, or a nanobody that binds the immune cell target (e.g., CD3, 4-1BB, CD28, CD137, CD89, CD16, CD25, CD13, CD29, CD44, CD71, CD73, CD90, CD105, CD166, CD27, GITR, TIGIT, LAG3, TCR, CD40L, OX40, PD-1, CTLA-4, or STRO-1).

Upon delivery, an immune cell (e.g., a T cell, a B cell, a macrophage, or a natural killer cell) may inactivate, inhibit, kill, or protect the PD-L1 positive cell. The function of the bispecific immune cell engager may depend on the type of immune cell recruited. In some embodiments, the immune cell (e.g., a regulatory T cell) may inhibit the PD-L1 positive cell (e.g., a PD-L1 positive T cell). For example, a bispecific immune cell engager comprising a CD25-binding agent may recruit a regulatory T cell to a PD-L1 positive T cell associated with an autoimmune response and inhibit the T cell.

A targeted immune cell (e.g., a T cell, a B cell, a macrophage, or a natural killer cell) can interact with the PD-L1-expressing cell via the immune cell binding moiety in the BiICE, producing an energetically favorable interface that enables a close proximity or narrow enough immune synapse of the immune cell engager to a cell, for example a targeted cancer cell. The immunological synapse may be narrow enough to enable signal exchange between the targeted cell, engaged by the PD-L1-binding peptide, and the targeted immune cell, engaged by the immune cell binding moiety, resulting in an immune response against the cancer cell. For example, bispecific PD-L1 binding peptide-CD3 BiICE molecules (e.g., comprising a PD-L1-binding CDP complexed with a CD3-binding moiety) can bind to both CD3 on the immune cell and PD-L1 on the target cell, producing an energetically favorable interface. The space between an immune cell and a target cell is referred to as an immune synapse, and in normal T cells it is primarily driven by a complex between the T cell receptor (TCR) on the T cell and a peptide-MHC complex on the target cell. In some embodiments, the immune synapse may function as a kinapse, or moving synapse. The width of this synapse is the distance between the cell membranes of the immune cell and the target cell. In some embodiments, such as in a TCR-driven synapse, the width of the synapse may be about 15 nm. In some embodiments, the synapse may have a width of from about 3 nm to about 25 nm, from about 5 nm to about 20 nm, or from about 10 nm to about 15 nm. In some embodiments, the synapse may have a width of about 3 nm, about 5 nm, about 8 nm, about 10 nm, about 13 nm, about 15 nm, about 18 nm, about 20 nm, about 23 nm, or about 25 nm. In some embodiments, the synapse may have a width of less than 3 nm, less than 5 nm, less than 8 nm, less than 10 nm, less than 13 nm, less than 15 nm, less than 18 nm, less than 20 nm, less than 23 nm, less than 25 nm, less than 30 nm, less than 35 nm, less than 40 nm, less than 45 nm, less than 50 nm, less than 55 nm, less than 60 nm, less than 65 nm, less than 70 nm, less than 75 nm, less than 80 nm, less than 85 nm, less than 90 nm, less than 95 nm, or less than 100 nm. A BiICE molecule may induce an immunological synapse of dimensions dependent on the size of the BiICE and binding location on the respective targets (e.g., the immune cell and the cancer cell). For example, larger molecules with more space between the binding entities will produce a larger synapse. The immunological synapse size can determine the efficacy of a T cell response, such as the rate of degranulation, activation and exhaustion dynamics, and T cell mobility within the tumor microenvironment. BiICE molecules containing smaller binding moieties, such as CDPs, can form immunological synapses that are smaller than can be achieved with bispecific molecules containing larger binding moieties like antibodies. In some embodiments, a smaller synapse formed between a T cell and a cancer cell may yield a more potent T cell killing response due to closer proximity of the T cell to the cancer cell. The interaction between a PD-L1-binding CDP and PD-L1 (e.g., between SEQ ID NO: 1 and PD-L1) may place the termini of the CDP close to the surface of PD-L1. In contrast, an scFv may bind to PD-L1 in an orientation such that the termini of the scFv may be as far as 5 nm away from the surface of PD-L1, resulting in an immunological synapse diameter of as much as 5 nm greater than an immunological synapse formed with a PD-L1-binding CDP. A 5 nm increase in synapse diameter may have substantial biochemical or physiological consequences since a typical immune cell synapse is about 15 nm. A narrower synapse, such as formed by a BiICE containing a PD-L1-binding CDP, may trigger a potent T cell killing response. In some embodiments, the immunological synapse of a PD-L1-binding peptide complex (e.g., a BiICE containing a PD-L1-binding CDP) is about 3 nm, about 5 nm, about 8 nm, about 10 nm, about 13 nm, about 15 nm, about 18 nm, about 20 nm, about 23 nm, or about 25 nm. In some embodiments, the immunological synapse of a PD-L1-binding peptide complex is no more than 3 nm, no more than 4 nm, no more than 5 nm, no more than 6 nm, no more than 7 nm, no more than 8 nm, no more than 9 nm, no more than 10 nm, no more than 13 nm, no more than 15 nm, no more than 18 nm, no more than 20 nm, no more than 23 nm, no more than 25 nm, no more than 30 nm, no more than 35 nm, no more than 40 nm, no more than 45 nm, no more than 50 nm, no more than 60 nm, no more than 65 nm, no more than 70 nm, no more than 75 nm, no more than 80 nm, no more than 85 nm, no more than 90 nm, no more than 95 nm, or no more than 100 nm.

### Chimeric Antigen Receptors

A PD-L1-binding peptide of the present disclosure (e.g., any one of SEQ ID NO: 1-SEQ ID NO: 118, SEQ ID NO: 435, SEQ ID NO: 436, or SEQ ID NO: 554-SEQ ID NO: 567) may be complexed with one or more components of a chimeric antigen receptor to form a PD-L1-binding chimeric antigen receptor. In some embodiments, a PD-L1-binding peptide may be linked or fused to a transmembrane domain, an intracytoplasmic domain, a heavy chain variable domain, a light chain variable domain, or combinations thereof, of a chimeric antigen receptor (CAR). In some embodiments, a PD-L1-binding peptide may replace a single chain variable fragment (scFv) of a chimeric antigen receptor to form a PD-L1-binding chimeric antigen receptor (PD-L1-binding CAR). For example, a PD-L1-binding CAR may comprise a PD-L1-binding peptide (e.g., any one of SEQ ID NO: 1-SEQ ID NO: 118, SEQ ID NO: 435, SEQ ID NO: 436, or SEQ ID NO: 554-SEQ ID NO: 567), a linker (e.g., SEQ ID NO: 154-SEQ ID NO: 241 or SEQ ID NO: 433), a transmembrane domain, and an intracellular costimulatory domain. In some embodiments, a nucleotide sequence encoding a PD-L1-binding chimeric antigen receptor may be expressed in an immune cell (e.g., a T cell). The PD-L1-binding chimeric antigen receptor may be expressed on the surface of the immune cell. In some embodiments, the PD-L1-binding chimeric antigen receptor may function by recruiting the T cell to a PD-L1 positive cell (e.g., a PD-L1 positive cancer cell) and killing or inactivating the PD-L1 positive cell.

### Peptide Oligonucleotide Complexes

In some embodiments, an active agent may comprise an oligonucleotide. A PD-L1-binding peptide (e.g., any one of SEQ ID NO: 1-SEQ ID NO: 118, SEQ ID NO: 435, SEQ ID NO: 436, or SEQ ID NO: 554-SEQ ID NO: 567) may be complexed with an oligonucleotide to form a peptide oligonucleotide complex, also referred to as a peptide-nucleotide agent conjugate, a peptide oligonucleotide complex, or a peptide target-binding agent complex, may comprise a peptide complexed with a nucleotide (e.g., an oligonucleotide). The peptide of the peptide oligonucleotide complex may comprise a PD-L1-binding peptide, as described herein. In some embodiments, the peptide may be a PD-L1-binding peptide (e.g., any one of SEQ ID NO: 1-SEQ ID NO: 118, SEQ ID NO: 435, SEQ ID NO: 436, or SEQ ID NO: 554-SEQ ID NO: 567). A PD-L1-binding peptide of a peptide oligonucleotide complex may mediate binding of the peptide oligonucleotide complex to PD-L1, which may facilitate endocytosis or transcytosis of the peptide oligonucleotide complex across a cell barrier or entry into a cancer cell or tissue. For example, a peptide oligonucleotide complex comprising a PD-L1-binding peptide may cross a cellular membrane, enabling interactions between the nucleotide of the peptide oligonucleotide complex and various cytosolic or nuclear components (e.g., genomic DNA, an ORF, mRNA, pre-mRNA, or DNA). In some embodiments, a peptide oligonucleotide complex comprising a PD-L1-binding peptide may cross a cellular membrane by being endocytosed into a cell. A PD-L1-binding peptide of a peptide oligonucleotide complex may be a pH-dependent PD-L1-binding peptide engineered to have higher binding affinity for PD-L1 at an extracellular pH (e.g., pH 7.4) and lower binding affinity at an endosomal pH (e.g., pH 5.5 or pH 6.5).

The nucleotide of the peptide oligonucleotide complex may be a target-binding agent comprising single stranded DNA, single stranded RNA, double stranded DNA, double stranded RNA, or a combination thereof. As used herein, the term “nucleotide” may refer to an oligonucleotide or polynucleotide molecule or to a single nucleotide base. For example, a nucleotide of a peptide complex may comprise a DNA or RNA oligonucleotide. In some embodiments, the nucleotide may be a small interfering RNA (siRNA), a micro RNA (miRNA, or miR), an anti-miR, an antisense RNA, an antisense oligonucleotide (ASO), a complementary RNA, a complementary DNA, an interfering RNA, a small nuclear RNA (snRNA), a spliceosomal RNA, an inhibitory RNA, a nuclear RNA, an oligonucleotide complementary to a natural antisense transcript (NAT), an aptamer, a gapmer, a splice blocker ASO, or a U1 adapter. For example, a nucleotide of the peptide oligonucleotide complex may comprise a sequence of any one of any one of SEQ ID NO: 366-SEQ ID NO: 396 or a sequence complementary to a portion of any sequence provided in SEQ ID NO: 397-SEQ ID NO: 430 or SEQ ID NO: 549 or an open reading frame listed in TABLE 17. In some embodiments, the nucleotide may be an siRNA that inhibits translation of a target mRNA by promoting degradation of the target mRNA. In some embodiments, the nucleotide may be an miRNA that inhibits translation of a target mRNA by promoting cleavage or destabilization of the target mRNA. In some embodiments, the nucleotide may be an aptamer that binds to a target protein, thereby inhibiting protein-protein interactions with the target protein, inhibiting enzymatic activity of the target protein, or activating the target protein.

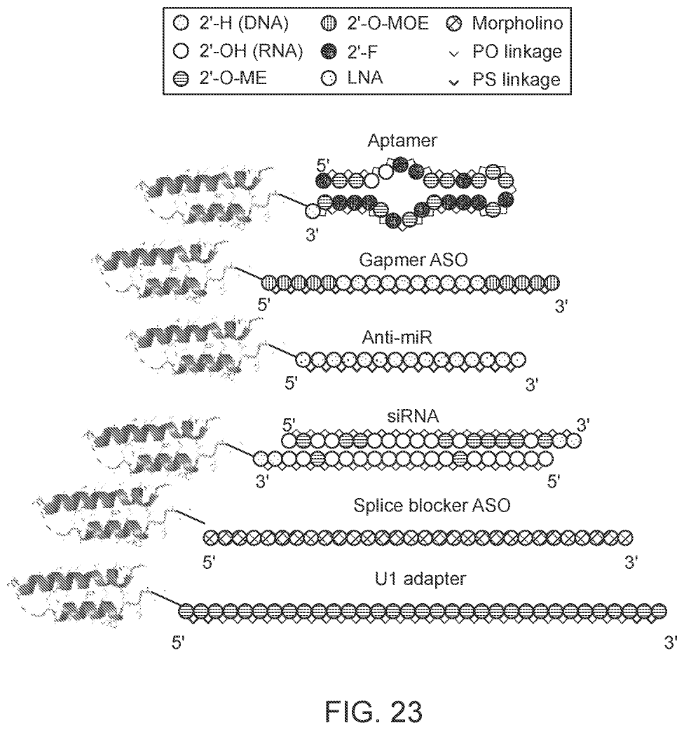

Examples of structures of various peptide oligonucleotide complexes (e.g., CDP-oligonucleotide complexes containing alternative and nonconventional bases) are illustrated in FIG. 23. Examples of oligonucleotides include an aptamer, a gapmer, an anti-miR, an siRNA, a splice blocker ASO, and a U1 adapter. The peptide portion of the peptide oligonucleotide complex (e.g., a CDP of a CDP-oligonucleotide complex) can be used to guide the nucleotide sequence (e.g., an oligonucleotide of a CDP-oligonucleotide complex) to a specific tissue, target, or cell.

In some embodiments, a peptide oligonucleotide complex binds PD-L1 with an affinity of no more than 10 nM, 5 nM, 1 nM, 800 pM, 600 pM, 500 pM, 400 pM, 300 pM, 250 pM, or 200 pM. In some embodiments, the affinity is identical or similar at pH 7.0 as at pH 7.4, identical or similar at pH 6.5 as at pH 7.4, identical or similar at pH 6.0 as at pH 7.4, or identical or similar at pH 5.5 as at pH 7.4. In some embodiments, the affinity is within ±nM, ±3 nM, ±5 nM, ±10 nM, ±30 pM, ±50 pM, ±100 pM, ±300 pM, ±500 pM, or ±1000 pM, when compared at pH 7.0 and pH 7.4, pH 6.5 and pH 7.4, pH 6.0 and pH 7.4, or pH 5.5 and pH 7.4. In some embodiments, the affinity is within 1-fold, 2-fold, 3-fold, 5-fold or 10-fold relative difference when compared at pH 7.0 and pH 7.4, pH 6.5 and pH 7.4, pH 6.0 and pH 7.4, or pH 5.5 and pH 7.4. In some aspects, the affinity of the peptide oligonucleotide complex binds the PD1 molecule is higher at a higher pH than at a lower pH. In some aspects, the higher pH is pH 7.4, pH 7.2, pH 7.0, or pH 6.8. In some aspects, the lower pH is pH 6.5, pH 6.0, pH 5.5, pH 5.0, or pH 4.5. In some aspects, the affinity of the peptide oligonucleotide complex for PD-L1 is higher at pH 7.4 than at pH 6.0. In some aspects, the affinity of the peptide oligonucleotide complex for PD-L1 is higher at pH 7.4 than at pH 5.5. In some aspects, the target binding peptide is capable of binding the target molecule with a dissociation constant (K_{D}) of no more than 100 nM, no more than 20 nM, no more than 10 nM, no more than 5 nM, no more than 2 nM, no more than 1 nM, no more than 0.5 nM, no more than 0.2 nM, no more than 1 nM, or no more than 0.1 nM at pH 7.4. In some aspects, the target binding peptide is capable of binding the target molecule with a dissociation constant (K_{D}) of no less than 1 nM, no less than 2 nM, no less than 5 nM, no less than 10 nM, no less than 20 nM, no less than 50 nM, no less than 100 nM, no less than 200 nM, or no less than 500 nM at pH 5.5. In some aspects, the affinity of the peptide oligonucleotide complex for PD-L1 at pH 7.4 is at least 1.25-fold, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 50-fold, at least 100-fold, at least 500-fold, at least 1000-fold, at least 5000-fold, or at least 10,000-fold greater than the affinity of the peptide oligonucleotide complex for PD-L1 at pH 5.5.

The peptide oligonucleotide complexes of the present disclosure may include nucleotide and nucleotide variants within the peptide oligonucleotide complex wherein the nucleotide portion is targeted to specific target molecule for modulation. Modulation of a target molecule may comprise degradation, inhibiting translation, decreasing expression, increasing expression, enhancing a binding interaction (e.g., a protein-protein interaction), or inhibiting a binding interaction (e.g., a protein-protein interaction). Disclosed herein are nucleotide sequences that may be used in the nucleotide portion of the peptide oligonucleotide complex, such as those targeting or complementary to nucleotides (e.g., DNA or RNA molecules) listed in SEQ ID NO: 397-SEQ ID NO: 430 or SEQ ID NO: 549, TABLE 10, or TABLE 17, or to nucleotides (e.g., DNA or RNA molecules) encoding the proteins listed in SEQ ID NO: 397-SEQ ID NO: 430 or SEQ ID NO: 549, TABLE 10, or TABLE 17, or otherwise described herein. Examples of nucleotide sequences that may be used in the nucleotide portion of the peptide oligonucleotide complex include SEQ ID NO: 366-SEQ ID NO: 396 and SEQ ID NO: 492-SEQ ID NO: 545. As disclosed herein, nucleic acid sequences, variants, and properties of the nucleic acids that are used in the nucleic acid portion of the peptide oligonucleotide complex may be referred to as nucleic acids of the present disclosure, nucleotides of the present disclosure, or like terminology. It may be understood that such nucleic acids or nucleotides are described in the context of the peptide oligonucleotide complexes disclosed, such as a nucleotide sequence comprising single stranded (ssDNA, ssRNA), double stranded (dsDNA, dsRNA), or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), oligonucleotide complementary to a natural antisense transcripts (NATs) sequences, siRNA, snRNA, aptamer, gapmer, anti-miR, splice blocker ASO, or U1 Adapter within the peptide oligonucleotide complex, with the accorded alterations, functions and uses described.

In some embodiments, the nucleotide sequence (e.g., a target binding agent capable of binding a target molecule) is single stranded (ssDNA, ssRNA), double stranded (dsDNA, dsRNA), or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter. Peptides according to the present disclosure can be conjugated to, linked to, or fused to such nucleotide sequences to make a peptide oligonucleotide complex. In addition, other active agents (e.g., small molecule, protein, or peptide active agents) as described herein can be conjugated to, linked to, complexed with, or fused to such nucleotide sequences, peptides or peptide oligonucleotide complex to form peptide oligonucleotide complex conjugates.

A nucleotide (e.g., a nucleotide of a peptide oligonucleotide complex) may be fully or partially reverse complementary to all or a portion of a target molecule (e.g., a target DNA or RNA sequence). In some embodiments, a target molecule expresses or encodes a protein (e.g., an mRNA encoding a protein associated with a disease). In some embodiments, a nucleotide may be fully or partially reverse complementary to a portion of an open reading frame encoding a gene or protein of interest. In some embodiments, a nucleotide may be reverse complementary to any portion of an RNA or open reading frame encoding a transcript or protein of interest. Examples of sequences that may serve as target molecules for the target binding nucleotides described herein are provided in SEQ ID NO: 397-SEQ ID NO: 430 and SEQ ID NO: 549 along any portion of its length. In some embodiments, a target molecule may comprise a fragment of any of the sequences provided in TABLE 17 along any portion of its length. In some embodiments, a target molecule may comprise a fragment of any of the sequences provided in SEQ ID NO: 397-SEQ ID NO: 430 or SEQ ID NO: 549. In some embodiments, a target molecule may comprise a sequence with one or more T residues replaced with U or one or more U residues replaced with T.

A number of technologies can be used to generate therapeutically active nucleotide sequences for use in peptide oligonucleotide complexes that include the PD-L1-binding peptides (e.g., SEQ ID NO: 1-SEQ ID NO: 118, SEQ ID NO: 435, SEQ ID NO: 436, or SEQ ID NO: 554-SEQ ID NO: 567) disclosed herein. Several have examples of molecules in the clinic or advanced clinical development and can be employed for the nucleotide portion within the peptide oligonucleotide complexes described herein. A nucleotide of a peptide oligonucleotide complex may bind to a target molecule (e.g., a target DNA, RNA, or protein) and modulate an activity of the target molecule. In this way, the nucleotide may function as a target-binding agent, also referred to as a targeted agent. Examples of nucleotides that may function as target-binding agents include nucleotide antisense RNAs, complementary RNAs, inhibitory RNAs, interfering RNAs, nuclear RNAs, antisense oligonucleotides, microRNAs, oligonucleotides complementary to natural antisense transcripts, small interfering RNAs, small nuclear RNAs, aptamers, gapmers, anti-miRs, splice blocker antisense oligonucleotides, and U1 adapters.

Nucleotides (e.g., oligonucleotides targeted to a specific sequence for its regulation) may enter into cells through complexation with a PD-L1-binding peptide to form a PD-L1-binding peptide oligonucleotide complex. The PD-L1-binding peptide oligonucleotide complex may then be endocytosed by PD-L1 or may enter the cell by other mechanisms. The oligonucleotide, with or without complex to the PD-L1-binding peptide (e.g., after linker cleavage), may exit the endosome or lysosome slowly over time through no active mechanism or through mechanisms of endosomal escape or through other mechanisms. The peptide oligonucleotide complex may exit the endosome or lysosome. A fragment or cleavage product of the peptide oligonucleotide complex may exit the endosome or lysosome. The oligonucleotide, the peptide oligonucleotide complex, or any fragment thereof may enter the cytosol and may enter the nucleus.

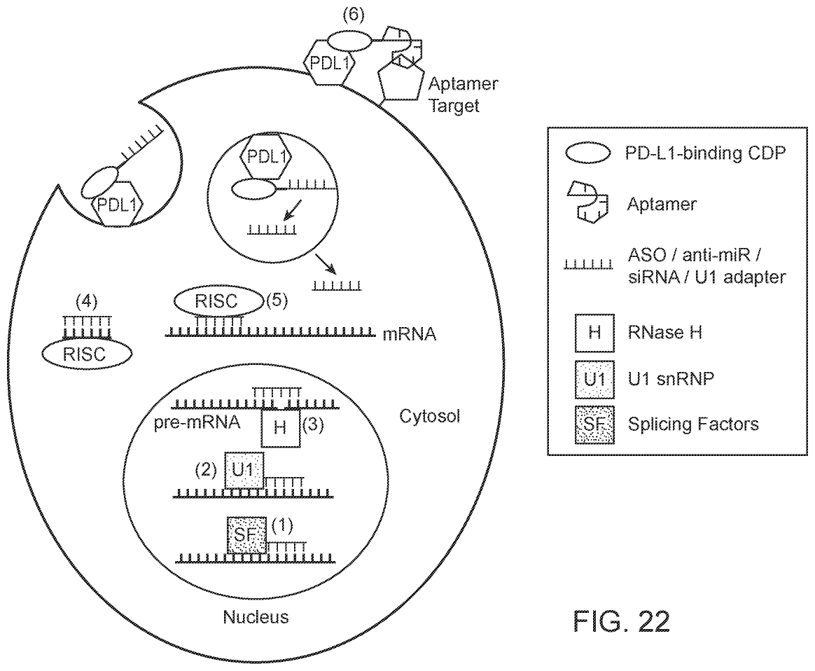

Possible mechanisms of action of oligonucleotides are illustrated in FIG. 22. In one embodiment, upon entry into the nucleus, oligonucleotides can (1) bind directly to mRNA structures and prevent the maturation (e.g., capping or splicing) of the targeted sequence, (2) modulate alternative splicing of a targeted sequence, (3) and recruit RNaseH1 to induce cleavage of a targeted sequence. In another embodiment, oligonucleotides in the cytoplasm can bind directly to the target mRNA and sterically block the ribosomal subunits from attaching and/or running along the mRNA transcript during translation hence resulting in lack of translation of the target sequence. In another embodiment, oligonucleotides can also be designed to (4) directly bind to microRNA (miRNA) sequences or natural antisense transcripts (NATs) sequences, each of the foregoing thereby prohibiting miRNAs and NATs from inhibiting their own specific RNA targets, which ultimately leads to reduced degradation or increased translation of one or more sequences themselves targeted by the miRNA or NAT. In another embodiment, siRNA (which may be targeted to a specific sequence and regulate expression of the target sequence) may alternatively be used to bind and regulate a targeted sequence in the cytoplasm (5), engaging an RNA-induced silencing complex (RISC), which is a multiprotein complex that incorporates one strand of a small interfering RNA (siRNA) or micro RNA (miRNA), using the siRNA or miRNA as a template for recognizing complementary mRNA of the targeted sequence. When it finds a complementary strand, its RNase domain cleaves the targeted sequence. In another embodiment, an aptamer (e.g., a nucleotide that modulates a specific protein or other target) may alternatively be used to bind and regulate a target (6). An aptamer (e.g., extracellular or intracellular) may function by directly binding and modulating activity of a protein target, for example by forming aptamer-protein interactions rather than through base pairing or hybridization interactions.

For example, conventional ASO, or antisense oligonucleotides, are typically 18-30 nucleotides (nt) in length. Several ASO therapeutic strategies exist, two of which (differing in their mechanism of target RNA interference) are further described. The first ASOs are sometimes called “Gapmers” because they have a central region with DNA-based-sugar nucleotides that are often (but not always) flanked by non-DNA-sugar nucleotides with greater resistance to nucleases. The DNA region, at least 4 nt in length but typically >6, causes a DNA/RNA hybrid that engages RNase H endonuclease to cleave the target RNA. Among clinically approved gapmers are fomivirsen and mipomersen. In some embodiments, a DNA region of a gapmer may comprise from about 4 to about 30, from about 4 to about 25, from about 4 to about 20, from about 4 to about 15, from about 4 to about 10, from about 6 to about 30, from about 6 to about 25, from about 6 to about 20, from about 6 to about 15, or from about 6 to about 10 nucleotide residues. In some embodiments, a non-DNA region of a gapmer may comprise about 1, 2, 3, 4, 5, 6, 7, 8, 9, or 10 nucleotide residues 3′ of the DNA region. In some embodiments, a non-DNA region of a gapmer may comprise about 1, 2, 3, 4, 5, 6, 7, 8, 9, or 10 nucleotide residues 5′ of the DNA region. Examples of gapmers are provided in TABLE 5.

TABLE 5
Exemplary Gapmer Sequences

| SEQ ID NO | Sequence | Target |
|---|---|---|
| SEQ ID NO: 383 | 5′-TGACTTGTCAAGTCATCCTT-3′ | MAPT |
| SEQ ID NO: 384 | 5′-GCAGGTTAAGTGATTAACCA-3′ | MAPT |
| SEQ ID NO: 385 | 5′-TCCTCTCCACAATTATTGAC-3′ | MAPT |
| SEQ ID NO: 386 | 5′-GACGTATTTAGGAGAGGAAG-3′ | MAPT |
| SEQ ID NO: 387 | 5′-CTGATACTATGCATGTGGAG-3′ | MAPT |
| SEQ ID NO: 388 | 5′-GCCGGGTCATTATTCTTTTT-3′ | MAPT |
| SEQ ID NO: 492 | 5′-GTTGGTACATAACGTTTTGA-3′ | CBP |
| SEQ ID NO: 493 | 5′-TGAATTCCAGGATAACCTGA-3′ | CBP |
| SEQ ID NO: 494 | 5′-CCCGAACACTAAGTGTTAAT-3′ | CBP |
| SEQ ID NO: 495 | 5′-ACCATGTACATGAATTCAAG-3′ | CBP |
| SEQ ID NO: 496 | 5′-AATCTTCTCGAGTTTCGTAT-3′ | CBP |
| SEQ ID NO: 497 | 5′-TATAGGAATTAGAGCGCTTG-3′ | CBP |
| SEQ ID NO: 498 | 5′-AAAGAAGGCTTCTTCTCTAG-3′ | CBP |
| SEQ ID NO: 499 | 5′-GGCTTCAGCCATTATGTATA-3′ | CBP |
| SEQ ID NO: 500 | 5′-GGGATACATTATAAGCTTGC-3′ | CBP |
| SEQ ID NO: 501 | 5′-TTGATTTGAGAATGTTCAGC-3′ | CBP |
| SEQ ID NO: 502 | 5′-GCTTGGGTATTTTTTGATCA-3′ | CBP |
| SEQ ID NO: 503 | 5′-TCCATGGGATTCTTTACGAT-3′ | CBP |
| SEQ ID NO: 504 | 5′-TGGGGTTAAATGAATTCATC-3′ | CBP |
| SEQ ID NO: 505 | 5′-CTTGTTTATGTAAACGCGAC-3′ | CBP |
| SEQ ID NO: 506 | 5′-TGGGTTACTTAAAGAAGTGG-3′ | CBP |
| SEQ ID NO: 507 | 5′-CCAATTGTGTTTTGAATTCC-3′ | CBP |
| SEQ ID NO: 508 | 5′-CAAAACGTTTTTCATGGTTC-3′ | CBP |
| SEQ ID NO: 509 | 5′-TTGATATCTGTAGGGAAGGT-3′ | CBP |
| SEQ ID NO: 510 | 5′-TAAAGTTAGCATTCATGCAG-3′ | CBP |
| SEQ ID NO: 511 | 5′-TAAAAGGCCTAATTCTCCTC-3′ | CBP |
| SEQ ID NO: 512 | 5′-CGGCCATTTTTAATTCTTTC-3′ | p300 |
| SEQ ID NO: 513 | 5′-GACAGCTGTTTATGTTTAGA-3′ | p300 |
| SEQ ID NO: 514 | 5′-CTGAGTATATGGTGAACCAT-3′ | p300 |
| SEQ ID NO: 515 | 5′-GTGAAATGATTTGTCGAGAA-3′ | p300 |
| SEQ ID NO: 516 | 5′-AGCATATGCAACTAGGTTTT-3′ | p300 |
| SEQ ID NO: 517 | 5′-GTGAATTCTGATGAAGAGCT-3′ | p300 |
| SEQ ID NO: 518 | 5′-TGTGCTACTAGTAGATGGAG-3′ | p300 |
| SEQ ID NO: 519 | 5′-CATCTTCACTTCCTGGGAAG-3′ | p300 |
| SEQ ID NO: 520 | 5′-TTTCCGGTTATATAACCAGG-3′ | p300 |
| SEQ ID NO: 521 | 5′-GTATTGTGCACAACTGTTTG-3′ | p300 |
| SEQ ID NO: 522 | 5′-ATCTGATGCATCTTTCTTCC-3′ | p300 |
| SEQ ID NO: 523 | 5′-GTGCACTTTTCTTTAAACAG-3′ | p300 |
| SEQ ID NO: 524 | 5′-TTTCATGATAGACTGCAGTC-3′ | p300 |
| SEQ ID NO: 525 | 5′-TCCATGGTGGCATATAGTTT-3′ | p300 |
| SEQ ID NO: 526 | 5′-CTTTGAAAAATTTGCACGTG-3′ | p300 |
| SEQ ID NO: 527 | 5′-TTTTGTAAGGCTTGTTGAGA-3′ | p300 |
| SEQ ID NO: 528 | 5′-GAGGTTTGAATTCAAGTCTG-3′ | p300 |
| SEQ ID NO: 529 | 5′-GTCTTTTAGGCTACGAAAGA-3′ | p300 |
| SEQ ID NO: 530 | 5′-TAGAAAATTTTCTGGAGCAC-3′ | p300 |
| SEQ ID NO: 531 | 5′-TACAACAGTTGAGGGTTTTT-3′ | p300 |

The second conventional ASO simply serves to bind to the target transcript, but not induce RNase degradation, so no DNA-based-sugars are used. Instead, binding is designed to disrupt processing into mature mRNA. One such activity relies on binding to the mRNA at or near splice sites to drive particular splice isoforms in the target RNA, resulting in modulating target RNA by disrupting mRNA splicing and resulting in exon skipping. These are commonly called “splice blocking” or “splice blocker” ASOs amongst other known names. One example is eteplirsen, designed to alter splicing of DMD (dystrophin) gene in Duchenne Muscular Dystrophy patients, correcting a mutation that would otherwise create a truncated and non-functional dystrophin by splicing out the mutant exons and creating a different truncated (but functional) protein to appear.

Another example is siRNA molecules which specifically interact with the canonical RNAi pathway (the RISC complex) to drive cleavage or steric blocking of hybridized transcripts; cleavage-vs-blocking depends on whether the match is perfect (cleavage) or imperfect but still stable (blocking). Length is typically a double-stranded RNA where the overlapping region is 19-22 and each strand has two extra nt at their 3′ ends. Chemistry is largely RNA-based-sugars, with some DNA-based sugars at the 3′ overhangs. Clinical examples include patisiran (targets TTR) and givosiran (targets ALAS1). In some embodiments, an overlapping region of a siRNA may comprise from about 10 to about 40, from about 10 to about 35, from about 10 to about 30, from about 10 to about 25, from about 10 to about 22, from about 10 to about 21, from about 10 to about 20, from about 15 to about 40, from about 15 to about 35, from about 15 to about 30, from about 15 to about 25, from about 15 to about 22, from about 15 to about 21, from about 15 to about 20, from about 17 to about 40, from about 17 to about 35, from about 17 to about 30, from about 17 to about 25, from about 17 to about 22, from about 17 to about 21, from about 17 to about 20, from about 18 to about 40, from about 18 to about 35, from about 18 to about 30, from about 18 to about 25, from about 18 to about 22, from about 18 to about 21, from about 18 to about 20, from about 19 to about 40, from about 19 to about 35, from about 19 to about 30, from about 19 to about 25, from about 19 to about 22, from about 19 to about 21, or from about 19 to about 20 nucleotide residues. In some embodiments, an overhang region may comprise about 1, 2, 3, 4, 5, 6, 7, 8, 9, or 10 nucleotide residues. Examples of siRNAs are provided in TABLE 6.

TABLE 6
Exemplary siRNA Sequences

| SEQ ID NO | Sequence | Target |
|---|---|---|
| SEQ ID NO: 389 | 3′-AAGUGAUUCGUUGUGUUAUAA-5′ | IL-23R |
| SEQ ID NO: 390 | 5′-AAUUCACUAAGCAACACAAUA-3′ | |
| SEQ ID NO: 391 | 3′-UAGAAGUGUACCUGUGUACAA-5′ | IL-23R |
| SEQ ID NO: 392 | 5′-AAAUCUUCACAUGGACACAUG-3′ | |
| SEQ ID NO: 393 | 3′-CGACGGAACGUUAGACUUGAA-5′ | IL-23R |
| SEQ ID NO: 394 | 5′-AAGCUGCCUUGCAAUCUGAAC-3′ | |
| SEQ ID NO: 395 | 3′-CCACCGUUCUUCAUGAACCAA-5′ | IL-23R |
| SEQ ID NO: 396 | 5′-AAGGUGGCAAGAAGUACUUGG-3′ | |
| SEQ ID NO: 532 | 3′-AUCAUUGAGACCGGUAUCGAA-5′ | CBP |
| SEQ ID NO: 533 | 5′-AAUAGUAACUCUGGCCAUAGC-3′ | |
| SEQ ID NO: 534 | 3′-GUCGUCUACUUCGUCGUCUAA-5′ | CBP |
| SEQ ID NO: 535 | 5′-AACAGCAGAUGAAGCAGCAGA-3′ | |
| SEQ ID NO: 536 | 3′-UUAGGUCCGUAGAUCCAAGAA-5′ | CBP |
| SEQ ID NO: 537 | 5′-AAAAUCCAGGCAUCUAGGUUC-3′ | |
| SEQ ID NO: 538 | 3′-ACAAUGAUCUCUUCUUCGGAA-5′ | CBP |
| SEQ ID NO: 539 | 5′-AAUGUUACUAGAGAAGAAGCC-3′ | |
| SEQ ID NO: 540 | 5′-AAUCUAUCUUCAGUAGCUUGU-3′ | p300 |
| SEQ ID NO: 541 | 5′-AAACAAGCUACUGAAGAUAGA-3′ | p300 |
| SEQ ID NO: 542 | 5′-AAUAAGUGGCAUCACGAGGUA-3′ | p300 |
| SEQ ID NO: 543 | 5′-AAUACCUCGUGAUGCCACUUA-3′ | p300 |
| SEQ ID NO: 544 | 5′-AAGUCUGGUAGAUGGCAACCU-3′ | p300 |
| SEQ ID NO: 545 | 5′-AAAGGUUGCCAUCUACCAGAC-3′ | p300 |

Another example are anti-miRs. Anti-miRs may function as steric blockers designed against miRNAs that would block a RISC complex loaded with a specific disease-associated miRNA without being subject to cleavage by the RISC complex RNase subunit. One clinical example is miravirsen, a 15-base oligo with a mixture of DNA and LNA sugars that targets miR-122 in hepatitis C patients. An anti-miR nucleotide may be of sufficient length to anneal specifically and stably to the target miR, but the length of the sequence may vary. For example, an anti-miR may have a length of up to about 21 nt, corresponding to the maximum size loaded into RISC. In some embodiments, an anti-miR nucleotide may comprise from about 10 to about 25, from about 10 to about 23, from about 10 to about 21, from about 10 to about 20, from about 10 to about 19, from about 10 to about 18, from about 13 to about 25, from about 13 to about 23, from about 13 to about 21, from about 13 to about 20, from about 13 to about 19, from about 13 to about 18, from about 15 to about 25, from about 15 to about 23, from about 15 to about 21, from about 15 to about 20, from about 15 to about 19, from about 15 to about 18, from about 16 to about 25, from about 16 to about 23, from about 16 to about 21, from about 16 to about 20, from about 16 to about 19, or from about 16 to about 18 nucleotide residues.

Another example is U1 adapters which have two parts. One anneals to the U1-snRNA of the U1-snRNP complex, and the other binds to the target RNA, bringing the U1-snRNP to the polyA site and inhibiting polyadenylation; absence of a polyA tail causes the mRNA to be degraded. The U1-binding region is at least 10 nt but up to 19 nt. Target binding region can be from about 15 nt to about 25 nt. Chemistry in early studies made heavy use of LNA and 2′-O-Methyl sugars. In some embodiments, a U1 binding region may comprise from about 10 to about 25, from about 10 to about 23, from about 10 to about 21, from about 10 to about 20, from about 10 to about 19, from about 10 to about 18, from about 13 to about 25, from about 13 to about 23, from about 13 to about 21, from about 13 to about 20, from about 13 to about 19, from about 13 to about 18, from about 15 to about 25, from about 15 to about 23, from about 15 to about 21, from about 15 to about 20, from about 15 to about 19, from about 15 to about 18, from about 16 to about 25, from about 16 to about 23, from about 16 to about 21, from about 16 to about 20, from about 16 to about 19, or from about 16 to about 18 nucleotide residues. In some embodiments, a target binding region may comprise from about 10 to about 40, from about 10 to about 35, from about 10 to about 30, from about 10 to about 25, from about 10 to about 22, from about 10 to about 21, from about 10 to about 20, from about 15 to about 40, from about 15 to about 35, from about 15 to about 30, from about 15 to about 25, from about 15 to about 22, from about 15 to about 21, from about 15 to about 20, from about 17 to about 40, from about 17 to about 35, from about 17 to about 30, from about 17 to about 25, from about 17 to about 22, from about 17 to about 21, from about 17 to about 20, from about 18 to about 40, from about 18 to about 35, from about 18 to about 30, from about 18 to about 25, from about 18 to about 22, from about 18 to about 21, from about 18 to about 20, from about 19 to about 40, from about 19 to about 35, from about 19 to about 30, from about 19 to about 25, from about 19 to about 22, from about 19 to about 21, or from about 19 to about 20 nucleotide residues.

Another example of a nucleotide of the present disclosure is an aptamer. Aptamers disrupt target activity using a mechanism that differs from other nucleotides described herein that form base pairing interactions with a target nucleotide. Aptamers are nucleic acids that form secondary structures (e.g., where a single strand of nucleic acid base-pairs with itself upon folding, creating loops in various locations). Aptamers may be screened for interaction with target proteins. Aptamers may have varied nucleotide chemistry and may include a mixture of conventional RNA and/or DNA sugars and modified sugars (e.g., 2′-O-Methyl (2′-O-Me) RNA or 2′-Fluoro (2′-F) RNA sugars). For example, one clinically approved aptamer, pegaptanib (a VEGF-binding aptamer), has a mixture of 2′-O-Methyl (2′-O-Me) RNA and 2′-Fluoro (2′-F) RNA sugars and regular RNA and DNA sugars. An aptamer sequence may be long enough to form a stable secondary structure (e.g., through intramolecular base pairing), but the length may vary. In some embodiments, an aptamer sequence may comprise from about 20 nt to about 40 nt. For example, experiments that identified pegaptanib used oligos of 20-40 nt in length. Shorter nucleotides (e.g., sequences shorter than about 40 nt) may be advantageous, as longer oligonucleotides may complicate nucleotide synthesis or engage the interferon response pathway. In some embodiments, an aptamer may comprise from about 15 to about 60, from about 15 to about 50, from about 15 to about 40, from about 15 to about 35, from about 15 to about 30, from about 20 to about 60, from about 20 to about 50, from about 20 to about 40, from about 20 to about 35, from about 20 to about 30, from about 25 to about 60, from about 25 to about 50, from about 25 to about 40, from about 25 to about 35, or from about 25 to about 30 nucleotide residues.

Nucleotides may be designed for use in the peptide nucleotide complexes of the present disclosure. In some embodiments, nucleotides that modify processing, translation, or other RNA functions (e.g., a gapmer, splice blocker, siRNA, anti-miR, or U1 adapter), have one or more of the following properties: (a) 8-50 nt in length, but preferably 12-30 nt in length. It is understood that any length of a nucleotide (nt) can be used within the foregoing ranges; (b) cross-species homology (e.g., by targeting highly-conserved motifs) is often a desirable feature but is not necessary for activity or clinical development; (c) avoidance of common SNPs in humans unless that SNP is involved in disease pathology (e.g., an allele-specific oligo) is often a desirable feature but is not necessary for activity or clinical development; (d) gene specificity (they have minimal homology to other sequences; for example, a sequence may have 3 or more mismatches to every other sequence). (e) avoid predicted secondary structures in both the oligo and the target region (there are software tools available to screen in silico for such secondary structure formation); (f) higher G/C content may be preferable, as G/C-rich sequences (e.g. CCAC, TCCC, GCCA) may be helpful for increasing affinity of the nucleotide to its target, whereas A/T-rich sequences (e.g. TAA) or runs of 4+G (GGGG) may exhibit low or result in structural (G-quadruplex) formation. An oligonucleotide sequence can be 60%, 70%, 75%, 80%, 85%, 90%, 91%, 92%, 93%, 94%, 95%, 96%, 97%, 98%, 99%, or 100% complementarity or match to the target sequence. In some situations, an oligonucleotide with 100% complementarity will result in the target RNA being degraded. In some situations, an oligonucleotide that is less than 100% complementarity may not lead to degradation of the target RNA but may prevent translation and production of the encoded protein.

In some embodiments, gapmers have one or more of the following properties: (a) 12-30 nt in length. It is understood that any length of a nucleotide (nt) can be used within the foregoing range. (b) target sites are anywhere in the pre-mRNA, including UTRs, exons, or introns (c) central DNA region: minimum of 4 contiguous DNA nucleotides, often 10 or more are used. No artificial substitutions at 2′ site (e.g. 2′-O-methyl [2′-O-ME] or 2′-O-methoxyethyl [2′-O-MOE]) are tolerated due to requirements of RNase H recognition. (d) flanking region: can be DNA- or RNA-based-sugars. 2′ substitutions such as 2′-O-ME or 2′-O-MOE are tolerated. Linked nucleic acids (LNA) and morpholino (phosphorodiamidate morpholino oligo) chemistry are also acceptable in flanking region. (e) Backbone can be natural phosphodiester (PO) or non-natural phosphorothioate (PS) linkages. A clinical example is fomivirsen, a 21 nt gapmer wherein the whole oligo is PS-backbone DNA. Another example is mipomersen, a 20 nt gapmer wherein the entire backbone is PS linkages, and the central region uses DNA sugar flanked by 2′-O-MOE modified RNA. For these two examples, all C bases are 5-methyl-C, though this is not a strict requirement for engagement of RNase H1. Similarly, thiophosphorodiamidate chemistries may be used.

In some embodiments, steric blockers have one or more of the following properties; (a) as the molecule does not need to engage RNase H or any other enzyme, backbone and sugar chemistry can be more varied, (g) target sites for the nucleotide are complementary to one or more splice sites in the target RNA. A clinical example is eteplirsen, a 30 nt splice blocking ASO wherein whole oligonucleotide uses morpholino (Phosphorodiamidate morpholino oligo) chemistry. Another clinical example is nusinersen, an 18 nt ASO, whose backbone is entirely PS linked and uses 2′-O-MOE RNA chemistry. All C bases are 5-methyl-C, though this is not a strict requirement for engagement of RNase H1. Similarly, thiophosphorodiamidate chemistries may be used.

In some embodiments, siRNA have one or more of the following properties: (a) can be between 15 and 25 nt in length (between 13 to 23 nt overlap respectively), or up to 25 nt (23 nt overlap) per strand, but 21 nt (19 nt overlap) is common. It is understood that any length of a nucleotide (nt) overlap can be used within the foregoing ranges; (b) complements a sequence typically but not exclusively of 21-nt length in the target mRNA that typically but not exclusively begins with “AA” (c) target sites are ideally found in the mature spliced mRNA as the RISC complex for RNA cleavage is primarily cytosolic; (d) preferably but not exclusively avoids sequences within 100 nt of the mRNA start site, as the transcript at start site is more likely to be occupied by RNA polymerase, (e) successful siRNA constructs typically have more G/C at 5′ end of sense strand, more A/T at 3′ end of sense strand, and are roughly 30-60% in G/C content.

In some embodiments, anti-miR (anti-miRNA) have one or more of the following properties: (a) a perfect match to target sequence (specifically the 5′ end of the guide strand of the miRNA); (b) length can vary and can even be greater than the length of the mature guide strand. Screening for effective anti-miR constructs may begin with the shortest sequence that achieves specificity (no off-target homology) and increase length from there to empirically determine ideal minimal length for strong miRNA inhibition; (c) 2′ sugar modifications (2′-O-Me, 2′-O-MOE, 2′-F) and LNA sugars are commonly used. Sugars can be a mixture. A clinical example of an anti-miR is miravirsen, which uses a mixture of DNA and LNA sugars (d) PS linkages in backbone are common. PS linkages may reduce affinity, but sugar modifications may increase affinity.

In some embodiments, aptamers have one or more of the following properties: (a) length of aptamers can vary widely, as there is no biological complex (e.g., RISC) they interact with to function. Although composed of nucleic acids, they are more protein-like in function (e.g., bind to a target protein, etc.). The minimum length may be determined empirically to maintain sufficient stability of intra-strand hybridization to fold into a secondary structure, the upper limit on size is limited only by pharmacology, as longer sequences have a higher risk of engaging inflammatory pathways. Aptamer screening typically begins with libraries of 20-40 nt in length (not including flanking regions required for library amplification during screening); (b) as they form interactions via secondary structure rather than base pairing interactions, there are few limitations for their base patterns, since secondary structures are not only desirable but essential to their function. Design may be empirical for each target; (c) selection is typically via Systematic Evolution of Ligands by EXponential Enrichment (SELEX): random or semi-random sequences between primer-binding flanking regions are exposed to a target of interest on a solid substrate. The pooled oligonucleotide mixture is rinsed from the substrate, leaving only sequences that interact with the target remaining, and then binding sequences are eluted and amplified by PCR. (d) Sugar modifications commonly used include 2′-fluoro (2′-F), 2′-O-MOE, and 2′-O-Me, though other chemistries including (but not limited to) LNA and unlocked-nucleic-acids (UNA) are also possible; (e) backbones are typically PO or PS, but other linkages such as methylphosphonate are possible. A clinical aptamer example, pegaptanib, is entirely PO backbone, but others in development use other linkages. (f) aptamer termini are typically capped with unnatural nucleotide chemistries (e.g. 3′ inverted thymidine) or biotinylated nucleotides to reduce susceptibility to nucleases; (g) because activity is not based on base-pairing, aptamers can be much more creative with chemical modifications of the bases themselves; these can include bases designed to induce covalent bonds with target proteins to permanently disable them; (h) such modifications are tested after selection of an active, high affinity aptamer, as unmodified bases are required for nucleic acid amplification during SELEX (i) if the target protein is extracellular, less considerations are necessary than for cell penetration capabilities.

In some embodiments, other general design considerations aimed at enhancing pharmacokinetic (PK) properties of the nucleotide, peptide, or peptide oligonucleotide complex include one or more of the following properties: (a) building in conjugation to moieties that reduce clearance or increase cellular uptake including cholesterol or other lipids, diacylglycerol, GalNAc, palmitoyl, PEG, an RGD motif, cell penetrating peptides or moieties (e.g., a PD-L1-binding peptide or cell penetrating peptide as described herein). Adding cholesterol to the peptide oligonucleotide complex can improve biodistribution to the target tissue, increase cellular uptake by endocytosis, and alter the serum pharmacokinetics.

The therapeutic activity and molecular method of the peptide oligonucleotide complex may depend on which target molecule (e.g., a DNA or RNA) that the nucleic acid complements, or in the case of an aptamer, which target molecule (e.g., protein or other macromolecule) it binds. Target choice can fall into one or more non-mutually exclusive categories such as tissue-target-based or disease-selective. Known targets have known mRNA and genomic sequences that can be used to design a variety of complementary nucleic acids for use in the peptide nucleotide complexes described herein depending on the activity (e.g., gene regulation, protein degradation, reduction of cancer cell activators) desired. Examples of targets are provided in SEQ ID NO: 397-SEQ ID NO: 430 or SEQ ID NO: 549, TABLE 10, and TABLE 17. For example, tissue-targeting may comprise selecting targets acting in the tissues where a PD-L1-binding peptide portion of the peptide oligonucleotide complex would preferentially access or accumulate. For example, serum proteins produced in the liver may be targeted, such as by a TTR to treat transthyretin-related amyloid diseases, or by various apolipoproteins to treat hypercholesterolemia or cardiovascular disease. Moreover, proteins produced in the lungs or lung tissue expressing PD-L1 may be targeted, used to target inflammatory cytokines or cytokine receptors to treat pulmonary disorders (e.g., COPD), or to downregulate receptors (e.g., ACE2) that determine tropism of airborne viruses (e.g., SARS-CoV-2).

Alternatively, targets can be selected that act in areas where the PD-L1-binding peptide accumulates (e.g., for example in PD-L1-expressing cancer cells), based on known expression of PD-L1 in the tissue or target cell type, or that act where an oligonucleotide that is not complexed with the PD-L1-binding peptide would otherwise be excluded, including tumors. Tumor targeting can be used for peptide oligonucleotide complexes of this disclosure as tumors often have high levels of PD-L1 and are often vascularized enough for rapid perfusion of serum-resident PD-L1-binding peptides and their oligonucleotide cargo. Targets for the peptide oligonucleotide complexes can include oncogenes, for example by designing the nucleic acid portion of the complex to target overexpressed genes or those for which the tumor is lacking a redundant ortholog (i.e., normal cells function by using X or Y, tumors do not express Y, so X is targeted). In addition, disease-selective targeting can be used to treat conditions where the transcript is selectively found in the diseased tissue, and preferentially accumulate there, to improve safety and reduce off-target effects. PD-L1 can also be expressed on tissues such as colon or other gastrointestinal tissue, skeletal muscle, adipose tissue, lymphoid tissue, soft tissue, placenta, seminal vesicles, tonsils, and resting or activated T cells, B cells, dendritic cells, and macrophages. Thus PD-L1 binding peptides of this disclosure can be used to deliver oligonucleotides or other active agents to those tissues.

The target-binding agent (e.g., a nucleotide of a peptide oligonucleotide complex) may be capable of binding the targets described in SEQ ID NO: 397-SEQ ID NO: 430 or SEQ ID NO: 549, TABLE 10, or TABLE 17, or to nucleotides (e.g., DNA or RNA molecules) encoding the proteins listed in SEQ ID NO: 397-SEQ ID NO: 430 or SEQ ID NO: 549, TABLE 10, or TABLE 17, or otherwise described herein. Examples of nucleotide sequences that may be used in the nucleotide portion of the peptide oligonucleotide complex include SEQ ID NO: 366-SEQ ID NO: 396 and SEQ ID NO: 492-SEQ ID NO: 545. It is understood that any oligonucleotide may be used that is complementary to a portion of the target DNA or RNA molecule. Such target binding agent may comprise a nucleotide sequence is single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter. Such oligos may be about 5 to 30 nt in length, 10 to 25 nt in length, 15 to 25 nt in length, 19 to 23 nt in length, or at least 10 nt in length, at least 15 nt in length, at least 20 nt in length, at least 25 nt in length, or at least 30 nt in length, at least 50 nt in length, at least 100 nucleotides in length across any portion of the target RNA. Examples of sequences to which such oligonucleotides may bind (e.g., are complementary to) include SEQ ID NO: 397-SEQ ID NO: 430 and SEQ ID NO: 546-SEQ ID NO: 549, or any genomic or ORF sequence referenced in TABLE 17. One of skill in the art can readily design or determine the length of the target binding agent and whether the target binding agent is complementary to the reference target RNA sequence, and can thus determine using the chemistry of RNA and DNA where such target binding agent will bind to such reference target RNA sequence for the designed length across any portion of the target RNA. Consequently, for any RNA target described herein, including for any of the targets or molecules encoding the targets described in TABLE 10, and SEQ ID NO: 397-SEQ ID NO: 430 and SEQ ID NO: 546-SEQ ID NO: 549, or any genomic or ORF sequence referenced in TABLE 17 such target binding agent of any nt length is described.

In some embodiments, a nucleotide binds to the target molecule with a melting temperature of not less than 37° C. and not more than 99° C. In some embodiments, a nucleotide binds to the target molecule with a melting temperature of not less than 40° C. and not more than 85° C., not less than 40° C. and not more than 65° C., not less than 40° C. and not more than 55° C., not less than 50° C. and not more than 85° C., not less than 60° C. and not more than 85° C., or not less than 55° C. and not more than 65° C.

In some embodiments, a nucleotide binds the target molecule with an affinity of not more than 500 nM, not more than 100 nM, not more than 50 nM, not more than 10 nM, not more than 1 nM, not more than 500 pM, not more than 400 pM, not more than 300 pM, not more than 200 pM, or not more than 100 pM. In some embodiments, a nucleotide binds the target molecule with an affinity of not more than 500 nM and not less than 100 pM, not more than 100 nM and not less than 200 pM, not more than 50 nM and not less than 300 pM, not more than 10 nM and not less than 400 pM, or not more than 1 nM and not less than 500 pM.

In some embodiments, a nucleotide comprises at least 90%, at least 92%, at least 93%, at least 94%, at least 95%, at least 96%, at least 97%, at least 98%, or at least 99% sequence identity to any one of SEQ ID NO: 366-SEQ ID NO: 396. In some embodiments, a nucleotide comprises a sequence of any one of SEQ ID NO: 366-SEQ ID NO: 396, any one of SEQ ID NO: 366-SEQ ID NO: 396 wherein U is replaced with T, or any one of SEQ ID NO: 366-SEQ ID NO: 396 wherein T is replaced with U. In some embodiments, a nucleotide comprises no more than 1, 2, 3, 4, or 5 base changes relative to a sequence of any one of SEQ ID NO: 366-SEQ ID NO: 396.

In some embodiments, a nucleotide is at least 60%, at least 70%, at least 80%, at least 90%, at least 92%, at least 93%, at least 94%, at least 95%, at least 96%, at least 97%, at least 98%, at least 99%, or 100% reverse complementary to the target molecule. In some embodiments, a nucleotide is 100% reverse complementary to the target molecule. In some embodiments, a nucleotide comprises no more than 1, 2, 3, 4, or 5 base pair mismatches upon binding to the target molecule. In some embodiments, a nucleotide comprises at least 1, 2, 3, 4, or 5 base pair mismatches upon binding to the target molecule.

In some embodiments, a nucleotide may modulate an activity of a target molecule. In some embodiments, modulating the activity of the target molecule comprises reducing expression of the target molecule, increasing the expression of the target molecule, reducing translation of the target molecule, degrading the target molecule, reducing a level of the target molecule, modifying the processing of the target molecule, modifying the splicing of the target molecule, inhibiting processing of the target molecule, reducing a level of a protein encoded by the target molecule, or blocking an interaction with the target molecule. In some embodiments, the expression of the target molecule is reduced by at least 10%, 25%, 30%, 40%, 50%, 60%, 75%, 80%, 90%, 95%, 99%, 99.5%, or 99.9%. In some embodiments, the translation of the target molecule is reduced by at least 10%, 25%, 30%, 40%, 50%, 60%, 75%, 80%, 90%, 95%, 99%, 99.5%, or 99.9%. In some embodiments, the expression of the target molecule is reduced by a factor of at least 2, 4, 8, 10, 15, 16, 20, 32, 50, 64, 100, 128, 200, 256, 500, 512, or 1000. In some embodiments, the translation of the target molecule is reduced by a factor of at least 2, 4, 8, 10, 15, 16, 20, 32, 50, 64, 100, 128, 200, 256, 500, 512, or 1000. In some embodiments, at least 10%, 25%, 30%, 40%, 50%, 60%, 75%, 80%, 90%, 95%, 99%, 99.5%, or 99.9% of the target molecule is degraded. In some embodiments, the level of the protein encoded by the target molecule is reduced by at least 10%, 25%, 30%, 40%, 50%, 60%, 75%, 80%, 90%, 95%, 99%, 99.5%, or 99.9%. In some embodiments, modifying the splicing of the target molecule increases a level of a protein encoded by the target molecule by at least 10%, 25%, 30%, 40%, 50%, 60%, 75%, 80%, 90%, 95%, 99%, 99.5%, or 99.9%.

A peptide oligonucleotide complex of the present disclosure may comprise a nucleotide complexed with a protein (e.g., a PD-L1-binding peptide). The nucleotide may comprise single stranded DNA, single stranded RNA, double stranded DNA, double stranded RNA, or combinations thereof. In some embodiments, a nucleotide of a peptide oligonucleotide complex may be non-naturally occurring, also referred to as an “engineered nucleotide”. In some embodiments, a nucleotide may comprise a naturally occurring sequence. A nucleotide may be exogenously expressed, enzymatically synthesized in vitro, or chemically synthesized. For example, a nucleotide may be expressed in a bacterial, yeast, or mammalian cell line and purified for use in a peptide oligonucleotide complex of the present disclosure. In another example, a nucleotide may be enzymatically synthesized in vitro using an RNA or DNA polymerase. In another example, a nucleotide may be chemically synthesized on a solid support using protected nucleotides.

One example of a chemical synthesis method that may be used to prepare a nucleotide for use in a peptide oligonucleotide complex of the present disclosure is phosphoramidite synthesis. Briefly, single nucleotide residues may be sequentially added from 3′ to 5′ to the growing nucleotide chain by repeating the steps of de-blocking (detritylation), coupling, capping, and oxidation. Phosphoramidite synthesis may be performed on a solid support such as controlled pore glass (CPG) or macroporous polystyrene (MPPS). Similarly, thiophosphorodiamidate may be used.

A nucleotide of a peptide oligonucleotide complex may bind to a target molecule (e.g., a target DNA, a target RNA, or a target protein). In some embodiments, binding of the oligonucleotide to the target molecule may alter an activity of the target molecule. For example, binding of an oligonucleotide (e.g., an siRNA, an miRNA, a gapmer, or a U1 adaptor) to a target mRNA or pre-mRNA may increase or decrease translation of the target mRNA or pre-mRNA. In another example, binding of a nucleotide to a target DNA may increase or decrease expression of a gene encoded by the target DNA. In another example, binding of a nucleotide to an RNA (such as a transcript, pre-RNA, unspliced RNA, nuclear RNA, complimentary sequence to a NAT, or mRNA) expressed from a target DNA such as a gene or ORF may increase or decrease expression of a gene encoded by the target DNA. In another example, binding of an oligonucleotide (e.g., an aptamer) to a target protein may increase or decrease activity (e.g., an enzymatic activity or a binding activity) of the target protein. In some embodiments, the target molecule may be associated with a disease or condition and increasing or decreasing the activity of the target molecule may treat the disease or condition.

A sequence of the oligonucleotide of a peptide oligonucleotide complex may be selected for its ability to bind to or modulate the activity of a target molecule. In some embodiments, an oligonucleotide may be reverse complementary to a target DNA or RNA molecule. For example, an siRNA oligonucleotide may be reverse complementary to a target RNA molecule. In some embodiments, am oligonucleotide may be partially reverse complementary (e.g., comprising one or more mis-matched base pairs) to a target DNA or RNA molecule. For example, an siRNA oligonucleotide may comprise a base mismatch relative to a target RNA molecule. In some embodiments, a sequence of the oligonucleotide may be selected for its annealing temperature relative to a target DNA or RNA molecule. A preferred annealing temperature may be achieved by selecting the length of the nucleotide, the degree of complementarity of the nucleotide to the target molecule, the chemistry of the nucleotides, or any combination thereof. Nucleotide sequence parameters (e.g., complementarity, annealing temperature, melting temperature, base mismatches, and binding affinity) may be calculated using any available software, such as ITD OligoAnalyzer and the like. In some embodiments, an oligonucleotide may adopt a secondary structure that binds to a target DNA, RNA, or protein molecule. For example, an aptamer may adopt a secondary structure to bind to a target protein. The aptamer sequence may be selected to adopt a secondary structure that binds to a target protein. Nucleotide secondary structure may be predicted using any available software, such as RNAfold and the like. In some embodiments, a nucleotide sequence may be determined experimentally by selecting for the ability to bind to a target molecule. For example, a nucleotide library may be contacted to a target molecule, and sequences that bind to the target molecule may be identified.

In some embodiments, a nucleotide comprises a G/C content of not less than 20% and not more than 80%. In some embodiments, a nucleotide comprises a G/C content of not less than 30% and not more than 65%. In some embodiments, the nucleotide comprises a G/C content of not less than 20%, not less than 25%, not less than 30%, not less than 35%, not less than 40%, not less than 45%, or not less than 50%. In some embodiments, the nucleotide comprises a G/C content of not more than 80%, not more than 75%, not more than 70%, not more than 65%, or not more than 50%. In some embodiments, a nucleotide comprises an A/T content or A/U content of not less than 20% and not more than 80%. In some embodiments, a nucleotide comprises an A/T content or A/U content of not less than 30% and not more than 65%. In some embodiments, the nucleotide comprises a A/U (or A/T, or combination of A/U and A/T) content of not less than 20%, not less than 25%, not less than 30%, not less than 35%, not less than 40%, not less than 45%, or not less than 50%. In some embodiments, the nucleotide comprises a A/U content (or A/T, or combination of A/U and A/T) of not more than 80%, not more than 75%, not more than 70%, not more than 65%, or not more than 50%. In some embodiments, a nucleotide has a length of no more than 1000 nt, 600 nt, 200 nt, 100 nt, 60 nt, 56 nt, 52 nt, 50 nt, 48 nt, 46 nt, 44 nt, 22 nt, 40 nt, 38 nt, 36, nt, 34 nt, 32 nt, 30 nt, or 24 nt. In some embodiments, a nucleotide has a length of from 24 to 100 nt, from 24 to 60 nt, from 24 to 50 nt, or from 36 to 50 nt. In some embodiments, a nucleotide has a length of about 42 nt.

In some embodiments, a nucleotide has a length of no more than 500 nt, 300 nt, 100 nt, 50 nt, 30 nt, 28 nt, 26 nt, 25 nt, 24 nt, 23 nt, 22 nt, 21 nt, 20 nt, 19 nt, 18, nt, 17 nt, 16 nt, 15 nt, or 12 nt. In some embodiments, a nucleotide has a length of from 12 to 50 nt, from 12 to 30 nt, from 12 to 25 nt, from 18 to 25 nt, from 18 to 25 nt, from 19 to 23 nt, or from 20 to 22 nt. In some embodiments, a nucleotide has a length of about 21 nt.

A peptide oligonucleotide complex of the present disclosure (e.g., a peptide oligonucleotide complex comprising a PD-L1-binding peptide and a nucleotide) may be further conjugated, linked, or fused to an active agent in addition to the nucleotide active agent (e.g., a target-binding agent capable of binding a target molecule). Such additional active agent may be complexed, fused, linked or conjugated to one or more of the peptide, nucleotide, or linker within the peptide oligonucleotide complex. In some embodiments, the active agent may be directly or indirectly linked to the peptide of the peptide oligonucleotide complex or the nucleotide of the peptide oligonucleotide complex. A peptide nucleic acid complex further comprising an additional active agent may be referred to as a peptide-active agent conjugate or a peptide construct.

The peptide oligonucleotide complexes of the present disclosure can also be used to deliver another active agent. Peptides according to the present disclosure can be conjugated to, linked to, or fused to an agent for use in the treatment of tumors and cancers or other diseases. For example, in certain embodiments, the peptides described herein are fused or conjugated to another molecule, such as an active agent that provides an additional functional capability. A peptide or nucleotide can be fused with an active agent through expression of a vector containing the sequence of the peptide with the sequence of the active agent. In various embodiments, the sequence of the peptide and the sequence of the active agent can be expressed from the same Open Reading Frame (ORF). In various embodiments, the sequence of the peptide and the sequence of the active agent can comprise a contiguous sequence. The peptide and the active agent can each retain similar functional capabilities in the peptide construct compared with their functional capabilities when expressed separately. In certain embodiments, examples of active agents can include other peptides.

As another example, in certain embodiments, the peptides or nucleotides described herein are attached to another molecule, such as an active agent that provides a functional capability. The active agent may be any active agent (e.g., therapeutic agent, detectable agent, or binding moiety) described herein. In some embodiments, the peptide or nucleotide is covalently or non-covalently linked to an active agent, e.g., directly or via a linker. Exemplary linkers suitable for use with the embodiments herein are discussed in further detail below.

### Modification of Peptides

A peptide can be modified (e.g., chemically modified, mutationally modified, or modified with a peptide) in one or more of a variety of ways. In some embodiments, the peptide can be mutated to add function, delete function, or modify the in vivo behavior. One or more loops between the disulfide linkages of a peptide (e.g., a PD-L1-binding peptide or peptide complex) can be modified or replaced to include active elements from other peptides (such as described in Moore and Cochran, Methods in Enzymology, 503, p. 223-251, 2012). In some embodiments, the peptides of the present disclosure (e.g., PD-L1-binding peptides or peptide complexes) can be further functionalized and multimerized by adding an additional functional domain. For example, an albumin-binding domain from a Finegoldia magna peptostreptococcal albumin-binding protein (SEQ ID NO: 245, MKLNKKLLMAALAGAIVVGGGVNTFAADEPGAIKVDKAPEAPSQELKLTKEEAEKAL KKEKPIAKERLRRLGITSEFILNQIDKATSREGLESLVQTIKQSYLKDHPIKEEKTEETPKY NNLFDKHELGGLGKDKGPGRFDENGWENNEHGYETRENAEKAAVKALGDKEINKSYT ISQGVDGRYYYVL SREEAETPKKPEEKKPEDKRPKMTIDQWLLKNAKEDAIAELKKAGI TSDFYFNAINKAKTVEEVNALKNEILKAHAGKEVNPSTPEVTPSVPQNHYHENDYANIG AGEGTKEDGKKENSKEGIKRKTAREEKPGKEEKPAKEDKKENKKKENTDSPNKKKKE KAALPEAGRRKAEILTLAAASLSSVAGAFISLKKRK). For example, an albumin-binding domain of SEQ ID NO: 243 (LKNAKEDAIAELKKAGITSDFYFNAINKAKTVEEVNALKNEILKA) can be added to a peptide of the present disclosure. In some embodiments, a peptide of the present disclosure can be functionalized with an albumin-binding domain that has been modified for improved albumin affinity, improved stability, reduced immunogenicity, improved manufacturability, or a combination thereof. For example, a peptide can be functionalized with a modified albumin-binding domain of SEQ ID NO: 244 (LKEAKEKAIEELKKAGITSDYYFDLINKAKTVEGVNALKDEILKA) having high thermostability and improved serum half-life compared to the albumin binding domain of SEQ ID NO: 243. The albumin-binding domain comprises a simple three-helical structure that would be unlikely to disturb the independent folding of the other peptide domains (e.g., CDP domains). In some embodiments, a functional domain (e.g., an albumin-binding domain) can increase the serum half-life of a peptide or peptide complex of the present disclosure. A functional domain (e.g., an albumin-binding domain) can be included in any orientation relative to the PD-L1-binding peptide. For example, a functional domain can be linked to the PD-L1-binding peptide. An additional functional domain can be linked to one or more peptides (e.g., a PD-L1-binding peptide or peptide complex) via a linker (e.g., a peptide linker of any one of SEQ ID NO: 154-SEQ ID NO: 241 or SEQ ID NO: 433).

Amino acids of a peptide or a peptide complex (e.g., a PD-L1-binding peptide or peptide complex) can also be mutated, such as to increase half-life, modify, add or delete binding behavior in vivo, add new targeting function, modify surface charge and hydrophobicity, or allow conjugation sites. N-methylation is one example of methylation that can occur in a peptide of the disclosure. In some embodiments, the peptide is modified by methylation on free amines. For example, full methylation can be accomplished through the use of reductive methylation with formaldehyde and sodium cyanoborohydride.

The peptides can be modified to add function, such as to graft loops or sequences from other proteins or peptides onto peptides of this disclosure. Likewise, domains, loops, or sequences from this disclosure can be grafted onto other peptides or proteins such as antibodies that have additional function.

In some embodiments, a PD-L1-binding peptide or peptide complex can comprise a tissue targeting domain and can accumulate in the target tissue upon administration to a subject. For example, PD-L1-binding peptides can be conjugated to, linked to, or fused to a molecule (e.g., small molecule, peptide, or protein) with targeting or homing function for a cell of interest or a target protein located on the surface or inside said cell. In some embodiments, PD-L1-binding peptides can be conjugated to, linked to, or fused to a molecule that extends the plasma and/or biological half-life, or modifies the pharmacodynamic (e.g., enhanced binding to a target protein) and/or pharmacokinetic properties (e.g., rate and mode of clearance) of the peptides, or any combination thereof.

A chemical modification can, for instance, extend the half-life of a peptide or change the biodistribution or pharmacokinetic profile. A chemical modification can comprise a polymer, a polyether, polyethylene glycol, a biopolymer, a polyamino acid, a fatty acid, a dendrimer, an Fc region, a simple saturated carbon chain such as palmitate or myristolate, or albumin. A polyamino acid can include, for example, a poly amino acid sequence with repeated single amino acids (e.g., poly glycine), and a poly amino acid sequence with mixed poly amino acid sequences (e.g., gly-ala-gly-ala (SEQ ID NO: 550)) that can or may not follow a pattern, or any combination of the foregoing.

The peptides of the present disclosure can be modified such that the modification increases the stability and/or the half-life of the peptides. The attachment of a hydrophobic moiety, such as to the N-terminus, the C-terminus, or an internal amino acid, can be used to extend half-life of a peptide of the present disclosure. The peptides can also be modified to increase or decrease the gut permeability or cellular permeability of the peptide. In some cases, the peptides of the present disclosure show high accumulation in glandular cells of the intestine, demonstrating applicability in the treatment and-or prevention of diseases or conditions of the intestines, such as Crohn's disease or more generally inflammatory bowel diseases. The peptide of the present disclosure can include post-translational modifications (e.g., methylation and/or amidation and/or glycosylation), which can affect, e.g., serum half-life. In some embodiments, simple carbon chains (e.g., by myristoylation and/or palmitylation) can be conjugated to, linked to, the fusion proteins or peptides. The simple carbon chains can render the fusion proteins or peptides easily separable from the unconjugated material. For example, methods that can be used to separate the fusion proteins or peptides from the unconjugated material include, but are not limited to, solvent extraction and reverse phase chromatography. Lipophilic moieties can extend half-life through reversible binding to serum albumin. Conjugated moieties can, e.g., be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be conjugated to, linked to, myristic acid (tetradecanoic acid) or a derivative thereof. In other embodiments, the peptides of the present disclosure can be coupled (e.g., conjugated, linked, or fused) to a half-life modifying agent. Examples of half-life modifying agents can include, but is not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, albumin, or a molecule that binds to albumin. In some embodiments, the half-life modifying agent can be a serum albumin binding peptide, for example SA21 (SEQ ID NO: 242, RLIEDICLPRWGCLWEDD). In some embodiments, a SA21 peptide can be conjugated or fused to the CDPs of the present disclosure (e.g., any of SEQ ID NO: 1-SEQ ID NO: 118, SEQ ID NO: 435, SEQ ID NO: 436, or SEQ ID NO: 554-SEQ ID NO: 567). Additionally, conjugation of the peptide to a near infrared dye, such as Cy5.5, or to an albumin binder such as Albu-tag can extend serum half-life of any peptide as described herein. In some embodiments, immunogenicity is reduced by using minimal non-human protein sequences to extend serum half-life of the peptide.

In some embodiments, the first two N-terminal amino acids (GS) of SEQ ID NO: 60-SEQ ID NO: 118, SEQ ID NO: 435, SEQ ID NO: 436, or SEQ ID NO: 554-SEQ ID NO: 567 serve as a spacer or linker in order to facilitate conjugation or fusion to another molecule, as well as to facilitate cleavage of the peptide from such conjugated to, linked to, or fused molecules. In some embodiments, the fusion proteins or peptides of the present disclosure can be conjugated to, linked to, or fused to other moieties that, e.g., can modify or effect changes to the properties of the peptides.

In some embodiments, peptides or peptide complexes of the present disclosure can also be conjugated to, linked to, or fused to other affinity handles. Other affinity handles can include genetic fusion affinity handles. Genetic fusion affinity handles can include 6×His (HHHHHH (SEQ ID NO: 248); immobilized metal affinity column purification possible), FLAG (DYKDDDDK (SEQ ID NO: 432); anti-FLAG immunoprecipitation), and “shorty” FLAG (DYKDE (SEQ ID NO: 431). In some embodiments, peptides or peptide complexes of the present disclosure can also be conjugated to, linked to, or fused to an expression tag or sequence to improve protein expression and/or purification.

Additionally, more than one peptide sequence (e.g., a peptide derived from a toxin or venom protein) can be present on, conjugated to, linked to, or fused with a particular peptide. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule or can substitute for a subsequence of the sequence that encodes the biomolecule.

A PD-L1-binding peptide of the present disclosure (e.g., any one of SEQ ID NO: 1-SEQ ID NO: 118, SEQ ID NO: 435, SEQ ID NO: 436, or SEQ ID NO: 554-SEQ ID NO: 567) may be modified with a cell penetrating peptide to form a cell penetrating PD-L1-binding peptide. The cell penetrating peptide may facilitate delivery of the PD-L1-binding peptide or peptide complex into a cell or across a cellular layer (e.g., across the blood brain barrier or across the endosome into a cytosol). In some embodiments, a cell penetrating PD-L1-binding peptide may be further complexed with an additional active agent to facilitate delivery of the additional active agent into a cell or across a cellular layer. This may enable delivery of active agents (e.g., therapeutic agents) to intracellular targets. Examples of cell penetrating peptides that may be used in combination with the PD-L1-binding peptides of the present disclosure are provided in TABLE 7.

TABLE 7
Exemplary Cell Penetrating Peptides

| SEQ ID NO | Name | Sequence |
|---|---|---|
| SEQ ID NO: 249 | CysTat | CYRKKRRQRRR |
| SEQ ID NO: 250 | S19-TAT | PFVIGAGVLGALGTGIGGIGRKKRRQRRR |
| SEQ ID NO: 251 | R8 | RRRRRRRR |
| SEQ ID NO: 252 | pAntp | RQIKIWFQNRRMKWKK |
| SEQ ID NO: 253 | Pas-TAT | FFLIPKGGRKKRRQRRR |
| SEQ ID NO: 254 | Pas-R8 | FFLIPKGRRRRRRRR |
| SEQ ID NO: 255 | PasFHV | FFLIPKGRRRRNRTRRNRRRVR |
| SEQ ID NO: 256 | Pas-pAntP | FFLIPKGRQIKIWFQNRRMKWKK |
| SEQ ID NO: 257 | F2R4 | FFRRRR |
| SEQ ID NO: 258 | B55 | KAVLGATKIDLPVDINDPYDLGLLLRHLRHHS |
| | | NLLANIGDPAVREQVLSAMQEEE |
| SEQ ID NO: 259 | auzurin | LSTAADMQGVVTDGMASGLDKDYLKPDD |
| SEQ ID NO: 260 | IMT-P8 | RRWRRWNRFNRRRCR |
| SEQ ID NO: 261 | BR2 | RAGLQFPVGRLLRRLLR |
| SEQ ID NO: 262 | OMOTAG1 | KRAHHNALERKRR |
| SEQ ID NO: 263 | OMOTAG2 | RRMKANARERNRM |
| SEQ ID NO: 264 | pVEC | LLIILRRRIRKQAHAHSK |
| SEQ ID NO: 265 | SynB3 | RRLSYSRRRF |
| SEQ ID NO: 266 | DPV1047 | VKRGLKLRHVRPRVTRMDV |
| SEQ ID NO: 267 | CY105Y | CSIPPEVKFNKPFVYLI |
| SEQ ID NO: 268 | Transportan | GWTLNSAGYLLGKINLKALAALAKKIL |
| SEQ ID NO: 269 | MTS | KGEGAAVLLPVLLAAPG |
| SEQ ID NO: 270 | hLF | KCFQWQRNMRKVRGPPVSCIKR |
| SEQ ID NO: 271 | PFVYLI | PFVYLI |
| SEQ ID NO: 272 | yBBR | VLDSLEFIASKL |
| SEQ ID NO: 273 | DRI-TAT31 | rrrqrrkkrgy |
| SEQ ID NO: 274 | cyclic | FΦRRRRQ |
| | heptapeptide | |
| | cyclo | |
| SEQ ID NO: 275 | Argₙ | Argₙ wherein n is a whole number |
| | | and can be 2, 3, 4, 5, 6, 7, 8, |
| | | 9, or 10 |
| SEQ ID NO: 276 | Tat peptide | YGRKKRRQRRR |
| SEQ ID NO: 277 | | GRKKRRQRRR |
| SEQ ID NO: 278 | | GRKKRRQRRRPQ |
| SEQ ID NO: 279 | DPV3 | RKKRRRESRKKRRRES |
| SEQ ID NO: 280 | C10H | RKGFYKRKQCKPSRGRKR |
| SEQ ID NO: 281 | VP22 | NAATATRGRSAASRPTQRPRAPARSASRPRRP |
| | | VQ |
| SEQ ID NO: 282 | TP10 | AGYLLGKINLKALAALAKKIL |
| SEQ ID NO: 283 | MAP | KLALKLALKALKAALKLA |
| SEQ ID NO: 284 | BPrPp | MVKSKIGSWILVLFVAMWSDVGLCKKRP |
| SEQ ID NO: 285 | ARF | MVRRFLVTLRIRRACGPPRVRV |
| SEQ ID NO: 286 | GALA | WEAALAEALAEALAEHLAEALAEALEALAA |
| SEQ ID NO: 287 | SAP | VRLPPPVRLPPPVRLPPP |
| SEQ ID NO: 288 | MPG | GLAFLGFLGAAGSTMGAWSQPKKKRKV |
| SEQ ID NO: 289 | Pep-1 | KETWWETWWTEWSQPKKKRKV |
| SEQ ID NO: 290 | [WR]4 | WRWRWRWR |
| SEQ ID NO: 291 | Ig(v) | MGLGLHLLVLAAALQGAKKKRKV |
| SEQ ID NO: 292 | K-FGF | AAVALLPAVLLAHLLAP |
| SEQ ID NO: 293 | Melittin | GIGAVLKVLTTGLPALISWIKRKRQQ |
| SEQ ID NO: 294 | gH625 | HGLASTLTRWAHYNALIRAF |
| SEQ ID NO: 295 | HIV-1 TAT | GRKKRRQRRRPPQ |
| | protein (48-60) | |
| SEQ ID NO: 296 | MPG HIV- | GALFLGFLGAAGSTMGAWSQPKKKRKV |
| | gp41/SV40 | |
| | T-antigen | |
| SEQ ID NO: 297 | R6W3 | RRWWRRWRR |
| SEQ ID NO: 298 | NLS | CGYGPKKKRKVGG |
| SEQ ID NO: 299 | 8-lysines | KKKKKKKK |
| SEQ ID NO: 300 | HRSV | RRIPNRRPRR |
| SEQ ID NO: 301 | AIP6 | RLRWR |
| SEQ ID NO: 302 | Pep-1 | KETWWETWWTEWSQPKKRKV |
| SEQ ID NO: 303 | MAP17 | QLALQLALQALQAALQLA |
| SEQ ID NO: 304 | VT5 | DPKGDPKGVTVTVTVTVTGKGDPKPD |
| SEQ ID NO: 305 | Bac7 | RRIRPRPPRLPRPRPRPLPFPRPG |
| SEQ ID NO: 306 | (PPR)n | PPRPPRPPR |
| SEQ ID NO: 307 | | PPRPPRPPRPPR |
| SEQ ID NO: 308 | | PPRPPRPPRPPRPPR |
| SEQ ID NO: 309 | | PPRPPRPPRPPRPPRPPR |
| SEQ ID NO: 310 | INF7 | GLFEAIEGFIENGWEGMIDGWYGC |
| SEQ ID NO: 311 | CADY | GLWRALWRLLRSLWRLLWRA |
| SEQ ID NO: 312 | Pep-7 | SDLWEMMMVSLACQY |
| SEQ ID NO: 313 | TGN | TGNYKALHPHNG |
| SEQ ID NO: 314 | Ku-70 | VPMLK |
| SEQ ID NO: 315 | CPP | RRRRRRGGRRRRRG |
| | (RRRRRRGGRR | |
| | RRRG) | |
| SEQ ID NO: 316 | SVS-1 | KVKVKVKVDPPTKVKVKVK |
| SEQ ID NO: 317 | L-CPP | LAGRRRRRRRRRK |
| SEQ ID NO: 318 | RLW | RLWMRWYSPRTRAYG |
| SEQ ID NO: 319 | K16ApoE | KKKKKKKKKKKKKKKKLRVRLASHLRKLRK |
| | | RLLRDA |
| SEQ ID NO: 320 | Angiopep-2 | TFFYGGSRGKRNNFKTEEY |
| SEQ ID NO: 321 | ACPP | EEEEEEEEPLGLAGRRRRRRRRN |
| SEQ ID NO: 322 | KAFAK | KAFAKLAARLYRKALARQLGVAA |
| SEQ ID NO: 323 | hCT (9-32) | LGTYTQDFNKFHTFPQTAIGVGAP |
| SEQ ID NO: 324 | VP22(version2) | DAATATRGRSAASRPTQRPRAPARSASRPRRP |
| | | VE |
| SEQ ID NO: 325 | MPG | GALFLGFLGAAGSTMGAWSQPKSKRKV |
| SEQ ID NO: 326 | hPP3 | KPKRKRRKKKGHGWSR |
| SEQ ID NO: 327 | PepNeg | SGTQEEY |
| SEQ ID NO: 328 | CPP | RRRRRGGRRRRRG |
| | (RRRRRGGRRR | |
| | RRG) | |
| SEQ ID NO: 329 | CM18-TAT | KWKLFKKIGAVLKVLTTG |
| SEQ ID NO: 330 | PTD4 | YARAAARQARA |
| SEQ ID NO: 331 | WaTx | MKYFTLALTLLFLLLINPCKDMNFAWAESSEK |
| | | VERASPQQAKYCYEQCNVNKVPFDQCYQMC |
| | | SPLERS |
| SEQ ID NO: 332 | maurocaline | GDCLPHLKLCKENKDCCSKKCKRRGTNIEKR |
| | | CR |
| SEQ ID NO: 333 | imperatoxin | GDCLPHLKRCKADNDCCGKKCKRRGTNAEK |
| | | RCR |
| SEQ ID NO: 334 | hadrucalcin | SEKDCIKHLQRCRENKDCCSKKCSRRGTNPEK |
| | | RCR |
| SEQ ID NO: 335 | hemicalcin | GDCLPHLKLCKADKDCCSKKCKRRGTNPEKR |
| | | CR |
| SEQ ID NO: 336 | opicalcin-1 | GDCLPHLKRCKENNDCCSKKCKRRGTNPEKR |
| | | CR |
| SEQ ID NO: 337 | opicalcin-2 | GDCLPHLKRCKENNDCCSKKCKRRGANPEKR |
| | | CR |
| SEQ ID NO: 338 | midkine (62-104) | CKYKFENWGACDGGTGTKVRQGTLKKARYN |
| | | AQCQETIRVTKPC |
| SEQ ID NO: 339 | MCoTI-II | SGSDGGVCPKILKKCRRDSDCPGACICRGNGY |
| | | CG |
| SEQ ID NO: 340 | chlorotoxin | MCMPCFTTDHQMARKCDDCCGGKGRGKCYG |
| | | PQCLCR |
| SEQ ID NO: 341 | calcin | MLICLF |

In some embodiments, a tissue targeting domain can comprise a transferrin receptor-binding (TfR-binding) peptide, such as SEQ ID NO: 350 (REGCASRCMKYNDELEKCEARMMSMSNTEEDCEQELEDLLYCLDHCHSQ), which can promote transcytosis across the blood-brain barrier and deliver the PD-L1-binding peptide to the central nervous system including brain tumors. In some embodiments, a TfR-binding peptide may be derived from a scaffold of SEQ ID NO: 351 (REGCASHCTKYKAELEKCEARVSSRSNTEETCVQELFDFLHCVDHCVSQ) or SEQ ID NO: 352 (GSREGCASHCTKYKAELEKCEARVSSRSNTEETCVQELFDFLHCVDHCVSQ). For example, CNS access via transcytosis across the BBB may be performed by binding a PD-L1-binding peptide complexed with a TfR-binding peptide to transferrin receptor (TfR) followed by recycling the complex to the cell surface. Some of the PD-L1-binding peptide/TfR-binding peptide complexes may access low pH early endosomes. The PD-L1-binding peptide or an additional active agent complex may be exposed to endosome upon endocytosis of TfR and may remain and not be degraded due to stability of the PD-L1-binding peptide. If the PD-L1-binding peptide/TfR-binding peptide complex includes additional cell penetration capabilities, the peptide may facilitate accelerated escape of the PD-L1-binding peptide or additional active agent from the endosomal compartment into the cytosol. Even without added cell penetration capabilities, the PD-L1-binding peptide or additional active agent may slowly leak out of endosomes and access the cytosol.

### Nucleotide Modifications

In some embodiments, the nucleic acid portion of a peptide oligonucleotide complex (e.g., an oligonucleotide of a PD-L1-binding peptide oligonucleotide complex) contains one or more bases within the nucleic acid molecule that are modified. Such modifications can occur whether the nucleic acid portion a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter. One or more bases in a given nucleotide sequence may be modified to increase in vivo stability, to increase resistance to enzymes such as nucleases, increase protein binding including to serum proteins, increase in vivo half-life, to modify the tissue biodistribution, or to modify how the immune system responds. The phosphonate, the ribose, or the base may be modified. In some aspects, the modification comprises a phosphorothioate modification, a phosphodiester modification, a thio-phosphoramidate modification, a methyl phosphonate modification, a phosphorodithioate modification, a methoxypropylphosphonate modification, a 5′-(E)-vinylphosphonate modification, a 5′methyl phosphonate modification, an (S)-5′-C-methyl with phosphate modification, a 5′-phosphorothioate modification, a peptide nucleic acid (PNA), a 2′-O methyl modification, a 2′-O-methoxyethyl (2′-O-Me) modification, a 2′-fluoro (2′-F) modification, a 2′-deoxy-2′-fluoro modification, a 2′arabino-fluor modification, a 2′-O-benyzl modification, a 2′-O-methyl-4-pyridine modification, a locked nucleic acid (LNA), an amino-LNA, a thio-LNA, an ENA, an amino ENA, a carbo-ENA, a (S)-cEt-bridged nucleic acid, an (S)-MOE, a bridged nucleic acid, a tricyclo-DNA, a morpholino nucleic acid (PMO), an unlocked nucleic acid (UNA), a glycol nucleic acid (GNA), a bridged nucleic acid (BNA), an ethyl (S)-cEt nucleic acid, a pseudouridine, a 2′-thiouridine, an N6′methyadenosine, a 5′-methylcytidine, a 5′-fluoro-2′-deoxyuridine, a N′ethylpiperidine 7′-EAA triazole modified adenine, an N-ethylpiperidine 6′-triazole modified adenine, a 6′-phenylpyrrolocytosine, a 2′,4′-difluorotoluyl ribonucleoside, a 5′nitroindole, a 5′ methyl, a 5′ phosphonate, an inverted A base, a 2′-H (deoxyribose), a 2′-OH (ribose), or any combination thereof. The oligonucleotide may be comprised entirely of a combination of 2′-O-Me and 2′-F modifications. Diastereomers or one or both stereoisomers may be used. Any of the stabilization chemistries or patterns, including STC, ESC, advanced, ESC, AD1-3, AD5, disclosed in Hu Signal Transduction and Targeted Therapy 2020,5:101 can be used. Pyrimidines can be 2′-fluoro-modified, which can increase stability to nucleases but can also increase immune system activation. The RNA backbone can be phosphorothioate-substituted (where the non-bridging oxygen is replaced with sulfur), which can increase resistance to nuclease digestion as well as altering the biodistribution and tissue retention and increasing the pharmacokinetics such as by increasing protein binding, but can also induce more immune stimulation. Methyl phosphonate modification of an RNA can also be used. 2′-Omethyl and 2′-F RNA bases can be used, which can protect against base hydrolysis and nucleases and increase the melting temperature of duplexes. Bridged, Locked, and other similar forms of Bridged Nucleic Acids (BNA, LNA, cEt) where any chemical bridge such as an N—O linkage between the 2′ oxygen and 4′ carbons in ribose can be incorporated to increase resistance to exo- and endonucleases and enhance biostability. These include BNA where an N—O linkage between the 2′ and 4′ carbons occur and where any chemical modification of the nitrogen (including but not limited to N—H, N—CH3, N-benzene) in the bridge can be added to increase stability RNA backbone or base modifications can be placed anywhere in the RNA sequence, at one, multiple, or all base locations. Optionally, phosophorothioate nucleic acid linkages may be used between the 2-4 terminal nucleic acids of one or both sequences. Optionally 2′F modified nucleic acids may be used at least at 2-4 positions, at least 5%, at least 10% at least 25% of internal positions, at least 50%, at least 75%, or up to 100% of internal positions, all internal positions or all positions. Optionally, one or more of 2′F base, an LNA base, a BNA base, an ENA base, a 2′O-MOE base, a morpholino base, a 2′OMe base, a 5′-Me base, a (S)-cEt base or combinations thereof may be used at least at 2-4 positions, at least 5%, at least 10% at least 25% of internal positions, at least 50%, at least 75%, or up to 100% of internal positions, all internal positions or all positions.

Modified bases can be used to increase in the in vivo half-life of the oligonucleotide. They can allow the oligonucleotide to remaining intact in the serum, endosome, cytosol, or nucleus, including for days, weeks, or months. This can allow ongoing activity, including if the oligonucleotide is slowly released from the endosome over days, weeks, or months within a given cell (such as described in Brown et al., Nucleic Acids Research, 2020, p 11827-11844).

In some embodiments, a nucleotide comprises at least one phosphorothioate linkage. In some embodiments, a peptide oligonucleotide complex comprises from 1 to 12 phosphorothioate linkages. In some embodiments, a nucleotide comprises at least one thiophosphoroamidate linkage. In some embodiments, a nucleotide comprises from 1 to 12 thiophosphoroamidate linkages. In some embodiments, a nucleotide comprises at least one modified base. In some embodiments, at least modified base comprises a 2′F base, an LNA base, a BNA base, an ENA base, a 2′O-MOE base, a 5′-Me base, a (S)-cEt base, a 2′OMe base, a morpholino base, or combinations thereof.

### Linkers

Peptides according to the present disclosure (e.g., PD-L1-binding peptides or peptide complexes) can be attached to another moiety (e.g., an additional active agent), such as a small molecule, a second peptide, a second CDP, a protein, a miniprotein, an antibody, an antibody fragment, an Fc, an Fc knob, an Fc hole, an aptamer, polypeptide, polynucleotide, a fluorophore, a radioisotope, a radionuclide chelator, a polymer, a biopolymer, a fatty acid, an acyl adduct, a chemical linker, a binding moiety, or sugar or other active agent or detectable agent described herein through a linker, or directly in the absence of a linker. In the absence of a linker, for example, an active agent or a detectable agent can be conjugated to, linked to, or fused to the N-terminus or the C-terminus of a peptide to create an active agent or detectable agent fusion peptide. In other embodiments, the link can be made by a peptide fusion via reductive alkylation. In some embodiments, a cleavable linker is used for in vivo delivery of the peptide, such as a linker that can be cleaved or degraded upon entry in a cell, endosome, or a nucleus. In some embodiments, in vivo delivery of a peptide requires a small linker that does not interfere with penetration of a cell or localization to a nucleus of a cell. A linker can also be used to covalently attach a peptide as described herein to another moiety or molecule having a separate function, such a targeting, cytotoxic, therapeutic, homing, imaging, or diagnostic functions.

A peptide can be directly attached to another molecule by a covalent attachment. For example, the peptide is attached to a terminus of the amino acid sequence of a larger polypeptide or peptide molecule, or is attached to a side chain, such as the side chain of a lysine, serine, threonine, cysteine, tyrosine, aspartic acid, a non-natural amino acid residue, or glutamic acid residue. The attachment can be via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond, or linker as described herein) can be used to link other molecules.

Attachment via a linker can involve incorporation of a linker moiety between the other molecule and the peptide. The peptide and the other molecule can both be covalently attached to the linker. The linker can be cleavable, labile, non-cleavable, stable, stable self-immolating, hydrophilic, or hydrophobic. As used herein, the term “non-cleavable” or “stable” (such as used in association with an amide, cyclic, or carbamate linker or as otherwise as described herein) is often used by a skilled artisan to distinguish a relatively stable structure from one that is more labile or “cleavable” (e.g., as used in association with cleavable linkers that may be dissociated or cleaved structurally by enzymes, proteases, self-immolation, pH, reduction, hydrolysis, certain physiologic conditions, or as otherwise described herein). It is understood that “non-cleavable” or “stable” linkers offer stability against cleavage or other dissociation as compared to “cleavable” linkers, and the term is not intended to be considered an absolute non-cleavable or non-dissociative structure under any conditions. Consequently, as used herein, a “non-cleavable” linker is also referred to as a “stable” linker. The linker can have at least two functional groups with one bonded to the peptide, the other bonded to the other molecule, and a linking portion between the two functional groups. Some example linkers are described in Jain, N., Pharm Res. 32(11): 3526-40 (2015), Doronina, S. O., Bioconj Chem. 19(10): 1960-3 (2008), Pillow, T. H., J Med Chem. 57(19): 7890-9 (2014), Dorywalksa, M., Bioconj Chem. 26(4): 650-9 (2015), Kellogg, B. A., Bioconj Chem. 22(4): 717-27 (2011), and Zhao, R. Y., J Med Chem. 54(10): 3606-23 (2011).

Non-limiting examples of the functional groups for attachment can include functional groups capable of forming an amide bond, an ester bond, an ether bond, a carbonate bond, a carbamate bond, or a thioether bond. Non-limiting examples of functional groups capable of forming such bonds can include amino groups; carboxyl groups; hydroxyl groups; aldehyde groups; azide groups; alkyne and alkene groups; ketones; hydrazides; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion can include alkylene, alkenylene, alkynylene, polyether, such as polyethylene glycol (PEG), hydroxy carboxylic acids, polyester, polyamide, polyamino acids, polypeptides, cleavable peptides, valine-citrulline, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, and ester groups.

In some cases, a linker can comprise a triazole group, such as any one of the heterocyclic compounds with molecular formula C₂H₃N₃, having a five-membered ring of two carbon atoms and three nitrogen atoms, optionally with a hydrogen atom bonded to N at any position in the ring, such as:

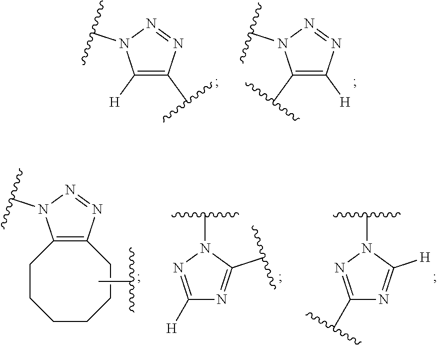

for example, a 1, 2, 3-Triazole (such as 1H1,2,3-Triazole, 2H1,2,3-Triazole, or 1-methyl-4,5,6,7,8,9-hexahydro-1H-cycloocta[d][1,2,3]triazole) or a 1,2,4-Triazole (such as 1H1,2,4-Triazole or 4H1,2,4-Triazole).

Additional non-limiting examples of linkers include linear or non-cyclic linkers such as:

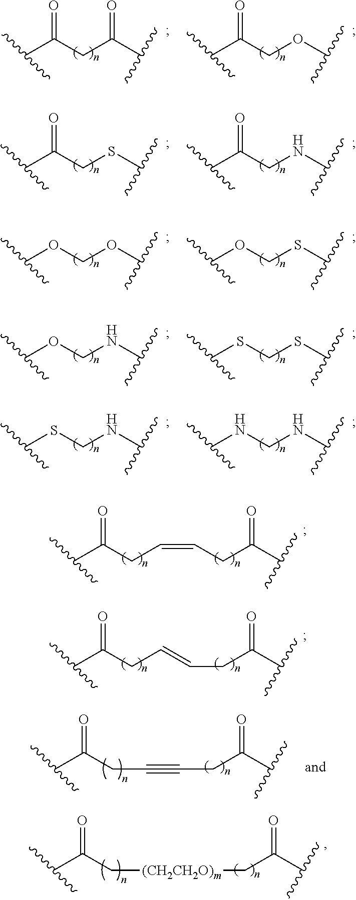

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50, or any linker as disclosed in Jain, N., Pharm Res. 32(11): 3526-40 (2015) or Ducry, L., Antibody Drug Conjugates (2013).

In some cases, a linker can comprise a cyclic group, such as an organic nonaromatic or aromatic ring, optionally with 3-10 carbons in the ring, optionally built from a carboxylic acid,

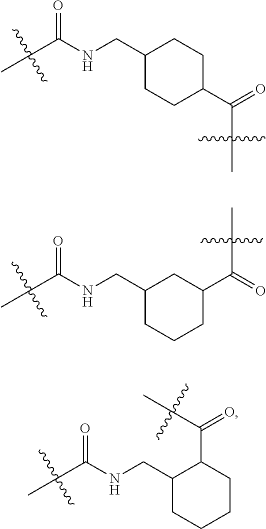 [SMILES: *C(=O)NCC1CCC(C(*)=O)CC1 ; *C(=O)NCC1CCCC(C(*)=O)C1 ; *C(=O)NCC1CCCCC1C(*)=O]

for example trans-4-(aminomethyl) cyclohexane carboxylic acid,

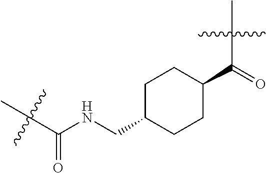 [SMILES: *C(=O)NC[C@H]1CC[C@H](C(*)=O)CC1]

or a substituted analog or a stereoisomer thereof. This linker can optionally be used to form a carbamate linkage. In some cases, a carbamate linkage can be more resistant to cleavage, such as by hydrolysis, enzymes such as esterases, or other chemical reactions, than an ester linkage.

In some cases, a linker can comprise a cyclic carboxylic acid, for example a cyclic dicarboxylic acid, for example one of the following groups: 1,4-cyclohexane dicarboxylic acid, 1,2-cyclohexane dicarboxylic acid, or 1,3-cyclohexane dicarboxylic acid, 1,1-cyclopentanediacetic acid,

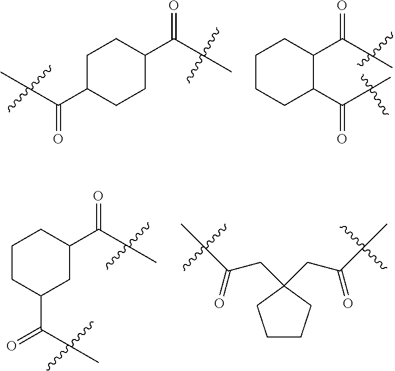 [SMILES: *C(=O)C1CCC(C(*)=O)CC1 ; *C(=O)C1CCCCC1C(*)=O ; *C(=O)C1CCCC(C(*)=O)C1 ; *C(=O)CC1(CC(*)=O)CCCC1]

or a substituted analog or a stereoisomer thereof. For example, the linker can comprise one of the following groups.

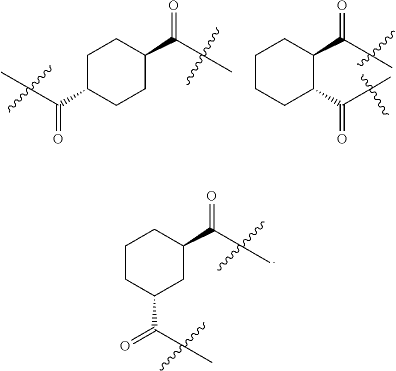 [SMILES: *C(=O)[C@H]1CC[C@H](C(*)=O)CC1 ; *C(=O)[C@@H]1CCCC[C@H]1C(*)=O ; *C(=O)[C@@H]1CCC[C@@H](C(*)=O)C1]

In some instances, the linker can optionally be used to form an ester linkage. In some cases, a cyclic ester linkage can be more sterically resistant to cleavage, such as by hydrolysis by water, enzymes such as esterases, or other chemical reactions, than a noncyclic or linear ester linkage.

In some cases, a linker can comprise an aromatic dicarboxylic acid, for example terephthalic acid, isophthalic acid, phthalic acid

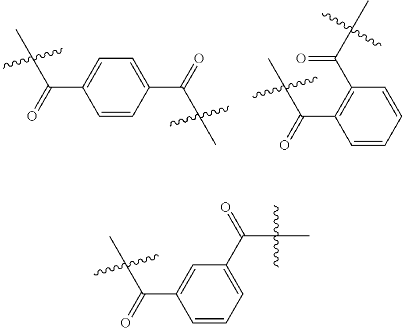 [SMILES: *C(=O)c1ccc(C(*)=O)cc1 ; *C(=O)c1ccccc1C(*)=O ; *C(=O)c1cccc(C(*)=O)c1]

or a substituted analog thereof.

In some cases, a linker can comprise a natural or non-natural amino acid, for example cysteine,

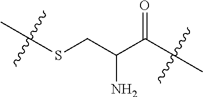 [SMILES: *SCC(N)C(*)=O]

or a substituted analog or a stereoisomer thereof. In some instances, a linker can comprise alanine (A, Ala); arginine (R, Arg); asparagine (N, Asn); aspartic acid (D, Asp); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val); or any plurality or combination thereof. In some embodiments, the non-natural amino acid can comprise one or more functional groups, e.g., alkene or alkyne, that can be used as functional handles.

In some cases, a linker can comprise one of the following groups:

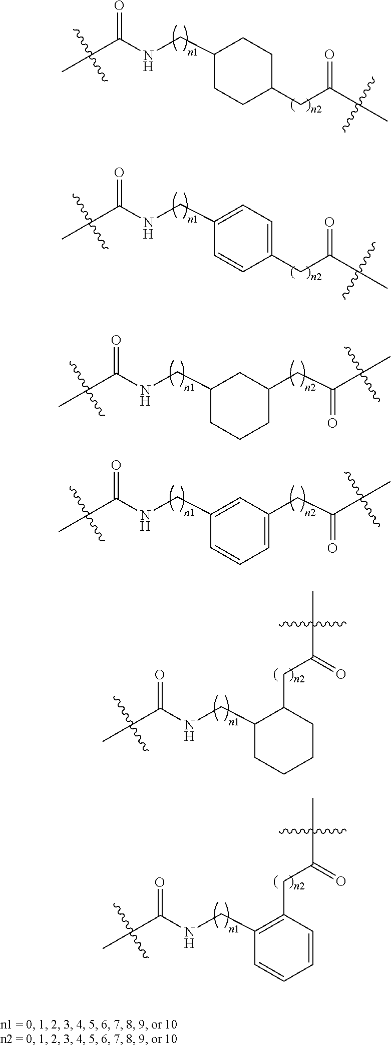

or a substituted analog or a stereoisomer thereof. In some instances the linker is selected from one of the following groups:

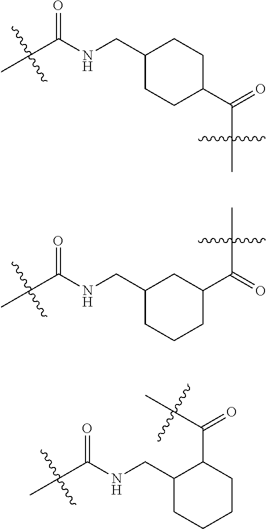 [SMILES: *C(=O)NCC1CCC(C(*)=O)CC1 ; *C(=O)NCC1CCCC(C(*)=O)C1 ; *C(=O)NCC1CCCCC1C(*)=O]

or a substituted analog or a stereoisomer thereof.

In some cases, a linker can comprise one of the following groups:

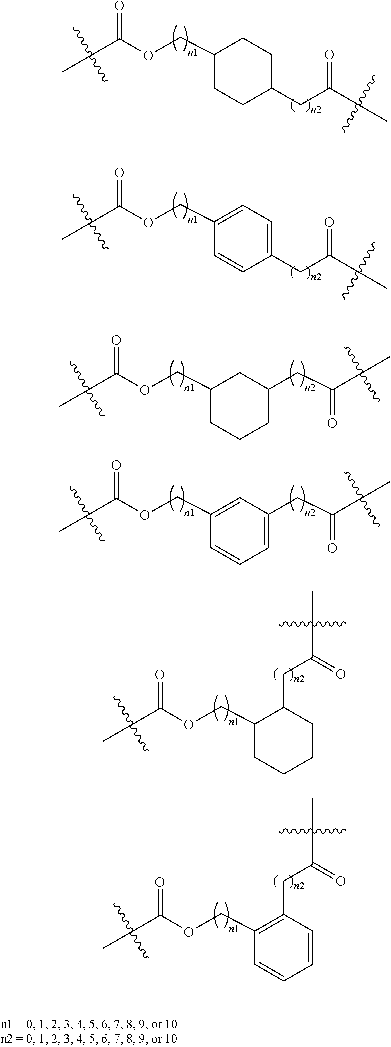

or a substituted analog or a stereoisomer thereof. In some instances, the linker is selected from one of the following groups:

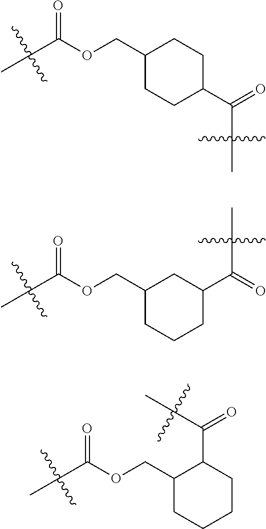 [SMILES: *C(=O)OCC1CCC(C(*)=O)CC1 ; *C(=O)OCC1CCCC(C(*)=O)C1 ; *C(=O)OCC1CCCCC1C(*)=O]

or a substituted analog or a stereoisomer thereof.

In some cases, a substituted analog or a stereoisomer is a structural analog of a compound disclosed herein, for which one or more hydrogen atoms of the compound can be substituted by one or more groups of halo (e.g., Cl, F, Br), alkyl (e.g., methyl, ethyl, propyl), alkenyl, alkynyl, arylalkyl, cycloalkyl, cycloalkylalkyl, heteroalkyl, heterocycloalkyl, or any combination thereof. In some cases, a stereoisomer can be an enantiomer, a diastereomer, a cis or trans stereoisomer, a E or Z stereoisomer, or a R or S stereoisomer.

Non-limiting examples of linear linkers include;

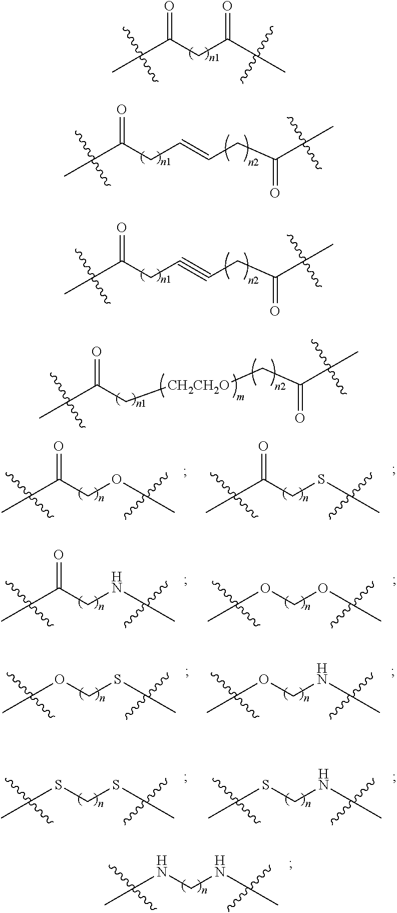

wherein each n1, or n2 or m is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some instances, the linker can comprise a linear dicarboxylic acid, e.g., one of the following groups: succinic acid, 2,3-dimethylsuccinic acid, glutaric acid, adipic acid, 2,5-dimethyladipic acid,

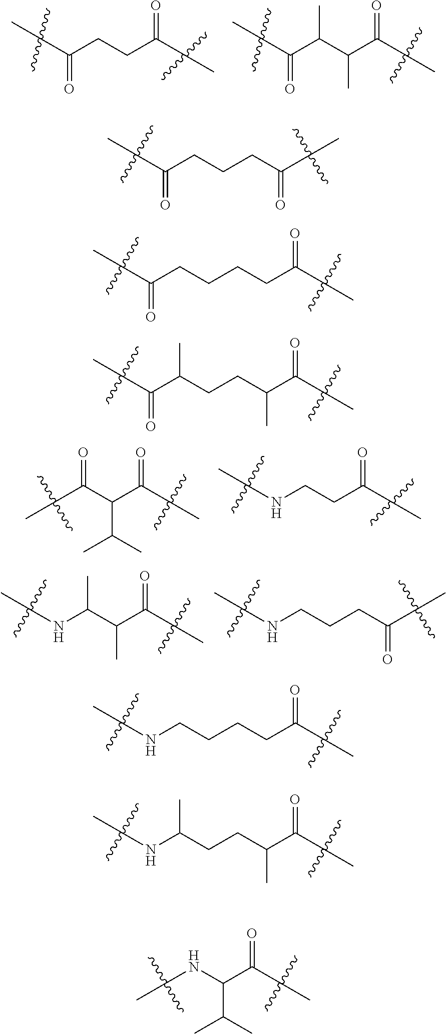

or a substituted analog or a stereoisomer thereof. In some cases, the linker can be used to form a carbamate linkage. In some embodiments, the carbamate linkage can be more resistant to cleavage, such as by hydrolysis, enzymes such as esterases, or other chemical reactions, than an ester linkage. In some cases, the linker can be used to form a linear ester linkage. In some embodiments, the linear ester linkage can be more susceptible to cleavage, such as by hydrolysis, enzymes such as esterases, or other chemical reactions, than a cyclic ester or carbamate linkage. Side chains such as methyl groups on the linear ester linkage can optionally make the linkage less susceptible to cleavage than without the side chains.

In some cases a linker can be a succinic linker, and a targeting agent (e.g., a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter) or other active agent or detectable agent can be attached to a peptide via an ester bond or an amide bond with two methylene carbons in between. In other cases, a linker can be any linker with both a hydroxyl group and a carboxylic acid, such as hydroxy hexanoic acid or lactic acid.

In some cases, a nucleotide (e.g., a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter), an active agent, or a detectable agent can be attached to a peptide using any one or more of the linkers shown below in TABLE 8. In some embodiments, a peptide, an additional active agent, or a detectable agent can be attached to a nucleotide any one or more of the linkers shown below in TABLE 8.

TABLE 8
Exemplary Linkers for Use in Peptide Conjugates or Complexes

| Compound | | |
|---|---|---|
| number | Chemical structure | Linker is based on: |
| 1 | 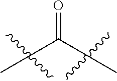 [SMILES: *C(*)=O] | Carbonic acid |
| 2 | 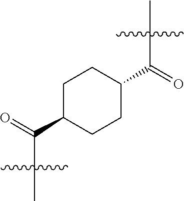 [SMILES: *C(=O)[C@H]1CC[C@H](C(*)=O)CC1] | trans-1,4- cyclohexanedicarboxylic acid |
| 3 | 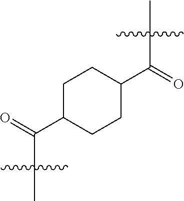 [SMILES: *C(=O)C1CCC(C(*)=O)CC1] | 1,4-cyclohexanedicarboxylic acid |
| 4 | 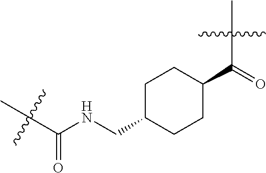 [SMILES: *C(=O)NC[C@H]1CC[C@H](C(*)=O)CC1] | trans-1-aminomethylamine- cyclohexane-4-carboxylic acid (carbamate) |
| 5 | 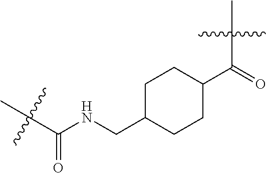 [SMILES: *C(=O)NCC1CCC(C(*)=O)CC1] | 1-aminomethylamine- cyclohexane-4-carboxylic acid |
| 6 | 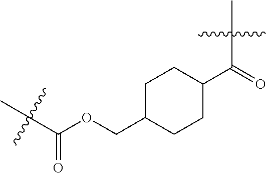 [SMILES: *C(=O)OCC1CCC(C(*)=O)CC1] | 4-(carboxyoxy)-methyl- cyclohexane-1-carboxylic acid |
| 7 | 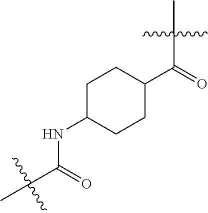 [SMILES: *C(=O)NC1CCC(C(*)=O)CC1] | 4-(carboxyamino)- cyclohexane-1-carboxylic acid |
| 8 | 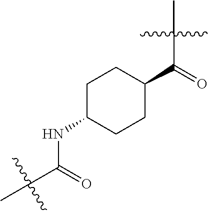 [SMILES: *C(=O)N[C@H]1CC[C@H](C(*)=O)CC1] | trans-4-(carboxyamino)- cyclohexane-1-carboxylic acid |
| 9 | 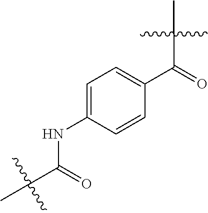 [SMILES: *C(=O)Nc1ccc(C(*)=O)cc1] | 4-(carboxyamino)benzoic acid |
| 10 | 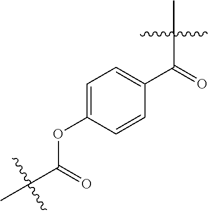 [SMILES: *C(=O)Oc1ccc(C(*)=O)cc1] | 4-(carboxyoxy)benzoic acid |
| 11 | 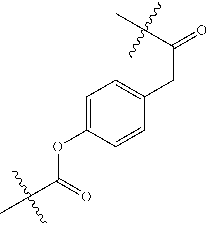 [SMILES: *C(=O)Cc1ccc(OC(*)=O)cc1] | 2-(4- (carboxyoxy)phenyl)acetic acid |
| 12 | 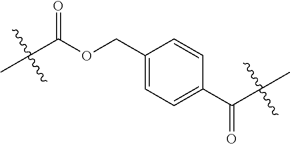 [SMILES: *C(=O)OCc1ccc(C(*)=O)cc1] | 4- ((carboxyoxy)methyl)benzoic acid |
| 13 | 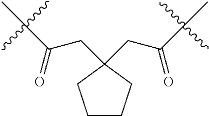 [SMILES: *C(=O)CC1(CC(*)=O)CCCC1] | 2,2′-(cyclopentane-1,1- diyl)diacetic acid |
| 14 | 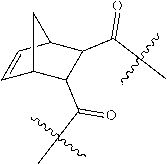 [SMILES: *C(=O)C1C2C=CC(C2)C1C(*)=O] | cis-5-norborene-endo-2,3- dicarboxylic acid |
| 15 | 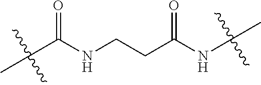 [SMILES: *NC(=O)CCNC(*)=O] | (3-amino-3- oxypropyl)carbamic acid |
| 16 | 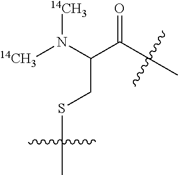 [SMILES: *SCC(C(*)=O)N([14CH3])[14CH3]] | [¹⁴C]-Cysteine |
| 17 | 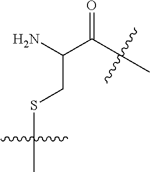 [SMILES: *SCC(N)C(*)=O] | Cysteine |
| 18 | 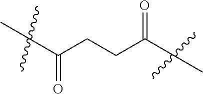 [SMILES: *C(=O)CCC(*)=O] | Succinic acid |
| 19 | 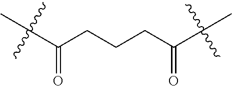 [SMILES: *C(=O)CCCC(*)=O] | Glutaric acid |
| 20 | 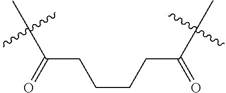 [SMILES: *C(=O)CCCCC(*)=O] | Adipic acid |
| 21 | 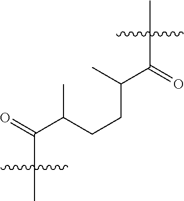 [SMILES: *C(=O)C(C)CCC(C)C(*)=O] | 2,5-dimethyladipic acid (DMA) |
| 22 | 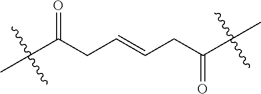 [SMILES: *C(=O)C/C=C/CC(*)=O] | trans-β-hydromuconic acid |
| 23 | 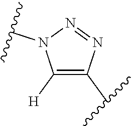 [SMILES: *c1cn(*)nn1] | 1,4-dimethyl-1H-1,2,3-triazole |
| 24 | 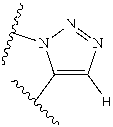 [SMILES: *c1cnnn1*] | 1,5-dimethyl-1H-1,2,3-triazole |
| 25 | 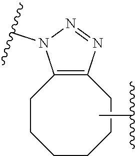 | 1-methyl-4,5,6,7,8,9- hexahydro-1H- cycloocta[d][1,2,3]triazole |
| 26 | 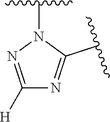 [SMILES: *c1ncnn1*] | 1,5-dimethyl-1H-1,2,4-triazole |
| 27 | 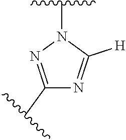 [SMILES: *c1ncn(*)n1] | 1,3-dimethyl-1H-1,2,4-triazole |

In some cases, an active agent is attached to a linker wherein a nucleophilic functional group (e.g., a hydroxyl group) of the active agent molecule acts as the nucleophile and replaces a leaving group on the linker moiety, thereby attaching it to the linker.

In other cases, an active agent is attached to a linker wherein a nucleophilic functional group (e.g., thiol group, amine group, etc.) of the linker replaces a leaving group on the active agent, thereby attaching it to the linker. Such leaving group (or functional group that may be converted into a leaving group) may be a primary alcohol to form a thioether bond, thereby attaching it to the linker. A primary alcohol can be converted into a leaving group such as a mesylate, a tosylate, or a nosylate in order to accelerate the nucleophilic substitution reaction.

The peptide-active agent complexes of the present disclosure (e.g., PD-L1-binding peptide complexes) can comprise an active agent (e.g., a therapeutic agent, a detectable agent, or an immune cell binding moiety), a linker, and/or a peptide of the present disclosure. A general connectivity between these three components can be active agent-linker-peptide, such that the linker is attached to both the active agent and the peptide. In many cases, the peptide is attached to a linker via an amide bond. Amide bonds can be relatively stable (e.g., in vivo) compared to other bonds described herein, such as esters, carbonates, etc. The amide bond between the peptide and the linker may thus provide advantageous properties due to its in vivo stability of the active agent is sought to be cleaved from a peptide-active agent-conjugate without the linker being attached to the active agent after such in vivo cleavage. Thus, in various cases, an active agent is attached to the linker-peptide moiety via linkages such as ester, carbonate, carbamate, etc., wherein the peptide or active agent is attached to the linker via an amide bond. This can allow for selective cleavage of the active agent-linker bond (as opposed to the linker-peptide bond) allowing the active agent to be released without a linker moiety attached to it after cleavage. The use of such different active agent-linker bonds or linkages can allow the modulation of active agent release in vivo, e.g., in order to achieve a therapeutic function while minimizing off-target effects (e.g., reduction in drug release during circulation).

The linker can be a cleavable or a stable linker. The use of a cleavable linker permits release of the conjugated moiety (e.g., a nucleotide targeting agent, a therapeutic agent, a detectable agent, or a combination thereof) from the peptide, e.g., after targeting to the target tissue or cell or subcellular compartment or after endocytosis. In some cases, the linker is enzyme cleavable, e.g., a valine-citrulline linker (SEQ ID NO: 217) that can be cleavable by cathepsin, or an ester linker that can be cleavable by esterase. In some embodiments, the linker contains a self-immolating portion. In other embodiments, the linker includes one or more cleavage sites for a specific protease, such as a cleavage site for matrix metalloproteases (MMPs), thrombin, urokinase-type plasminogen activator, or cathepsin (e.g., cathepsin K).

Thus, in some cases, a peptide-active agent complexes of the present disclosure can comprise one or more, about two or more, about three or more, about five or more, about ten or more, or about 15 or more amino acids that can form an amino acid sequence cleavable by an enzyme. Such enzymes can include proteinases. A peptide-active agent complex can comprise an amino acid sequence that can be cleaved by a Cathepsin, a Chymotrypsin, an Elastase, a Subtilisin, a Thrombin I, or a Urokinas, or any combination thereof.

Alternatively or in combination, the cleavable linker can be cleaved, dissociated, or broken by other mechanisms, such as via pH, reduction, or hydrolysis. Hydrolysis can occur directly due to water reaction, or be facilitated by an enzyme, or be facilitated by presence of other chemical species. A hydrolytically labile linker, (amongst other cleavable linkers described herein) can be advantageous in terms of releasing active agents from the peptide. For example, an active agent in a conjugate form with the peptide may not be active, but upon release from the conjugate after targeting to the target tissue or cell or subcellular compartment, the active agent is active. The cleaved active agent may retain the chemical structure of the active agent before cleavage or may be modified. In some embodiments, a stable linker may optionally not cleave in buffer over extended periods of time (e.g., hours, days, or weeks). In some embodiments, a stable linker may optionally not cleave in body fluids such as plasma or synovial fluid over extended periods of time (e.g., hours, days, or weeks). In some embodiments, a stable linker optionally may cleave, such as after exposure to enzymes, reactive oxygen species, other chemicals or enzymes that may be present in cells (such as macrophages), cellular compartments (such as endosomes and lysosomes), inflamed areas of the body (such as inflamed joints), or tissues or body compartments. In some embodiments, a stable linker may optionally not cleave in vivo but present an active agent that is still active when conjugated to, linked to, or fused to the peptide.

The rate of hydrolysis of the linker (e.g., a linker of a peptide conjugate) can be tuned. For example, the rate of hydrolysis of linkers with unhindered esters may be faster compared to the hydrolysis of linkers with bulky groups next an ester carbonyl. A bulky group can be a methyl group, an ethyl group, a phenyl group, a ring, or an isopropyl group, or any group that provides steric bulk. In another example, the rate of hydrolysis can be faster with hydrophilic groups, such as alcohols, acids, or ethers, or near an ester carbonyl. In another example, hydrophobic groups present as side chains or by having a longer hydrocarbon linker can slow cleavage of the ester. In some embodiments, cleavage of a carbamate group can also be tuned by hindrance, hydrophobicity, and the like. In another example, using a less labile linker, such as a carbamate rather than an ester, can slow the cleavage rate of the linker. In some cases, the steric bulk can be provided by the drug itself, such as by ketorolac when conjugated via its carboxylic acid. The rate of hydrolysis of the linker can be tuned according to the residency time of the conjugate in the target tissue or cell or subcellular compartment, according to how quickly the peptide accumulates in the target tissue or cell or subcellular compartment, or according to the desired time frame for exposure to the active agent in the target tissue or cell or subcellular compartment. For example, when a peptide is cleared from the target tissue or cell or subcellular compartment relatively quickly, the linker can be tuned to rapidly hydrolyze. In contrast, for example, when a peptide has a longer residence time in the target tissue or cell or subcellular compartment, a slower hydrolysis rate can allow for extended delivery of an active agent. This can be important when the peptide is used to deliver a drug to the target tissue or cell or subcellular compartment (e.g., a tumor cell or a tumor tissue). “Programmed hydrolysis in designing paclitaxel prodrug for nanocarrier assembly” Sci Rep 2015, 5, 12023 Fu et al., provides an example of modified hydrolysis rates. In some embodiments, rates of cleavage can vary by species, body compartment, and disease state. For instance, cleavage by esterases may be more rapid in rat or mouse plasma than in human plasma, such as due to different levels of carboxyesterases. In some embodiments, a linker may be tuned for different cleavage rates for similar cleavage rates in different species.

In some cases, a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

In some embodiments, the linker can release the active agent in an unmodified form. In other embodiments, the active agent can be released with chemical modification. In still other embodiments, catabolism can release the active agent still linked to parts of the linker and/or peptide.

The linker can be a stable linker or a cleavable linker. In some embodiments, the stable linker can slowly release the conjugated moiety by an exchange of the conjugated moiety onto the free thiols on serum albumin. In some embodiments, the use of a cleavable linker can permit release of the conjugated moiety (e.g., an active agent) from the peptide, e.g., after administration to a subject in need thereof. In other embodiments, the use of a cleavable linker can permit the release of the conjugated therapeutic from the peptide. In some cases, the linker is enzyme cleavable, e.g., a valine-citrulline linker (SEQ ID NO: 217). In some embodiments, the linker contains a self-immolating portion. In other embodiments, the linker includes one or more cleavage sites for a specific protease, such as a cleavage site for matrix metalloproteases (MMPs), thrombin, cathepsins, peptidases, or beta-glucuronidase. Alternatively or in combination, the linker is cleavable by other mechanisms, such as via pH, reduction, or hydrolysis.

The rate of hydrolysis or reduction of the linker can be fine-tuned or modified depending on an application. For example, the rate of hydrolysis of linkers with unhindered esters can be faster compared to the hydrolysis of linkers with bulky groups next to an ester carbonyl. A bulky group can be a methyl group, an ethyl group, a phenyl group, a ring, or an isopropyl group, or any group that provides steric bulk. In some cases, the steric bulk can be provided by the drug itself, such as by ketorolac when conjugated, linked, or fused via its carboxylic acid. The rate of hydrolysis of the linker can be tuned according to the residency time of the conjugate or fusion in the target location. For example, when a peptide is cleared from a tumor, or the brain, relatively quickly, the linker can be tuned to rapidly hydrolyze. When a peptide has a longer residence time in the target location, a slower hydrolysis rate would allow for extended delivery of an active agent.

The rate of hydrolysis of the linker (e.g., a linker of a peptide conjugate) can be measured. Such measurements can include determining free active agent in plasma, or synovial fluid, or other fluid or tissue of a subject in vivo and/or by incubating a linker or a peptide conjugate comprising a linker of the present disclosure with a buffer (e.g., PBS) or blood plasma from a subject (e.g., rat plasma, human plasma, etc.) or synovial fluid or other fluids or tissues ex vivo. The methods for measuring hydrolysis rates can include taking samples during incubation or after administration and determine free active agent, free peptide, or any other parameter indicate of hydrolysis, including also measuring total peptide, total active agent, or conjugated active agent-peptide. The results of such measurements can then be used to determine a hydrolysis half-life of a given linker or peptide conjugate comprising the linker. A hydrolysis half-life of a linker can differ depending on the plasma or fluid or species or other conditions used to determine such half-life. This can be due to certain enzymes or other compounds present in a certain plasma (e.g., rat plasma). For instance, different fluids (such as plasma or synovial fluid) can contain different amounts of enzymes such as esterases, and these levels of these compounds can also vary depending on species (such as rat versus human) as well as disease state (such as normal versus arthritic).

The conjugates of the present disclosure can be described as having a modular structure comprising various components, wherein each of the components (e.g., peptide, linker, active agent and/or detectable agent) can be selected dependently or independently of any other component. For example, a conjugate for use in the treatment of pain can comprise a PD-L1-binding peptide of the present disclosure (e.g., those having the amino acid sequence of any one of SEQ ID NO: 1-SEQ ID NO: 118, SEQ ID NO: 435, SEQ ID NO: 436, or SEQ ID NO: 554-SEQ ID NO: 567), a linker (e.g., any linker described in TABLE 8 or TABLE 9, SEQ ID NO: 154-SEQ ID NO: 241, SEQ ID NO: 433, or otherwise described) and an active agent (e.g., a therapeutic agent, a detectable agent, or an immune cell binding moiety). The linker, for example, can be selected and/or modified to achieve a certain active agent release (e.g., a certain release rate) via a certain mechanism (e.g., via hydrolysis, such as enzyme and/or pH-dependent hydrolysis) at the target site (e.g., in the brain) and/or to minimize systemic exposure to the active agent. During the testing of a conjugate any one or more of the components of the conjugate can be modified and/or altered to achieve certain in vivo properties of the conjugate, e.g., pharmacokinetic (e.g., clearance time, bioavailability, uptake and retention in various organs) and/or pharmacodynamic (e.g., target engagement) properties. Thus, the conjugates of the present disclosure can be modulated to prevent, treat, and/or diagnose a variety of diseases and conditions, while reducing side effects (e.g., side effects that occur if such active agents are administered alone (i.e., not conjugated to a peptide)).

In some embodiments, the non-natural amino acid can comprise one or more functional groups, e.g., alkene or alkyne, that can be used as functional handles. For example, a multiple bond of such functional groups can be used to add one or more molecules to the conjugate. The one or more molecules can be added using various synthetic strategies, some of which may include addition and/or substitution chemistries. For example, an addition reaction using a multiple bond can comprise the use of hydrobromic acid, wherein the bromine can act as a leaving group and thus be substituted with various moieties, e.g., active agents, detectable agents, agents that can modify or alter the pharmacokinetic (e.g., plasma half-life, retention and/or uptake in central nervous system (CNS) or elsewhere) and/or pharmacodynamic (e.g., hydrolysis rate such as an enzymatic hydrolysis rate) properties of the conjugate.

In some embodiments, a conjugate as described herein comprises one or more non-natural amino acid and/or one or more linkers. Such one or more non-natural amino acid and/or one or more linkers can comprise one or more functional groups, e.g., alkene or alkyne (e.g., non-terminal alkenes and alkynes), which can be used as functional handles. For example, a multiple bond of such functional groups can be used to add one or more molecules to the conjugate. The one or more molecules can be added using various synthetic strategies, some of which may include addition and/or substitution chemistries, cycloadditions, etc. For example, an addition reaction using a multiple bond can comprise the use of hydrogen bromide (e.g., via hydrohalogenation reactions), wherein the bromide substituent, once attached, can act as a leaving group and thus be substituted with various moieties comprising a nucleophilic functional groups, e.g., active agents, detectable agents, agents. As another example, a multiple bond can be used as a functional handle in a cycloaddition reaction. Cycloaddition reactions can comprise 1,3-dipolar cycloadditions, [2+2]-cycloadditions (e.g., photocatalyzed), Diels-Alder reactions, Huisgen cycloadditions, nitrone-olefin cycloadditions, etc. Such cycloaddition reactions can be used to attached various functional groups, functional moieties, active agents, detectable agents, and so forth to the conjugate. For example, a 1,3-dipolar cycloaddition reaction can be used to attach a molecule to a conjugate, wherein the molecule comprises a 1,3-dipole that can react with, e.g., an alkyne to form a 5-membered ring, thereby attaching said molecule to the conjugate.

The addition of such agents or molecules (e.g., via nucleophilic or electrophilic addition followed by nucleophilic substitution) can have various application. For example, attaching such molecule or agent can modify or alter the pharmacokinetic (e.g., plasma half-life, retention and/or uptake in CNS or biodistribution) and/or pharmacodynamic (e.g., hydrolysis rate such as an enzymatic hydrolysis rate) properties of the conjugate. Attaching such molecule or agent can also alter (e.g., increase) the depot effect of a conjugate, or provide functionality for in vivo tracking, e.g., using fluorescence or other types of detectable agents.

In some embodiments, a conjugate of the present disclosure can comprise a linker comprising one or more of the following groups:

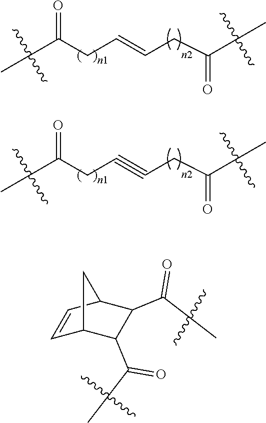

or a substituted analog or a stereoisomer thereof, wherein each n1 and n2 is independently a value from 1 to 10. Such a group can be used as a handle to attach one or more molecules to a conjugate, e.g., to alter the pharmacokinetic (e.g., plasma half-life, retention and/or uptake in central nervous system (CNS) or elsewhere) and/or pharmacodyna via nucleophilic or electrophilic addition followed by nucleophilic substitution mic properties of the conjugate. Functionalization of such a group can occur using one or more multiple bonds (e.g., double bonds, triple bonds, etc.) of the groups. Such functionalization can comprise addition and/or substitution chemistries. For example, a functional group of a linker, such as a double bond, can be converted into a single bond (e.g., via an addition reaction such as a nucleophilic addition reaction), wherein one or both of the carbon atoms of the newly formed single bond can have a leaving group (e.g., a bromine) attached to them. Such a leaving group can then be used (e.g., via nucleophilic substitution reaction) to attach a specific molecule (e.g., an active agent, a detectable agent, etc.) to that carbon atom(s) of the linker.

As another example, a multiple bond can be used as a functional handle in a cycloaddition reaction. Cycloaddition reactions can comprise 1,3-dipolar cycloadditions, [2+2]-cycloadditions (e.g., photocatalyzed), Diels-Alder reactions, Huisgen cycloadditions, nitrone-olefin cycloadditions, etc. Such cycloaddition reactions can be used to attached various functional groups, functional moieties, active agents, detectable agents, and so forth to the conjugate. For example, a 1,3-dipoalr cycloaddition reaction can be used to attach a molecule to a conjugate, wherein the molecule comprises a 1,3-dipole that can react with, e.g., an alkyne to form a 5-membered ring, thereby attaching said molecule (e.g., active agent, detectable agent, etc.) to the conjugate. In some cases, molecules may be attached to a conjugate to e.g., modulate the half-life, increase the depot effect, or provide new functionality of a conjugate, such as fluorescence for tracking.

### Peptide Linkers

The peptides of the presented disclosure (e.g., PD-L1-binding peptides or peptide complexes) can be linked or fused in numerous ways. For example, a PD-L1-binding peptide can be linked or fused to an active agent (e.g., a therapeutic agent, an immune cell binding moiety, an Fc, or an albumin-binding peptide) via a peptide linker to form a PD-L1-binding peptide complex. In some embodiments, a peptide linker does not disturb the independent folding of peptide domains (e.g., of a PD-L1-binding peptide). In some embodiments, a peptide linker does not negatively impact manufacturability (synthetic or recombinant) of the peptide complex (e.g., the PD-L1-binding peptide complex). In some embodiments, a peptide linker does not impair post-synthesis chemical alteration (e.g. conjugation of a fluorophore or albumin-binding chemical group) of the peptide complex (e.g., the PD-L1-binding peptide complex).

In some embodiments, a peptide linker can connect the C-terminus of a first peptide (e.g., a PD-L1-binding peptide) to the N-terminus of a second peptide (e.g., an active agent peptide). In some embodiments, a peptide linker can connect the C-terminus of the second peptide (e.g., an active agent peptide) to the N-terminus of a third peptide (e.g., a PD-L1-binding peptide).

In some embodiments, a linker can comprise a Tau-theraphotoxin-Hs1a, also known as DkTx (double-knot toxin), extracted from a native knottin-knottin dimer from Haplopelma schmidti (e.g., SEQ ID NO: 166). The linker can lack structural features that would interfere with dimerizing independently functional proteins or peptides (e.g., a PD-L1-binding peptide and an immune cell targeting agent). In some embodiments, a linker can comprise a glycine-serine (Gly-Ser or GS) linker (e.g., SEQ ID NO: 154-SEQ ID NO: 165 or SEQ ID NO: 194-SEQ ID NO: 199). Gly-Ser linkers can have minimal chemical reactivity and can impart flexibility to the linker. Serines can increase the solubility of the linker or the peptide complex, as the hydroxyl on the side chain is hydrophilic. In some embodiments, a linker can be derived from a peptide that separates the Fc from the Fv domains in a heavy chain of human immunoglobulin G (e.g., SEQ ID NO: 167). In some embodiments, a linker derived from a peptide from the heavy chain of human IgG can comprise a cysteine to serine mutation relative to the native IgG peptide.

In some embodiments, peptides of the present disclosure can be dimerized using an immunoglobulin heavy chain Fc domain. These Fc domains can be used to dimerize functional domains (e.g., a PD-L1-binding peptide and an immune cell targeting agent), either based on antibodies or other otherwise soluble functional domains. In some embodiments, dimerization can be homodimeric if the Fc sequences are native. In some embodiments, dimerization can be heterodimeric by mutating the Fc domain to generate a “knob-in-hole” format where one Fc CH3 domain contains novel residues (knob) designed to fit into a cavity (hole) on the other Fc CH3 domain. A first peptide domain (e.g., a PD-L1-binding peptide) can be coupled to the knob, and a second peptide domain (e.g., an immune cell targeting agent) can be coupled to the hole. Knob+knob dimers can be highly energetically unfavorable. A purification tag can be added to the “knob” side to remove hole+hole dimers and select for knob+hole dimers.

The peptides of the present disclosure (e.g., the PD-L1-binding peptides) can be linked to another peptide (e.g., an active agent peptide) at the N-terminus or C-terminus. In some embodiments, one or more peptides can be linked or fused via a peptide linker (e.g., a peptide linker comprising a sequence of any of SEQ ID NO: 154-SEQ ID NO: 241 or SEQ ID NO: 433). For example, a PD-L1-binding peptide can be fused to an active agent via a peptide linker of any of SEQ ID NO: 154-SEQ ID NO: 241 or SEQ ID NO: 433. A peptide linker (e.g., a linker connecting a PD-L1-binding peptide and an active agent) can have a length of about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45, or about 50 amino acid residues. A peptide linker can have a length of from about 2 to about 5, from about 2 to about 10, from about 2 to about 20, from about 3 to about 5, from about 3 to about 10, from about 3 to about 15, from about 3 to about 20, from about 3 to about 25, from about 5 to about 10, from about 5 to about 15, from about 5 to about 20, from about 5 to about 25, from about 10 to about 15, from about 10 to about 20, from about 10 to about 25, from about 15 to about 20, from about 15 to about 25, from about 20 to about 25, from about 20 to about 30, from about 20 to about 35, from about 20 to about 40, from about 20 to about 45, from about 20 to about 50, from about 3 to about 50, from about 3 to about 40, from about 3 to about 30, from about 10 to about 40, from about 10 to about 30, from about 50 to about 100, from about 100 to about 200, from about 200 to about 300, from about 300 to about 400, from about 400 to about 500, or from about 500 to about 600 amino acid residues.

In some embodiments, a peptide can be appended to the N-terminus of any peptide of the present disclosure following an N-terminal GS dipeptide and preceding, for example, a GGGS (SEQ ID NO: 154) spacer. In some embodiments, a peptide (e.g., an active agent) can be appended to either the N-terminus or C-terminus of any peptide disclosed herein (e.g., a PD-L1-binding peptide) using a peptide linker such as GₓS_{y} (SEQ ID NO: 155) peptide linker, wherein x and y independently are any whole number, such as 1, 2, 3, 4, 5, 6, 7, 8, 9, 10, 12, 16, or 20 and the G and S residues are arranged in any order. In some embodiments, the peptide linker comprises (GS)x (SEQ ID NO: 156), wherein x can be any whole number, such as 1, 2, 3, 4, 5, 6, 7, 8, 9, and 10. In some embodiments, the peptide linker comprises GGSSG (SEQ ID NO: 157), GGGGG (SEQ ID NO: 158), GSGSGSGS (SEQ ID NO: 159), GSGG (SEQ ID NO: 160), GGGGS (SEQ ID NO: 161), GGGS (SEQ ID NO: 154), GGS (SEQ ID NO: 162), GGGSGGGSGGGS (SEQ ID NO: 163), or a variant or fragment thereof or any number of repeats and combinations thereof. Additionally, KKYKPYVPVTTN (SEQ ID NO: 166) from DkTx, and EPKSSDKTHT (SEQ ID NO: 167) from human IgG₃ can be used as a peptide linker or any number of repeats and combinations thereof. In some embodiments, the peptide linker comprises GGGSGGSGGGS (SEQ ID NO: 164) or a variant or fragment thereof or any number of repeats and combinations thereof.

In some embodiments, a linker of the present disclosure can comprise a cleavable or stable linker moiety. In some embodiments, cleavable linkers of the present disclosure can include, for example, protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers (e.g., esterase cleavable linker), ultrasound-sensitive linkers, and X-ray cleavable linkers. In some embodiments, the linker is not a cleavable linker.

A linker can comprise multiple amino acids. A linker can comprise 1, 2, 3, 4, 5, 6, 7, 8, 9, 10, 20 or more amino acids. A linker can comprise any of the linkers in the below TABLE 9 (where X=6-azidohexanoic acid and Z=citrulline). In some cases, an active can be attached to a peptide using any one or more of the linkers shown below in TABLE 9. In some embodiments, the peptide linker comprises a linker of any of SEQ ID NO: 154-SEQ ID NO: 241 or SEQ ID NO: 433. Examples of peptide linkers compatible with the peptide complexes of the present disclosure are provided in TABLE 9. It is understood that any of the foregoing linkers or a variant or fragment thereof can be used with any number of repeats or any combinations thereof. It is also understood that other peptide linkers in the art or a variant or fragment thereof can be used with any number of repeats or any combinations thereof.

TABLE 9
Exemplary Peptide Linkers

| SEQ ID NO | Sequence |
|---|---|
| SEQ ID NO: 154 | GGGS |
| SEQ ID NO: 155 | GₓS_{y} |
| SEQ ID NO: 156 | (GS)x |
| SEQ ID NO: 157 | GGSSG |
| SEQ ID NO: 158 | GGGGG |
| SEQ ID NO: 159 | GSGSGSGS |
| SEQ ID NO: 160 | GSGG |
| SEQ ID NO: 161 | GGGGS |
| SEQ ID NO: 162 | GGS |
| SEQ ID NO: 163 | GGGSGGGSGGGS |
| SEQ ID NO: 164 | GGGSGGSGGGS |
| SEQ ID NO: 165 | GGGGSGGGGSGGGGS |
| SEQ ID NO: 166 | KKYKPYVPVTTN |
| SEQ ID NO: 167 | EPKSSDKTHT |
| SEQ ID NO: 168 | AGSGGSGGSGGSPVPSTPPTNSSSTPPTPSPSPVPSTPPTNSSSTPPTP |
| | SPSPVPSTPPTNSSSTPPTPSPSAS |
| SEQ ID NO: 169 | AGSGGSGGSGGSPVPSTPPTPSPSTPPTPSPSPVPSTPPTNSSSTPPTP |
| | SPSPVPSTPPTPSPSTPPTPSPSAS |
| SEQ ID NO: 170 | AGSGGSGGSGGSPVPSTPPTPSPSTPPTPSPSGGSGNSSGSGGSPVPS |
| | TPPTPSPSTPPTPSPSAS |
| SEQ ID NO: 171 | AGSGGSGGSGGSPVPSTPPTPSPSTPPTPSPSPVPSTPPTPSPSTPPTP |
| | SPSPVPSTPPTPSPSTPPTPSPSAS |
| SEQ ID NO: 172 | AGSGGSGGSGGSPVPSTPPTPSPSTPPTPSPSIQRTPKIQVYSRHPAE |
| | NGKSNFLNCYVSGFHPSDIEVDLLKNGERIEKVEHSDLSFSKDWSF |
| | YLLYYTEFTPTEKDEYACRVNHVTLSQPKIVKWDRDPVPSTPPTPS |
| | PSTPPTPSPSAS |
| SEQ ID NO: 173 | AGSGGSGGSGGSPVPSTPPTPSPSTPPTPSPSIQRTPKIQVYSRHPAE |
| | NGKSNFLNCYVSGFHPSDIEVDLLKNGERIEKVEHSDLSFSKDWSF |
| | YLLYYTEFTPTEKDEYACRVNHVTLSQPKIVKWDRDGGSGGSGG |
| | SGGSAS |
| SEQ ID NO: 174 | AGPVPSTPPTPSPSTPPTPSPSIQRTPKIQVYSRHPAENGKSNFLNCY |
| | VSGFHPSDIEVDLLKNGERIEKVEHSDLSFSKDWSFYLLYYTEFTP |
| | TEKDEYACRVNHVTLSQPKIVKWDRDGGSGGSGGSGGSIQRTPKI |
| | QVYSRHPAENGKSNFLNCYVSGFHPSDIEVDLLKNGERIEKVEHS |
| | DLSFSKDWSFYLLYYTEFTPTEKDEYACRVNHVTLSQPKIVKWDR |
| | DGGSGGSGGSGAS |
| SEQ ID NO: 175 | AGSGGSGGSGGSPVPSTPPTPSPSTPPTPSPSIQRTPKIQVYSRHPAE |
| | NGKSNFLNCYVSGFHPSDIEVDLLKNGERIEKVEHSDLSFSKDWSF |
| | YLLYYTEFTPTEKDEYACRVNHVTLSQPKIVKWDRDGGSGGSGG |
| | SGGSIQRTPKIQVYSRHPAENGKSNFLNCYVSGFHPSDIEVDLLKN |
| | GERIEKVEHSDLSFSKDWSFYLLYYTEFTPTEKDEYACRVNHVTLS |
| | QPKIVKWDRDPVPSTPPTPSPSTPPTPSPSAS |
| SEQ ID NO: 176 | AGSGGSGGSGGSPVPSTPPTPSPSTPPTPSPSQIFVKTLTGKTITLEV |
| | EPSDTIENVKAKIQDKEGIPPDQQRLIFAGKQLEDGRTLSDYNIQKE |
| | STLHLVLRLRGGGGSGGSGGSGGSAS |
| SEQ ID NO: 177 | AGSGGSGGSGGSPVPSTPPTPSPSTPPTPSPSDGRYSLTYIYTGLSK |
| | HVEDVPAFQALGSLNDLQFFRYNSKDRKSQPMGLWRQVEGMED |
| | WKQDSQLQKAREDIFMETLKDIVEYYNDSNGSHVLQGRFGCEIEN |
| | NRSSGAFWKYYYDGKDYIEFNKEIPAWVPFDPAAQITKQKWEAE |
| | PVYVQRAKAYLEEECPATLRKYLKYSKNILDRQDPPSVVVTSHQA |
| | PGEKKKLKCLAYDFYPGKIDVHWTRAGEVQEPELRGDVLHNGNG |
| | TYQSWVVVAVPPQDTAPYSCHVQHSSLAQPLVVPWEASPVPSTPP |
| | TPSPSTPPTPSAS |
| SEQ ID NO: 178 | AGSGGSGGSGGSGGSGGSGGSGGSDGRYSLTYIYTGLSKHVEDVP |
| | AFQALGSLNDLQFFRYNSKDRKSQPMGLWRQVEGMEDWKQDSQ |
| | LQKAREDIFMETLKDIVEYYNDSNGSHVLQGRFGCEIENNRSSGAF |
| | WKYYYDGKDYIEFNKEIPAWVPFDPAAQITKQKWEAEPVYVQRA |
| | KAYLEEECPATLRKYLKYSKNILDRQDPPSVVVTSHQAPGEKKKL |
| | KCLAYDFYPGKIDVHWTRAGEVQEPELRGDVLHNGNGTYQSWV |
| | VVAVPPQDTAPYSCHVQHSSLAQPLVVPWEASGGSGGSGGSGGS |
| | DGRYSLTYIYTGLSKHVEDVPAFQALGSLNDLQFFRYNSKDRKSQ |
| | PMGLWRQVEGMEDWKQDSQLQKAREDIFMETLKDIVEYYNDSN |
| | GSHVLQGRFGCEIENNRSSGAFWKYYYDGKDYIEFNKEIPAWVPF |
| | DPAAQITKQKWEAEPVYVQRAKAYLEEECPATLRKYLKYSKNIL |
| | DRQDPPSVVVTSHQAPGEKKKLKCLAYDFYPGKIDVHWTRAGEV |
| | QEPELRGDVLHNGNGTYQSWVVVAVPPQDTAPYSCHVQHSSLAQ |
| | PLVVPWEASPVPSTPPTPSPSTPPTPSPSAS |
| SEQ ID NO: 179 | AGSGNSSGSGGSGGSGNSSGSGGSPVPSTPPTPSPSTPPTPSPSAS |
| SEQ ID NO: 180 | KLSGGGGSGGGGSGGGGSAEAWYNLGNAYYKQGDYQKAIEYYQ |
| | KALELDPNNAEAWYNLGNAYYKQGDYQKAIEYYQKALELDPNN |
| | AEAWYNLGNAYYKQGDYQKAIEDYQKALELDPNNLQRSAGGGG |
| | SGGGGSGGGGAS |
| SEQ ID NO: 181 | KLSGGGGSGGGGSGGGGSAEAWYNLGNAYYKQGDYQKAIEYYQ |
| | KALELDPNNAEAWYNLGNAYYKQGDYQKAIEYYQKALELDPNN |
| | AEAWYNLGNAYYKQGDYQKAIEDYQKALELDPNNLQAEAWKN |
| | LGNAYYKQGDYQKAIEYYQKALELDPNNASAWYNLGNAYYKQ |
| | GDYQKAIEYYQKALELDPNNAKAWYRRGNAYYKQGDYQKAIED |
| | YQKALELDPNNRSRSAGGGGSGGGGSGGGGAS |
| SEQ ID NO: 182 | KLSGGGGSGGGGSGGGGSAEAWYNLGNAYYKQGDYQKAIEYYQ |
| | KALELDPNNAEAWYNLGNAYYKQGDYQKAIEYYQKALELDPNN |
| | AEAWYNLGNAYYKQGDYQKAIEDYQKALELDPNNLQAEAWKN |
| | LGNAYYKQGDYQKAIEYYQKALELDPNNASAWYNLGNAYYKQ |
| | GDYQKAIEYYQKALELDPNNAKAWYRRGNAYYKQGDYQKAIED |
| | YQKALELDPNNRSAEAWYNLGNAYYKQGDYQKAIEYYQKALEL |
| | DPNNAEAWYNLGNAYYKQGDYQKAIEYYQKALELDPNNAEAW |
| | YNLGNAYYKQGDYQKAIEDYQKALELDPNNLQRSAGGGGSGGG |
| | GSGGGGAS |
| SEQ ID NO: 183 | KLSGGGGSGGGGSGGGGSAEAWYNLGNAYYKQGDYQKAIEYYQ |
| | KALELDPNNAEAWYNLGNAYYKQGDYQKAIEYYQKALELDPNN |
| | AEAWYNLGNAYYKQGDYQKAIEDYQKALELDPNNLQAEAWKN |
| | LGNAYYKQGDYQKAIEYYQKALELDPNNASAWYNLGNAYYKQ |
| | GDYQKAIEYYQKALELDPNNAKAWYRRGNAYYKQGDYQKAIED |
| | YQKALELDPNNRSAEAWYNLGNAYYKQGDYQKAIEYYQKALEL |
| | DPNNAEAWYNLGNAYYKQGDYQKAIEYYQKALELDPNNAEAW |
| | YNLGNAYYKQGDYQKAIEDYQKALELDPNNLQAEAWKNLGNAY |
| | YKQGDYQKAIEYYQKALELDPNNASAWYNLGNAYYKQGDYQK |
| | AIEYYQKALELDPNNAKAWYRRGNAYYKQGDYQKAIEDYQKAL |
| | ELDPNNRSAGGGGSGGGGSGGGGAS |
| SEQ ID NO: 184 | GGGGSAS |
| SEQ ID NO: 185 | GGGGSGGGGSAS |
| SEQ ID NO: 186 | GGGGSGGGGSGGGGSAS |
| SEQ ID NO: 187 | GGGGSGGGGSGGGGSGGGGSGGGGSAS |
| SEQ ID NO: 188 | GGGGSGGGGSGGGGSGGGGSGGGGSGGGGSAS |
| SEQ ID NO: 189 | AGGGSGGGGSGGGGSGGGGSGGGGSGGGGSGGGGSAS |
| SEQ ID NO: 190 | AGGGSGGGGSGGGGSGGGGSGGGGSGGGGSGGGGSGGGGSAS |
| SEQ ID NO: 191 | AGGGSGGGGSGGGGSGGGGSGGGGSGGGGSGGGGSGGGGSGGG |
| | GSAS |
| SEQ ID NO: 192 | G |
| SEQ ID NO: 193 | A |
| SEQ ID NO: 194 | GS |
| SEQ ID NO: 195 | GA |
| SEQ ID NO: 196 | GSS |
| SEQ ID NO: 197 | GSSS |
| SEQ ID NO: 198 | GSSSS |
| SEQ ID NO: 199 | (GSSS)_{y} where y = 1-10 |
| SEQ ID NO: 200 | VA |
| SEQ ID NO: 201 | VZ |
| SEQ ID NO: 202 | GVZG |
| SEQ ID NO: 203 | FK |
| SEQ ID NO: 204 | VK |
| SEQ ID NO: 205 | GVAG |
| SEQ ID NO: 206 | GVZGG |
| SEQ ID NO: 207 | XG |
| SEQ ID NO: 208 | XGG |
| SEQ ID NO: 209 | XA |
| SEQ ID NO: 210 | XAA |
| SEQ ID NO: 211 | XGS |
| SEQ ID NO: 212 | XGA |
| SEQ ID NO: 213 | XGVZG |
| SEQ ID NO: 214 | XGVAG |
| SEQ ID NO: 215 | XGVZGG |
| SEQ ID NO: 216 | Val-Arg |
| SEQ ID NO: 217 | Val-Cit |
| SEQ ID NO: 218 | Phe-Lys |
| SEQ ID NO: 219 | Met-Lys |
| SEQ ID NO: 220 | Asn-Lys |
| SEQ ID NO: 221 | Ile-Pro |
| SEQ ID NO: 222 | Gly-Ile |
| SEQ ID NO: 223 | Gly-Leu |
| SEQ ID NO: 224 | Gly-Tyr |
| SEQ ID NO: 225 | Gly-Met |
| SEQ ID NO: 226 | Met-Ile |
| SEQ ID NO: 227 | Ala-Ile |
| SEQ ID NO: 228 | Pro-Ile |
| SEQ ID NO: 229 | Gly-Pro-Gln-Gly-Ile-Ala-Gly-Gln |
| SEQ ID NO: 230 | Gly-Pro-Gln-Gly-Ile-Phe-Gly-Gln |
| SEQ ID NO: 231 | Gly-Pro-Gln-Gly-Ile-Trp-Gly-Gln |
| SEQ ID NO: 232 | Gly-Pro-Gln-Gly-Ile-Leu-Gly-Gln |
| SEQ ID NO: 233 | Gly-Pro-Gln-Gly-Ile-Arg-Gly-Gln |
| SEQ ID NO: 234 | Gly-Pro-Leu-Gly-Ile-Ala-Gly-Gln |
| SEQ ID NO: 235 | Gly-Pro-Met-Gly-Ile-Ala-Gly-Gln |
| SEQ ID NO: 236 | Gly-Pro-Tyr-Gly-Ile-Ala-Gly-Gln |
| SEQ ID NO: 237 | GSVAGS |
| SEQ ID NO: 238 | GGGGSVAGGGGS |
| SEQ ID NO: 239 | GGGGSGGGGSVAGGGGSGGGGS |
| SEQ ID NO: 240 | GGGGSGGGGSPLGLAGGGGGSGGGGS |
| SEQ ID NO: 241 | AEAAAKEAAAKAVAAEAAAKEAAAKA |
| SEQ ID NO: 433 | GGGSGGGS |

A linker can provide a minimum distance between the PD-L1-binding peptide and the active agent, such that the active agent does not inhibit or prevent binding of the PD-L1-binding peptide to PD-L1. Similarly, linker can provide a minimum distance between the PD-L1-binding peptide and the active agent (e.g., an additional binding moiety), such that the PD-L1-binding peptide does not inhibit or prevent binding of the active agent to its target (e.g., an immune cell target). A linker can be long enough to avoid steric hindrance of the active agent inhibiting binding of the peptide to PD-L1. A linker can be longer than the shortest distance of the N-terminal amine in the peptide to PD-L1 when bound. A linker can be longer than a salt bridge, which can be 2-4 angstroms long. A linker can be at least 5, 10, 20, 40 or more angstroms long. A linker can comprise at least 1, at least 2, at least 3, at least 5, at least 10, at least 15, at least 20, and least 25, or more carbon, oxygen, nitrogen, sulfur, and/or phosphorous atoms in the linker backbone between the peptide and the oligonucleotide. A linker can include 1, 2, 3, 4, 5, 10, 15, 20 or more amino acids. A linker can include 1, 2, 3, 4, 5, 10, 20 or more nucleotide bases.

A peptide or peptide complex according to the present disclosure may be attached to another moiety such as a small molecule, a second peptide, a second CDP, a protein, a miniprotein, a cytokine, a cytokine-receptor chain complex, an antibody, an antibody fragment, an Fc, an Fc knob, an Fc hole, an aptamer, polypeptide, polynucleotide, a fluorophore, a radioisotope, a radionuclide chelator, a polymer, a biopolymer, a fatty acid, an acyl adduct, a binding moiety, a chemical linker, or sugar, immune-oncology agent, or other active agent described herein through a linker, or directly in the absence of a linker.

A peptide or peptide complex can be conjugated a nucleotide, an active agent, or a detectable agent via a linker that can be described with the formula Peptide-A-B-C-active agent, wherein the linker is A-B-C. A can be a stable amide link to an amine or carboxylic acid on the peptide and the linker and can be achieved via a tetrafluorophenyl (TFP) ester, an NHS ester, or an ATT group (thiazolidine-thione). A can be a stable carbamate linker such as that formed by reacting an amine on the peptide with an imidazole carbamate active intermediate formed by reaction of CDI with a hydroxyl on the linker. A can be a stable secondary amine linkage such as that formed by reductive alkylation of the amine on the peptide with an aldehyde or ketone group on the linker. A can be a stable thioether linker formed using a maleimide or bromoacetamide in the linker with a thiol in the peptide, a triazole linker, a stable oxime linker, or an oxacarboline linker. A can comprise a triazole. B can comprise (—CH2-)ₓ-, with or without branching a short PEG (—CH₂CH₂O—)ₓ (x is 1-20), or a short polypeptide such as GGGSGGGS (SEQ ID NO: 433), Val-Ala (SEQ ID NO: 200), Val-Cit (SEQ ID NO: 217), Val-Cit-PABC, Gly-Ile (SEQ ID NO: 222), Gly-Leu (SEQ ID NO: 223), other spacers, or no spacer. C can be a disulfide bond, an amide bond, a triazole bond, carbamate, a carbon-carbon single double or triple bond, or an ester bond to a thiol, an amine, a hydroxyl, or carboxylic acid on the active agent. C can be a thioether formed between a maleimide on the linker and a sulfhydroyl on the active agent, a secondary or tertiary amine, a carbamate, or other stable bond. In some embodiments, C can refer to the “cleavable” or “stable” part of the linker. In other embodiments, A and/or B can also be the “cleavable” or stable part. In some embodiments, A can be amide, carbamate, thioether via maleimide or bromoacetamide, triazole, oxime, or oxacarboline. Any linker chemistry described in “Current ADC Linker Chemistry,” Jain et al., Pharm Res, 2015 DOI 10.1007/s11095-015-1657-7 or in Bioconjugate Techniques, 3^{rd} edition, by Greg Hermanson can be used.

### Methods of Delivery and Treatment Using Peptides and Peptide Complexes

In some embodiments, the PD-L1-binding peptides of the present disclosure can induce a biologically relevant response. In some embodiments, the biologically relevant response can be induced after intravenous, subcutaneous, peritoneal, intracranial, intrathecal, intratumoral, or intramuscular dose, and in some embodiments, after a single intravenous, subcutaneous, peritoneal, intracranial, or intramuscular dose. In some embodiments, the PD-L1-binding peptides or PD-L1-binding peptide complexes can be used alone or in combination with various other classes of therapeutic compounds used to treat various diseases or conditions including cancers or immunological disorders (e.g., autoimmune diseases).

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case can be determined using techniques, such as a dose escalation study.

The methods, compositions, and kits of this disclosure can comprise a method to prevent, treat, arrest, reverse, or ameliorate the symptoms of a condition. The treatment can comprise treating a subject (e.g., an individual, a domestic animal, a wild animal, or a lab animal afflicted with a disease or condition) with a peptide or peptide complex of the disclosure. The disease can be a cancer or tumor. The disease can be an autoimmune disorder. In treating the disease, the peptide can contact the tumor or cancerous cells or an immune cell. The subject can be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment can be provided to the subject for more than 1 day, 1 week, 1 month, 6 months, 12 months, 2 years or more after clinical onset of disease. Treatment can be provided to the subject for less than 1 day, 1 week, 1 month, 6 months, 12 months, or 2 years after clinical onset of the disease. Treatment can also include treating a human in a clinical trial. A treatment can comprise administering to a subject a pharmaceutical composition, such as one or more of the pharmaceutical compositions described throughout the disclosure. A treatment can comprise a once daily dosing, twice a day dosing, dosing every other day, dosing every third day, dosing every week, dosing every other week, dosing every month, dosing every three months, or dosing every six months. A treatment can comprise delivering a peptide of the disclosure to a subject, either intravenously, subcutaneously, intramuscularly, by inhalation, dermally, topically, by intra-articular injection, orally, sublingually, intrathecally, transdermally, intranasally, via a peritoneal route, intratumorally (e.g., directly into a tumor such as via injection), directly into the brain (e.g., via and intracerebral ventricle route), or directly onto a joint, e.g. via topical, intra-articular injection route. A treatment can comprise administering a peptide-active agent complex to a subject, either intravenously, subcutaneously, intramuscularly, by inhalation, by intra-articular injection, dermally, topically, orally, intrathecally, transdermally, intransally, parenterally, orally, via a peritoneal route, nasally, sublingually, or directly onto cancerous tissues. Intravenous dosing can be bolus or can be by infusion.

### PD-L1 and PD-1 Inhibition

A PD-L1-binding peptide of the present disclosure (e.g., any one of SEQ ID NO: 1-SEQ ID NO: 118, SEQ ID NO: 435, SEQ ID NO: 436, or SEQ ID NO: 554-SEQ ID NO: 567) or a PD-L1-binding peptide complex may be administered to a subject (e.g., a human or non-human animal subject) to inhibit PD-L1 activity in the subject. PD-L1 activity may be associated with immunosuppression, T cell exhaustion, or immune function, and inhibiting PD-L1 may reduce immunosuppression, reduce T cell exhaustion, restore immune function, or a combination thereof. Inhibition of PD-L1 may be beneficial in diseases such as cancer in which PD-L1 positive cancer cells may evade a host immune response by inhibiting interactions between PD-L1 on the cancer cell and PD-1 on a host T cell. In some embodiments, inhibiting PD-L1 (e.g., by administering a PD-L1-binding peptide) may enhance a host immune response against the cancer cell, thereby treating the cancer. In some embodiments, inhibiting PD-L1 (e.g., by administering a PD-L1-binding peptide) may enhance a host immune response in the case of chronic infection, where T cell exhaustion may be a problem, or in sepsis or other acute infections.

The PD-L1-binding peptides of the present disclosure may inhibit PD-L1 by blocking interactions between PD-L1 and PD-1. For example, SEQ ID NO: 1 binds PD-L1 at the PD-1 binding interface, preventing PD-1 from accessing the binding interface. In some embodiments, the PD-L1 binding peptides of the present disclosure may inhibit PD-L1 by binding to and stabilizing PD-L1 in an inactive conformation.

Administration of a PD-L1-binding peptide or peptide complex may be used in a method of treating cancer by binding to and inhibiting PD-L1 upon administration to a subject. Inhibition of PD-L1 may reduce T cell exhaustion and enhance a host immune response against the cancer. The PD-L1-binding peptides described herein may be used to treat any PD-L1 positive cancer. Examples of cancers that may be treated by administering a PD-L1-binding peptide or peptide complex include melanoma, non-small cell lung cancer, small cell lung cancer, renal cancer, esophageal cancer, oral cancer, hepatocellular cancer, ovarian cancer, cervical cancer, colorectal cancer, lymphoma, bladder cancer, liver cancer, gastric cancer, breast cancer, pancreatic cancer, prostate cancer, Merkel cell carcinoma, mesothelioma, or brain cancer (e.g., glioblastoma, astrocytoma, meningioma, metastatic brain cancer, or primary brain cancer).

### Active Agent Delivery

A PD-L1-binding peptide complex of the present disclosure (e.g., a complex comprising a peptide of any one of SEQ ID NO: 1-SEQ ID NO: 118, SEQ ID NO: 435, SEQ ID NO: 436, or SEQ ID NO: 554-SEQ ID NO: 567 complexed with an active agent) may be used in a method of delivering an active agent to a cell, region, or tissue of interest in a subject. Upon administration, the PD-L1-binding peptide complex may target and bind to PD-L1 (e.g., on a PD-L1 positive cell) and deliver the active agent to the cell, tissue, or region containing the PD-L1. Targeted delivery of active agents to PD-L1 positive cells, tissues, or regions may increase the therapeutic window of the active agent compared to administration of the active agent alone because targeted delivery may result in an increased concentration of active agent at the target cell, tissue, or region compared to surrounding tissue. This may reduce negative off target effects, decrease the dosage needed to produce a therapeutic effect, or both.

A PD-L1-binding peptide complex may be administered to delivery an active agent to any PD-L1 positive cell, region, or tissue. For example, a PD-L1-binding peptide complex may deliver an active agent to a cancer cell, an immune cell, or a pancreatic beta cell. Examples of active agents that may be delivered to a PD-L1 positive cell may include immune cell targeting agents, immune cells (e.g., a T cell, a B cell, a macrophage, a natural killer cell, a fibroblast, a regulatory T cell, a regulatory immune cell, a neural stem cell, or a mesenchymal stem cell), anti-cancer agents, chemotherapeutic agents, radiotherapy agents, proinflammatory cytokines, or oligonucleotides.

Chemotherapeutic or anti-cancer agents may function by killing or inhibiting proliferation of a target cancer cell (e.g., a PD-L1 positive cancer cell). Examples of chemotherapeutics or anti-cancer agents that may be complexed with a PD-L1-binding peptide of the present disclosure include antineoplastic agents, cytotoxic agents, tyrosine kinase inhibitors, mTOR inhibitors, retinoids, or anti-cancer antibodies. Proinflammatory cytokines may function by stimulating an immune response against a target (e.g., a PD-L1 positive cancer cell). Examples of proinflammatory cytokines that may be complexed with a PD-L1-binding peptide of the present disclosure include TNFα, IL-2, TL-6, IL-12, TL-15, IL-21, or IFNγ. Anti-inflammatory agents may function by inhibiting an inflammatory response in or around the target (e.g., by inhibiting a cyclooxygenase enzyme or stimulating a glucocorticoid receptor). Examples of anti-inflammatory agents that may be complexed with a PD-L1-binding peptide of the present disclosure include anti-inflammatory cytokines, steroids, glucocorticoids, corticosteroids, or nonsteroidal anti-inflammatory drugs (NSAIDs).

Oligonucleotides may function by modulating alternative splicing of the target sequence, dictating the location of a polyadenylation site of the target sequence, inhibiting translation of the target sequence, inhibiting binding of the target sequence to a secondary target sequence, recruiting RISC to the target sequence, recruiting RNaseH1 to the target sequence, inducing cleavage of the target sequence, or regulating the target sequence upon binding of the oligonucleotide to the target sequence. In some embodiments, the oligonucleotide may comprise an oncolytic viral vector, an mRNA, an miRNA, or an siRNA.

In some embodiments, a PD-L1-binding peptide complex may be used to deliver an active agent to treat a disease or condition associated with PD-L1. For example, a PD-L1-binding peptide complex may be administered to treat a cancer (e.g., melanoma, skin cancer, non-small cell lung cancer, small cell lung cancer, non-small-cell lung carcinoma, renal cancer, esophageal cancer, oral cancer, hepatocellular cancer, ovarian cancer, cervical cancer, colorectal cancer, colon cancer, rectal cancer, head and neck cancer, lymphoma, bladder cancer, liver cancer, gastric cancer, stomach cancer, breast cancer, pancreatic cancer, prostate cancer, Merkel cell carcinoma, mesothelioma, or brain cancer, including primary brain cancer or metastatic brain cancer, a PDL1-expressing cancer, a primary cancer, a metastatic cancer, gastric cancer, squamous cell carcinoma, urothelial carcinoma, or cervical cancer), an autoimmune disease (e.g., rheumatoid arthritis, atherosclerosis, ischemia-reperfusion injury, colitis, psoriasis, lupus, inflammatory bowel disease, Crohn's disease, ulcerative colitis, multiple sclerosis, type 1 diabetes, or neuroinflammation), hyperglycemia, type 2 diabetes, infection, or neuronal injury. In some embodiments, treatment of cancer may comprise delivering an anti-cancer agent or immune stimulating agent to a PD-L1 positive cancer cell. In some embodiments, treatment of an autoimmune disorder may comprise delivery of an anti-inflammatory agent or immunosuppressive agent to a PD-L1 positive immune cell, thereby reducing an autoimmune response in the subject. In some embodiments, treatment of hyperglycemia may comprise delivering a protective agent to a pancreatic beta cell, thereby preventing onset of type 1 diabetes.

### Nucleotide and Oligo Delivery

A peptide (e.g., a PD-L1-binding peptide) may be linked, conjugated, complexed with, or fused to a nucleotide via various chemistries resulting in peptide oligonucleotide complexes that may form either a cleavable or stable linkage to deliver the oligonucleotide to a cell. For example, in some embodiments, a PD-L1-binding peptide may bind to PD-L1 on the surface of cells, which may then be taken up via endocytosis into the early endosome. The nucleotide and peptide in the PD-L1-binding peptide oligonucleotide complex may either remain together (stable) or be cleaved apart (cleavable). If the linkage is stable, the PD-L1-binding peptide oligonucleotide complex may recycle back to the cell surface. Some of the PD-L1-binding peptide oligonucleotide complex may access low pH early endosomes. Once the nucleotides within the peptide oligonucleotide complex are exposed to endosome, they may remain and not be degraded due to stabilization chemistry such as by the oligonucleotide (backbone, sugar, linkage, etc.) variations described herein. If the PD-L1-binding peptide includes additional cell penetration capabilities, the peptide may facilitate accelerated escape of the oligonucleotide from the endosomal compartment into the cytosol. Even without added cell penetration capabilities, the oligonucleotides may slowly leak out of endosomes and access the cytosol.

Cleavage away from the PD-L1 binder peptide within the peptide oligonucleotide complex may be advantageous in order to avoid repeated cycling to the cell surface or to facilitate endosomal escape of the oligonucleotide, in which case cleavable linkers may be used between the oligonucleotide and the peptide. The nucleotides within or cleaved from the peptide oligonucleotide complex may traffic, either actively or by passive diffusion, between the cytosol and nucleus. Some of the nucleotides within the peptide oligonucleotide complex can function within the nucleus of a cell, including gapmers, ASO splice blockers, and U1 adapters. Others function within the cytosol, including siRNA and anti-miRs. Aptamers are unique in that they do not function through hybridization or base paring interactions with nucleic acid targets. Instead, aptamers form secondary structures to bind to proteins or other macromolecules. Aptamers may function wherever the target protein or macromolecule is located. For example, if the target is on the surface of cells, cell penetration via endosomal accumulation may not be necessary, and it may be advantageous for linkers to be cleavable or non-cleavable depending on PD-L1-binding peptide trafficking and stability.

The nucleotide portion of the peptide oligonucleotide complexes described herein may target specific RNAs (e.g., mRNAs or pre-mRNAs) from genes expressed in cancer and other diseases. For example, the nucleotide sequence in the complex may be complementary to any target provided in SEQ ID NO: 397-SEQ ID NO: 430 or SEQ ID NO: 549, TABLE 10, or TABLE 17. The nucleotide sequence in the complex may be complementary to the target RNA, or in the case of an aptamer, may bind a target protein or other macromolecule. The a nucleotide sequence may be single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter.

In some embodiments, a target of the nucleotide in a peptide oligonucleotide complex may be a gastrointestinal target, such as a gene with pro-inflammatory, extracellular matrix-modifying, or immune cell recruitment functionality. Peptide oligonucleotide complexes described herein (e.g., a peptide oligonucleotide complex comprising a PD-L1-binding peptide and a nucleotide that binds a gene target mRNA) that target gastrointestinal gene targets may be used to treat various gastrointestinal disorders, including inflammatory bowel disease (IBD), ulcerative colitis, and Crohn's disease.

In some embodiments, a target of the nucleotide in a peptide oligonucleotide complex may be a cancer target, such as a gene involved in oncogenic signaling, anti-apoptotic genes, pro-inflammatory signaling genes, protein homeostasis genes, developmental regulatory genes, or adapter protein genes that initiate downstream cell growth signaling. For example, targeting an over-expressed growth factor like HER2 can be challenging, but HER2 and other RTK (e.g., EGFR, ERBB3) signaling depends on adapter proteins like Grb2 to initiate cell growth signaling downstream. Knockdown of Grb2 can halt signaling in a way that is difficult to mutationally compensate as Grb2 loss is epistatic to HER2 activity. Cancer cells are typically under low levels of proteotoxic stress, as they are growing so quickly that their protein folding machinery struggles to keep up, so targeting protein homeostasis genes, such as heat shock proteins (HSPs), hypoxia-sensing proteins (e.g., HIF), and upregulators of the heat shock response, may reduce proteotoxic stress by helping to fold or stabilize proteins during folding. In some embodiments, a pro-inflammatory cytokine may be delivered via an mRNA in a peptide oligonucleotide complex, or an antisense construct targeting an anti-inflammatory signal may be delivered. Delivery of a pro-inflammatory signal or reduction of an anti-inflammatory signal may help to recruit B cells, T cells, macrophages, or other immune infiltrates to a tumor microenvironment. Peptide oligonucleotide complexes described herein (e.g., a peptide oligonucleotide complex comprising a PD-L1-binding peptide and a nucleotide that binds a gene target mRNA) that target cancer gene targets may be used to treat various cancers, including solid tumors. Developmental regulators, such as transcription factors involved in early cell fate and pluripotency, and chromatin remodeling enzymes, may be targeted to specifically harm de-differentiated cells which may be present in advanced tumors and associated with a more mobile and/or mitotic cell state. A peptide oligonucleotide construct targeting a cancer target may treat or prevent cancer by reducing oncogenic signaling, reducing target over-expression, reducing oncogenic antisense activity (e.g., miRNAs targeting tumor suppressors), and/or eliminating the source of the oncogenic signaling cascade.

Examples of gene targets (e.g., gastrointestinal, or cancer gene targets) are provided in TABLE 10.

TABLE 10
Examples of Disease-Specific Gene Targets

| Type/Target | Target Gene | Associated Disorders |
|---|---|---|
| Gastrointestinal | Pro-inflammatory genes | IBD, Ulcerative Colitis, Crohn's Disease |
| Gastrointestinal | ECM-modifying genes | IBD, Ulcerative Colitis, Crohn's Disease |
| Gastrointestinal | Immune cell recruitment genes | IBD, Ulcerative Colitis, Crohn's Disease |
| Gastrointestinal | TNF-α | IBD, Ulcerative Colitis, Crohn's Disease |
| Gastrointestinal | ICAM-1 | IBD, Ulcerative Colitis, Crohn's Disease |
| Gastrointestinal | NF-κB | IBD, Ulcerative Colitis, Crohn's Disease |
| Gastrointestinal | NF-κB subunits | IBD, Ulcerative Colitis, Crohn's Disease |
| Gastrointestinal | NF-κB p65 subunit | IBD, Ulcerative Colitis, Crohn's Disease |
| Gastrointestinal | Smad7 | IBD, Ulcerative Colitis, Crohn's Disease |
| Gastrointestinal | Carbohydrate Sulfotransferase | IBD, Ulcerative Colitis, Crohn's Disease |
| | 15 (CHST15) | |
| Gastrointestinal | IL-23 | IBD, Ulcerative Colitis, Crohn's Disease |
| Gastrointestinal | IL-12 | IBD, Ulcerative Colitis, Crohn's Disease |
| Gastrointestinal | IL-17 | IBD, Ulcerative Colitis, Crohn's Disease |
| Cancer Cells | Insulin-like Growth Factor 1 | Cancers, Solid Tumors |
| | (IGF-1) | |
| Cancer Cells | IGF-1 Receptor | Cancers, Solid Tumors |
| Cancer Cells | Androgen Receptor | Cancers, Solid Tumors |
| Cancer Cells | EGFR | Cancers, Solid Tumors |
| Cancer Cells | EGFR Isoforms | Cancers, Solid Tumors |
| Cancer Cells | EGFRvIII Isoform | Cancers, Solid Tumors |
| Cancer Cells | ERBB3 | Cancers, Solid Tumors |
| Cancer Cells | HER2 | Cancers, Solid Tumors |
| Cancer Cells | GRB2 | Cancers, Solid Tumors |
| Cancer Cells | KRAS | Cancers, Solid Tumors |
| Cancer Cells | Mutant KRAS | Cancers, Solid Tumors |
| Cancer Cells | KRAS-G12D | Cancers, Solid Tumors |
| Cancer Cells | MYC | Cancers, Solid Tumors |
| Cancer Cells | YAP1 | Cancers, Solid Tumors |
| Cancer Cells | Heat Shock Proteins | Cancers, Solid Tumors |
| Cancer Cells | HSP90 | Cancers, Solid Tumors |
| Cancer Cells | HSP70 | Cancers, Solid Tumors |
| Cancer Cells | HSP27 | Cancers, Solid Tumors |
| Cancer Cells | Hypoxia-sensing Proteins | Cancers, Solid Tumors |
| Cancer Cells | HIF1A | Cancers, Solid Tumors |
| Cancer Cells | HIF2A | Cancers, Solid Tumors |
| Cancer Cells | Pro-Inflammatory Cytokines | Cancers, Solid Tumors |
| Cancer Cells | IL-12 | Cancers, Solid Tumors |
| Cancer Cells | IL-12 mRNA | Cancers, Solid Tumors |
| Cancer Cells | TGF1B | Cancers, Solid Tumors |
| Cancer Cells | miR-21 mRNA | Cancers, Solid Tumors |
| Cancer Cells | MDM2 | Cancers, Solid Tumors |
| Cancer Cells | BCL2 | Cancers, Solid Tumors |
| Cancer Cells | FOXP3 | Cancers, Solid Tumors |
| Cancer Cells | DNMT1 | Cancers, Solid Tumors |
| Cancer Cells | HDACs | Cancers, Solid Tumors |
| Cancer Cells | Myb | Cancers, Solid Tumors |
| Cancer Cells | Fos | Cancers, Solid Tumors |
| Cancer Cells | Jun | Cancers, Solid Tumors |
| Blasts | NUP98-KDM5A | Erythroleukemia |
| | NTRK1 | |
| | JAK2, K-/N-RAS | |
| Blasts | JAK-STAT pathway; | Acute myeloid leukemia |
| | Hedgehog pathway; | |
| | PI3K/AKT pathway; | |
| | RAF/MEK/ERK pathway; | |
| | mTOR pathway | |
| varies | JAK-STAT pathway | Myeloproliferative neoplasms |
| | HDAC | |
| | MDM2, LSD1, CALR | |
| Uroepithelial | Viral sequences | BK virus |
| cells | | |
| Varies | Viral sequences | MS |
| Microglia, | Proinflammatory mediators, | Parkinson's Disease |
| astrocytes | such as IL1b, IL6, TNFa, | |
| | IFNg, chemokines | |
| Neurons | LRRK2 | Parkinson's Disease |
| Myocytes | Myostatin | Muscular disorders |
| Eye | HCMV | CMV retinitis |
| Eye | VEGF-165 | Neovascular age-related macular |
| | | degeneration |
| Liver | APOB | Hypercholesterolemia |
| Spinal cord | SMN2 | SMA |
| Muscle | DMD | DMD |
| Muscle | DMD | DMD |
| Muscle | DMD | DMD |
| Muscle | DMD | DMD |
| Kidney | miR-17 | ADPKD |
| Muscle | DMPK | DM1 |
| Liver | TTR | Transthyretin-mediated amyloidosis |
| Liver | TTR | Transthyretin-mediated amyloidosis |
| Liver | TTR | Transthyretin-mediated amyloidosis |
| Liver | TTR | Transthyretin-mediated amyloidosis |
| Liver | APOC-III | Familial chylomicronemia syndrome |
| Liver | SERPINC1 | Hemophilia A + B |
| Liver, kidney | HAO1 | Primary hyperoxaluria type 1 |
| Liver | ALAS1 | Acute hepatic porphyria |
| Eye | LCA10 | Leber's congenital amaurosis type 10 |
| Liver | ApoA | CVD |
| Liver | PCSK9 | CVD |
| Liver | APOC-III | CVD |
| GI | ICAM-1 | UC |
| Platelet cells | Factor XI | Clotting disorder |
| Pancreas | GCGR | Type II diabetes |
| Liver | DGAT2 | NASH |
| Cancer Cells | HSP27 | Cancer, solid tumors |
| Cancer Cells | STAT3 | Cancer |
| Cancer Cells | AR | Cancer |
| Cancer Cells | miR-155 | Cancer |
| Cancer Cells | GRB2 | Cancer, AML |
| Cancer Cells | TGFB2 | Cancer, glioma, prostate cancer |
| Muscle | CD49d | MS, DMD |
| | GHR | Acromegaly |
| | HBV surface antigen | Hepatitis B |
| Muscle | DNM2 | Centronuclear myopathy |
| Lungs | ENAC | Cystic fibrosis |
| | ANGPTL3 | Dyslipidemia |
| | AGT | Hypertension |
| Kidney | Factor B | Primary IgA nephropathy |
| | Kallikrein B1 | Hereditary angioedema |
| Eye | RHO | Retinitis pigmentosa |
| Eye | USH2A | Retinitis pigmentosa |
| Skin | COL7A1 | Dystrophic epidermolysis bullosa |
| Kidney | miR-21 | Alport syndrome |
| Skin | IL17RA | Psoriasis |

In some embodiments, an oligonucleotide may target a gene for downregulation. For example, PvRBSA, PvRBP2b, PfEMP1, pfmdr1, pfgchl, GPX4, SLC7a11, alpha-synuclein, PD-L1, NUP98-KDM5A, NTRK1, JAK2, K-/N-RAS, the JAK-STAT pathway, the Hedgehog pathway, the PI3K/AKT pathway, the RAF/MEK/ERK pathway, the mTOR pathway, HDAC, MDM2, LSD1, CALR, PKC, NF-κB, HSP90, HIV Tat, TNF-α, CCR2, CCR5, TAR (tat), RRE (rev), vpr, U5 leader, Nef, Gag, Vif, Env, IL1b, IL6, TNFα, IFNg, LRRK2, or Myostatin may be targeted for downregulation.

An example of an antagomir that may be complexed with a PD-L1-binding peptide to target a gene includes cobomarsen. An example of an aptamer that may be complexed with a PD-L1-binding peptide to target a gene includes pegaptanib. Examples of gapmers that may be complexed with a PD-L1-binding peptide to target a gene include fomivirsen, mipomersen, inotersen, volanesorsen, tofersen, tominersen, pelacarsen, alicaforsen, apatorsen, and trabedersen. Examples of siRNAs that may be complexed with a PD-L1-binding peptide to target a gene include patisiran, vutrisiran, revusiran, fitusiran, lumasiran, givosiran, and inclisiran. Examples of splice blockers that may be complexed with a PD-L1-binding peptide to target a gene include nusinersen, eteplirsen, golodirsen, viltolarsen, casimersen, and sepofarsen. An example of a translation blocker that may be complexed with a PD-L1-binding peptide to target a gene includes prexigebersen.

Any targets for the nucleic acid portion of the peptide oligonucleotide complex described herein can be used in conjunction with a U1 adapter to degrade targeted mRNAs. The target recognition (or complementary nucleic acid to the target mRNA) portion directs the peptide oligonucleotide complex to the targeted mRNA selected for degradation, while the U1 portion prevents the addition of polyA to the mRNA resulting in degradation of the targeted mRNA. U1 adapters can comprise any nucleotide sequence complementary to the ssRNA component of the U1 small nuclear ribonucleoprotein (U1 snRNP). In some embodiments, the U1 adapter sequences engage the U1 snRNP near its poly A site. In some embodiments, the length of the U1 adapter is 15 to 25 nt in length, or about 20 nt in length. In some embodiments, the U1 adapter is above 40% in its G/C content. Exemplary U1 adapters are shown in TABLE 11, in conjunction with a target nucleic acid “target recognition” portion which comprises a nucleotide sequence is single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, or splice blocker ASO. The 10-19 nt U1 Adapter is italicized.

TABLE 11
Examples of Target Recognition
Constructs with U1 Adapters

| 5′-Nucleic Acid complementary | |
|---|---|
| to Target mRNA-U1 Adapter-3′ | SEQ ID NO: |
| 5′-[Target_recognition]UCCC | SEQ ID NO: 366 |
| CUGCCAGGUAAGUAU-3′ [19 nt] | |
| 5′-[Target_recognition]CCCU | SEQ ID NO: 367 |
| GCCAGGUAAGUAU-3′ [17 nt] | |
| 5′-[Target_recognition]CUGC | SEQ ID NO: 553 |
| CAGGUAAGUAU-3′ [15 nt] | |
| 5′-[Target_recognition]UGCC | SEQ ID NO: 368 |
| AGGUAAGUAU-3′ [14 nt] | |
| 5′-[Target_recognition]GCCA | SEQ ID NO: 370 |
| GGUAAGUAU-3′ [13 nt] | |
| 5′-[Target_recognition]CCAG | SEQ ID NO: 371 |
| GUAAGUAU-3′ [12 nt] | |
| 5′-[Target_recognition]CAGG | SEQ ID NO: 372 |
| UAAGUAU-3′ [11 nt] | |
| 5′-[Target_recognition]CAGG | SEQ ID NO: 373 |
| UAAGUA-3′ [10 nt] | |

Exemplary U1 adapters include: UCCCCUGCCAGGUAAGUAU (SEQ ID NO: 366); CCCUGCCAGGUAAGUAU (SEQ ID NO: 367); CUGCCAGGUAAGUAU (SEQ ID NO: 368); UGCCAGGUAAGUAU (SEQ ID NO: 369); GCCAGGUAAGUAU (SEQ ID NO: 370); CCAGGUAAGUAU (SEQ ID NO: 371); CAGGUAAGUAU (SEQ ID NO: 372); and CAGGUAAGUA (SEQ ID NO: 373).

### Detectable Labeling and Imaging

A PD-L1-binding peptide complex of the present disclosure (e.g., a complex comprising a peptide of any one of SEQ ID NO: 1-SEQ ID NO: 118, SEQ ID NO: 435, SEQ ID NO: 436, or SEQ ID NO: 554-SEQ ID NO: 567 complexed with a detectable agent) may be used in a method of labeling a cell, region, or tissue of interest in a subject. Upon administration, the PD-L1-binding peptide complex may target and bind to PD-L1 (e.g., on a PD-L1 positive cell) and deliver the detectable agent to the cell, tissue, or region containing the PD-L1. The detectable agent of the peptide complex may produce a detectable signal and may be used to label a cell or tissue (e.g., a PD-L1 positive cell or tissue). In some embodiments, producing a detectable signal may comprise emitting a fluorescent light (e.g., a visible, ultraviolet, or infrared light), emitting electromagnetic radiation, absorbing electromagnetic radiation (e.g., light or X-rays), producing a contrast signal, producing an electron spin signal, emitting radiation, producing a magnetic signal, or combinations thereof. Examples of detectable agents that may be complexed with a PD-L1-binding peptide of the present disclosure include fluorophores, near-infrared dyes, contrast agents, nanoparticles, metal-containing nanoparticles, metal chelates, X-ray contrast agents, PET agents, radionuclides, or radionuclide chelators.

Delivery of a detectable agent to a PD-L1 positive region may be used in a method of diagnosing a disease or condition in a subject, for example a condition associated with PD-L1. For example, a PD-L1-binding peptide complex comprising a detectable agent may be administered to a subject who has or is suspected of having cancer. The peptide complex may target and bind to PD-L1 positive cancer cells, thereby labeling the PD-L1 positive cancer cells. The presence and location of the detectable agent may be imaged to diagnose the cancer.

### Immune Cell Recruitment

A PD-L1-binding peptide complex of the present disclosure (e.g., a complex comprising a peptide of any one of SEQ ID NO: 1-SEQ ID NO: 118, SEQ ID NO: 435, SEQ ID NO: 436, or SEQ ID NO: 554-SEQ ID NO: 567 complexed with an immune cell targeting agent) may be used in a method of recruiting an immune cell to a PD-L1 positive cell, tissue, or region. The PD-L1-binding peptide complex may comprise a bispecific immune cell engager. Upon administration, the bispecific immune cell engager may bind to a PD-L1 positive cell via the PD-L1-binding peptide and to an immune cell (e.g., a T cell, a B cell, a macrophage, a natural killer cell, a fibroblast, a regulatory T cell, a regulatory immune cell, a neural stem cell, or a mesenchymal stem cell) via the immune cell targeting agent. The bispecific immune cell engager may recruit the immune cell to the PD-L1 positive cell. In some embodiments, recruitment of the immune cell may stimulate an immune response against the target cell. For example, recruitment of a T cell, an NK cell, a macrophage, a fibroblast, a regulatory immune cell, a neural stem cell, or a mesenchymal stem cell to a PD-L1 positive cancer cell may induce a host immune response against the cancer cell or otherwise therapeutically modulate the microenvironment around PD-L1-positive tissue. In some embodiments, recruitment of the immune cell may inhibit an immune response against the target cell. For example, recruitment of a regulatory T cell (T_{reg}) to a pancreatic beta cell may protect the pancreatic beta cell and prevent the onset of type 1 diabetes. Type 1 diabetes occurs when T cells engage with insulin-producing pancreatic beta cells and attack them as an autoimmune disorder. Recruitment of T_{reg} cells to islets via dual engagement of PD-L1+beta cells and T_{reg} cells may reduce this autoimmune destruction of beta cells. In another example, recruitment of a regulatory T cell or a natural killer cell to a T cell involved in an autoimmune response may inhibit the autoimmune response and treat an autoimmune disorder. Regulatory T cells recruited to inflammatory tissue may produce anti-inflammatory signaling, thereby reducing inflammation at the site of the inflammatory tissue. Producing anti-inflammatory signaling at the site of PD-L1-positive pancreatic islet cells being invaded by activated T cells may delay, slow progression of, or reverse type 1 diabetes. In some embodiments, a BiICE comprising a PD-L1-binding peptide (e.g., a peptide of any one of SEQ ID NO: 1-SEQ ID NO: 118, SEQ ID NO: 435, SEQ ID NO: 436, or SEQ ID NO: 554-SEQ ID NO: 567) may be used in a method of recruiting a regulatory T cell or a natural killer cell to a PD-L1 positive cell, tissue, or region complexed with a regulatory T cell- or a natural killer cell-binding moiety. Recruitment of a neural stem cell may treat a site or neuronal injury. Recruitment of anti-inflammatory, regulatory immune cells (e.g. Tregs) can be helpful in chronic infection or sepsis or acute infection. A bispecific immune cell engager utilizing a PD-L1-binding peptide may also be administered to treat a cancer (e.g., melanoma, skin cancer, non-small cell lung cancer, small cell lung cancer, renal cancer, esophageal cancer, oral cancer, hepatocellular cancer, ovarian cancer, cervical cancer, colorectal cancer, colon cancer, rectal cancer, head and neck cancer, lymphoma, bladder cancer, liver cancer, gastric cancer, stomach cancer, breast cancer, pancreatic cancer, prostate cancer, Merkel cell carcinoma, mesothelioma, or brain cancer, including primary brain cancer or metastatic brain cancer, a PDL1-expressing cancer, a primary cancer, a metastatic cancer), an autoimmune or inflammatory disease (e.g., rheumatoid arthritis, atherosclerosis, ischemia-reperfusion injury, colitis, psoriasis, lupus, inflammatory bowel disease, Crohn's disease, ulcerative colitis, multiple sclerosis, type 1 diabetes, or neuroinflammation), hyperglycemia, type 2 diabetes, infection, or neuronal injury.

### CAR T-Cell Therapy

A PD-L1-binding peptide complex of the present disclosure (e.g., chimeric antigen receptor comprising a PD-L1-binding peptide of any one of SEQ ID NO: 1-SEQ ID NO: 118, SEQ ID NO: 435, SEQ ID NO: 436, or SEQ ID NO: 554-SEQ ID NO: 567) may be used in a method of recruiting a T cell to a PD-L1 positive cell, tissue, or region. For example, a PD-L1-binding chimeric antigen receptor (CAR) may be used in a CAR T-cell therapy. The PD-L1-binding chimeric antigen receptor may be expressed in a T cell (e.g., a T cell collected from a subject), and T cells expressing the CAR may be administered back to the subject. The PD-L1-binding peptide of the CAR may deliver the T cell to a PD-L1 positive cell of the subject (e.g., a PD-L1 positive cancer cell). The CAR T-cell may stimulate an immune response against the PD-L1 positive cell. In some embodiments, a PD-L1-binding CAR may be administered to treat a cancer (e.g., melanoma, skin cancer, non-small cell lung cancer, small cell lung cancer, renal cancer, esophageal cancer, oral cancer, hepatocellular cancer, ovarian cancer, cervical cancer, colorectal cancer, colon cancer, rectal cancer, head and neck cancer, lymphoma, bladder cancer, liver cancer, gastric cancer, stomach cancer, breast cancer, pancreatic cancer, prostate cancer, Merkel cell carcinoma, mesothelioma, or brain cancer, including primary brain cancer or metastatic brain cancer, a PDL1-expressing cancer, a primary cancer, a metastatic cancer).

### Peptide Stability

A peptide of the present disclosure can be stable in various biological or physiological conditions, such as physiologic extracellular pH, endosomal or lysosomal pH, or reducing environments inside a cell, in the cytosol, in a cell nucleus, or endosome or a tumor. For example, any peptide or peptide complex comprising any of SEQ ID NO: 1-SEQ ID NO: 118, SEQ ID NO: 435, SEQ ID NO: 436, or SEQ ID NO: 554-SEQ ID NO: 567 can exhibit resistance to reducing agents, proteases, oxidative conditions, or acidic conditions.

In some cases, biologic molecules (such as peptides and proteins) can provide therapeutic functions, but such therapeutic functions are decreased or impeded by instability caused by the in vivo environment. (Moroz et al. Adv Drug Deliv Rev 101:108-21 (2016), Mitragotri et al. Nat Rev Drug Discov 13(9):655-72 (2014), Bruno et al. Ther Deliv (11):1443-67 (2013), Sinha et al. Crit Rev Ther Drug Carrier Syst. 24(1):63-92 (2007), Hamman et al. BioDrugs 19(3):165-77 (2005)). For instance, the GI tract can contain a region of low pH (e.g. pH˜1), a reducing environment, or a protease-rich environment that can degrade peptides and proteins. Proteolytic activity in other areas of the body, such as the mouth, eye, lung, intranasal cavity, joint, skin, vaginal tract, mucous membranes, and serum, can also be an obstacle to the delivery of functionally active peptides and polypeptides. Additionally, the half-life of peptides in serum can be very short, in part due to proteases, such that the peptide can be degraded too quickly to have a lasting therapeutic effect when administering reasonable dosing regimens. Likewise, proteolytic activity in cellular compartments such as lysosomes and reduction activity in lysosomes and the cytosol can degrade peptides and proteins such that they can be unable to provide a therapeutic function on intracellular targets. Therefore, peptides that are resistant to reducing agents, proteases, and low pH can be able to provide enhanced therapeutic effects or enhance the therapeutic efficacy of co-formulated or conjugated, linked, or fused active agents in vivo.

Additionally, oral delivery of drugs can be desirable in order to target certain areas of the body (e.g., disease in the GI tract such as colon cancer, irritable bowel disorder, infections, metabolic disorders, and constipation) despite the obstacles to the delivery of functionally active peptides and polypeptides presented by this method of administration. For example, oral delivery of drugs can increase compliance by providing a dosage form that is more convenient for patients to take as compared to parenteral delivery. Oral delivery can be useful in treatment regimens that have a large therapeutic window. Therefore, peptides that are resistant to reducing agents, proteases, and low pH can allow for oral delivery of peptides without nullifying their therapeutic function.

Peptide Resistance to Reducing Agents. PD-L1-binding peptides or peptide complexes of this disclosure can contain one or more cysteines, which can participate in disulfide bridges that can be integral to preserving the folded state of the peptide. Exposure of peptides to biological environments with reducing agents can result in unfolding of the peptide and loss of functionality and bioactivity. For example, glutathione (GSH) is a reducing agent that can be present in many areas of the body, in the blood, and inside cells and can reduce disulfide bonds. As another example, a peptide can become reduced during trafficking of a peptide across the gastrointestinal epithelium after oral administration. A peptide can become reduced upon exposure to various parts of the GI tract. The GI tract can be a reducing environment, which can inhibit the ability of therapeutic molecules with disulfide bonds to have optimal therapeutic efficacy, due to reduction of the disulfide bonds. A peptide can also be reduced upon entry into a cell, such as after internalization by endosomes or lysosomes or into the cytosol, or other cellular compartments. Reduction of the disulfide bonds and unfolding of the peptide can lead to loss of functionality or affect key pharmacokinetic parameters such as bioavailability, peak plasma concentration, bioactivity, and half-life. Reduction of the disulfide bonds can also lead to loss of functionality due to increased susceptibility of the peptide to subsequent degradation by proteases, resulting in rapid loss of intact peptide after administration. In some embodiments, a peptide that is resistant to reduction can remain intact and can impart a functional activity for a longer period of time in various compartments of the body and in cells, as compared to a peptide that is more readily reduced.

In certain embodiments, the peptides of this disclosure can be analyzed for the characteristic of resistance to reducing agents to identify stable peptides. In some embodiments, the peptides of this disclosure can remain intact after being exposed to different molarities of reducing agents such as 0.00001 M-0.0001 M, 0.0001 M-0.001 M, 0.001 M-0.01 M, 0.01 M-0.05 M, 0.05 M-0.1 M, or 0.1 M to 0.2 M for 15 minutes or more. In some embodiments, the reducing agent used to determine peptide stability can be dithiothreitol (DTT), Tris(2-carboxyethyl)phosphine HCl (TCEP), 2-Mercaptoethanol, (reduced) glutathione (GSH), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a reducing agent. In some embodiments, peptides are completely resistant to GSH reducing conditions and are partially resistant to degradation in DTT reducing conditions. In some embodiments, peptides described herein can withstand or are resistant to degradation in physiological reducing conditions.

Peptide Resistance to Proteases. The stability of peptides of this disclosure can be determined by resistance to degradation by proteases. Proteases, also referred to as peptidases or proteinases, are enzymes that can degrade peptides and proteins by breaking bonds between adjacent amino acids. Families of proteases with specificity for targeting specific amino acids can include serine proteases, cysteine proteases, threonine proteases, aspartic proteases, glutamic proteases, and asparagine proteases. Additionally, metalloproteases, matrix metalloproteases, elastase, carboxypeptidases, Cytochrome P450 enzymes, and cathepsins can also digest peptides and proteins. Proteases can be present at high concentration in blood, in mucous membranes, lungs, skin, the GI tract, the mouth, nose, eye, and in compartments of the cell. Misregulation of proteases can also be present in various diseases such as rheumatoid arthritis and other immune disorders. Degradation by proteases can reduce bioavailability, biodistribution, half-life, and bioactivity of therapeutic molecules such that they are unable to perform their therapeutic function. In some embodiments, peptides that are resistant to proteases can better provide therapeutic activity at reasonably tolerated concentrations in vivo.

In some embodiments, peptides of this disclosure can resist degradation by any class of protease. In certain embodiments, peptides of this disclosure resist degradation by pepsin (which can be found in the stomach), trypsin (which can be found in the duodenum), serum proteases, or any combination thereof. In some embodiments, the proteases used to determine peptide stability can be pepsin, trypsin, chymotrypsin, or any combination thereof. In certain embodiments, peptides of this disclosure can resist degradation by lung proteases (e.g., serine, cysteinyl, and aspartyl proteases, metalloproteases, neutrophil elastase, alpha-1 antitrypsin, secretory leucoprotease inhibitor, and elafin), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a protease.

Peptide Stability in Acidic Conditions. Peptides of this disclosure can be administered in biological environments that are acidic. For example, after oral administration, peptides can experience acidic environmental conditions in the gastric fluids of the stomach and gastrointestinal (GI) tract. The pH of the stomach can range from about 1-4 and the pH of the GI tract ranges from acidic to normal physiological pH descending from the upper GI tract to the colon. In addition, the vagina, late endosomes, and lysosomes can also have acidic pH values, such as less than pH 7. These acidic conditions can lead to denaturation of peptides and proteins into unfolded states. Unfolding of peptides and proteins can lead to increased susceptibility to subsequent digestion by other enzymes as well as loss of biological activity of the peptide. In certain embodiments, the peptides of this disclosure can resist denaturation and degradation in acidic conditions and in buffers, which simulate acidic conditions. In certain embodiments, peptides of this disclosure can resist denaturation or degradation in buffer with a pH less than 1, a pH less than 2, a pH less than 3, a pH less than 4, a pH less than 5, a pH less than 6, a pH less than 7, or a pH less than 8. In some embodiments, peptides of this disclosure remain intact at a pH of 1-3. In certain embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH less than 1, a pH less than 2, a pH less than 3, a pH less than 4, a pH less than 5, a pH less than 6, a pH less than 7, or a pH less than 8. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH of 1-3. In other embodiments, the peptides of this disclosure can be resistant to denaturation or degradation in simulated gastric fluid (pH 1-2). In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to simulated gastric fluid. In some embodiments, low pH solutions such as simulated gastric fluid can be used to determine peptide stability.

In some embodiments, the peptides described herein are resistant to degradation in vivo, in the serum of a subject, or inside a cell. In some embodiments, the peptides are stable at physiological pH ranges, such as about pH 7, about pH 7.5, between about pH 5 to 7.5, between about 6.5 to 7.5, between about pH 5 to 8, or between about pH 5 to 7. In some embodiments, the peptides described herein are stable in acidic conditions, such as less than or equal to about pH 5, less than or equal to about pH 3, or within a range from about 3 to about 5. In some embodiments, the peptides are stable in conditions of an endosome or lysosome, or inside a nucleus.

Peptide Stability at High Temperatures. Peptides of this disclosure can be administered in biological environments with high temperatures. For example, after oral administration, peptides can experience high temperatures in the body. Body temperature can range from 36° C. to 40° C. High temperatures can lead to denaturation of peptides and proteins into unfolded states. Unfolding of peptides and proteins can lead to increased susceptibility to subsequent digestion by other enzymes as well as loss of biological activity of the peptide. In some embodiments, a peptide of this disclosure can remain intact at temperatures from 25° C. to 100° C. High temperatures can lead to faster degradation of peptides. Stability at a higher temperature can allow for storage of the peptide in tropical environments or areas where access to refrigeration is limited. In certain embodiments, 5%-100% of the peptide can remain intact after exposure to 25° C. for 6 months to 5 years. 5%-100% of a peptide can remain intact after exposure to 70° C. for 15 minutes to 1 hour. 5%-100% of a peptide can remain intact after exposure to 100° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 25° C. for at least 6 months to 5 years. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 70° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 100° C. for 15 minutes to 1 hour.

### Methods of Manufacture

Various expression vector/host systems can be utilized for the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides, peptide complexes, or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV)), or transformed with recombinant plasmid expression vectors (e.g., Ti plasmid) containing the aforementioned nucleic acid sequence, or animal cell systems infected with recombinant virus expression vectors (e.g., adenovirus, vaccinia virus, lentivirus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secrete minimal amounts of proteolytic enzymes.

The PD-L1-binding peptides or peptide complexes of this disclosure can be advantageously made by a single recombinant expression system, with no need for chemical synthesis or modifications. For example, a PD-L1-binding peptide or peptide complex can be expressed in CHO cells, yeast, pichia, E. coli, or other organisms.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, peptides can also be synthesized in a cell-free system prior to extraction using a variety of known techniques employed in protein and peptide synthesis.

In some cases, a host cell produces a peptide that has an attachment point for a cargo molecule (e.g., a therapeutic agent). An attachment point could comprise a lysine residue, an N-terminus, a cysteine residue, a cysteine disulfide bond, a glutamic acid or aspartic acid residue, a C-terminus, or a non-natural amino acid. The peptide could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly. Peptide fragments can be then be joined together enzymatically or synthetically.

In other aspects, the peptides of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000).

Nucleic acids, including RNA and DNA polynucleotides, used in the peptide nucleotide complexes described therein can also be produced using the methods described in U.S. Pat. No. 9,279,149, and is incorporated herein by reference. In some embodiments, RNA or DNA polynucleotides are synthesized by enzymatic/PCR methods. For example, RNA polynucleotides can be synthesized using an enzyme, such as a nucleotidyl transferase (e.g., E. coli poly(A) polymerase or E. coli poly(U) polymerase), which can add RNA nucleotides to the 3′ end. Alternatively, E. coli poly(U) polymerase can be used. A 3′ unblocked reversible terminator ribonucleotide triphosphates (rNTPs) can be used during polynucleotide synthesis. Alternatively, 3′blocked, 2′blocked, or 2′-3′ blocked rNTPs can be used alongside either enzyme described above. RNA or DNA polynucleotides can also be synthesized using standard solid-phase synthesis techniques and phosphoramidite-based methods or thiophosphorodiamidate methods. RNA or DNA polynucleotides of the present disclosure can be prepared by conventional solid phase oligonucleotide synthesis. For example any method of solid-phase synthesis can be employed including, but not limited to methods described, as shown at https://www.atdbio.com/content/17/Solid-phase-oligonucleotide-synthesis, and in Albericio (Solid-Phase Synthesis: A practical guide, CRC Press, 2000), Lambert et al. (Oligonucleotide Synthesis: Solid-Phase Synthesis, DNA, DNA Sequencing, RNA, Small Interfering RNA, Nucleoside, Nucleic Acid, Nucleotide, Phosphoramidite, Sense, Betascript Publishing, 2010), and Guzaev, A. P. et al. (Current Protocols in Nucleic Acid Chemistry. 2013; 53:3.1:3.1.1-3.1.60), each of which are incorporated herein by reference. Solid supports such as CPG or polystyrene can be used. Protected 2′-deoxynucleosides (dA, dC, dG, and T), ribonucleosides (A, C, G, and U), or chemically modified nucleosides, such as LNA or BNA can be used. Phosphoramidite chemistry can be used by cycling through the following steps: detritylation of the support-bound 3′-nucleoside, activation and coupling, capping, and oxidation. At the end of synthesis, the protected nucleotide can be cleaved from the support and then deprotected. The product can be purified by HPLC. Protecting groups used in solid-phase synthesis of RNA polynucleotides can include t-butyldimethylsilyl (TBDMS) or tri-iso-propylsilyloxymethyl (TOM). The RNA or DNA polynucleotides can have a modified backbone to enhance stability. Additionally, non-natural or modified bases can be used to serve as unique functional handles for subsequent chemical conjugation. In some embodiments, modification of the 5′ and or 3′ ends of the RNA or DNA can be performed to result in desired functional groups, stability, or activity. In some embodiments, the functional handles comprise modified bases including one or more modified uridine, modified guanosine, modified cytidine, or modified adenosine base of the RNA. An example of such modified base is a uridine with an extended amine. Nucleic acids, including a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), an oligonucleotide complementary to a natural antisense transcripts (NATs) sequences, siRNA, snRNA, aptamer, gapmer, anti-miR, splice blocker ASO, or U1 Adapter can be made using such methods. It may be advantageous to manufacture the oligonucleotide and the peptide by synthetic methods and then conjugate them together, with improved purity, safety, and cost of goods. Oligonucleotides, including modified oligonucleotides, may be manufactured by any of the methods disclosed in Glazier et al. Chemical synthesis and biological application of modified oligonucleotides. Bioconjugate Chemistry, 2020, 31, 1213-1233.

In some embodiments, the peptides of this disclosure can be more stable during manufacturing. For example, peptides of this disclosure can be more stable during recombinant expression and purification, resulting in lower rates of degradation by proteases that are present in the manufacturing process, a higher purity of peptide, a higher yield of peptide, or any combination thereof. In some embodiments, the peptides can also be more stable to degradation at high temperatures and low temperatures during manufacturing, storage, and distribution. For example, in some embodiments peptides of this disclosure can be stable at 25° C. In other embodiments, peptides of this disclosure can be stable at 70° C. or higher than 70° C. In some embodiments, peptides of this disclosure can be stable at 100° C. or higher than 100° C.

### Pharmaceutical Compositions

A pharmaceutical composition of the disclosure can be a combination of any peptide as described herein with other chemical components, such as carriers, stabilizers, diluents, dispersing agents, suspending agents, thickening agents, antioxidants, solubilizers, buffers, osmolytes (e.g., sugars, disaccharides and sugar alcohols), salts, surfactants, amino acids, encapsulating agents, bulking agents, cryoprotectants, and/or excipients. The pharmaceutical composition facilitates administration of a peptide described herein to an organism. In some cases, the pharmaceutical composition comprises factors that extend half-life of the peptide and/or help the peptide to penetrate the target cells. In some embodiments, a pharmaceutical composition comprises a cell modified to express and secrete a PD-L1-binding peptide or peptide complex of the present disclosure.

Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intratumoral, intranasal, and topical administration. A pharmaceutical composition can be administered in a local or systemic manner, for example, via injection of the peptide described herein directly into an organ, optionally in a depot.

Parenteral injections can be formulated for bolus injection, infusion, or continuous infusion. The pharmaceutical compositions can be in a form suitable for parenteral injection as a sterile suspension, solution, or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptide-antibody complexes described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such peptide-antibody complexes described herein to allow for the preparation of highly concentrated solutions.

Alternatively, the peptide described herein can be lyophilized or in powder form for re-constitution with a suitable vehicle, e.g., sterile pyrogen-free water, before use. In some embodiments, a purified peptide is administered intravenously. A peptide described herein can be administered to a subject in order to home, target, migrate to, or be directed to a CNS cell, a brain cell, a cancerous cell, or a tumor. In some embodiments, a peptide can be conjugated to, linked to, or fused to another peptide that provides a targeting function to a specific target cell type in the central nervous system or across the blood brain barrier. Exemplary target cells include a CNS cell, erythrocyte, an erythrocyte precursor cell, an immune cell, a stem cell, a muscle cell, a brain cell, a thyroid cell, a parathyroid cell, an adrenal gland cell, a bone marrow cell, an appendix cell, a lymph node cell, a tonsil cell, a spleen cell, a muscle cell, a liver cell, a gallbladder cell, a pancreas cell, a cell of the gastrointestinal tract, a glandular cell, a kidney cell, a urinary bladder cell, an endothelial cell, an epithelial cell, a choroid plexus epithelial cell, a neuron, a glial cell, an astrocyte, or a cell associated with a nervous system.

A peptide of the disclosure can be applied directly to an organ, or an organ tissue or cells, such as brain or brain tissue or cells, during a surgical procedure. The recombinant peptide described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives.

In practicing the methods of treatment or use provided herein, therapeutically effective amounts of a peptide described herein can be administered in pharmaceutical compositions to a subject suffering from a condition, such as a condition that affects the immune system. In some embodiments, the subject is a mammal such as a human or a primate. A therapeutically effective amount can vary widely depending on the severity of the disease, the age and relative health of the subject, the potency of the compounds used, and other factors.

In some embodiments, a peptide is cloned into a viral or non-viral expression vector. Such expression vector can be packaged in a viral particle, a virion, or a non-viral carrier or delivery mechanism, which is administered to patients in the form of gene therapy. In other embodiments, patient cells are extracted and modified to express a peptide capable of binding PD-L1 ex vivo before the modified cells are returned back to the patient in the form of a cell-based therapy, such that the modified cells will express the peptide once transplanted back in the patient.

Pharmaceutical compositions can be formulated using one or more physiologically acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, buffer exchanging the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically acceptable salt form.

Methods for the preparation of peptide described herein comprising the compounds described herein include formulating peptide described herein with one or more inert, pharmaceutically acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Pharmaceutical compositions can also include permeation or absorption enhancers (Aungst et al. AAPS J. 14(1):10-8. (2012) and Moroz et al. Adv Drug Deliv Rev 101:108-21. (2016)). Permeation enhancers can facilitate uptake of molecules from the GI tract into systemic circulation. Permeation enhancers can include salts of medium chain fatty acids, sodium caprate, sodium caprylate, N-(8-[2-hydroxybenzoyl]amino)caprylic acid (SNAC), N-(5-chlorosalicyloyl)-8-aminocaprylic acid (5-CNAC), hydrophilic aromatic alcohols such as phenoxyethanol, benzyl alcohol, and phenyl alcohol, chitosan, alkyl glycosides, dodecyl-2-N,N-dimethylamino propionate (DDAIPP), chelators of divalent cations including EDTA, EGTA, and citric acid, sodium alkyl sulfate, sodium salicylate, lecithin-based, or bile salt-derived agents such as deoxycholates.

Compositions can also include protease inhibitors including soybean trypsin inhibitor, aprotinin, sodium glycocholate, camostat mesilate, vacitracin, or cyclopentadecalactone.

### Kits

In one aspect, peptides described herein can be provided as a kit. In another embodiment, peptide complexes described herein can be provided as a kit. In another embodiment, a kit comprises amino acids encoding a peptide described herein, a vector, a host organism, and an instruction manual. In some embodiments, a kit includes written instructions on the use or administration of the peptides

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

### EXAMPLES

The invention is further illustrated by the following non-limiting examples.

### Example 1

### Manufacture of Peptides

This example describes the manufacture of the peptides and peptide complexes described herein (e.g., any one of SEQ ID NO: 1-SEQ ID NO: 118, SEQ ID NO: 435, SEQ ID NO: 436, or SEQ ID NO: 554-SEQ ID NO: 567). Peptides derived from proteins were generated in mammalian cell culture using a published methodology. (A. D. Bandaranayke, C. Correnti, B. Y. Ryu, M. Brault, R. K. Strong, D. Rawlings. 2011. Daedalus: a robust, turnkey platform for rapid production of decigram quantities of active recombinant proteins in human cell lines using novel lentiviral vectors. Nucleic Acids Research. (39)21, e143).

The peptide sequence was reverse-translated into DNA, synthesized, and cloned in-frame with siderocalin using standard molecular biology techniques (M. R. Green, Joseph Sambrook. Molecular Cloning. 2012 Cold Spring Harbor Press). The resulting construct was packaged into a lentivirus, transduced into HEK-293 cells, expanded, isolated by immobilized metal affinity chromatography (IMAC), cleaved with tobacco etch virus (TEV) protease, and purified to homogeneity by reverse-phase chromatography. Following purification, each peptide was lyophilized and stored frozen.

### Example 2

### Construction of a Cystine Dense Peptide Screening Library

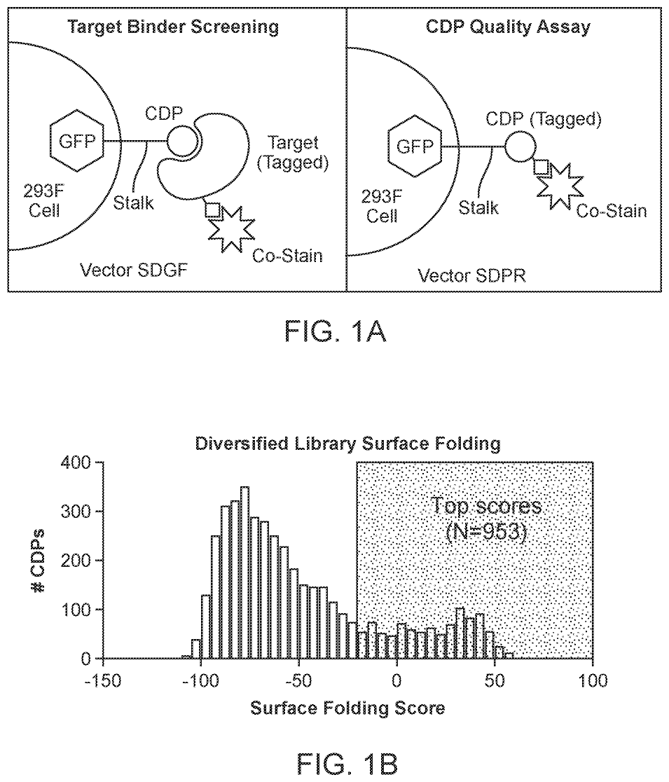

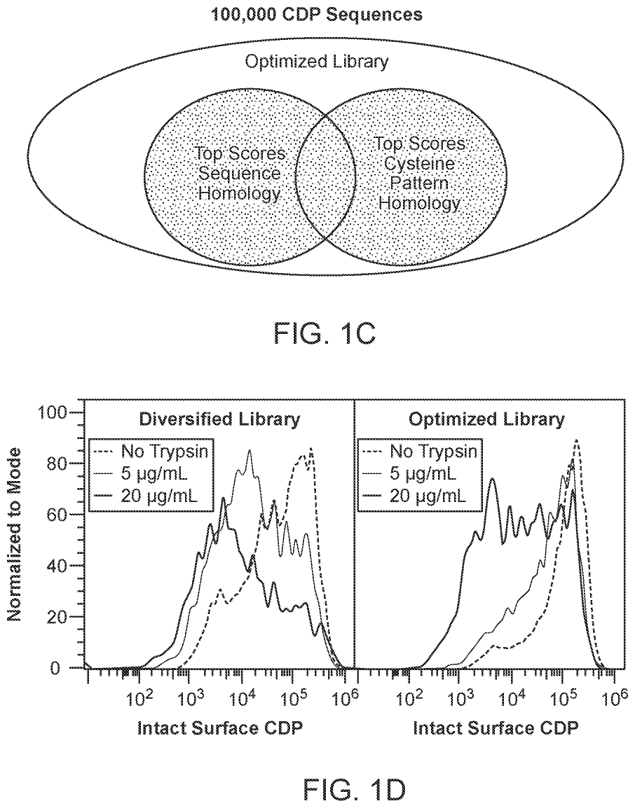

This example describes construction of a cystine dense peptide screening library using surface folding models and protease resistance data. Surface display may serve as a highly efficient method for screening peptides for target engagement, and mammalian surface display may be particularly useful for cystine dense peptides (CDPs) that utilize a secretory pathway that is capable of folding cysteine-rich surface proteins. A mammalian surface display system used to screen the improved cystine dense peptide library is illustrated in FIG. 1A. Under conditions where the CDP was not tagged but the cells were stained with an affinity-tagged (e.g., biotinylated) target protein and labeled with a fluorescent co-stain (e.g., streptavidin), cells expressing target-binding CDPs were sorted and grown over several rounds to enrich for sequences of interest (left, “Vector SDGF”). Under conditions where the CDP itself is tagged (e.g., 6×His (SEQ ID NO: 248)), the fluorescent co-stain (e.g., anti-6×His) was used to detect intact CDPs on the surface of the cell (right, “Vector SDPR”).

A CDP library was narrowed to a subset of CDP scaffolds (N=953) that scored especially highly in a quantitative surface folding assay. A composite surface folding score incorporating surface expression and protease resistance was used to assess the surface folding of the members of the large CDP library, as shown in FIG. 1B. The top scoring CDPs represented cystine scaffolds predicted to have high surface expression, protease resistance, or both. A database of 96,000 CDP sequences, gleaned from public sequence databases (Swiss-Prot and TrEMBL), was searched to identify peptides with structural or sequence homology to the identified 953 CDP scaffolds. An additional 7,940 CDPs were identified and combined with the 953 identified CDP scaffolds to create an optimized library containing 8,893 CDP members, as illustrated in FIG. 1C.

To determine whether homologs of top-performing CDPs improve library performance, the optimized library (optimized CDP library) and the original library (diversified CDP library) were cloned into a SDPR surface display lentivector with a C-terminal 6×His tag (SEQ ID NO: 248) and tested as a pool by low multiplicity of infection (MOI of 1) transduction, as shown in FIG. 1D. Cells were treated with 5 μg/mL trypsin, 20 μg/mL trypsin, or PBS (to trypsin) for 5 minutes, reduced with 10 mM DTT for 5 minutes, and stained with an anti-6×His antibody. In cells treated with PBS (no trypsin), anti-His staining was representative of total surface protein levels. In trypsin-treated cells, anti-His staining was representative of trypsin resistance since CDPs that have been cleaved by trypsin will release their His tag upon reduction with DTT and will not confer staining to their cell. The PBS-treated samples showed 2.14 times higher staining in the optimized library than in the diversified library, indicating better surface expression of the optimized pool. Trypsin treatment revealed similar improvements of the optimized library over the diversified library. Since only 11% of the optimized library represented top scoring diversified sequences, the higher staining of the optimized library, both without and with trypsin treatment, indicated that homology to a top scoring CDP was an effective means of identifying untested sequences that can be expected to demonstrate high surface expression and protease resistance similar to the top scoring CDPs.

### Example 3

### Structural Analysis of an Optimized Cystine Dense Peptide Library

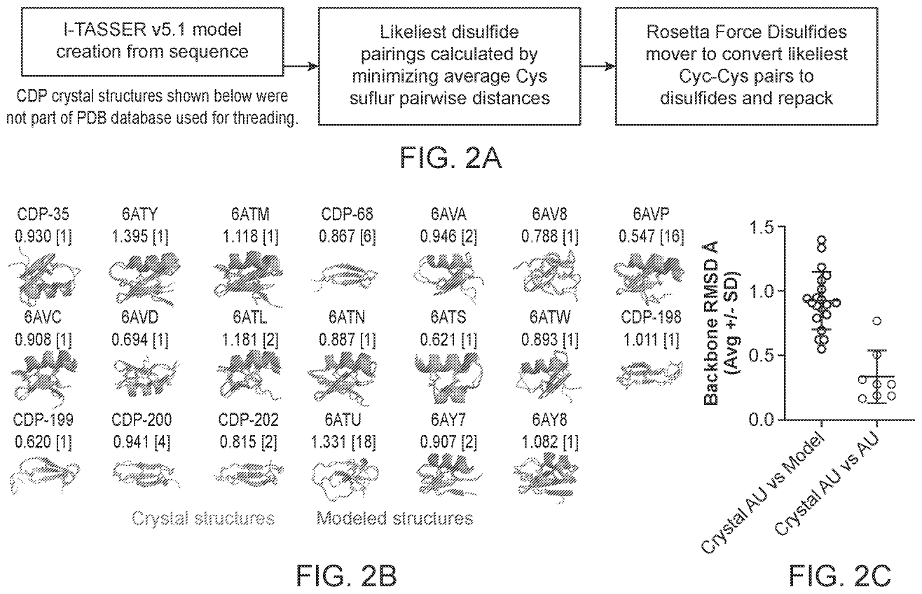

This example describes computational modeling to perform structural analysis of an optimized cystine dense peptide (CDP) library. Structures of the optimized library members were modeled and compared to known crystal structures to facilitate both hypothesis-driven docking simulations and mutational structure-activity relationship (SAR). Protein structural modeling tools I-TASSER and Rosetta were used in combination to model the structures of CDP library members. I-TASSER has previously been used with high success rates in small scaffolds with complex folding. Rosetta protein modeling software was selected for one of their programs, “ForceDisulfides,” which can convert cysteine pairs with beta carbons in close proximity into disulfides in the model. As illustrated in FIG. 2A, the structural modeling pipeline used I-TASSER version 5.1 modeling software to create a structural model from a CDP sequence. The model was used to determine the likeliest disulfide pairings by minimizing the average pairwise distances between bonded cysteine sulfur atoms. Using the ForceDislufides program, Rosetta relaxed the disulfide-bonded structures with an all-atom refinement and repacking algorithm to minimize steric clashes.

The structural modeling pipeline was applied to CDPs that had been previously crystallized, and the models were compared to the experimentally obtained crystal structures. PyMol protein visualization software was used to align backbone atoms of the crystal structures and computational models. Alignments of the CDP structures are provided in FIG. 2B. The structures aligned with an average root-mean-square deviation (RMSD) of 0.924 Å, as shown in FIG. 2C. While this RMSD was greater than the 0.333 Å average RMSD when asymmetric units (AUs) of crystals were aligned to each other, an average RMSD of <1 Å compares well with the ˜1.5-2.2 Å RMSD values typically seen as evidence of successful modeling of de novo miniprotein designs. The pipeline was then applied to the remaining members of the optimized library. Of 8893 members modeled, 4298 structures passed the set confidence thresholds (I-TASSER C-score>0 and Rosetta energy score<80).

### Example 4

### Pre-Screening of Target-Compatible Scaffolds Using Low-Resolution Docking

This example describes using low-resolution docking of cystine dense peptide structural models for in silico pre-screening of target-compatible scaffolds. The CDP structural library, generated as described in EXAMPLE 3, was used to predict favorable binding to targets of clinical interest. Because high-resolution docking simulations are computationally expensive at large scale (thousands of binder candidates with no a priori interface knowledge), low-resolution RosettaDock scripts were used. Low-resolution simulations run faster than high-resolution simulations by converting side chains to a single large pseudo-atom, or centroid, that simplifies energetic calculations by eliminating rotamer packing. This conversion was utilized to predict favorable docking regions where high-scoring docks clustered (using DBSCAN) and to rank scaffolds for docking compatibility to targets of interest, allowing the generation of focused sub-libraries from the high diversity optimized library, identified in EXAMPLE 2. To generate the models, a target protein and CDP of interest are fed into RosettaDock in Low Resolution mode, performing at least 2000 runs. For each dock, the center of mass (CoM) of the CDP was identified and the docking interaction scored. The centers of mass from the top 100-200 docks were analyzed with DBSCAN to identify clusters of high-scoring docks, and the center of each cluster was defined as a possible peptide docking site.

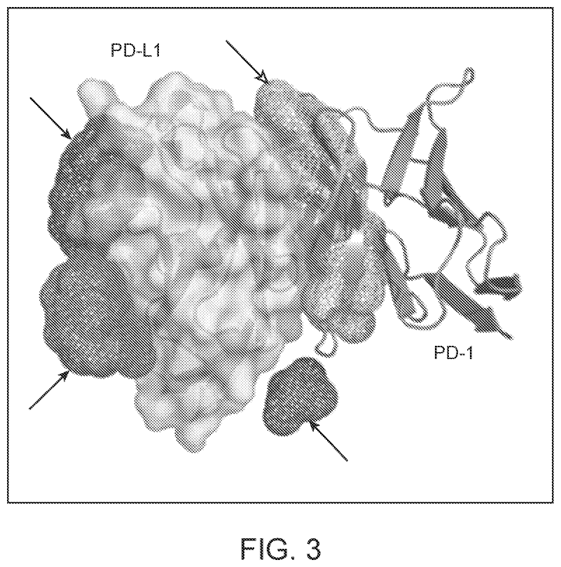

To test in silico pre-screening of the optimized CDP library, the 4298-member optimized model library was docked against domains seen in a high-resolution co-crystal structures of PD-L1 with PD-1 (PDB ID No: 4ZQK). After docking all 4298 CDP scaffolds to the target, common target regions for CDP docking on the assembled candidate docking sites were identified using DBSCAN clustering. As shown in FIG. 3, four clusters were identified, one of which was located at the PD-L1:PD-1 interface and could represent a cluster of binders from which PD-L1 inhibitor candidates may be identified.

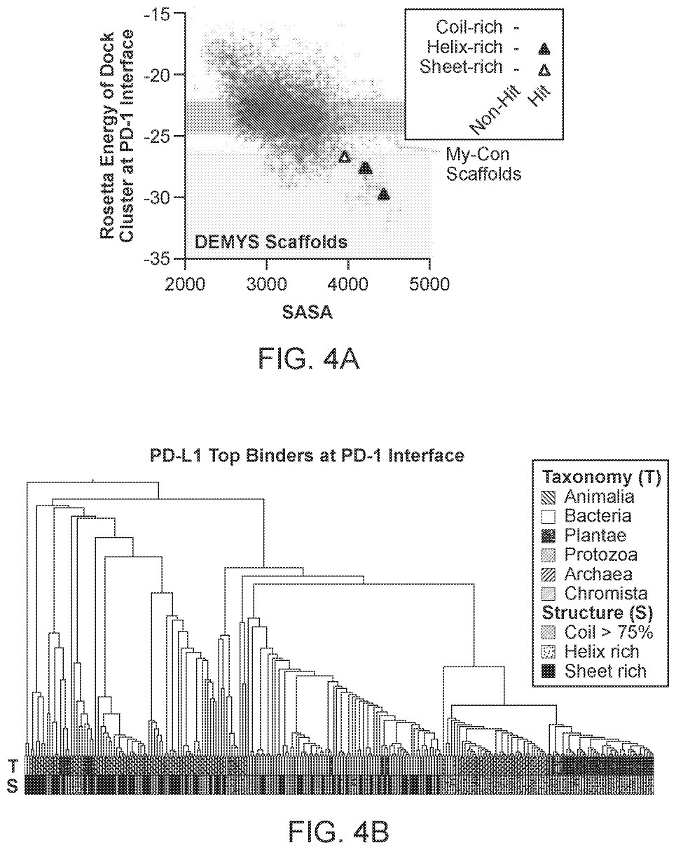

This whole-library docking was used to select sub-libraries enriched for scaffolds with favorable surface shape/energetics for target binding to PD-L1 at the PD-1 binding interface. A sub-library for high-scoring PD-L1 scaffolds predicted to dock at the PD-1 interface is shown in FIG. 4A and FIG. 4B. FIG. 4A shows Rosetta energy of docking for the high-scoring PD-L1 scaffolds plotted against the solvent accessible surface area (SASA). Scaffolds are color-coded based on the dominant structural element in the scaffold. The lighter shaded region represents scaffolds that were used for docking-enriched Met/Tyr scanning (DEMYS) library generation, described in greater detail in EXAMPLE 5. Even with <300 members, the scaffold library still possesses structural and taxonomical diversity, as seen in the phylogenetic tree shown in FIG. 4B.

### Example 5

### Identification of PD-L1-Binding Cystine Dense Peptides

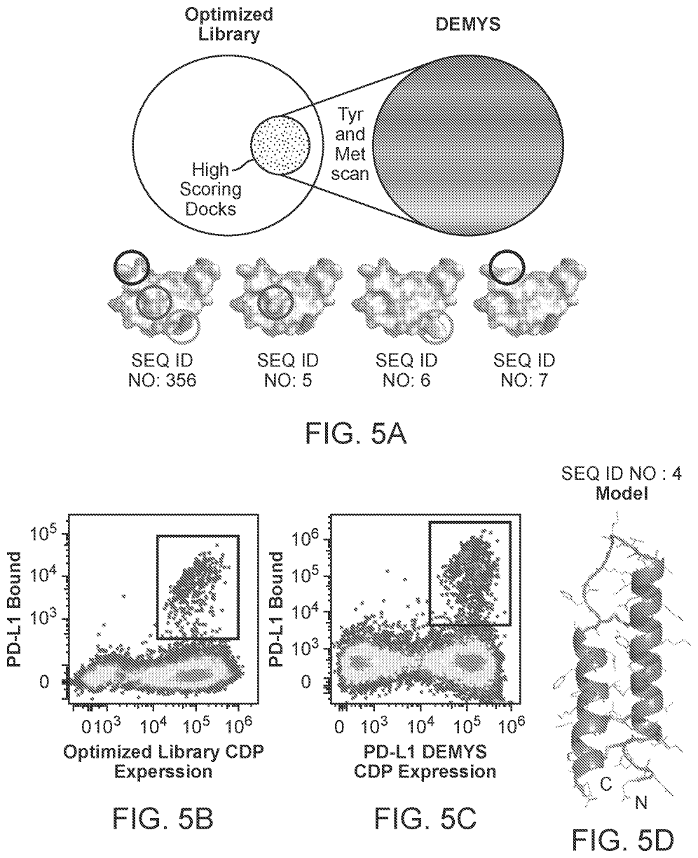

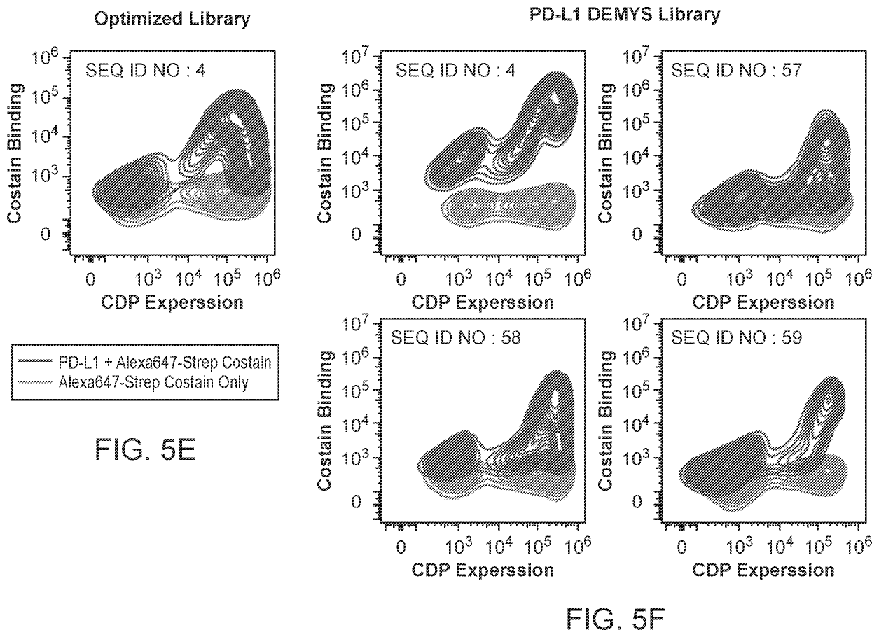

This example describes identification of PD-L1-binding cystine dense peptides using docking-enriched methionine (M)-tyrosine (Y) scanning (DEMYS). The surface display assay and library generation techniques described in EXAMPLE 2-EXAMPLE 4 were implemented as a high throughput screening platform to identify PD-L1-binding CDPs. The limited sub-library of potential PD-L1-binding scaffolds permitted further diversification using tyrosine and methionine scanning to create hydrophobic patches that can seed novel protein:protein interactions. Tyrosine (Tyr) and methionine (Met) were chosen as they are aromatic and aliphatic residues, respectively, that contain a polar atom, avoiding the extreme hydrophobic character of similarly sized phenylalanine (Phe) and leucine (Leu) residues that could impact solubility. The docking-enriched M-Y scan (DEMYS) strategy for combining sub-library selection of docking-capable scaffolds with tyrosine and methionine scanning is illustrated in FIG. 5A. Scaffolds from the optimized model library that scored well in low-resolution target docking were selected and further diversified by Met/Tyr scanning of hydrophilic surface residues. A sample scaffold, color coded by hydrophobicity such that lighter shading indicates carbon atoms not contacting a polar atom, intermediate shading indicates acidic atoms, darker shading indicates basic atoms, and the remaining atoms are shown in white, is shown in its parental (WT) form and with three example Met or Tyr mutations (D18M, R26Y, and R36M). This sample scaffold was used to illustrate Met/Tyr scanning as a means of seeding or expanding hydrophobic patches for novel protein:protein interactions.

DEMYS was implemented to identify PD-L1-binding peptides that bound at the PD-1 binding interface. A DEMYS library targeted to the PD-L1:PD-1 interface was created. Both the optimized library, generated as described in EXAMPLE 4, and PD-L1:PD-1 DEMYS libraries were screened via SDGF mammalian display, as described in EXAMPLE 2, to identify PD-L1 binding CDPs. The optimized mammalian display screen, shown in FIG. 5B, yielded a validated binding scaffold, corresponding to SEQ ID NO: 4. Validation of SEQ ID NO: 4 binding to PD-L1 is shown in FIG. 5E. The DEMYS library mammalian display screen, shown in FIG. 5C, yielded hits representing four distinct parental CDP scaffolds, corresponding to SEQ ID NO: 3, SEQ ID NO: 57, SEQ ID NO: 58, and SEQ ID NO: 59, derived from SEQ ID NO: 353 (EEDCKVHCVKEWAAYKACAERIKSDTTGQAHCSGQYFDFWKCVDHCAAP, corresponding to SEQ ID NO: 354 (GSEEDCKVHCVKEWAAYKACAERIKSDTTGQAHCSGQYFDFWKCVDHCAAP) without an N-terminal GS), SEQ ID NO: 355 (EESCKPQCVKAWLEYQACAERVEKDESGEAHCTGQYFDYWHCVDKCAAK), SEQ ID NO: 356 (ARTCESQSHRFKGPCVSDTNCASVCRTERFSGGHCRGFRRRCLCTKHC), and SEQ ID NO: 357 (EERCKPQCVKSLYEYEKCVKRVENDDTGHKHCTGQYFDYWSCIDKCVAS), respectively. The highest staining of the four DEMYS hits, corresponding to SEQ ID NO: 3, was a variant of SEQ ID NO: 4, containing the same cystine scaffold. The remaining three hits, corresponding to SEQ ID NO: 57, SEQ ID NO: 58, and SEQ ID NO: 59, represented three distinct cystine scaffolds. Validation of SEQ ID NO: 2, SEQ ID NO: 57, SEQ ID NO: 58, and SEQ ID NO: 59 binding to PD-L1 are shown in FIG. 5F. Three of the four DEMYS hits were highly helical in nature and had high solvent-accessible surface area (SASA). While the optimized library as a whole contained high-SASA CDPs of a variety of structures (coil-rich, sheet-rich, or helix-rich), M-Y scanning focused on helix-rich structures based on docking enrichment results. The M-Y scan of the helix-rich CDPs from the second-generation library yielded multiple PD-L1-binding hits. The I-TASSER/Rosetta modeled scaffold for SEQ ID NO: 4, whose scaffold was identified as a PD-L1 binding hit in both screens, is shown in FIG. 5D.

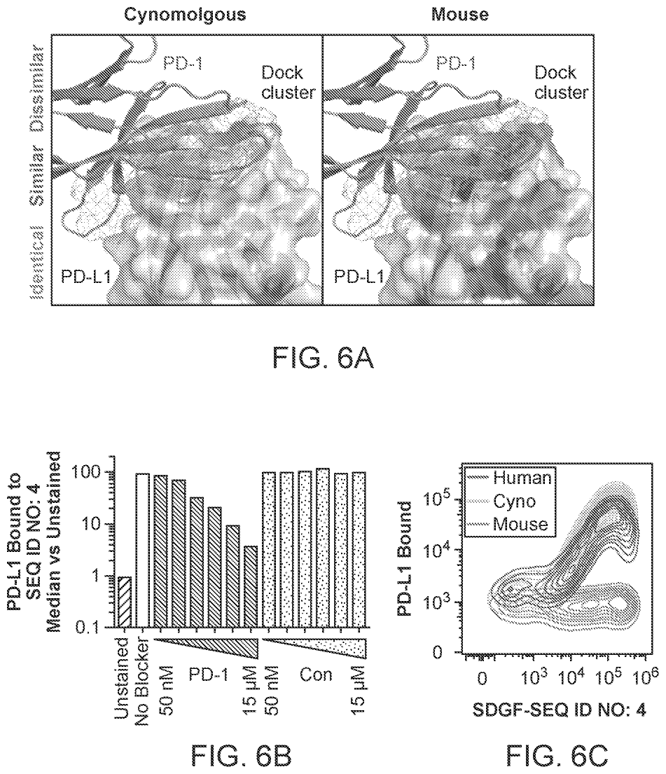

As predicted by the library's derivation from top scoring scaffolds at the PD-L1:PD-1 interface, it was observed that high concentrations of PD-1-Fc can disrupt PD-L1 binding to cells surface-expressing SEQ ID NO: 4. Furthermore, the cluster of high-scoring docks predicted to disrupt PD-1 binding is found on a surface of PD-L1 with high homology to cynomolgus monkeys, but poor murine homology, as shown in FIG. 6A. In silico docking was used to predict binding of identified PD-L1-binding CDPs to PD-L1. The optimized library top 200 predicted PD-L1-binders (cyan mesh) docked at the PD-1 interface and are shown superimposed upon a surface rendition of PD-L1 (PDB ID No: 4ZQK), color-coded for cynomolgus monkey (left) and mouse (right) homology. PD-1 bound to PD-L1 is shown as a ribbon structure. The ability of SEQ ID NO: 4 to disrupt PD-1 interactions with PD-L1 was demonstrated using a competition assay, as shown in FIG. 6B. HEK 293F cells surface-expressing SEQ ID NO: 4 via SDGF demonstrated a reduction in staining when PD-1 competitor Fc fusion was added at either 50 nM, 150 nM, 500 nM, 1.5 μM, 5 μM, or 15 μM concentrations, but no difference in staining when control Fc fusion protein competitor is used at comparable concentrations. In flow staining assays, shown in FIG. 6C, cells surface-expressing SEQ ID NO: 4 bound both human and cynomolgus PD-L1, but no interactions with murine PD-L1 were observed. These results provided further validation of the modeled interface PD-L1-CDP binding interface. These results also demonstrate that SEQ ID NO: 4 binds at a site that is competitive with PD-1 and also demonstrate that SEQ ID NO: 4 binds to both human and cynomolgus PD-1.

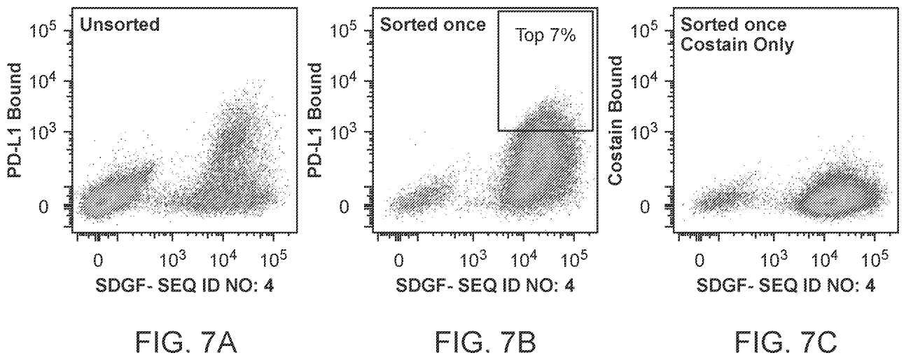

Site-saturation mutagenesis (SSM) was used to develop a high-affinity variant of SEQ ID NO: 4. Cells displaying CDP variants of SEQ ID NO: 4 were stained with fluorescent PD-L1, and flow sorted based on binding to PD-L1, as shown in FIG. 7A. Sorted cells were regrown and pooled, as shown in FIG. 7B. Flow sorting of sorted cells stained with co-stain only (no PD-L1) is shown in FIG. 7C.

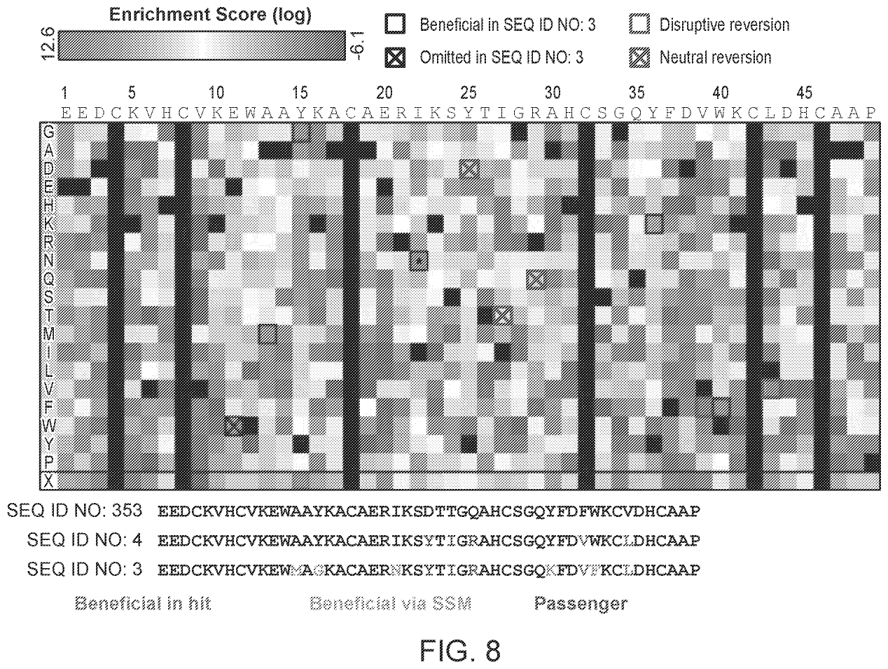

After two rounds of staining, flow sorting, and regrowth, the resulting variant pool was sequenced to identify enriched and depleted variants, as shown in FIG. 8. Enriched variants represented amino acid substitutions that likely improve target binding (either directly at the interface, or indirectly by improving folding and stability), while depletion indicated amino acid variants that disrupt binding capability. As shown in FIG. 8, “Beneficial in SEQ ID NO: 3” indicates those point mutations that were combined and included in SEQ ID NO: 3 to increase affinity of SEQ ID NO: 3 for PD-L1 relative to SEQ ID NO: 4. “Omitted in SEQ ID NO: 3” indicates a point mutation that appeared beneficial in isolation but, when combined with other beneficial point mutations, became disruptive to binding. This point mutation was omitted in SEQ ID NO: 3. “Disruptive reversions” indicate point mutations that, when reverted to the parent amino acid, reduced binding, indicating that those point mutations in SEQ ID NO: 4 were beneficial to PD-L1 binding. “Neutral reversions” indicate reversions to the parental amino acid present in SEQ ID NO: 353 that do not impact binding to PD-L1, showing that these mutations were acquired during library generation but were not instrumental to SEQ ID NO: 4 binding to PD-L1. Such neutral reversions are also referred to as passenger mutations.

Enrichment results shown in FIG. 8 are provided in tabular form in TABLE 12. Relative enrichment of each amino acid variant (in columns) is shown relative to the amino acid at the corresponding position in SEQ ID NO: 4 (in rows). Positive values represented amino acid substitutions that likely improve target binding, while negative values indicated amino acid variants that disrupt binding capability.

TABLE 12
Relative Enrichment of Amino Acid Variants Binding to PD-L1

| SEQ ID | | Amino acid Variant | | | | | | | | | | | | | | | | | | |
|---|---|---|---|---|---|---|---|---|---|---|---|---|---|---|---|---|---|---|---|---|
| NO: 4 | | G | D | E | H | K | R | N | Q | S | T | A | M | I | L | V | F | W | Y | P |
| 01 | E | −3.49 | −3.54 | −3.02 | | −0.96 | −1.64 | −3.87 | −4.09 | −2.99 | −2.41 | −3.97 | 3.90 | −4.59 | −2.29 | 2.34 | −2.51 | 2.06 | −2.43 | −3.44 |
| 02 | E | 2.83 | −0.54 | −1.39 | | −3.12 | −1.09 | −4.81 | −3.54 | −4.50 | −0.73 | −3.00 | −3.37 | −3.95 | 1.39 | −3.72 | 0.67 | −3.73 | −4.03 | −1.75 |
| 03 | D | −1.18 | −4.54 | | 0.84 | −3.02 | −4.65 | −3.81 | −0.44 | −4.65 | 1.38 | −4.43 | −4.48 | −2.37 | −4.78 | −4.21 | −3.25 | −5.00 | −1.91 | −3.91 |
| 04 | C | | | | | | | | | | | | | | | | | | | |
| 05 | K | −0.51 | −5.36 | −4.12 | −4.90 | −4.48 | | 0.67 | −2.96 | −3.41 | −4.24 | −4.32 | −0.97 | −5.39 | −4.16 | 2.54 | −4.27 | −3.00 | −4.24 | −4.72 |
| 06 | V | −0.11 | −5.07 | −5.17 | 2.95 | −3.56 | −3.55 | −4.11 | −2.95 | 1.18 | 0.36 | −5.79 | 1.71 | −2.71 | 0.16 | | −3.31 | −4.05 | −3.04 | −6.12 |
| 07 | H | −4.80 | −0.19 | 2.53 | 1.31 | | 1.79 | 3.32 | 2.29 | 3.11 | 1.74 | −3.87 | −4.41 | −3.14 | −1.13 | −4.69 | −3.56 | −1.48 | 0.76 | −2.63 |
| 08 | C | | | | | | | | | | | | | | | | | | | |
| 09 | V | −4.50 | −4.79 | 1.38 | −3.00 | −4.00 | −6.05 | −5.90 | −3.08 | −4.74 | −5.91 | −5.72 | 0.91 | −4.82 | −3.41 | | −4.59 | −4.65 | −5.14 | −5.54 |
| 10 | K | −0.48 | 2.30 | −5.21 | −1.50 | −2.60 | | 2.29 | −4.35 | 1.35 | 2.67 | 3.39 | −1.71 | −3.24 | −3.83 | −4.53 | −5.87 | −5.16 | −3.83 | −2.89 |
| 11 | E | −2.36 | 3.33 | 2.78 | | 4.85 | −1.77 | −2.14 | −0.82 | 1.51 | 3.05 | 5.09 | 3.93 | 3.89 | 1.55 | 5.74 | −0.78 | 8.78 | −2.55 | −2.94 |
| 12 | W | −1.69 | 0.20 | −2.38 | 0.94 | −0.65 | −1.55 | −0.09 | −2.56 | 3.71 | 0.56 | −0.09 | −2.29 | −1.76 | −5.60 | −1.95 | −1.86 | | −5.60 | 2.49 |
| 13 | A | −0.68 | | −0.96 | 0.06 | −1.29 | 2.57 | −0.08 | 4.49 | 3.53 | −0.58 | 1.71 | 10.78 | 10.00 | 11.08 | 10.51 | −0.65 | 2.61 | −1.67 | −1.77 |
| 14 | A | 1.58 | | −2.11 | 2.21 | −0.17 | 0.38 | −1.65 | 3.07 | −0.78 | −4.52 | 0.46 | −3.92 | −4.77 | −3.90 | −4.95 | −2.96 | −4.41 | 2.30 | −0.91 |
| 15 | Y | 12.23 | 9.87 | 0.81 | 4.66 | 8.32 | 10.21 | 8.82 | 10.33 | 10.90 | 9.18 | 6.92 | 2.29 | 2.95 | 3.31 | 2.68 | −0.43 | 0.20 | | −0.08 |
| 16 | K | −4.47 | −4.90 | 3.16 | −1.46 | −3.14 | | 2.26 | 0.91 | −4.05 | −4.37 | −4.75 | −1.29 | −4.42 | 0.47 | 1.89 | −4.68 | −3.31 | −0.94 | −4.21 |
| 17 | A | 3.38 | | 2.88 | −0.77 | −1.95 | −4.05 | −4.32 | −3.91 | −5.16 | −1.47 | 2.96 | −3.43 | −4.92 | 0.76 | −5.84 | −3.50 | 1.07 | −0.88 | 1.70 |
| 18 | C | | | | | | | | | | | | | | | | | | | |
| 19 | A | 1.67 | | −1.41 | 0.59 | 1.77 | 1.78 | −0.84 | 5.69 | −3.37 | 1.19 | −0.36 | −3.76 | −5.07 | −5.31 | −5.86 | 0.01 | −3.65 | −5.74 | 4.27 |
| 20 | E | 2.11 | 0.63 | 3.09 | | −5.81 | −1.50 | −5.48 | 1.10 | −4.78 | −4.07 | −4.36 | −4.93 | −5.98 | −5.60 | −1.15 | −5.60 | −5.56 | −5.00 | 3.67 |
| 21 | R | 3.23 | −4.53 | 1.12 | −0.81 | 2.68 | 0.08 | | 2.19 | 3.41 | 2.68 | 4.85 | −0.41 | −3.27 | −4.58 | 2.12 | −4.55 | −2.65 | 6.09 | −0.23 |
| 22 | I | 6.28 | −1.27 | 9.88 | 7.55 | 0.32 | 3.59 | −3.27 | 10.64 | −2.32 | 1.60 | 1.98 | 0.12 | | −2.18 | −1.95 | 1.98 | −0.04 | 1.66 | 8.45 |
| 23 | K | 2.47 | −0.05 | 4.02 | 1.11 | 2.54 | | 1.06 | 1.78 | 0.10 | 0.38 | −3.45 | −3.60 | −5.72 | −2.33 | −4.53 | −5.41 | −3.88 | 0.54 | 2.95 |
| 24 | S | 5.33 | 1.05 | 1.86 | −3.17 | 2.30 | −0.54 | −2.97 | 2.75 | 1.48 | | −3.06 | −5.01 | −1.25 | −3.48 | 0.11 | 2.39 | −3.74 | −3.16 | 3.28 |
| 25 | Y | −3.50 | 3.43 | 4.40 | −4.18 | 0.64 | −3.20 | −4.07 | 2.78 | 5.16 | 1.13 | 2.46 | 2.70 | 2.30 | −1.55 | 2.90 | −1.23 | −4.86 | | 0.96 |
| 26 | T | 0.86 | 1.72 | 5.54 | −4.16 | −3.06 | 0.99 | 4.12 | −2.48 | −0.24 | −4.85 | | −4.52 | −0.32 | −5.19 | −5.05 | −3.37 | 2.17 | 3.49 | −3.71 |
| 27 | I | 0.68 | 0.11 | −2.95 | 3.85 | −4.70 | −4.81 | −4.72 | 1.49 | 1.73 | −1.99 | 2.87 | −2.68 | | −5.11 | 2.80 | −2.02 | −4.66 | −4.55 | 0.37 |
| 28 | G | | −3.36 | 0.45 | −0.20 | −0.64 | −2.77 | −5.14 | 0.98 | 0.64 | −0.72 | 4.94 | −4.69 | −5.03 | 1.80 | 0.41 | 1.39 | −5.62 | −4.64 | 2.88 |
| 29 | R | 2.29 | 2.31 | −1.47 | 1.45 | −2.71 | 0.17 | | 1.70 | 4.61 | 3.73 | 1.35 | −1.90 | 3.15 | −2.77 | −2.85 | −5.15 | −0.34 | 3.09 | 2.56 |
| 30 | A | −4.55 | | 2.90 | −3.24 | −5.02 | −5.12 | −4.62 | 2.56 | −4.58 | −2.64 | −1.97 | −1.40 | 2.60 | −0.62 | −4.80 | −3.74 | −3.97 | −0.42 | −2.65 |
| 31 | H | 4.13 | −4.73 | 2.85 | 5.02 | | −2.48 | −1.77 | 2.43 | −2.80 | −1.57 | −2.56 | −0.89 | −2.34 | −5.42 | −5.04 | 1.89 | 3.50 | −0.59 | −4.93 |
| 32 | C | | | | | | | | | | | | | | | | | | | |
| 33 | S | 1.08 | 1.42 | −4.00 | 3.23 | −0.62 | −5.43 | −1.02 | 1.69 | −0.29 | | −0.56 | −5.30 | −2.00 | −3.12 | −1.46 | −5.88 | −0.68 | 1.30 | 2.68 |
| 34 | G | | −4.71 | −2.05 | −5.02 | −2.55 | −4.85 | −4.82 | −4.10 | −1.19 | −2.19 | −4.08 | 2.40 | −4.43 | 4.21 | −1.94 | −3.83 | −5.67 | −5.03 | −4.07 |
| 35 | Q | 0.87 | 2.42 | 0.24 | −0.46 | −3.46 | 0.64 | −0.65 | 2.57 | | −4.24 | −0.31 | −4.60 | −4.95 | −1.15 | −2.28 | −3.49 | 1.79 | −4.31 | −1.43 |
| 36 | Y | −0.19 | −1.76 | −0.07 | 3.54 | 3.67 | 8.65 | −1.25 | 3.02 | 2.30 | 3.66 | 4.09 | −0.70 | −2.02 | 4.76 | 0.51 | 1.65 | −5.01 | | 7.33 |
| 37 | F | 4.37 | −2.34 | −4.93 | 2.28 | −5.29 | −3.85 | −1.85 | −4.56 | 2.46 | −3.69 | −5.09 | −4.15 | −2.82 | −4.72 | −4.17 | | −3.83 | −4.08 | −5.11 |
| 38 | D | −2.16 | −2.63 | | −4.64 | −5.89 | 5.47 | −5.02 | −0.66 | −4.72 | −4.37 | −4.36 | −4.16 | −4.64 | −4.44 | −4.78 | −4.34 | −1.46 | −1.48 | −4.37 |
| 39 | V | 0.01 | −5.12 | −4.63 | −5.06 | −3.31 | −3.35 | −3.62 | −0.87 | −3.26 | −3.80 | −4.44 | −0.42 | −3.60 | −2.69 | | −4.85 | −4.18 | −3.50 | 3.07 |
| 40 | W | 2.35 | −0.23 | −3.57 | −4.59 | 3.94 | −4.22 | 0.81 | −2.04 | −3.53 | −3.12 | −2.26 | −2.48 | −1.43 | −1.28 | 2.59 | 12.61 | | −3.02 | 0.21 |
| 41 | K | −0.11 | 2.08 | 5.01 | −0.51 | −4.70 | | −2.05 | −0.69 | −0.36 | −3.57 | −2.53 | −1.29 | −4.11 | −0.61 | −4.12 | −4.33 | −5.70 | −3.98 | −4.98 |
| 42 | C | | | | | | | | | | | | | | | | | | | |
| 43 | L | −2.13 | −5.49 | −4.25 | 1.01 | −0.07 | −3.59 | −3.00 | −5.51 | 4.69 | −4.83 | −3.23 | −1.55 | −4.79 | | −4.41 | 0.95 | −1.03 | −4.45 | −3.20 |
| 44 | D | −1.47 | −5.27 | | 1.49 | −0.41 | −3.08 | 1.75 | −2.62 | −4.77 | 2.54 | 0.67 | 1.59 | −0.75 | 1.52 | −5.47 | 0.64 | −5.17 | −4.57 | −0.44 |
| 45 | H | 3.33 | 1.89 | 4.43 | 4.07 | | 0.26 | 0.29 | −2.81 | −1.52 | −1.42 | −3.84 | 0.73 | −0.72 | 1.67 | −5.60 | −4.12 | −4.20 | −2.27 | 2.86 |
| 46 | C | | | | | | | | | | | | | | | | | | | |
| 47 | A | 1.63 | | −0.18 | −2.26 | −2.30 | 1.70 | −3.87 | −4.32 | −2.89 | 1.66 | −0.79 | −5.25 | 1.74 | 1.70 | −3.03 | −3.81 | −5.62 | −3.71 | −5.22 |
| 48 | A | 2.29 | | 0.56 | 0.25 | −1.20 | −0.32 | 1.99 | −3.81 | 0.85 | −3.63 | 1.83 | −2.02 | −5.01 | −4.41 | −0.85 | 0.76 | −0.36 | −4.22 | −4.32 |
| 49 | P | −4.02 | 1.61 | −3.12 | −3.88 | 1.84 | −3.92 | −3.36 | 1.50 | 6.30 | 2.18 | −0.52 | −4.04 | −1.41 | −4.81 | −3.91 | 2.21 | −0.43 | −4.62 | |

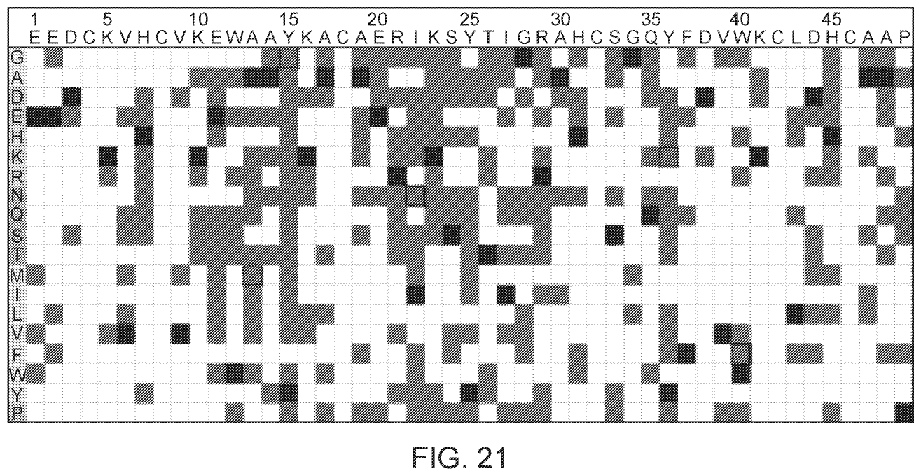

The heatmap shown in FIG. 8 was recolored in FIG. 21 to identify each amino acid that facilitated PD-L1 binding. As seen in FIG. 21, amino acid substitutions relative to SEQ ID NO: 4 determined to have a neutral or beneficial effect are colored in red, and residues of the original SEQ ID NO: 4 sequence are colored black. Amino acids shaded in either dark gray or black are considered to facilitate binding to PD-L1. For example, V6 of SEQ ID NO: 4 has five amino acid substitutions, E, Q, S, M, or L, that were either neutral or beneficial to PD-L1 binding. Therefore, any of E, Q, S, M, L, or V at site six of the CDP facilitate binding to PD-L1. The consensus sequences SEQ ID NO: 358-SEQ ID NO: 363 were identified based on this enrichment data.

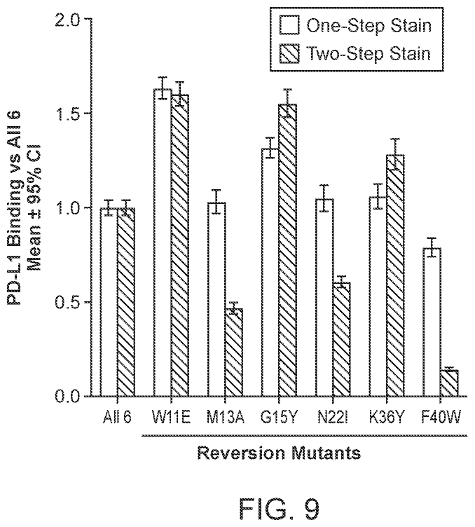

Six highly enriched amino acid substitutions (E11W, A13M, Y15G, 122N, Y36K, and W40F) were combined (SEQ ID NO: 8) and evaluated against both SEQ ID NO: 4 and each single reversion variant (SEQ ID NO: 9-SEQ ID NO: 14, corresponding to reversion mutants WI IE, M13A, G15Y, N22I, K36Y, and F40W, respectively), as shown in FIG. 9, to see if all mutations were synergistic. Increased PD-L1 staining was indicative of higher affinity for PD-L1. The highest-staining variant contained all except the E11W mutation, so SEQ ID NO: 3, corresponding to SEQ ID NO: 4 with amino acid substitutions A13M, Y15G, 122N, Y36K, and W40F, was selected as the affinity matured variant. SSM was also used to evaluate the mutations found in SEQ ID NO: 4, relative to the parent scaffold (SEQ ID NO: 353). SEQ ID NO: 4 differed from the SEQ ID NO: 353 by five amino acid substitutions (D25Y, T27I, Q29R, F39V, and V43L). The SSM enrichment/depletion data showed that two of these substitutions (F39V and V43L) were beneficial to PD-L1 binding, as the respective reversion mutations were depleted. The other three reversions (D25Y, T27I, and Q29R) were not depleted, showing that the Q29R, F39V, and V43L substitutions found in SEQ ID NO: 4 are likely to be benign passenger mutations.

### Example 6

### Stability and Binding Affinity of a High-Affinity PD-L1-Binding Cystine Dense Peptide

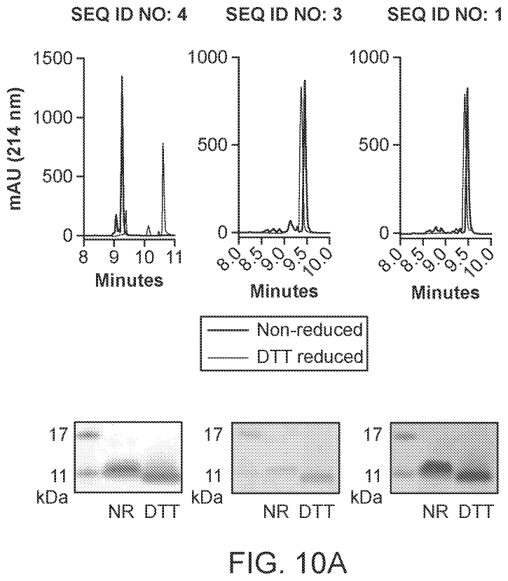

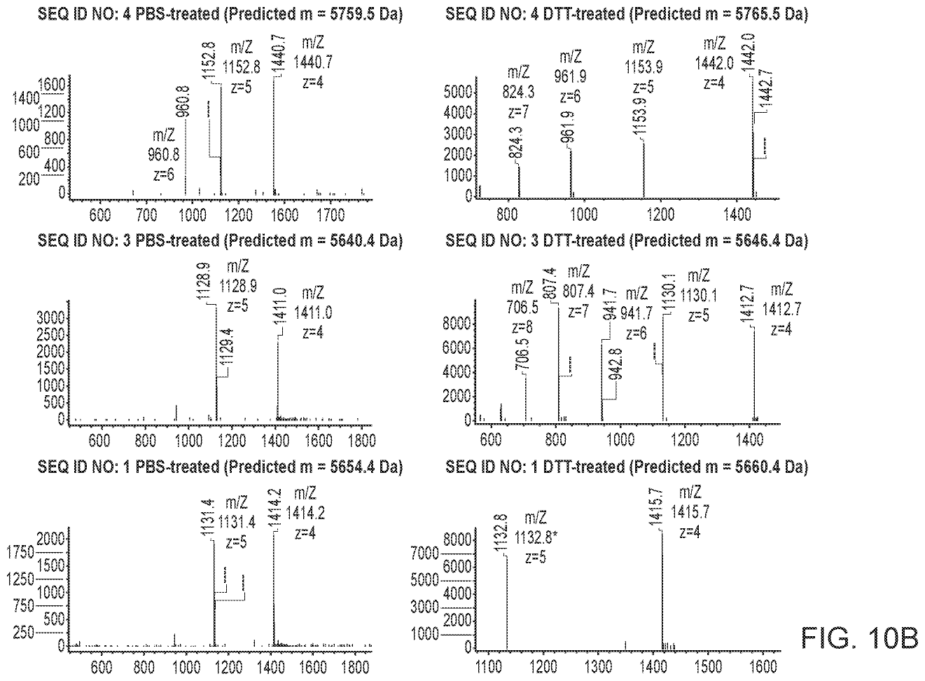

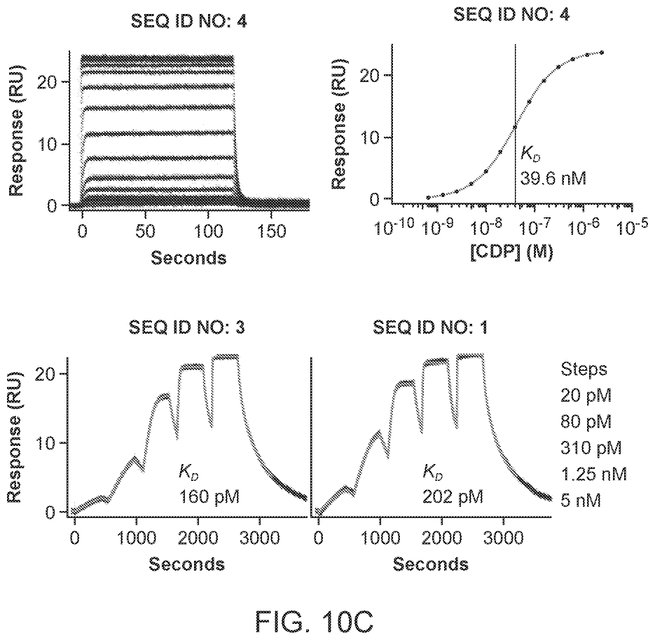

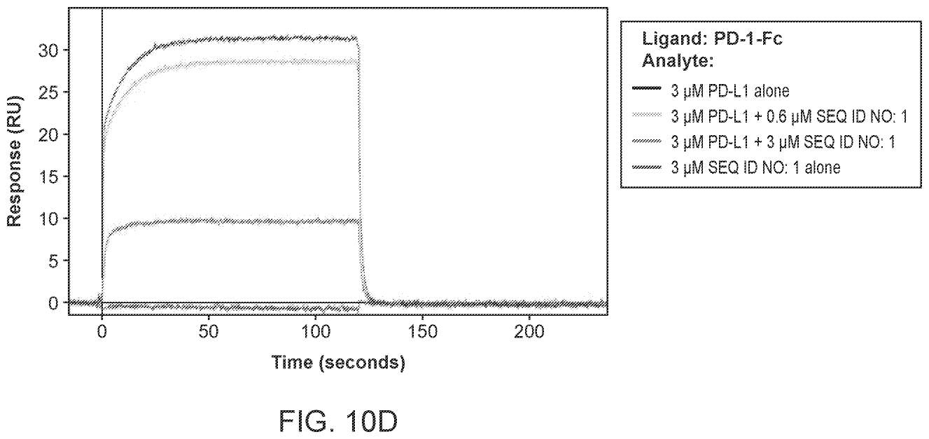

This example describes characterization of the stability, purity, folding, and binding affinity of a high-affinity PD-L1-binding cystine dense peptide. CDPs were produced as soluble molecules as described in EXAMPLE 1. Three recombinant CDPs were evaluated for PD-L1 binding: SEQ ID NO: 4, SEQ ID NO: 3, and SEQ ID NO: 1 which contained a single N22Q amino acid substitution relative to SEQ ID NO: 3. The N22Q substitution present in SEQ ID NO: 1 removes a canonical N-linked glycosite acquired during affinity maturation. All three CDPs were produced at high yields and high homogeneity, as evaluated by reversed phase high-performance liquid chromatography (RP-HPLC, top) and SDS-PAGE (bottom) in FIG. 10A. While SEQ ID NO: 3 contained a canonical N-linked glycosite, there was minimal evidence of glycosylation, which might be seen as a minor peak indicated by an arrow in the top middle panel. Smearing consistent with extensive glycosylation is not visible in the SDS-PAGE gel (bottom). Exposure of the CDPs to high concentrations of reducing agent (10 mM DTT, measured by RP-HPLC in FIG. 10A) showed that the non-matured hit, SEQ ID NO: 4, demonstrated a substantial mobility shift in RP-HPLC consistent with losing cystine-stabilized tertiary structure. The affinity matured variants SEQ ID NO: 3 and SEQ ID NO: 1, however, demonstrated only a subtle mobility shift in RP-HPLC. Confirmation from the MS data (FIG. 10B) that the DTT-treated samples were ˜6 Da heavier than the PBS-treated samples, indicated that both SEQ ID NO: 3 and SEQ ID NO: 1 variants were indeed reduced by DTT but retained the majority of their tertiary structure. This shows that some or all of the mutations acquired during affinity maturation contribute to cystine-independent tertiary structure stabilization. The mobility shift observed upon boiling in LDS sample buffer with 10 mM DTT (bottom, FIG. 10A) shows CDP folding that is mediated by disulfides. Full gels are provided in FIG. 18. The mass spectrometry data, shown in FIG. 10B, in which the m/z values for all three CDPs are consistent with oxidized cysteines, further confirms CDP folding.

Binding to PD-L1 of the three recombinant CDPs was evaluated by surface plasmon resonance (SPR), as shown in FIG. 10C. The SPR analysis demonstrated improved affinity upon maturation, as seen by the higher affinity of SEQ ID NO: 3 and SEQ ID NO: 1 as compared to SEQ ID NO: 4. Specifically, SEQ ID NO: 4 bound PD-L1 with an equilibrium dissociation constant (K_{D}) of 39.6±0.3 nM, whereas SEQ ID NO: 3 and SEQ ID NO: 1 bound PD-L1 with K_{D}=160±1 pM and 202±2 pM, respectively, demonstrating an approximately 200-fold improvement in K_{D} upon affinity maturation of SEQ ID NO: 4. Further SPR analysis was performed to measure competition of SEQ ID NO: 1 with the PD-L1-binding domain of PD-1, shown in bold and underline in the full-length sequence of PD-1

(MQIPQAPWPVVWAVLQLGWRPGWFLDSPDRPWNPPTFSPALLVVT
EGDNATFTCSFSNTSESFVLNWYRMSPSNQTDKLAAFPEDRSQPGQ
DCRFRVTQLPNGRDFHMSVVRARRNDSGTYLCGAISLAPKAQIKES
LRAELRVTERRAEVPTAHPSPSPRPAGQFQTLVVGVVGGLLGSLVL
LVWVLAVICSRAARGTIGARRTGQPLKEDPSAVPVFSVDYGELDFQ
WREKTPEPPVPCVPEQTEYATIVFPSGMGTSSPARRGSADGPRSAQ
PLRPEDGHCSWPL, SEQ ID NO: 349)

or PD-L1, as shown in FIG. 10D. This data confirmed that SEQ ID NO: 1 competes with PD-1 and that competition was not an artifact of cell surface staining.

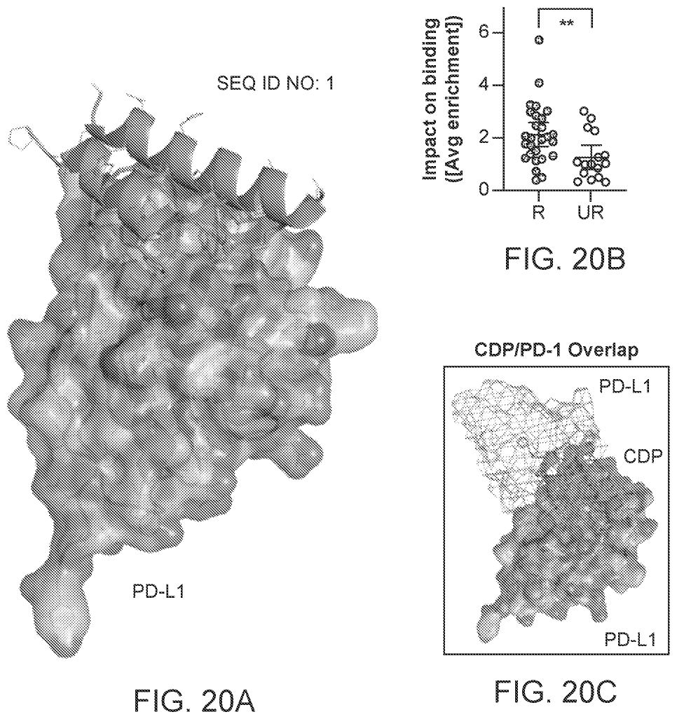

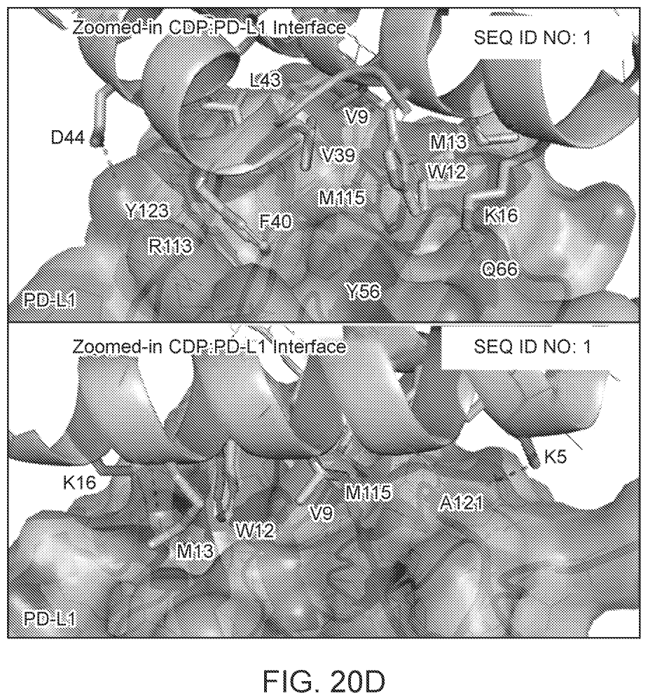

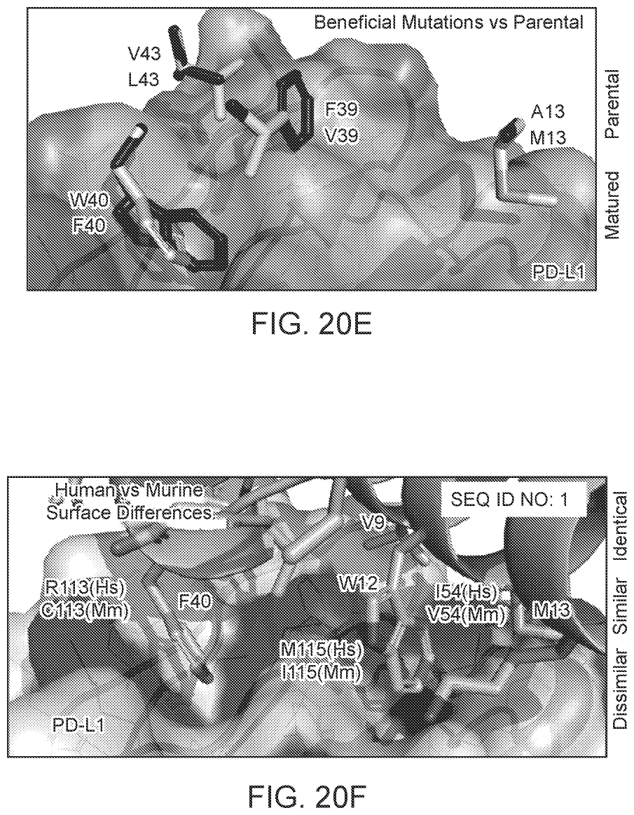

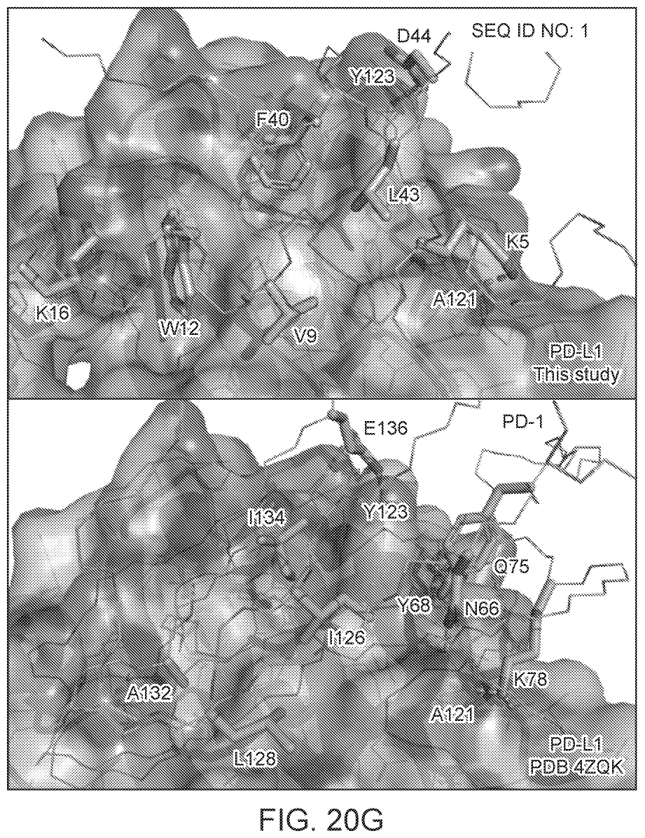

The N22Q variant CDP (SEQ ID NO: 1) was co-crystalized with PD-L1 to confirm the CDP binding site and visualize the surface interactions with PD-L1, as shown in FIG. 20A. SEQ ID NO: 1, a variant that eliminated a canonical N-linked glycosite acquired during affinity maturation, was produced as a soluble molecule as described in EXAMPLE 1 and was co-crystalized with PD-L1. A portion of the CDP, from A19 through Q35, was unresolved in the 2.0 Å structure. Enrichment analysis was performed to determine the impact of amino acid substitutions at residues that were resolved in the crystal structure relative to residues that were unresolved in the crystal structure. The average SSM enrichment scores of unresolved residues were less extreme (deviated less from 0) than seen with resolved residues, as shown in FIG. 20B, showing the specific side chain identities of unresolved residues were less important to high affinity binding. The portion that did resolve matched the model from E1 through C18 and from D38 through A48. K36 and F37 resolved but were not part of the D38-A48 helix.

The resolved portion had an interface surface area of 620 Å² as assessed by PISA (PDBe PISA v1.52), which was similar to the observed interface surface area of PD-L1 with PD-1 (622 Å², PDB 4ZQK). The CDP's location on PD-L1 fell squarely within both the PD-1 occupancy space, as shown in FIG. 20C, showing the in silico low-resolution docking enrichment was predictive of the interface of this hit. A closer look at the interface, shown in FIG. 20D and in FIG. 20G, revealed that the CDP makes use of many of the same interaction sites as PD-1. Both K5 of SEQ ID NO: 1 and K78 of PD-1 made a salt bridge with the A121 backbone oxygen of PD-L1, while both D44 of SEQ ID NO: 1 and E136 of PD-1 similarly formed a salt bridge with Y123 of PD-L1. F40 of SEQ ID NO: 1 sat in pocket formed by Y56, R113, M115, and Y123 of PD-L1, making hydrophobic contacts (M115), herringbone ring stacking interactions (two Y's), and a cation-pi interaction (R113). This pocket was also occupied by I134 of PD-1. Furthermore, V9, W12, and L43 of SEQ ID NO: 1 also shared sites of hydrophobic interactions used by L128, A132, and I126 of PD-1, respectively. The interface-adjacent mutations that differentiated SEQ ID NO: 1 from its parental scaffold would be expected to disrupt binding when reverted to the parental side chains, as illustrated in FIG. 20E. The hydrophobic interactions of both M13 and L43 with the surface of PD-L1 would be lost in the parental A13 and V43; the pocket occupied by F40 would have to distort to accommodate the parental W40, altering the interface elsewhere; and parental F39 does not neatly fit against the surface as V39 does. Finally, analysis of the human/mouse and human/cynomolgus monkey (cyno) homology on the PD-L1 surface revealed that the interaction site contained several non-homologous side chains between human and mice, as shown in FIG. 20F. The human/cyno homology is perfect at this interface, which matches the cross-reactivity data. SEQ ID NO: 4 is not cross-reactive with murine PD-L1 but does bind cyno PD-L1.

### Example 7

### Incorporation of a PD-L1-Binding Peptide into a Bispecific Immune Cell Engager

This example describes incorporation of a high-affinity PD-L1-binding cystine dense peptide into a bispecific immune cell engager (BiICE) to produce a highly effective anti-tumor agent. A PD-L1-binding CDP that disrupts the PD-L1:PD-1 interface could serve as an immuno-oncology drug, inhibiting a checkpoint signal commonly communicated by tumor cells to infiltrating T cells. Using such a PD-L1-binding CDP in a bispecific immune cell engager (BiICE) format, designed to engage both CD3 on T cells and PD-L1 on cancer cells, could further transform PD-L1 from a signal on tumor cells that protects them from immune attack into a signal on tumor cells that encourages attack from the immune system by encouraging activated T cells to engage and kill PD-L1-overexpressing cells. To test this, SEQ ID NO: 2, corresponding to SEQ ID NO: 4 with an N22D substitution, was cloned into a heterodimeric Fc fusion construct containing a set of mutations facilitating knob-and-hole heterodimerization. This PD-L1-binding CDP Fc fusion construct was paired, using knob-and-hole heterodimerization, with an Fc fusion to a CD3-engaging scFv to form a PD-L1-binding CDP/scFv bispecific immune cell engager (CS-BiICE). The CDP-based BiICE (“CS-BiICE”) was formed from a first fusion protein (METDTLLLWVLLLWVPGSTGDYKDEGGSEEDCKVHCVKEWMAGKACAERDKSYTIG RAHCSGQKFDVFKCLDHCAAPGGGGSGGGGSGGGGSEPKSSDKTHTCPPCPAPELLGG PSVFLFPPKPKDTLMISRTPEVTCVVVDVSSHEDPEVKFNWYVDGVEVHNAKTKPREEQ YASTYRVVSVLTVLHQDWLNGKEYKCKVSNKALGAPIEKTISKAKGQPREPQVYTLPP SRDELTKNQVSLSCAVKGFYPSDIAVEWESNGQPENNYKTTPPVLDSDGSFFLVSKLTV DKSRWQQGNVFSCSVMHEALHNHYTQKSLSLSPGK; SEQ ID NO: 342) containing a PD-L1-binding CDP (SEQ ID NO: 2) with an N-terminal signal peptide (METDTLLLWVLLLWVPGSTG; SEQ ID NO: 247; “SP”) and FLAG tag (DYKDEGGS; SEQ ID NO: 246) fused to an Fc “hole” sequence via a linker and a second fusion protein (METDTLLLWVLLLWVPGSTGEVQLVESGGGLVQPGGSLKL SCAASGFTFNKYAMNW VRQAPGKGLEWVARIRSKYNNYATYYADSVKDRFTISRDDSKNTAYLQMNNLKTEDT AVYYCVRHGNFGNSYISYWAYWGQGTLVTVSSGGGGSGGGGSGGGGSQTVVTQEPSL TVSPGGTVTLTCGSSTGAVTSGNYPNWVQQKPGQAPRGLIGGTKFLAPGTPARFSGSLL GGKAALTLSGVQPEDEAEYYCVLWYSNRWVFGGGTKLTVLGGGGSEPKSSDKTHTCP PCPAPELLGGPSVFLFPPKPKDTLMISRTPEVTCVVVDVSHEDPEVKFNWYVDGVEVHN AKTKPREEQYASTYRVVSVLTVLHQDWLNGKEYKCKVSNKALGAPIEKTISKAKGQPR EPQVYTLPPSRDELTKNQVSLWCLVKGFYPSDIAVEWESNGQPENNYKTTPPVLDSDGS FFLYSKLTVDKSRWQQGNVFSCSVMHEALHNHYTQKSLSLSPGKGGGGSHHHHHH; SEQ ID NO: 347) containing an anti-CD3 single-chain fragment variable (scFv) fused to an Fc “knob” sequence with an N-terminal signal peptide and C-terminal 6×His tag (HHHHHH; SEQ ID NO: 248). The “hole” sequence heterodimerizes with the “knob” sequence.

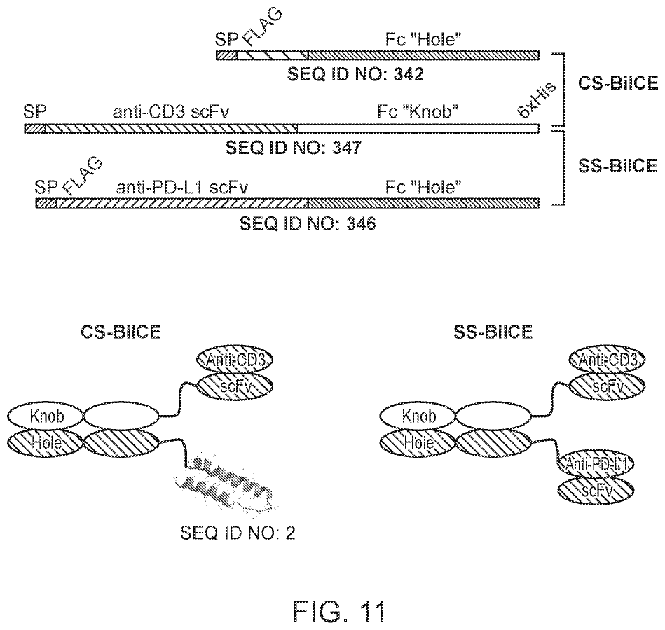

A comparator molecule was also constructed. The comparator molecule, referred to as SS-BiICE, contained an anti-PD-L1 scFv in place of the PD-L1-binding CDP but was otherwise the same as the CS-BiICE molecule. The scFv-based BiICE (“SS-BiICE”) was formed from a first fusion protein (METDTLLLWVLLLWVPGSTGDYKDEGGSDIVLTQSPATLSLSPGERATLSCRATESVE YYGTSLVQWYQQKPGQPPKLLIYAASSVDSGVPSRFSGSGSGTDFTLTINSLEAEDAAT YFCQQSRRVPYTFGQGTKLEIKGGGGSGGGGSGGGGSEVQLVQSGAEVKKPGASVKM SCKASGYTFTSYVMHWVKQAPGQRLEWIGYVNPFNDGTKYNEMFKGRATLTSDKSTS TAYMELSSLRSEDTAVYYCARQAWGYPWGQGTLVTVSSGGGGSEPKSSDKTHTCPPCP APELLGGPSVFLFPPKPKDTLMISRTPEVTCVVVDVSSHEDPEVKFNWYVDGVEVHNAK TKPREEQYASTYRVVSVLTVLHQDWLNGKEYKCKVSNKALGAPIEKTISKAKGQPREP QVYTLPPSRDELTKNQVSLSCAVKGFYPSDIAVEWESNGQPENNYKTTPPVLDSDGSFF LVSKLTVDKSRWQQGNVFSCSVMHEALHNHYTQKSLSLSPGK; SEQ ID NO: 346) containing an anti-PD-L1 scFc comprising SEQ ID NO: 343 (EVQLVQSGAEVKKPGASVKMSCKASGYTFTSYVMHWVKQAPGQRLEWIGYVNPFND GTKYNEMFKGRATLTSDKSTSTAYMELSSLRSEDTAVYYCARQAWGYPWGQGTLVTV SS) and SEQ ID NO: 344 (DIVLTQSPATLSLSPGERATLSCRATESVEYYGTSLVQWYQQKPGQPPKLLIYAASSVD SGVPSRFSGSGSGTDFTLTINSLEAEDAATYFCQQSRRVPYTFGQGTKLEIK) with an N-terminal signal peptide and FLAG tag fused to an Fc “hole” sequence and a second fusion protein (SEQ ID NO: 347) containing an anti-CD3 single-chain fragment variable (scFv) fused to an Fc “knob” sequence with an N-terminal signal peptide and C-terminal 6×His tag (SEQ ID NO: 248). Schematics of the CDP-containing CS-BiICE and the comparator SS-BiICE are illustrated in FIG. 11.

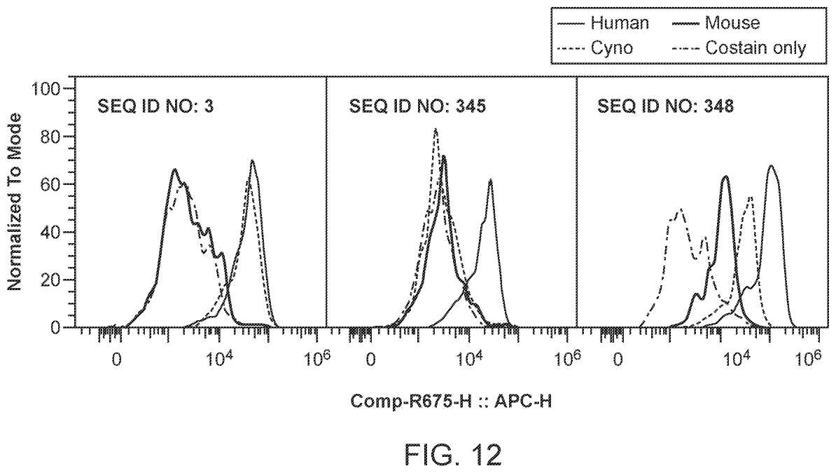

Like SEQ ID NO: 4, SEQ ID NO: 3, which contains a single D22N amino acid substitution relative to SEQ ID NO: 2, and the anti-PD-L1 scFv also lacked murine cross-reactivity, as shown in FIG. 12. Cross-reactivity of SEQ ID NO: 3, anti-PD-L1 scFv (DIVLTQSPATLSLSPGERATLSCRATESVEYYGTSLVQWYQQKPGQPPKLLIYAASSVD SGVPSRFSGSGSGTDFTLTINSLEAEDAATYFCQQSRRVPYTFGQGTKLEIKGGGGSGGG GSGGGGSEVQLVQSGAEVKKPGASVKMSCKASGYTFTSYVMHWVKQAPGQRLEWIG YVNPFNDGTKYNEMFKGRATLTSDKSTSTAYMELSSLRSEDTAVYYCARQAWGYPWG QGTLVTVSS; SEQ ID NO: 345), and an scFv derived from atezolizumab (EVQLVESGGGLVQPGGSLRLSCAASGFTFSDSWIHWVRQAPGKGLEWVAWISPYGGS TYYADSVKGRFTISADTSKNTAYLQMNSLRAEDTAVYYCARRHWPGGFDYWGQGTL VTVSSGGGGSGGGGSGGGGSDIQMTQSPSSLSASVGDRVTITCRASQDVSTAVAWYQQ KPGKAPKLLIYSASFLYSGVPSRFSGSGSGTDFTLTISSLQPEDFATYYCQQYLYHPATFG QGTKVEIK; SEQ ID NO: 348) to human, cynomolgus monkey (“Cyno”), or mouse PD-L1 was determined by measuring staining of cells expressing the PD-L1 binding moieties with fluorescently labeled PD-L1 orthologs. SEQ ID NO: 3 demonstrated binding to both human and cynomolgus PD-L1 but not to murine PD-L1. The anti-PD-L1 scFv (SEQ ID NO: 345) demonstrated binding to only human PD-L1. The scFv derived from atezolizumab (SEQ ID NO: 348) demonstrated binding to all three PD-L1 orthologs, where the highest binding is to human and the lowest binding is to mouse. Both scFv domains were of the VHVL orientation with a [GGGGS]₃ (SEQ ID NO: 165) linker joining VH and VL domains. The observed cross-reactivity of the scFv derived from atezolizumab to murine PD-L1 makes it a poor in vivo comparator in murine studies to SEQ ID NO: 3 and related variants as a BiICE component due to the possibility of on-target, off-tumor T-cell engagement. Unlike the scFv derived from atezolizumab, the anti-PD-L1 scFv did not show cross-reactivity to murine PD-L1. For this reason, the anti-PD-L1 scFv was selected as the comparator to SEQ ID NO: 3 and related variants to eliminate on-target off-tumor toxicity as a variable.

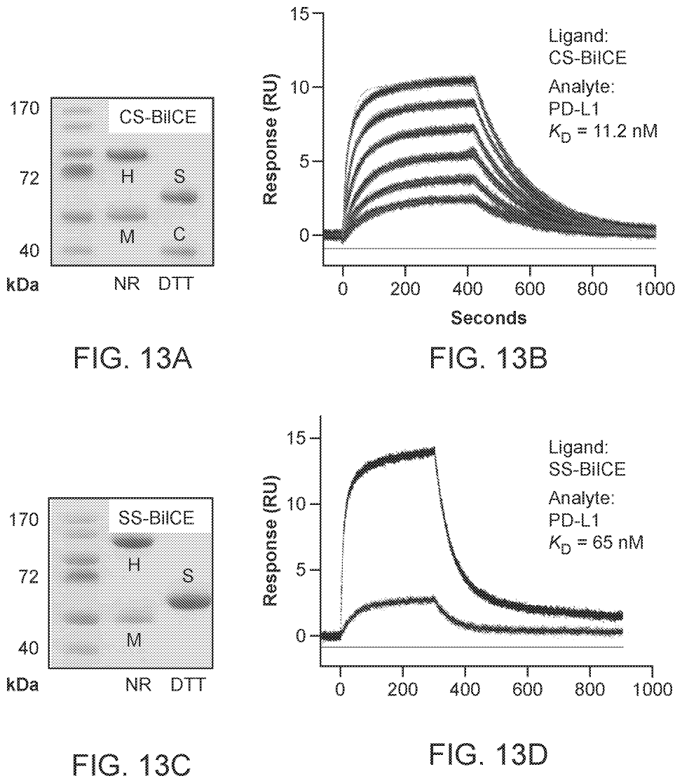

CS-BiICE and SS-BiICE molecules were produced as recombinant proteins as described in EXAMPLE 1 and purified by immobilized metal affinity chromatography (IMAC). SDS-PAGE gels of the purified CS-BiICE and SS-BiICE molecules are shown in FIG. 13A and FIG. 13C, respectively. Heterodimeric (H) species, representing a complex of anti-CD3-Fc knob (SEQ ID NO: 347) and PD-L1-binding-Fc hole (SEQ ID NO: 342 in CS-BiICE and SEQ ID NO: 346 in SS-BiICE), and anti-CD3-scFv-Fc monomer (M) were seen under non-reducing (NR) conditions for both CS-BiICE and SS-BiICE. The anti-CD3 monomer band was consistent with the band observed when anti-CD3-Fc knob was expressed alone. The presence of the monomeric band was due to the higher transduction efficiency for SEQ ID NO: 347 than SEQ ID NO: 346 or SEQ ID NO: 342. The heterodimer band was consistent with the molecular weight for the CS-BiICE and SS-BiICE heterodimers under non-reducing conditions in which disulfides join the two Fc halves. In the reduced lanes (“DTT”) of FIG. 13A and FIG. 13C, “S” band ran consistent with molecular weight of an scFv-Fc fusion, while the “C” band ran consistent with molecular weight of a CDP-Fc fusion and was only observed in the CS-BiICE sample. The presence of anti-CD3-scFv-Fc monomer did not affect T cell killing performance. Under reducing conditions (DTT), separate CDP-Fc and scFv-Fc species were seen for CS-BiICE due to their different sizes, while the scFv-Fc species appeared as a single band for SS-BiICE due to the similar molecular sizes of the two species. Binding affinity of CS-BiICE and SS-BiICE in vitro to PD-L1 was measured using SPR, as shown in FIG. 13B and FIG. 13D, respectively. Both CS-BiICE and SS-BiICE demonstrated binding in vitro to PD-L1. SPR of the CS-BiICE (FIG. 13B) demonstrated a reduction in overall PD-L1 affinity upon incorporation into a BiICE scaffold relative to the PD-L1-binding CDP alone (K_{D}=11.2 nM for CS-BiICE versus 202 pM for SEQ ID NO: 1). This reduction in affinity was likely due to a reduced association constant, as the dissociation rate of CS-BiICE was actually slower than that of the PD-L1-binding CDP alone (k_{d}=6.06×10⁻³ s⁻¹ for CS-BiICE versus 1.96×10⁻² s⁻¹ for SEQ ID NO: 1), while the association constant of the CS-BiICE was much lower (kₐ=4.07×10⁵ M⁻¹s⁻¹ for CS-BiICE versus 9.73×10⁷ M⁻¹s⁻¹ for SEQ ID NO: 1). In the context of an immunological synapse, there is expected to be high local receptor concentration, so a reduced on-rate may not be detrimental to performance. SPR of the SS-BiICE (FIG. 13D) demonstrated that the PD-L1-engaging scFv of the SS-BiICE promoted binding to PD-L1 at K_{D}=65 nM. Both the on-rate (kₐ=1.71×10⁵ M⁻¹s⁻¹) and the off-rate (k_{d}=1.10×10⁻² s⁻¹) of SS-BiICE were slightly worse than that of CS-BiICE. This demonstrates that SEQ ID NO: 2, which contains a single Q22D substitution relative to SEQ ID NO: 1, can be assembled with a CD3-binding moiety in an Fc fusion format, and that the assembled molecule binds PD-L1 and with a higher affinity than a comparator molecule using a PD-L1-binding scFv.

### Example 8

### Induction of T-Cell Killing Using a PD-L1-Binding Bispecific Immune Cell Engager

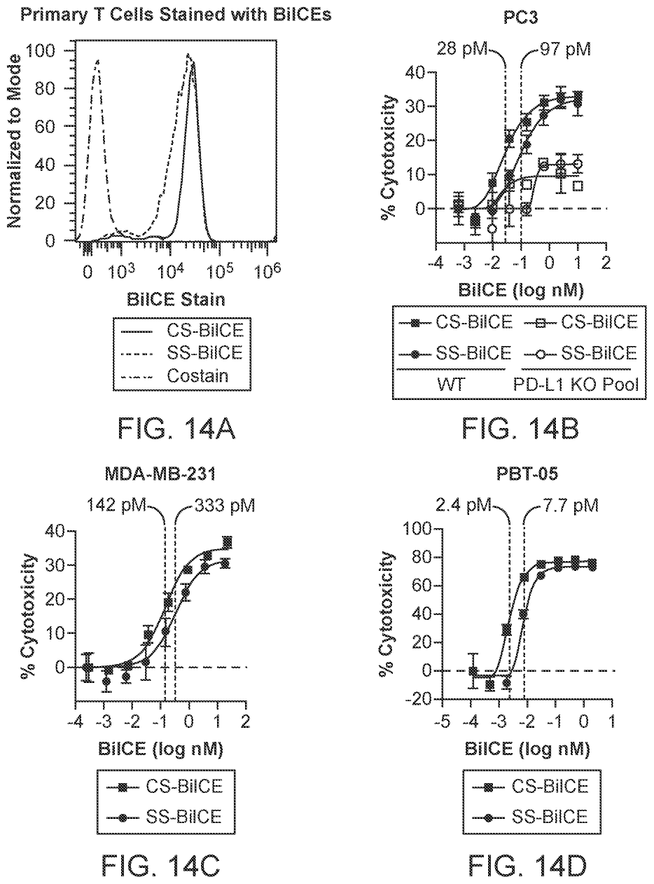

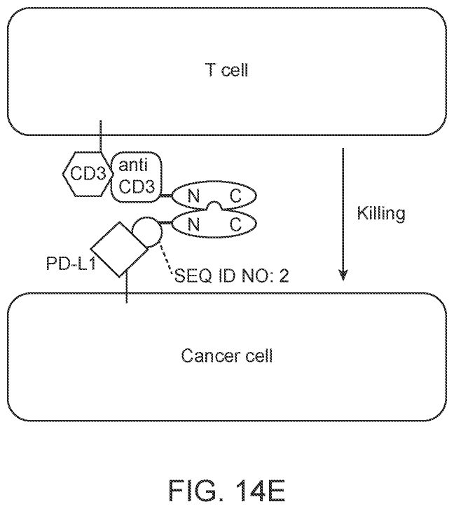

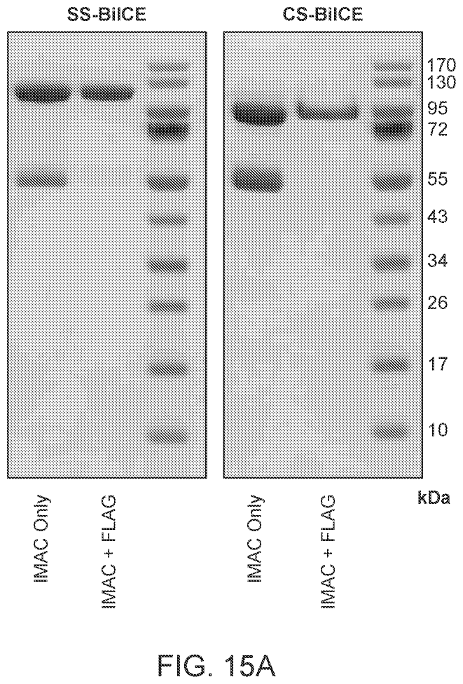

This example describes induction of T-cell killing using a bispecific immune cell engager (BiICE) molecule containing a PD-L1-binding CDP. The BiICE molecules, prepared and validated as described in EXAMPLE 7, demonstrated binding to primary T cells purified from human patient derived peripheral blood mononuclear cells (PBMCs, FIG. 14A). In vitro T-cell killing (TCK) assays using cancer cells incubated with activated T cells (ATCs) showed that PD-L1-binding BiICEs, CS-BiICE (SEQ ID NO: 342 heterodimerized with SEQ ID NO: 347) and SS-BiICE (SEQ ID NO: 346 heterodimerized with SEQ ID NO: 347), potently induced T-cell killing against three human cancer lines: prostate cancer PC3 (FIG. 14B), triple negative breast cancer MDA-MB-231 (FIG. 14C), and patient-derived pediatric brain tumor PBT-05 (FIG. 14D). The EC50 on PC3 cells was 28 pM for CS-BiICE and 97 pM for SS-BiICE, the EC50 on MDA-MB-231 cells was 142 pM for CS-BiICE and 333 pM for SS-BiICE, and the EC50 on PBT-05 cells was 2.4 pM for CS-BiICE and 7.7 pM for SS-BiICE. In each case, the CS-BiICE was more potent than the SS-BiICE. An example tumor-killing mechanism using a BiICE is illustrated in FIG. 14E.

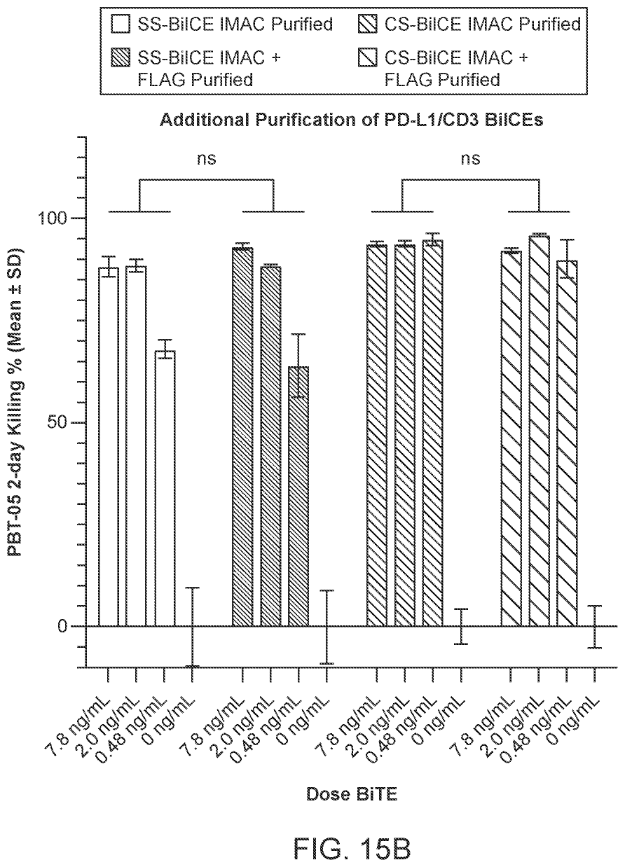

These assays also demonstrated that PD-L1-engagement is required for maximal activity, as pooled PD-L1 knockout PC3 cells demonstrated substantially less T-cell killing upon BiICE incubation (FIG. 14B); the small activity observed is likely related to remaining PD-L1-positive cells in the knockout pool. Since it was observed that preparations of both CS-BiICE and SS-BiICE contained impurities representing monomeric CD3-engaging scFv-Fc, a T-cell killing assay was performed using extra-purified BiICE preparations to further validate that T-cell killing activity was PD-L1-dependent. The PD-L1-engaging arm of each molecule also contains a short FLAG tag (DYKDE, SEQ ID NO: 431), so anti-FLAG-M1 affinity purification of bispecific BiICEs away from the anti-CD3 scFv-Fc monomers was performed. SDS-PAGE gels of IMAC only and IMAC plus FLAG extra-purified BiICE preparations are provided in FIG. 15A. Extra-purified BiICE preps that underwent both IMAG and FLAG purification were compared with the IMAC-purified preps in a TCK assays with PBT-05 cells, as shown in FIG. 15B. The presence of the CD3-engaging scFv-Fc monomer did not impact performance, showing the molecules' activities are dependent on bispecific engagement of both targets, and that the impurities found in the preps do not confer TCK activity. This data demonstrates that a bispecific T-cell engager built using a PD-L1-binding CDP of this disclosure and a CD3 binding moiety can cause T-cell mediated human cancer cell killing in vitro, and that is was more potent than a bispecific T-cell engager built using a PD-L1 binding CDP.

### Example 9

### In Vivo Treatment of Cancer Using a PD-L1-Binding Bispecific Immune Cell Engager

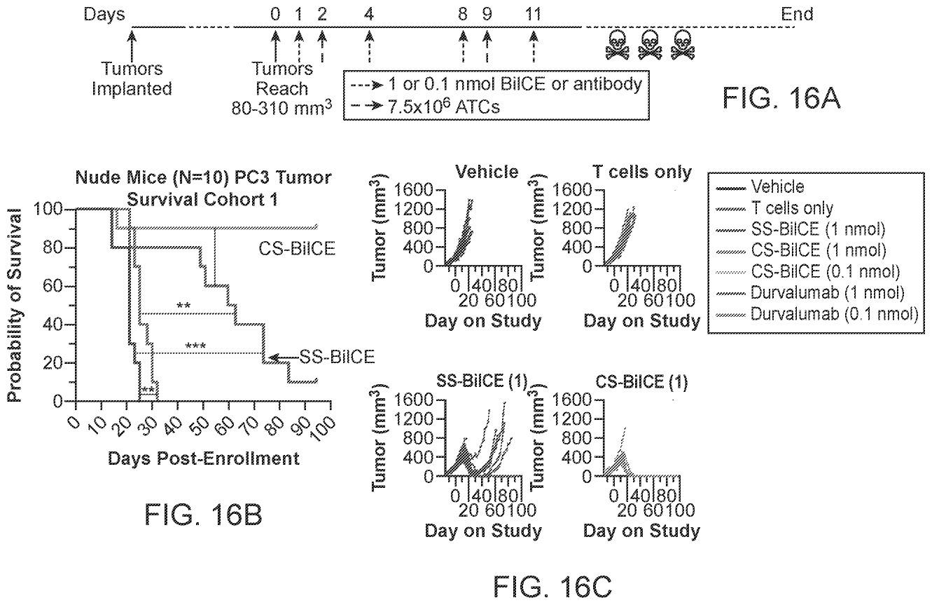

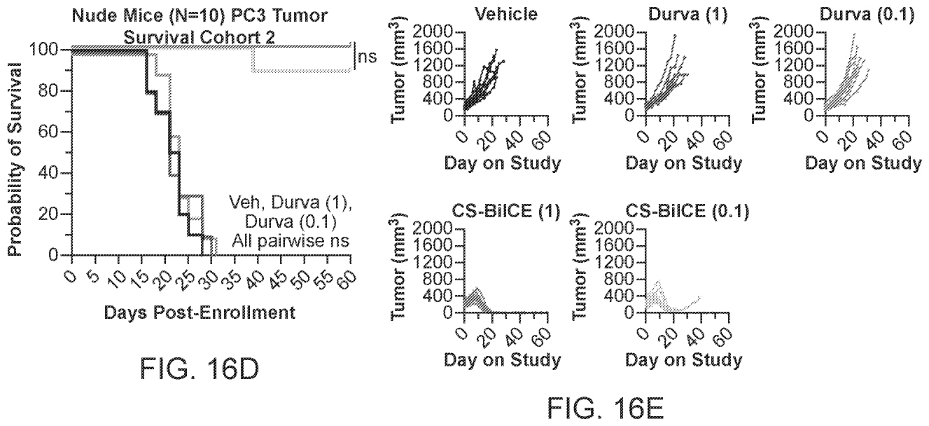

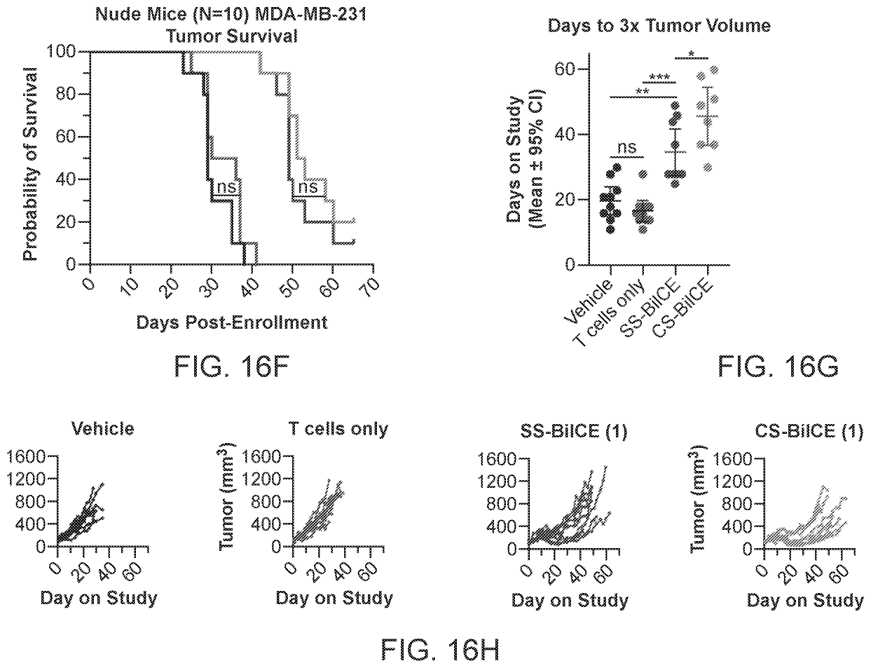

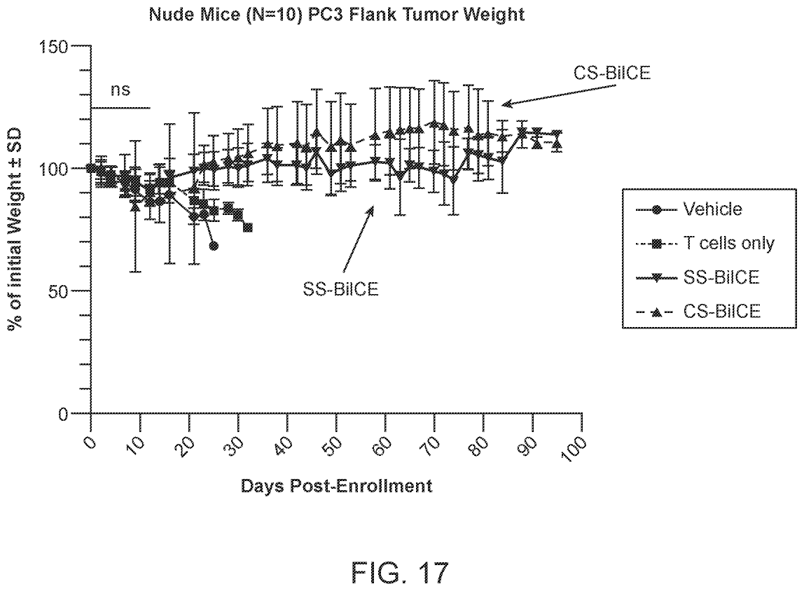

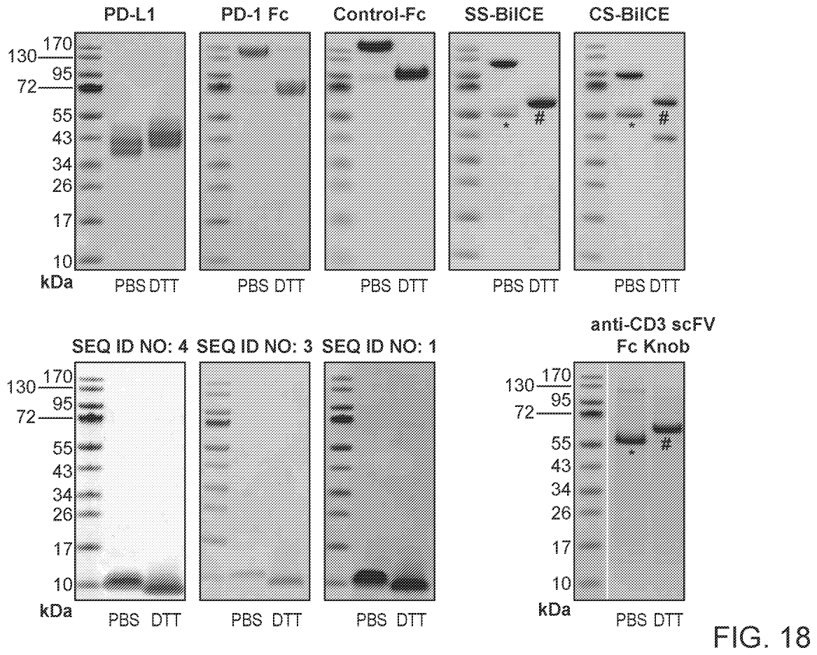

This example describes in vivo treatment of cancer using a bispecific immune cell engager (BiICE) molecule containing a PD-L1-binding CDP. As a preclinical proof of concept, the BiICE molecules, prepared and validated as described in EXAMPLE 7, were tested in mice carrying flank tumors. Nude mice carrying flank tumors, with masses of 100-200 mm³ upon enrollment, were treated with activated human T cells (ATCs, 7.5×10⁶ per dose) and 1 nmol doses of either CS-BiICE or SS-BiICE (1 nmol=100 μg SS-BiICE, 80 μg CS-BiICE per dose). T cells were activated using a T cell activation kit containing microspheres capable of binding and crosslinking CD3 and CD28 simultaneously. The two-week treatment included four BiICE injections (on days 1, 4, 8, and 11) and two ATC infusions (on days 2 and 7), as shown in the experimental timeline provided in FIG. 16A. Tumor size was measured over time. Mice were removed from study and euthanized when tumors reached 1500 mm³, if tumors developed an open ulcer on their surface, or if body weight dropped to 80% of the starting value. PC3 and MDA-MB-231 flank tumors were tested. PC3-bearing mice treated with ATCs and SS-BiICE lived almost 2.5× longer (median survival 61.5 days post-enrollment) than ATC-only mice (median survival 25 days), who themselves outlived vehicle-only mice (median survival 20 days), as shown in FIG. 16B. However, 9 out of 10 PC3-bearing mice treated with ATCs and SS-BiICE eventually required euthanasia based on tumor regrowth. With ATC and CS-BiICE treatment, only 1 of 10 mice with PC3 tumors required euthanasia; the other nine survived until culling at the conclusion of the experiment on Day 95 due to apparent complete elimination of the tumors. No overt signs of toxicity (weight loss or observed behavioral changes) were apparent in either BiICE treatment group. Weight trends of the mice treatment groups over the course of the study are shown in FIG. 17. Tumor volume of PC3 tumors were measured over the course of the study, as shown in FIG. 16C. These results demonstrated that 8 of 10 SS-BiICE mice responded to treatment, but of the 8 responders, 7 eventually saw re-growth of their tumors. In contrast, in the 9 of 10 CS-BiICE responders, all 9 demonstrated complete tumor clearance. A second cohort of PC3 flank tumor-bearing mice, as shown in FIG. 16D and FIG. 16E, validated the CS-BiICE elimination, while also demonstrating that anti-PD-L1 antibody durvalumab has no effect in this setting, showing that the BiICE effect is not simply due to inhibition of PD-L1, and that reduction in dose of CS-BiICE by 10 fold produced similar tumor elimination effects. These results indicated that CS-BiICE is at least 10 times as potent as SS-BiICE, because a 1/10 dose (0.1 nmol) of CS-BiICE eliminated PC3 tumors while a full SS-BiICE dose (1 nmol) did not.

In MDA-MB-231 tumors, for which the BiICEs demonstrated lower potency in vitro, both BiICEs substantially increased lifespan, as shown in FIG. 16F; median survival of 33 days in ATC-only mice was extended to 49 and 52 days with ATC+SS-BiICE and ATC+CS-BiICE, respectively, though no mice demonstrated complete MDA-MB-231 tumor clearance. Comparing the BiICEs in MDA-MB-231 flank tumors, CS-BiICE treatment did not significantly extend survival relative to SS-BiICE treatment, though CS-BiICE treatment did generate slower tumor growth kinetics than SS-BiICE (FIG. 1611), as measured by how long after enrollment it took for tumors to triple in volume (FIG. 16G). In both tumor models and with both PD-L1-engaging modalities, PD-L1/CD3 BiICE treatment presented substantial tumor mass reduction and survival enhancement, with CS-BiICE either subtly (MDA-MB-231 tumors) or substantially (PC3 tumors) out-performing SS-BiICE.

### Example 10

### Treatment of Cancer Using a PD-L1-Binding Bispecific Immune Cell Engager

This example describes treatment of cancer using a PD-L1-binding bispecific immune cell engager. A PD-L1-binding cystine-dense peptide of any one of SEQ ID NO: 1-SEQ ID NO: 118, SEQ ID NO: 435, SEQ ID NO: 436, or SEQ ID NO: 554-SEQ ID NO: 567 is complexed with a T cell-targeting agent to form a bispecific immune cell engager. The T cell-targeting agent binds to CD3, 4-1BB, CD137, or CD28. The bispecific immune cell engager is administered to a human subject with cancer. Upon administration, the bispecific immune cell engager recruits a T cell to a PD-L1 positive cancer cell by binding to the PD-L1 positive cancer cell through the PD-L1-binding CDP and binding to the T cell through the T cell-targeting agent. The recruited T cell targets and kills the PD-L1 positive cancer cell, thereby treating the cancer.

### Example 11

### Treatment of an Autoimmune Disorder Using a PD-L1-Binding Bispecific Immune Cell Engager

This example describes treatment of an autoimmune disorder using a PD-L1-binding bispecific immune engager. A PD-L1-binding cystine-dense peptide of any one of SEQ ID NO: 1-SEQ ID NO: 118, SEQ ID NO: 435, SEQ ID NO: 436, or SEQ ID NO: 554-SEQ ID NO: 567 is complexed with an immune cell-targeting agent that binds a regulatory T cell or a mesenchymal stem cell to form a bispecific immune cell engager. The immune cell-targeting agent binds to CD25, CD13, CD29, CD44, CD71, CD73, CD90, CD105, CD166, CD27, GITR, TIGIT, LAG3, TCR, CD40L, OX40, PD-1, CTLA-4, or STRO-1. The bispecific immune cell engager is administered to a human subject with an autoimmune disorder. Upon administration, the bispecific immune cell engager recruits regulatory T cell or mesenchymal stem cell to a PD-L1 positive cell such as a pancreatic beta cell by binding to the PD-L1 positive cell through the PD-L1-binding CDP and binding to the regulatory T cell or mesenchymal stem cell through the immune cell-targeting agent. The recruited regulatory T cell or mesenchymal stem cell targets PD-L1 rich cell such as a pancreatic islets to protect them from autoimmune T-cell killing, thereby treating the autoimmune disorder, such as type 1 diabetes.

### Example 12

### Treatment of a Cancer Using a PD-L1-Binding Peptide

This example describes treatment of a cancer using a PD-L1-binding peptide. A PD-L1-binding cystine-dense peptide of any one of SEQ ID NO: 1-SEQ ID NO: 118, SEQ ID NO: 435, SEQ ID NO: 436, or SEQ ID NO: 554-SEQ ID NO: 567 is administered to a subject having cancer. Upon administration to the subject, the PD-L1-binding peptide targets and binds to PD-L1 on a PD-L1 positive cancer cell. The PD-L1-binding peptide binds at a site overlapping with the PD-1 binding interface on PD-L1, preventing PD-L1 from binding, and inhibiting PD-L1. Binding and inhibiting of PD-L1 reduces immunosuppression, reduces T cell exhaustion, and restores immune function within the cancer cell microenvironment, thereby treating the cancer.

### Example 13

### Treatment of a Cancer Using a PD-L1-Binding Peptide Complexed with an Anti-Cancer Agent

This example describes treatment of a cancer using a PD-L1-binding peptide complexed with an anti-cancer agent. A PD-L1-binding cystine-dense peptide of any one of SEQ ID NO: 1-SEQ ID NO: 118, SEQ ID NO: 435, SEQ ID NO: 436, or SEQ ID NO: 554-SEQ ID NO: 567 is complexed with an anti-cancer agent. The PD-L1-binding peptide anti-cancer agent complex is administered to a subject with cancer. Upon administration to the subject, the PD-L1-binding peptide targets and binds to PD-L1 on a PD-L1 positive cancer cell and delivers the anti-cancer agent to the cancer cell. The anti-cancer agent kills the cancer cell, thereby treating the cancer.

### Example 14

### Imaging of a Cancer Using a PD-L1-Binding Peptide Complexed with a Detectable Agent

This example describes imaging of a cancer using a PD-L1-binding peptide complexed with a detectable agent. A PD-L1-binding cystine-dense peptide of any one of SEQ ID NO: 1-SEQ ID NO: 118, SEQ ID NO: 435, SEQ ID NO: 436, or SEQ ID NO: 554-SEQ ID NO: 567 is complexed with a detectable agent. The PD-L1-binding peptide detectable agent complex is administered to a subject with cancer or suspected to have cancer. Upon administration to the subject, the PD-L1-binding peptide targets and binds to PD-L1 on a PD-L1 positive cancer cell and delivers the detectable agent to the cancer cell. The detectable agent labels the cancer cell, and the presence or absence of the detectable agent in a region of the subject suspected to have cancer is detected, thereby imaging the cancer.

### Example 15

### Treatment of a Cancer Using a PD-L1-Binding Chimeric Antigen Receptor

This example describes treatment of a cancer using a PD-L1-binding chimeric antigen receptor. A chimeric antigen receptor comprising a PD-L1-binding cystine-dense peptide of any one of SEQ ID NO: 1-SEQ ID NO: 118, SEQ ID NO: 435, SEQ ID NO: 436, or SEQ ID NO: 554-SEQ ID NO: 567 in place of a single chain variable fragment (scFv), a transmembrane domain, and an intracellular domain is expressed in a T cell collected from a subject having cancer. T cells expressing the chimeric antigen receptor are administered to the subject. Upon administration, the PD-L1-binding peptide of the chimeric antigen receptor targets and binds to a PDL-L1 positive cancer cell and delivers the T cell to the cancer cell. The T cell kills the cancer cell, thereby treating the cancer.

### Example 16

### Engineering PD-L1-Binding Peptides for pH-Dependent Binding

This example describes development and in vitro testing of PD-L1-binding peptides or peptide complexes capable of pH-dependent dissociation from PD-L1, for example, at endosomal pH (e.g., pH 5.5).

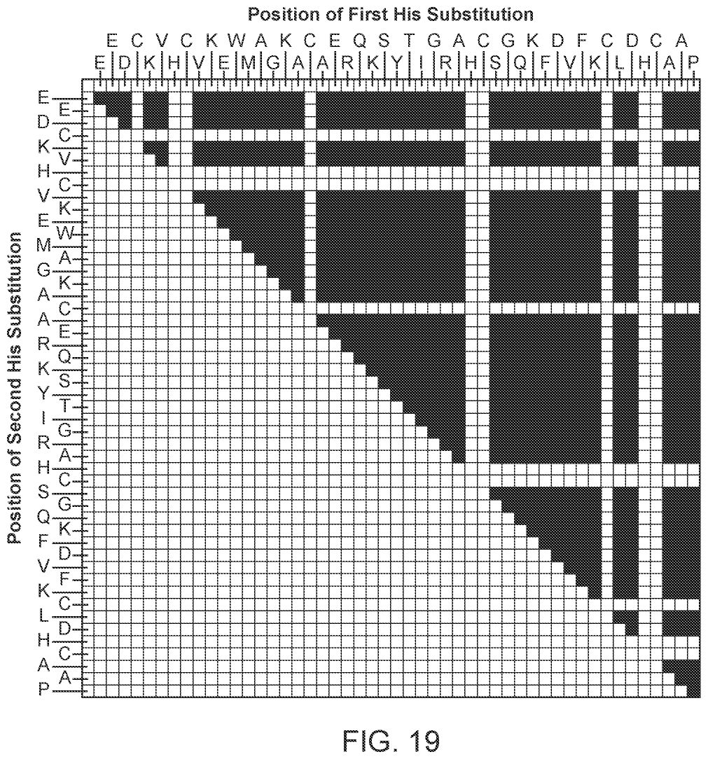

Imparting pH-dependent binding to a target-engaging domain (CDP or otherwise) can done in a variety of ways, an example of which is provided here. Here, a library of variants was designed containing histidine substitutions. Histidine residues were introduced because, of all of the natural amino acids, His is the only one with a side chain whose charge changes significantly between neutral (e.g., pH 7.4) and acidic (e.g., pH<6) endosomal conditions. This change of charge can alter binding, either directly (introducing a positive charge at low pH that could result in charge repulsion of nearby cationic groups) or indirectly (the change in charge imparts a subtle change in the binder's structure, disrupting a protein-protein interface) as the pH changes, for example from a physiologic extracellular environment to an endosomal environment as the endosome acidifies. In its simplest form, this could be executed by generating double-His doped libraries, where, for a CDP, every non-Cys, non-His residue could be substituted with a His one- or two-at-a-time. FIG. 19 shows a high-affinity PD-L1-binding CDP sequence (SEQ ID NO: 1, EEDCKVHCVKEWMAGKACAERQKSYTIGRAHCSGQKFDVFKCLDHCAAP) above and to the side of a His substitution matrix. Each black box represents a first and second site in which His could be substituted. Those purely along the top-left to bottom-right diagonal represent single His substitutions. Each black box represents a variant with one or two native-to-His substitutions, representing 821 peptide variants to be screened. A variant library containing the parental sequence and variants with one or two native-to-His substitutions was generated and tested.

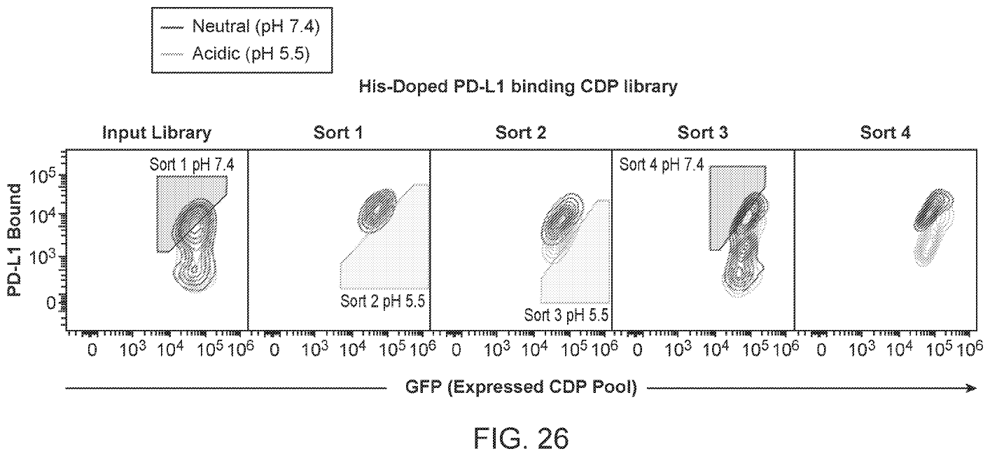

The resulting histidine-enriched PD-L1-binding peptides were evaluated for their PD-L1 binding in comparative binding experiments at various pH levels or ranges. A variant library of PD-L1-binding peptides was expressed via mammalian surface display, with each variant containing zero, one or two His substitutions. These variants were tested for maintenance of binding under extracellular pH (such as pH 7.4), and for reduced binding under endosomal pH (such as pH 5.5). Sequential screening was performed, as shown in FIG. 26. The input library was initially screened for PD-L1 binding at pH 7.4, and strong binders were selected (shaded area). The second and third rounds of screening (“Sort 1” and “Sort 2,” respectively) were performed at pH 5.5 to mimic endosomal pH, and the weak binders were collected (shaded area). The final round of screening (“Sort 3”) was performed at pH 7.4, and strong binders were selected. Differential binding at pH 7.4 and pH 5.5 was observed following screening (“Sort 4”).

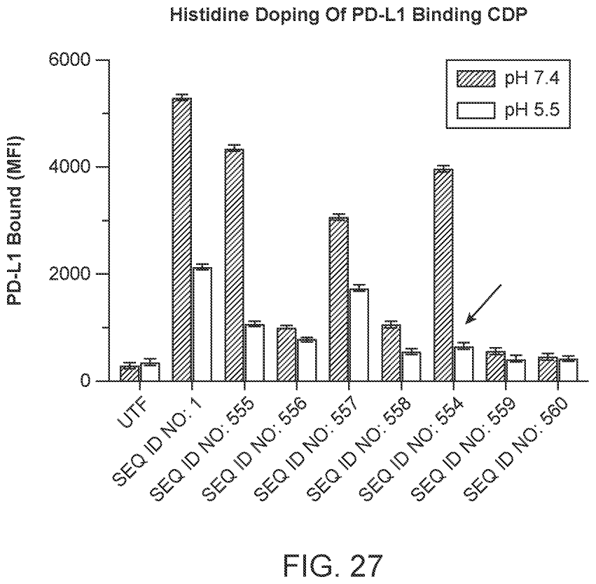

Variants of SEQ ID NO: 1 containing histidine substitutions at one, two, or three of E2H, M13H, and K16H amino acid positions were identified in the pooled screen as pH-dependent binders of PD-L1. pH-dependent binding was validated by measuring PD-L1 binding at pH 7.4 and pH 5.5 to cells surface expressing single variants, as shown in FIG. 27. Peptides containing substitutions at E2H (EHDCKVHCVKEWMAGKACAERQKSYTIGRAHCSGQKFDVFKCLDHCAAP, SEQ ID NO: 555), M13H (EEDCKVHCVKEWHAGKACAERQKSYTIGRAHCSGQKFDVFKCLDHCAAP, SEQ ID NO: 556), K16H (EEDCKVHCVKEWMAGHACAERQKSYTIGRAHCSGQKFDVFKCLDHCAAP, SEQ ID NO: 557), E2H and M13H (EHDCKVHCVKEWHAGKACAERQKSYTIGRAHCSGQKFDVFKCLDHCAAP, SEQ ID NO: 558), E2H and K16H (EHDCKVHCVKEWMAGHACAERQKSYTIGRAHCSGQKFDVFKCLDHCAAP, SEQ ID NO: 554), M13H and K16H (EEDCKVHCVKEWHAGHACAERQKSYTIGRAHCSGQKFDVFKCLDHCAAP, SEQ ID NO: 559), or E2H, M13H, and K16H (EHDCKVHCVKEWHAGHACAERQKSYTIGRAHCSGQKFDVFKCLDHCAAP, SEQ ID NO: 560) were compared to SEQ ID NO: 1. The variant corresponding to SEQ ID NO: 554, containing substitutions at E2H and K16H, showed strong binding to PD-L1 at pH 7.4 and substantial loss of binding at pH 5.5 (black arrow). The other variants and the parent peptide showed varying levels of PD-L1 binding at pH 7.4 and at pH 5.5, with varying degrees of pH dependence to the binding.

### Example 17

### Extension of Peptide Plasma Half-Life Using Serum Albumin-Binding Peptide Complexes

This example demonstrates a method of extending the serum or plasma half-life of a peptide using serum albumin-binding peptide complexes as disclosed herein. A peptide or peptide complex having a sequence of any one of SEQ ID NO: 1-SEQ ID NO: 118, SEQ ID NO: 435, SEQ ID NO: 436, or SEQ ID NO: 554-SEQ ID NO: 567 or SEQ ID NO: 119-SEQ ID NO: 153 is modified in order to increase its plasma half-life. The peptide and the serum half-life extending moiety are fused recombinantly, chemically synthesized as a single fusion, separately recombinantly expressed and conjugated, or separately chemically synthesized and conjugated. Fusing the peptide to a serum albumin-binding peptide extends the serum half-life of the peptide complex. The peptide or peptide complex is conjugated to a serum albumin-binding peptide, such as SA21 (SEQ ID NO: 242). Optionally, the peptide fused to SA21 has a sequence of any one of SEQ ID NO: 1, SEQ ID NO: 2, or SEQ ID NO: 342. Optionally, the peptide fused to SA21 is linked to SA21 via a peptide linker. The linker links two separately functional CDPs to incorporate serum half-life extension function into the peptide or peptide complex. The linker enables SA21 to cyclize without steric impediment from either member of the peptide complex. Alternatively, conjugation of the peptide to albumin, an albumin binder, such as Albu-tag, or a fatty acid, such as a C₁₄-C₁₅ fatty acid or palmitic acid, is used to extend plasma half-life. Plasma half-life is also optionally extended as a result of reduced immunogenicity by using minimal non-human protein sequences.

### Example 18

### Treatment of a CNS Cancer Using a PD-L1-Binding Peptide

This example describes treatment of a cancer using a PD-L1-binding peptide. A PD-L1-binding cystine-dense peptide of any one of SEQ ID NO: 1-SEQ ID NO: 118, SEQ ID NO: 435, SEQ ID NO: 436, or SEQ ID NO: 554-SEQ ID NO: 567 is fused with a TfR-binding peptide such as SEQ ID NO: 350, optionally using a linker such as SEQ ID NO: 154-SEQ ID NO: 241 or SEQ ID NO: 433. The PD-L1-binding CDP-TfR-binding peptide complex is administered to a subject having cancer. Upon administration to the subject, the PD-L1-binding CDP-TfR-binding peptide complex crosses the blood brain barrier and binds to PD-L1 on a PD-L1 positive primary or metastatic cancer cell in the brain. The PD-L1-binding CDP-TfR-binding peptide complex binds at a site overlapping with the PD-1 binding interface on PD-L1, preventing PD-L1 from binding, and inhibiting PD-L1. Binding and inhibiting of PD-L1 reduces immunosuppression, reduces T cell exhaustion, and restores immune function within the cancer cell microenvironment, thereby treating the cancer.

### Example 19

### Synthesis of a Peptide Oligonucleotide Complex for Antisense Therapy

A gene targeted for silencing in order to address a disease is identified and the desired single-stranded antisense oligonucleotide sequence is designed and synthesized based on the target coding or complementary sequence. The antisense oligonucleotide is conjugated to any PD-L1 binding peptide disclosed herein, including peptides of any one of SEQ ID NO: 1-SEQ ID NO: 118, SEQ ID NO: 435, SEQ ID NO: 436, or SEQ ID NO: 554-SEQ ID NO: 567 by any of the methods disclosed herein, for example, in accordance with EXAMPLE 26-EXAMPLE 31, such as with a cleavable or stable linker. Optionally, a nucleotide (including the backbone) is modified, such as to increase in vivo stability, to increase resistance to enzymes such as nucleases, increase protein binding including to serum proteins, increase in vivo half-life, to modify the tissue biodistribution, or to reduce immune system activation.

Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 20

### Synthesis of a Peptide-Oligonucleotide Conjugate for RNAi Therapy

A gene targeted for silencing in order to address a disease is identified and the desired double-stranded RNAi sequence is designed and synthesized based on the target coding or complementary sequence. The sense or the antisense oligonucleotide of the RNAi is conjugated to any PD-L1-binding peptide disclosed herein, including a peptide of any one of SEQ ID NO: 1-SEQ ID NO: 118, SEQ ID NO: 435, SEQ ID NO: 436, or SEQ ID NO: 554-SEQ ID NO: 567 by any of the methods disclosed herein, for example, in accordance with EXAMPLE 26-EXAMPLE 31, such as with a cleavable or stable linker. Optionally the peptide is conjugate to the sense (passenger) strand of the oligonucleotide. Optionally, a nucleotide (including the backbone) is modified, such as to increase in vivo stability, to increase resistance to enzymes such as nucleases, increase protein binding including to serum proteins, increase in vivo half-life, to modify the tissue biodistribution, or to reduce immune system activation. The sense and antisense strands are hybridized together, either before or after the conjugation.

Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 21

### Synthesis of a Peptide-Oligonucleotide Conjugate for U1 Adaptor Therapy

A gene targeted for silencing in order to address a disease is identified and the desired oligonucleotide sequence for U1 adaptor therapy is designed and synthesized based on the target coding or complementary sequence. The oligonucleotide is conjugated to any to any PD-L1-binding peptide disclosed herein, including peptide of SEQ ID NO: 1-SEQ ID NO: 118, SEQ ID NO: 435, SEQ ID NO: 436, or SEQ ID NO: 554-SEQ ID NO: 567 by any of the methods disclosed herein, for example, in accordance with EXAMPLE 26-EXAMPLE 31, such as with a cleavable or stable linker. Optionally, a nucleotide (including the backbone) is modified, such as to increase in vivo stability, to increase resistance to enzymes such as nucleases, increase protein binding including to serum proteins, increase in vivo half-life, to modify the tissue biodistribution, or to reduce immune system activation.

Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 22

### Synthesis of a Peptide-Oligonucleotide Conjugate for Aptamer Therapy

An aptamer sequence that interacts with a target molecule is selected to address a disease is identified against the target and synthesized. The aptamer oligonucleotide is conjugated to any PD-L1 binding peptide disclosed herein, including any peptide of SEQ ID NO: 1-SEQ ID NO: 118, SEQ ID NO: 435, SEQ ID NO: 436, or SEQ ID NO: 554-SEQ ID NO: 567 by any of the methods disclosed herein, for example, in accordance with EXAMPLE 26-EXAMPLE 31, such as with a cleavable or stable linker. Optionally, a nucleotide (including the backbone) is modified, such as to increase in vivo stability, to increase resistance to enzymes such as nucleases, increase protein binding including to serum proteins, increase in vivo half-life, to modify the tissue biodistribution, or to reduce immune system activation.

Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 23

### Conjugation of an Oligonucleotide and a Peptide Using Click Chemistry

An alkyne or azide group is installed in an oligonucleotide, such as by adding a hexynyl group to the 5′ end or the 3′ end of the oligonucleotide, installation of a 5-Octadiynyl dU, installation of a DIBO at the 5′ end using, which is optionally installed using a DIBO phosphoramidite, or installation of an azide group by use of an NHS ester reaction linking an azide group to a dT base. An azide or an alkyne group is installed on a peptide, such as by incorporating an N-terminal 6-azidohexanoic acid, an azidohomoalanine residue, or homopropargyl glycine residue. Optionally, the alkyne group comprises a strained ring such as strained cyclooctyne ring, such as DIBO. The oligonucleotide is conjugated to any PD-L1 binding peptide disclosed herein, including any peptide of SEQ ID NO: 1-SEQ ID NO: 118, SEQ ID NO: 435, SEQ ID NO: 436, or SEQ ID NO: 554-SEQ ID NO: 567. The oligonucleotide and the peptide are conjugated together by combining an azide group on one with the alkyne group on the other using a copper-catalyzed azide-alkyne cycloaddition or a strain-promoted azide-alkyne cycloaddition to form a triazole bond.

Any peptide oligonucleotide complexes of the present disclosure may be so modified with an alkyne or azide group and are described. Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 24

### Conjugation of an RNAi Sequence and a Peptide Using Click Chemistry

An alkyne group within a strained cyclooctyne is installed on an oligonucleotide, optionally linked to either the 5′ or the 3′ end of a sense or antisense strand. Optionally the strained cyclooctyne is DIBO, which is optionally installed on the 5′ end using a DIBO phosphoramidite. An azide group is installed on a peptide. Optionally, a 6-azidohexanoyl group is added to the N-terminus of any one of SEQ ID NO: 1-SEQ ID NO: 118, SEQ ID NO: 435, SEQ ID NO: 436, or SEQ ID NO: 554-SEQ ID NO: 567, optionally with a linker in between the 6-azidohexanoyl group and the peptide. Optionally, the peptide is prepared as a TFA salt form. The alkyne-containing oligonucleotide and the azide-containing peptide are contacted together, such as in a buffer, solution, or solvent. The azide and the alkyne react to form a triazole bond that links the oligonucleotide and the peptide. The sense and antisense strands of the RNAi are hybridized together, either before or after the conjugation reaction.

Any peptide oligonucleotide complexes of the present disclosure may be modified to include an alkyne group within a strained cyclooctyne and are described. Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 25

### Conjugation of a U1 Adapter Sequence and a Peptide Using Click Chemistry

An alkyne group within a strained cyclooctyne is installed on an oligonucleotide, optionally linked to either the 5′ or the 3′ end of a sequence, designed for U1 adapter therapy. Optionally the strained cyclooctyne is DIBO, which is optionally installed on the 5′ end using a DIBO phosphoramidite. An azide group is installed on a peptide. Optionally, the peptide is prepared as a TFA salt form. The alkyne-containing oligonucleotide and the azide-containing peptide are contacted together, such as in a buffer, solution, or solvent. The azide and the alkyne react to form a triazole bond that links the oligonucleotide and the peptide.

Any peptide oligonucleotide complexes of the present disclosure may be modified with an alkyne group within a strained cyclooctyne are described. Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 26

### Installation of a Thiol Group, an Amine Group, or an Aldehyde Group in an Oligonucleotide

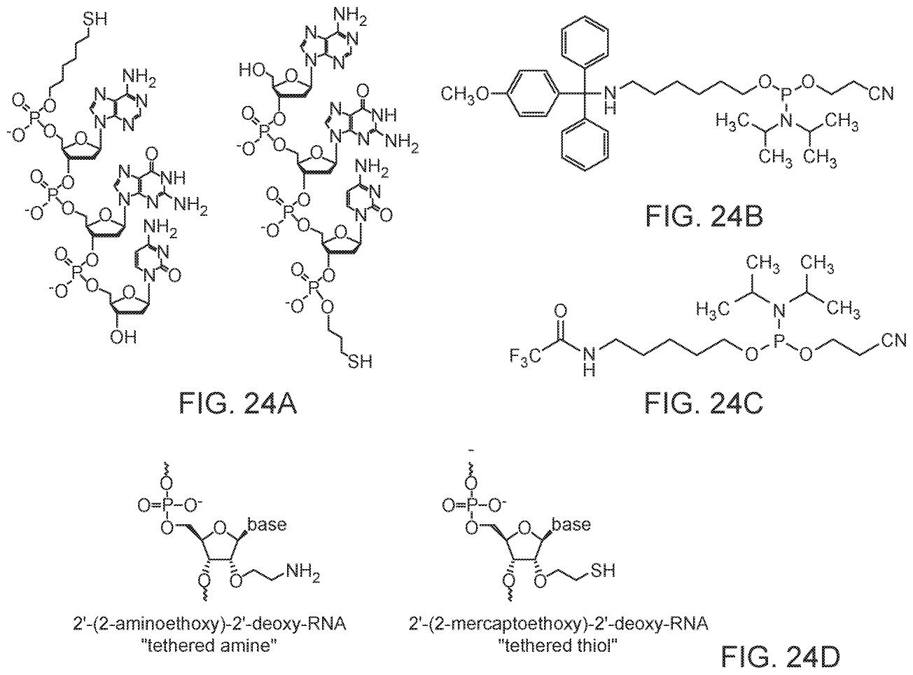

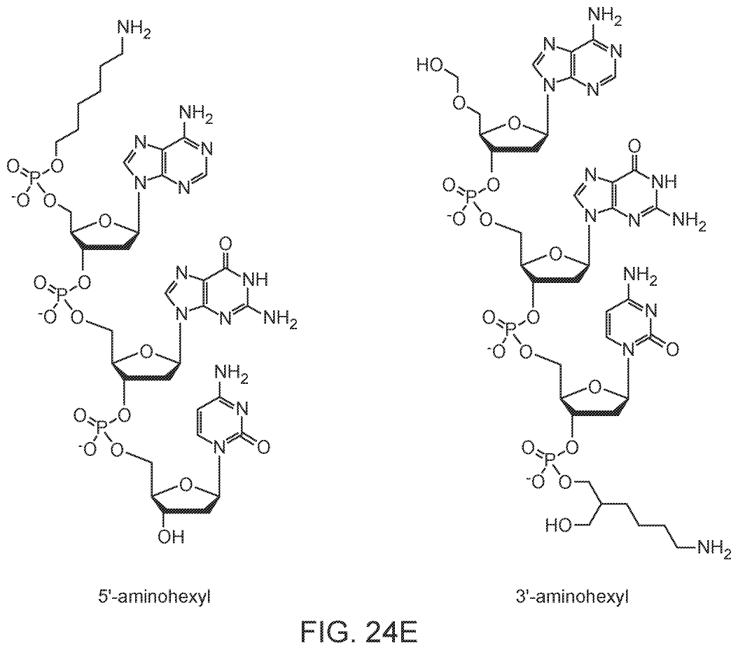

This example describes incorporation of a thiol group, an amine group, or an aldehyde group in RNA or DNA or any oligonucleotide. FIG. 24A-FIG. 24E illustrates incorporation or addition of these groups on RNA or DNA. A thiol group is added on an oligonucleotide, using EDC and imidazole to activate the 5′ phosphate group to a phosphorylimidazolide, and by subsequently reacting the resulting product with cystamine. This is followed by reduction with dithiothreitol (DTT) to form a phosphoramidite linkage to a free thiol group. A thiol group is, alternatively, added on an oligonucleotide by incorporating a phosphoramidite that contains a thiol during solid-phase phosphoramidite oligonucleotide synthesis, at either the 5′-end or the 3′-end of the oligonucleotide as shown in FIG. 24A-FIG. 24E. The phosphoramidite used during synthesis can have a protecting group on the thiol during synthesis that is removed during cleavage, purification, and workup. FIG. 24A illustrates structures of oligonucleotides containing a 5′-thiol (thiohexyl; C6) modification (left), and a 3′-thiol (C3) modification (right), as shown at https://www.atdbio.com/content/50/Thiol-modified-oligonucleotides.

An amine group is added on RNA or DNA by incorporating a phosphoramidite during synthesis that contains a protected amino group that is later deprotected. FIG. 24B illustrates an MMT-hexylaminolinker phosphoramidite. FIG. 24C illustrates a TFA-pentylaminolinker phosphoramidite, as shown at https://www.sigmaaldrich.com/catalog/product/sigma/m01023hh?lang=en&region=US.

Alternatively, thiol or amine containing oligonucleotide residues are included within the sequence at any chosen location in RNA or DNA, such as described by Jin et al. (J Org Chem. 2005 May 27; 70(11):4284-99). FIG. 24D illustrates RNA residues incorporating amine or thiol residues, as presented in Jin et al. (J Org Chem. 2005 May 27; 70(11):4284-99). Also, an oligonucleotide residue that contains a phosphorothioate group within the phosphodiester backbone (where a sulfur atom replaces a non-bridging oxygen in the phosphate backbone of the oligonucleotide) provides a reactive group that is similarly used for conjugation to a thiol group. Use of the phosphorothioate containing residues can also make the RNA more resistant to nuclease degradation.

FIG. 24E illustrates oligonucleotides with aminohexyl modifications at the 5′ (left) and 3′ ends (right).

Aldehyde functional groups can be incorporated at the 3′ end of RNA by using periodate oxidation to convert the diol into two aldehyde groups.

Other methods of incorporating or modifying functional groups are carried out using techniques set forth in Bioconjugate Techniques, by Greg Hermanson, 3rd edition.

Any peptide oligonucleotide complexes of the present disclosure may be modified with a thiol group, an amine group, or an aldehyde group and are described. Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 27

### Generation of Cleavable Linkers Between an Oligonucleotide with a Peptide

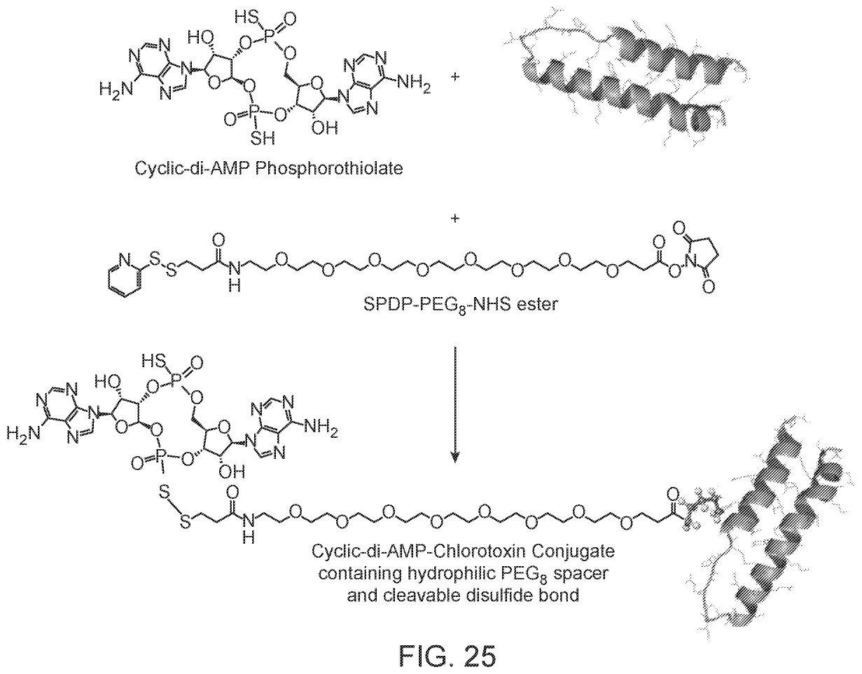

This example describes generation of cleavable linkers between an oligonucleotide with any one of peptides of SEQ ID NO: 1-SEQ ID NO: 118, SEQ ID NO: 435, SEQ ID NO: 436, or SEQ ID NO: 554-SEQ ID NO: 567. A disulfide linker is generated by combining a thiol-containing oligonucleotide with a peptide comprising a free thiol group. The thiol is incorporated on the peptide using Traut's reagent, SATA, SPDP or other appropriate reagents on a reactive amine (such as a heterobifunctional SPDP and NHS ester linker with the N-terminus or a lysine residue), or by incorporating a free cysteine residue in the peptide, as shown in FIG. 25. The disulfide linker is cleaved in the reducing environment of the cytoplasm or in the endosomal/lysosomal pathway.

An ester linkage is generated by combining a free hydroxyl group (such as on the 3′ end of an oligonucleotide) with a carboxylic acid group on the peptide (such as from the C-terminus, an aspartic acid, glutamic acid residue, or introduced via a linker to a lysine residue or the N-terminus) such as via Fisher esterification or via use of an acyl chloride. The ester linker is cleaved by hydrolysis, which is accelerated by the lower pH of endosomes and lysosomes, or by enzymatic esterase cleavage.

An oxime or hydrazone linkage is generated by combining an aldehyde group on the oligonucleotide with a peptide that has been functionalized with an aminooxy group (to form an oxime linkage) or a hydrazide group (to form a hydrazone linkage). The stability or lability of an oxime or hydrazone linkage is tailored by neighboring groups (Kalia et al., Angew Chem Int Ed Engl. 2008; 47(39):7523-6), and hydrolytic cleavage is accelerated in acidic compartments such as the endosome/lysosome.

A hydrazide group is incorporated on a peptide by reacting adipic acid dihydrazide or carbohydrazide with carboxylic acid groups in the C-terminus or in aspartic or glutamic acid residues. An aminooxy group is incorporated on a peptide by reacting the N-terminus or a lysine residue with a heterobifunctional molecule containing an NHS ester on one end and a phthalimidoxy group on the other end, followed by cleavage with hydrazine. The reaction is, optionally, catalyzed by addition of aniline.

The cleavage rate of any linker is tuned, for example, by modifying the electron density in the vicinity of the cleavable link or by sterically affecting access to the cleavage site (e.g., by adding bulky groups, such as methyl groups, ethyl groups, or cyclic groups).

Cleavable linkers are, alternatively, generated using methods set forth in Bioconjugate Techniques, by Greg Hermanson, 3rd edition.

Installation of a thiol, amine, or aldehyde groups in RNA or DNA, as a functional handle, is carried out as described above in EXAMPLE 26.

Any peptide oligonucleotide complexes of the present may contain a cleavable linker and are described. Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 28

### Generation of Stable Linkers Between an Oligonucleotide and a Peptide

This example describes generation of a stable linkers between RNA, DNA, or any oligonucleotide, with any one of peptides of SEQ ID NO: 1-SEQ ID NO: 118, SEQ ID NO: 435, SEQ ID NO: 436, or SEQ ID NO: 554-SEQ ID NO: 567. A stable linker through a secondary amine is generated by reductive amination, achieved by combining an aldehyde-containing oligonucleotide with the amine at the N-terminus of a peptide or in a lysine residue, followed by reduction with sodium cyanoborohydride.

A stable amide linkage is generated by combining an amine group on an oligonucleotide with the carboxylate at the C-terminus of a peptide or in an aspartic acid or glutamic acid residues.

A stable carbamate linkage is generated by activating a hydroxyl group in an oligonucleotide with carbonyldiimidazole (CDI) or N,N′-disuccinimidyl carbonate (DSC) and subsequently reacted with a peptide's N-terminus or lysine residue.

A maleimide linker is created by combining a thiol-containing oligonucleotide with a maleimide functionalized peptide. The peptide is functionalized using an NHS-X-maleimide heterobifunctional agent on a reactive amine in the peptide, wherein X is any linker. A maleimide linker is used as a stable linker or as a slowly cleavable linker, which is cleaved by exchange with other thiol-containing molecules in biological fluids. The maleimide linker is also stabilized by hydrolyzing the succinimide moiety of the linker using various substituents, including those described in Fontaine et al., Bioconjugate Chem., 2015, 26 (1), pp 145-152.

Other methods of incorporating, adding, or modifying functional groups in polynucleotides, for example, are carried out using techniques set forth in Bioconjugate Techniques, by Greg Hermanson, 3rd edition.

Installation of a thiol, amine, or aldehyde groups in an oligonucleotide, as a functional handle, is carried out as described above in EXAMPLE 26.

Any peptide oligonucleotide complexes of the present disclosure may contain a stable linker and are described. Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 29

### Generation of an Enzyme Cleavable Linkage Between an Oligonucleotide and a Peptide

This example describes generation of an enzyme cleavable linkage between RNA, DNA, or any oligonucleotide, and any one of peptides of SEQ ID NO: 1-SEQ ID NO: 118, SEQ ID NO: 435, SEQ ID NO: 436, or SEQ ID NO: 554-SEQ ID NO: 567. An enzymatically cleavable linkage is generated between an oligonucleotide and a peptide. The conjugate with a cleavable linkage is administered in vitro or in vivo and is cleaved by enzymes in the cells or body, releasing the oligonucleotide. The enzyme is present in the endosome/lysosome, the cytosol, the cell surface, or is upregulated in the tumor microenvironment or the tissue microenvironment. These enzymes include, but are not limited to, cathepsins (such as all those listed in Kramer et al., Trends Pharmacol Sci. 2017 October; 38(10):873-898) such as cathepsin B, glucoronidases including beta-glucuronidase, hyaluronidase and matrix metalloproteases, such as MMP-1, 2, 7, 9, 13, or 14 (Kessenbrock et al., Cell. 2010 Apr. 2; 141(1): 52-67). Cathepsin or MMPs cleave amino acid sequences of any one of SEQ ID NO: 200, SEQ TD NO: 204, or SEQ ID NO: 216-SEQ TD NO: 241 or SEQ TD NO: 433, shown below in TABLE 14 (see also Nagase, Hideaki. “Substrate specificity of MMPs.” Matrix Metalloproteinase Inhibitors in Cancer Therapy. Humana Press, 2001. 39-66; Dal Corso et al., Bioconjugate Chem., 2017, 28 (7), pp 1826-1833; Dal Corso et al., Chemistry—A European Journal 21.18 (2015): 6921-6929; Doronina et al., Bioconjug Chem. 2008 October; 19(10):1960-3). Glucuronidase linkers include any one of those described in Jeffrey et al., Bioconjugate Chem., 2006, 17 (3), pp 831-840.

TABLE 14
Enzymatically Cleavable Linkers

| | | May be |
|---|---|---|
| SEQ ID NO | Sequence | Cleaved By |
| SEQ ID NO: 200 | Val-Ala | Cathepsin |
| SEQ ID NO: 204 | Val-Lys | Cathepsin |
| SEQ ID NO: 216 | Val-Arg | Cathepsin |
| SEQ ID NO: 217 | Val-Cit | Cathepsin |
| SEQ ID NO: 218 | Phe-Lys | Cathepsin |
| SEQ ID NO: 219 | Met-Lys | Cathepsin |
| SEQ ID NO: 220 | Asn-Lys | Cathepsin |
| SEQ ID NO: 221 | Ile-Pro | Cathepsin |
| SEQ ID NO: 222 | Gly-Ile | MMP |
| SEQ ID NO: 223 | Gly-Leu | MMP |
| SEQ ID NO: 224 | Gly-Tyr | MMP |
| SEQ ID NO: 225 | Gly-Met | MMP |
| SEQ ID NO: 226 | Met-Ile | MMP |
| SEQ ID NO: 227 | Ala-Ile | MMP |
| SEQ ID NO: 228 | Pro-Ile | MMP |
| SEQ ID NO: 229 | Gly-Pro-Gln-Gly- | MMP |
| | Ile-Ala-Gly-Gln | |
| SEQ ID NO: 230 | Gly-Pro-Gln-Gly- | MMP |
| | Ile-Phe-Gly-Gln | |
| SEQ ID NO: 231 | Gly-Pro-Gln-Gly- | MMP |
| | Ile-Trp-Gly-Gln | |
| SEQ ID NO: 232 | Gly-Pro-Gln-Gly- | MMP |
| | Ile-Leu-Gly-Gln | |
| SEQ ID NO: 233 | Gly-Pro-Gln-Gly- | MMP |
| | Ile-Arg-Gly-Gln | |
| SEQ ID NO: 234 | Gly-Pro-Leu-Gly- | MMP |
| | Ile-Ala-Gly-Gln | |
| SEQ ID NO: 235 | Gly-Pro-Met-Gly- | MMP |
| | Ile-Ala-Gly-Gln | |
| SEQ ID NO: 236 | Gly-Pro-Tyr-Gly- | MMP |
| | Ile-Ala-Gly-Gln | |
| SEQ ID NO: 237 | GSVAGS | Cathepsin |
| SEQ ID NO: 238 | GGGGSVAGGGGS | Cathepsin |
| SEQ ID NO: 239 | GGGGSGGGGSVAGGGG | Cathepsin |
| | SGGGGS | |
| SEQ ID NO: 240 | GGGGSGGGGSPLGLAG | |
| | GGGGSGGGGS | |
| SEQ ID NO: 241 | AEAAAKEAAAKAVAAE | |
| | AAAKEAAAKA | |

A Val-Cit-PABC enzymatically cleavable linker, such as described in Jain et al., Pharm Res. 2015 November; 32(11):3526-40, is created by conjugating the PABC end to an amine group on the oligonucleotide. The valine end is further linked to the peptide, for example, by generating an amide bond to the C-terminus of the peptide. A spacer on either side of the molecule is optionally incorporated in order to facilitate steric access of the enzyme to the Val-Cit linkage (SEQ ID NO: 217). The linkage to the peptide is, alternatively, generated by activating the N-terminus of the peptide with SATA and creating a thiol group, which is subsequently reacted to a maleimidocaproyl group attached to the N-terminus of the Val-Cit pair (SEQ ID NO: 217). Upon cleavage by cathepsin B, the self-immolative PABC group spontaneously eliminates, releasing the amine-containing oligonucleotide with no further chemical modifications. Other amino acid pairs include Glu-Glu, Glu-Gly, and Gly-Phe-Leu-Gly (SEQ ID NO: 551).

Installation of a thiol, amine, or aldehyde group in RNA or DNA, as a functional handle, is carried out as described above in EXAMPLE 26.

Any peptide oligonucleotide complexes of the present disclosure may contain an enzyme cleavable linker and are described. Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 30

### Conjugation of an Oligonucleotide and a Peptide

This example describes conjugation of an oligonucleotide to a peptide of the present disclosure. The peptide is SEQ ID NO: 1. The N-terminus of SEQ ID NO: 1 is conjugated via reductive amination to 4-formyl-PBA. The PBA-containing peptide is complexed to the 3′ diol group of the oligonucleotide to form a boronate ester.

Alternatively, the oligonucleotide has a thiol-containing or phosphorothioate-containing nucleotide residue included in the sequence, during synthesis. The N-terminus of SEQ ID NO: 1 is modified with SATA (with subsequent deprotection using hydroxylamine) to form a thiol group.

Alternatively, The N-terminus of SEQ ID NO: 1) is modified with SPDP-PEG₄-NHS ester to form a protected thiol group, with a flexible hydrophilic PEG spacer. The two thiol groups in the modified oligonucleotide and SEQ ID NO: 1 are combined to form a cleavable disulfide bond. Alternatively, The N-terminus of SEQ ID NO: 1 is modified with bromoacetamido-PEG₄-TFP ester to form an amide bond, and then reacted with the thiol group within the oligonucleotide, to form a stable thioether bond.

Alternatively, the oligonucleotide has an amine-containing nucleotide included in the sequence, during synthesis. The N-terminus of SEQ ID NO: 1 is modified with SATA to form a thiol group. A maleimidocaproyl-Val-Cit-PABC linker is conjugated to the amine in the oligonucleotide and to the thiol in SEQ ID NO: 1.

Alternatively, the oligonucleotide is conjugated to the N-terminus of SEQ ID NO: 1 via reductive amination after oxidation of the 3′ diols to form a secondary amine conjugate.

Alternatively, the oligonucleotide has the 3′ end oxidized to aldehydes via periodate oxidation. The aldehyde is then reacted with the peptide of SEQ ID NO: 1, which is functionalized with an aminooxy group on the N-terminus to form a cleavable oxime bond.

Alternatively, a dsRNA is used. The 3′ end of the sense strand is synthesized with a thiol modification as shown in FIG. 24A-FIG. 24E. The N-terminus of SEQ ID NO: 1 is modified with bromoacetamido-PEG₄-TFP ester to form an amide bond, and then reacted with the thiol group within the dsRNA to form a stable thioether bond. Alternatively, the 5′ end of the sense strand or amino terminated nucleotides serves as the site of modification.

Alternatively, the peptide is any one of SEQ ID NO: 1-SEQ ID NO: 118, SEQ ID NO: 435, SEQ ID NO: 436, or SEQ ID NO: 554-SEQ ID NO: 567.

Alternatively, rather than using the N-terminus of the peptide, a lysine residue in the peptide is used. Optionally, one or more or all of the lysine residues are mutated to arginine residues so only one, or no, lysine residues are available for amine-based reactions.

Installation of a thiol, amine, or aldehyde groups in RNA or DNA, as a functional handle, is carried out as described above in EXAMPLE 26.

Optionally, the oligonucleotide is synthesized using any one or more modified bases in order to alter the stability, tissue biodistribution, immune reactivity, or any other property of the oligonucleotide.

Any peptide oligonucleotide complexes of the present disclosure (e.g., including an oligonucleotide of any one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17.

### Example 31

### Surface Plasmon Resonance (SPR) Analysis of Peptide Binding Interactions

This example illustrates surface plasmon resonance (SPR) analysis of peptide-oligonucleotide conjugate binding interactions with PD-L1.

A peptide-oligonucleotide conjugate (also referred to herein as a peptide oligonucleotide complex) is constructed by combining any of the PD-L1-binding peptides of this disclosure with an oligonucleotide. Optionally, the oligonucleotide is designed for RNase H-engaging antisense, splice-blocking antisense, siRNA, anti-miR, U1 adapter, or aptamer therapy. Optionally, a stable or cleavable linker is used between the peptide and the oligonucleotide.

The peptide-oligonucleotide conjugate is subjected to SPR (surface plasmon resonance) analysis. The affinity of the peptide-oligonucleotide to PD-L1 is measured by SPR, using either a PD-L1 moiety or the peptide-oligonucleotide conjugate as the analyte. Optionally the PD-L1 is human, murine, rat, canine, or non-human primate (e.g. cynomolgus or rhesus). The kₒₙ, k_{off}, and/or K_{D} of the peptide-oligonucleotide conjugate for PD-L1 is measured. Optionally the kₒₙ, k_{off}, and K_{D} of the peptide alone (not conjugated to the oligonucleotide) to PD-L1 is also measured. The K_{D} of the peptide-oligonucleotide conjugate to PD-L1 is found to be adequate to bind to the desired target cell and drive endocytic uptake or transcytosis of the peptide-oligonucleotide conjugate. The K_{D} of the peptide-oligonucleotide conjugate to the PD-L1 is optionally found to be less than 1 μM, less than 100 nM, less than 10 nM, less than 1 nM, or less than 0.5 nM. Optionally, the K_{D} of the peptide-oligonucleotide conjugate to the PD-L1 is found to be similar to the K_{D} of the peptide alone, such as within 100-fold, 10-fold, 5-fold, or 2-fold of each other. Optionally, the k_{off} of the peptide-oligonucleotide conjugate is found to be sufficient to allow peptide-oligonucleotide conjugate uptake into the endosome and release from PD-L1 prior to recycling or release from PD-L1 after transcytosis. In some cases, an increased PD-L1-binding affinity can correspond to a reduced transcytosis function, wherein in some cases, an increased PD-L1-binding affinity does not correspond to a change in transcytosis function compared to the reference peptide. It is assumed that the ratio of kₒₙ/k_{off} can affect the transcytosis function of a peptide, and thus modulation of kₒₙ and/or k_{off} can be used to generate PD-L1-binding peptides with optimal PD-L1 binding affinity and transcytosis function. Optionally, the linker between the peptide and the oligonucleotide and/or the oligonucleotide or peptide sequence are changed such that the kₒₙ, k_{off}, and/or K_{D} of the modified peptide-oligonucleotide conjugate to PD-L1 is closer to the desired values. Optionally, different peptide-oligonucleotide conjugates are compared, and the peptide-oligonucleotide conjugate with the most desirable kₒₙ, k_{off}, and/or K_{D} is selected for further use.

### Example 32

### Extension of Peptide Oligonucleotide Complex Plasma Half-Life

This example demonstrates a method of extending the serum or plasma half-life of a peptide as disclosed herein. A peptide oligonucleotide complex having a peptide sequence of any one of SEQ ID NO: 1-SEQ ID NO: 118, SEQ ID NO: 435, SEQ ID NO: 436, or SEQ ID NO: 554-SEQ ID NO: 567 is modified (such as modified in the peptide, the oligonucleotide, the linker, or either end) in order to increase its plasma half-life. Conjugation of the peptide oligonucleotide complex to a near infrared dye, such as Cy5.5 is used to extend serum half-life of the peptide construct. Alternatively, conjugation of the peptide oligonucleotide complex to an albumin binder, such as Albu-tag or a C₁₄-C₁₈ fatty acid, is used to extend plasma half-life. Optionally, plasma half-life is extended as a result of reduced immunogenicity by using minimal non-human protein sequences.

### Example 33

### Cell-Penetrating Peptide Oligonucleotide Complex Fusions

This example describes peptide fusions with additional cell penetrating peptides. A PD-L1-binding peptide oligonucleotide complex of the present disclosure is chemically conjugated or recombinantly expressed as a fusion to an additional cell penetrating peptide moiety. Any peptide oligonucleotide complexes of the present disclosure (e.g., including an oligonucleotide sequence of one of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17. The additional cell penetrating peptide moiety is one or multiple Arg residues, such as an RRRRRRRR (SEQ ID NO: 251) sequence conjugated to, linked to, or fused at the N-terminus or C-terminus, or a Tat peptide with the sequence YGRKKRRQRRR (SEQ ID NO: 276) that is conjugated to, linked to, or fused to the N-terminus or C-terminus of any PD-L1-binding peptide of the present disclosure. Alternatively, the additional cell penetrating peptide moiety is selected from maurocaline, imperatoxin, hadrucalcin, hemicalcin, oplicalin-1, opicalcin-2, midkine (62-104), MCoTI-II, or chlorotoxin, which is fused to the N-terminus or C-terminus of any PD-L1-binding peptide of the present disclosure. Alternatively, the additional cell penetrating peptide moiety is selected from TAT such as CysTAT (SEQ ID NO: 249), S19-TAT (SEQ ID NO: 250), R8 (SEQ ID NO: 251), pAntp (SEQ ID NO: 252), Pas-TAT (SEQ ID NO: 253), Pas-R8 (SEQ ID NO: 254), Pas-FHV (SEQ ID NO: 255), Pas-pAntP (SEQ ID NO: 256), F2R4 (SEQ ID NO: 257), B55 (SEQ ID NO: 258), azurin (SEQ ID NO: 259), IMT-P8 (SEQ ID NO: 260), BR2 (SEQ ID NO: 261), OMOTAG1 (SEQ ID NO: 262), OMOTAG2 (SEQ ID NO: 263), pVEC (SEQ ID NO: 264), SynB3 (SEQ ID NO: 265), DPV1047 (SEQ ID NO: 266), C105Y (SEQ ID NO: 267), transportan (SEQ ID NO: 268), MTS (SEQ ID NO: 269), hLF (SEQ ID NO: 270), PFVYLI (SEQ ID NO: 271), or yBBR (SEQ ID NO: 272), which is fused to the N-terminus or C-terminus of any PD-L1-binding peptide of the present disclosure. Alternatively, the additional cell penetrating peptide moiety is fused to the N-terminus or C-terminus of any PD-L1-binding peptide of the present disclosure by a linker. The linker is selected from GGGSGGGSGGGS (SEQ ID NO: 163), KKYKPYVPVTTN (SEQ ID NO: 166) (linker from DkTx), or EPKSSDKTHT (SEQ ID NO: 167) (linker from human IgG3), or any other linker. Alternatively, the PD-L1-binding peptide, the additional cell penetrating peptide moiety, and, optionally, the linker are joined by other means. For example, the other means includes, but is not limited to, chemical conjugation at any location, fusion of the additional cell penetrating peptide moiety and/or the linker to the C-terminus of the PD-L1-binding peptide, co-formulation with liposomes, or other methods.

Cell-penetrating peptide fusions or conjugates are administered to a subject in need thereof. The subject is a human or animal and has a disease, such as a brain cancer or other brain condition. Upon administration, the additional cell-penetrating peptides promote crossing the cellular membranes to access intracellular compartments. Alternatively, upon administration, the PD-L1-binding peptides promote endocytosis into cells expressing PD-L1 and the PD-L1-binding peptides and/or the additional cell penetrating peptides promote release of the oligonucleotide into the cytoplasm or other subcellular compartments.

### Example 34

### Peptide Oligonucleotide Complexes to Promote Nuclear Localization

This example describes peptide complexes to promote nuclear localization. The peptide and the oligonucleotide of a PD-L1-binding peptide oligonucleotide complex of the present disclosure are recombinantly expressed or chemically synthesized and then conjugated together with a linker. The peptide within the peptide oligonucleotide complex is selected from any sequence of SEQ ID NO: 1-SEQ ID NO: 118, SEQ ID NO: 435, SEQ ID NO: 436, or SEQ ID NO: 554-SEQ ID NO: 567. Any peptide oligonucleotide complexes of the present disclosure (e.g., including an oligonucleotide sequence of any of SEQ ID NO: 366-SEQ ID NO: 396 or SEQ ID NO: 492-SEQ ID NO: 545, linked or conjugated to SEQ ID NO: 1-SEQ ID NO: 118, SEQ ID NO: 435, SEQ ID NO: 436, or SEQ ID NO: 554-SEQ ID NO: 567) are described. Any peptide oligonucleotide complexes of the present disclosure can have oligonucleotides complementary to any target in TABLE 10, or to any of SEQ ID NO: 397-SEQ ID NO: 430 or SEQ ID NO: 546-SEQ ID NO: 549, or to any genomic or ORF sequence provided in TABLE 17. The peptide oligonucleotide complexes are conjugated to, linked to, or fused to a nuclear localization signal, such as a four-residue sequence of K-K/R-X-K/R (SEQ ID NO: 434), wherein X can be any amino acid, or a variant thereof (Lange et al, J Biol Chem. 2007 Feb. 23; 282(8):5101-5). The complexes are administered to a subject in need thereof. The subject is a human or animal and has a disease, such as cancer. Upon administration, PD-L1-binding peptides promote uptake by a PD-L1 expressing cell and the nuclear localization signal promotes trafficking to the nucleus and or the PD-L1-binding peptides promote endocytosis into cells expressing PD-L1.

### Example 35

### PD-L1-Binding Peptide Oligonucleotide Complex for pH-Dependent Endosomal Delivery

This example describes development and in vitro testing of PD-L1-binding peptide oligonucleotide complexes capable of pH-dependent dissociation from PD-L1, for example, at endosomal pH (e.g., pH 5.5).

One or more additional histidine residues are introduced into the sequence of PD-L1-binding peptides within the peptide oligonucleotide complex (e.g., any one of SEQ ID NO: 1-SEQ ID NO: 118, SEQ ID NO: 435, SEQ ID NO: 436, or SEQ ID NO: 554-SEQ ID NO: 567) complexed with an oligonucleotide. The resulting histidine-enriched PD-L1-binding peptide oligonucleotide complexes are evaluated for their PD-L1 binding in comparative binding experiments at various pH levels or ranges. Peptides with high PD-L1 binding affinity at physiological pH but a significantly reduced binding affinity at lower pH levels such as endosomal pH of 5.5 are selected for cellular binding, uptake, and intra-endosomal or intra-vesicular release experiments.

PD-L1-binding peptide oligonucleotide complexes with high endosomal delivery capabilities are identified and characterized. These results demonstrate that the PD-L1-binding peptide oligonucleotide complexes of the present disclosure can exhibit, or can be modified to exhibit pH-dependent PD-L1 binding kinetics that allows intra-vesicular release of PD-L1-binding peptide oligonucleotide complexes and PD-L1-binding peptide oligonucleotide complex comprising one or more active agents for endosomal and/or intracellular delivery. Higher levels of the peptide oligonucleotide complex may be delivered to or accumulate in the endosome due to dissociation from PD-L1 prior to PD-L1 recycling back to the cell surface.

In order to improve the intracellular delivery functions, the PD-L1-binding peptide oligonucleotide complexes as described herein are optionally modified to comprise a motif that facilitates low-pH endosomal release or escape of the peptide oligonucleotide complex or are constructed with a cleavable linker.

Cellular uptake and release experiments demonstrate that the PD-L1-binding peptide oligonucleotide complexes that comprise a motif for low-pH endosomal escape show are present in the cytosol at higher concentrations compared to peptides that do not comprise the motif for low-pH endosomal escape. This data demonstrates that the PD-L1-binding oligonucleotide complexes of the present disclosure can be successfully modified for enhanced intra-vesicular and intra-cellular delivery, including to subcellular compartments, while retaining their PD-L1 binding capabilities. These peptide oligonucleotide complexes can optionally be used in combination with various therapeutic and/or compounds for treatment and/or diagnosis of diseases and conditions.

### Example 36

### Comparison of Dose Toxicity of a PD-L1-Binding Peptide Oligonucleotide Complex to a PD-L1-Binding Antibody Oligonucleotide Complex

This example describes the comparison of the dose toxicity of a PD-L1-binding peptide oligonucleotide complex of this disclosure to anti-PD-L1 antibody oligonucleotide complex when administered to a murine subject. Optionally, the oligonucleotide targeting agent targets a gene that encodes for BACE. An anti-PD-L1 antibody oligonucleotide complex is administered to a subject at doses of 5 mg/kg, 25 mg/kg or 50 mg/kg, corresponding to molar doses per 25 g mouse mass of about 0.84 nmol, 4.2 nmol, and 8.4 nmol, respectively, as described in Couch, et al, 2013 (Couch et al, Sci Transl Med. 2013 May 1; 5(183):183ra57). A PD-L1-binding peptide oligonucleotide complex of this disclosure is administered to a subject at doses of about 31 mg/kg, corresponding to a molar concentration of about 100 nmol per 25 g mouse mass. Alternatively, a PD-L1-binding peptide oligonucleotide complex of this disclosure is administered to a subject at doses of 0.84 nmol, 4.2 nmol and 8.4 nmol or 100 nmol. Subjects receiving 31 mg/kg, or about 100 nmol per 25 g mouse mass, of the PD-L1-binding peptide oligonucleotide complex show effective pharmacodynamic and pharmacokinetic properties without signs of distress or toxicity over the course of at least 24 hours. Meanwhile subjects receiving 5 mg/kg, or about 0.84 nmol per 25 g mouse mass, of the anti-PD-L1 antibody oligonucleotide complex show reduced therapeutic efficacy, such as reduced pharmacodynamic amyloid beta inhibition, as compared to the subjects receiving 25 or 50 mg/kg, or 4.2 or 0.4 nmol per 25 kg mouse mass. The 25 or 50 mg/kg, or 4.2 or 0.4 nmol per 25 kg mouse mass, doses of the anti-PD-L1 antibody induce lethargy, distress, and hemolysis, or reduced reticulocyte count or other toxicities, within at least 30 minutes of administration. The results demonstrate that the therapeutic window (the dosage above which a therapeutic pharmacodynamic response is seen but below which toxicity is observed) is wider for peptide-based therapeutics than for antibody-based therapeutics. The results further demonstrate that PD-L1-binding peptide oligonucleotide complex-based therapeutics show less off-target binding and lower immune response as compared to PD-L1-binding antibody-based therapeutics, due to the smaller protein lengths (approximately 50 amino acids) providing fewer epitopes for an adaptive immune response and smaller surface area.

### Example 37

### PD-L1-Binding Peptide Oligonucleotide Complex Using siRNA for Treatment of Type 2 Diabetes

This example describes treatment of type 2 diabetes using a PD-L1-binding peptide nucleotide complex described herein. The transcription factor FoxO1 is required for beta cell identity and optimal insulin production. Its inhibition (via acetylation) by transcription cofactors p300 and/or cyclic AMP response element-binding (CREB) protein (CBP) results in de-differentiation of beta cells and reduction of insulin production capacity. While whole-body disruption of p300 or CBP would be harmful as they are not specific to pancreatic beta cells, targeting a p300 or CBP antisense construct to pancreatic beta cells via conjugation to a PD-L1-binding peptide could ameliorate Type 2 diabetes symptoms or progression. This example demonstrates this approach for targeting CBP, but an equivalent strategy targeting p300 is viable. The nucleic acid portion of the peptide oligonucleotide complex comprises siRNA targeting the CBP transcript. Short sequences in the CBP mRNA are identified (e.g., 21 nt sequences in the CBP mRNA), beginning with AA and ending in TT (or UU in RNA) that are between 30-60% G/C in content and complementary sequence to the CBP mRNA used in the complex. For example, any 21 mer complementary across the CBP mRNA that has imperfect complementarity (e.g., no more than 85% complementarity, or having at least 3 to 4 mismatches) or no to low complementarity (e.g., no more than 75%, 65%, 50%, or 30% complementarity) relative to other sequences in transcriptome (to reduce off target effects) may be used, with an optimal length that fits into RISC complex (e.g., a 21 mer+/−up to 5 nt).

The siRNA may bind a target molecule of any one of SEQ ID NO: 546-SEQ ID NO: 549. Duplex structures (e.g., dsRNA) for modulating CBP mRNA can include: SEQ ID NO: 532-SEQ ID NO: 539, provided in TABLE 13 describes exemplary CBP siRNAs which are four siRNA pairs. It is understood to that within each pair of complimentary sequences described (e.g., SEQ ID NO: 532 and SEQ ID NO: 533, SEQ ID NO: 534 and SEQ ID NO: 535, etc.) are together part of the same complex and are partial reverse complements to one another.

TABLE 13
Examples of CBP siRNAs

| SEQ ID NO | Sequence |
|---|---|
| SEQ ID NO: 532 | 3′-AUCAUUGAGACCGGUAUCGAA-5′ |
| SEQ ID NO: 533 | 5′-AAUAGUAACUCUGGCCAUAGC-3′ |
| SEQ ID NO: 534 | 3′-GUCGUCUACUUCGUCGUCUAA-5′ |
| SEQ ID NO: 535 | 5′-AACAGCAGAUGAAGCAGCAGA-3′ |
| SEQ ID NO: 536 | 3′-UUAGGUCCGUAGAUCCAAGAA-5′ |
| SEQ ID NO: 537 | 5′-AAAAUCCAGGCAUCUAGGUUC-3′ |
| SEQ ID NO: 538 | 3′-ACAAUGAUCUCUUCUUCGGAA-5′ |
| SEQ ID NO: 539 | 5′-AAUGUUACUAGAGAAGAAGCC-3′ |

Flanking ˜2-3 nucleotides are joined by phosphodiester (PO) or phosphorothioate (PS) linkages. All other backbones are PO linkages. Sugar chemistries are RNA, either regular (—OH) or 2′ modified (such as 2′-O-Me, 2′-F).

The PD-L1-binding peptide and the oligonucleotide of the peptide oligonucleotide complex are each expressed recombinantly or chemically synthesized and then conjugated together with a linker. Optionally, the linker is cleavable. Optionally, the PD-L1-binding peptide has reduced affinity for PD-L1 at pH lower than 7.4. The nucleic acid portion of the peptide oligonucleotide complex is, targeted against any portion of the CBP mRNA (e.g., NCBI Refseq ID NM_001079846.1 CREBBP [organism=Homo sapiens] [GeneID=1387], SEQ ID NO: 546), or a functional fragment thereof. Similarly, the siRNA may bind a target molecule of SEQ ID NO: 548, or a functional fragment thereof. The PD-L1-binding peptide oligonucleotide complex is administered to a subject in need thereof. The PD-L1-binding peptide oligonucleotide complex is administered intravenously, subcutaneously, intramuscularly, by suppository, or orally. The subject is a human or an animal. After administration, the PD-L1-binding peptide oligonucleotide complex accumulates in pancreatic beta cells and the CBP mRNA is degraded. The PD-L1-binding peptide nucleotide complex ameliorates the type 2 diabetes, and reduced symptoms of type 2 diabetes are exhibited. In patients these symptoms may include increased thirst, frequent urination, increased hunger, unintended weight loss, fatigue, blurred vision, slow-healing sores, frequent infections, numbness or tingling in the hands or feet, diabetic retinopathy, kidney disease (nephropathy), diabetic neuropathy, and macrovascular problems.

This data demonstrates that the PD-L1-binding peptide oligonucleotide complexes of the present disclosure effectively treat type 2 diabetes.

### Example 38

### PD-L1-Binding Peptide Oligonucleotide Complex Using a Gapmer for Treatment of Type 2 Diabetes

This example describes treatment of type 2 diabetes disease using a PD-L1-binding peptide nucleotide complex described herein. The transcription factor FoxO1 is required for beta cell identity and optimal insulin production. Its inhibition (via acetylation) by transcription cofactors p300 and/or CBP results in de-differentiation of beta cells and reduction of insulin production capacity. While whole-body disruption of p300 or CBP would be harmful as they are not specific to pancreatic beta cells, targeting a p300 or CBP antisense construct to pancreatic beta cells via conjugation to a PD-L1-binding peptide could ameliorate Type 2 diabetes symptoms or progression. This example will demonstrate this approach for targeting p300, but an equivalent strategy targeting CBP may be used. The nucleic acid portion of the peptide oligonucleotide complex comprises a gapmer targeting the p300 gene. Short sequences in the p300 mRNA are identified (e.g., 20 nt sequences in the p300 mRNA), that are greater than 40% G/C in content and complementary sequence to p300 mRNA used in the complex. For example, any 20 mer complementary to the p300 mRNA that has imperfect complementarity (e.g., no more than 85% complementarity, or having at least 3 to 4 mismatches) or no to low complementarity (e.g., no more than 75%, 65%, 50%, or 30% complementarity) relative to other sequences in transcriptome (to reduce off target effects) may be used (e.g., a 20 mer only found in the p300 gene).

Single stranded structures (e.g., ssRNA or ssDNA) for modulating p300 mRNA can include any one of SEQ ID NO: 512-SEQ ID NO: 531, provided in TABLE 5.

Any of SEQ ID NO: 512-SEQ ID NO: 531 may be synthesized as the corresponding RNA sequence, with U substituted for T. The gapmer may bind a target molecule of any one of the p300 transcript sequences derived from its open reading frame (NCBI Refseq ID NG_009817.1), which could include sequences found in its mature transcripts including NCBI Refseq IDs NM_001429.4 Homo sapiens E1A binding protein p300 (EP300), transcript variant 1, mRNA or NM_001362843.2 Homo sapiens E1A binding protein p300 (EP300), transcript variant 2, mRNA, SEQ ID NO: 548, or SEQ ID NO: 549.

For this example, one could construct ASOs with full backbone PS linkages, where all C bases are 5-methyl-C. For this example, the middle 10 nt are DNA sugars and the flanking 5 nt on each side are 2′O-MOE RNA sugars.

The peptide and the oligonucleotide of the PD-L1-binding peptide oligonucleotide complex of the disclosure are each expressed recombinantly or chemically synthesized and then conjugated together via a linker. Optionally the linker is cleavable. Optionally, the PD-L1 binding peptide has reduced affinity for PD-L1 at pH lower than 7.4. The nucleic acid portion of the peptide oligonucleotide complex is, targeted against any portion of the p300 pre-mRNA sequence derived from its open reading frame (NCBI Refseq NG_009817.1), or a functional fragment thereof including its mature transcripts such as NCBI Refseq IDs NM_001429.4 Homo sapiens E1A binding protein p300 (EP300), transcript variant 1, mRNA or NM_001362843.2 Homo sapiens E1A binding protein p300 (EP300), transcript variant 2, mRNA. The PD-L1-binding peptide oligonucleotide complex is administered to a subject in need thereof. The PD-L1-binding peptide nucleotide complex is administered direct intracranial, intravenously, subcutaneously, intramuscularly, orally, or intrathecally. The subject is a human or an animal. After administration, the PD-L1-binding peptide oligonucleotide complex accumulates in pancreatic beta cells and the CBP mRNA is degraded. The PD-L1-binding peptide nucleotide complex ameliorates the type 2 diabetes: Reduced symptoms of type 2 diabetes are exhibited. In patients these symptoms may include increased thirst, frequent urination, increased hunger, unintended weight loss, fatigue, blurred vision, slow-healing sores, frequent infections, numbness or tingling in the hands or feet, diabetic retinopathy, kidney disease (nephropathy), diabetic neuropathy, and macrovascular problems.

This data demonstrates that the PD-L1-binding peptide oligonucleotide complexes of the present disclosure effectively treat type 2 diabetes.

### Example 39

### PD-L1-Binding Peptide Oligonucleotide Complex Using an Anti-miR for Treatment of Solid Tumor

This example describes treatment of Cancers (e.g., glioblastoma multiforme (GBM), pancreatic cancer, breast cancer, colon cancer, lung cancer, head and neck cancer) using a PD-L1-binding peptide nucleotide complex described herein. Healthy tissues can express tumor suppressor genes such as PDCD4 and PTEN which control cell growth and apoptosis. The miRNA, miR-21 is a repressor of several such tumor suppressor genes, including PDCD4 and PTEN. The reduction of miR-21 hence can have utility in cancers (e.g., GBM, pancreatic cancer, breast cancer, colon cancer, lung cancer, or head and neck cancer) by restoring proper expression of tumor suppressor genes and enabling tumor suppression systems to work. The nucleic acid portion of the peptide nucleotide complex comprises an anti-miR targeting the miR-21 (i.e., anti-miR-21).

Mature miRNA guide strand of miR-21 is as follows: 5′-UAGCUUAUCAGACUGAUGUUGA-3′ (SEQ ID NO: 397). The anti-miR nucleotide may bind a target molecule of SEQ ID NO: 397. Base pairing to an anti-miR sequence would be as follows to generate a complementary anti-MIR-21 nucleic acid:

TABLE 15
Example of MIR-21 miRNA and Anti-miR Base Pairing

| SEQ ID NO: | Sequence | Name |
|---|---|---|
| SEQ ID NO: 397 | 3′-AGUUGUAGUCA | miRNA |
| | GACUAUUCGAU-5′ | |
| SEQ ID NO: 374 | 5′-UCAACAUCAGU | Full length |
| | CUGAUAAGCUA-3′ | anti-miR |

The optimal anti-miRNA must match at the seed region, typically sites 2-7 from the miRNA's 5′ end. Hence, truncations to test (to minimize length while maintaining potency) will truncate from the 5′ end of the anti-miR to maintain the 3′ end matching to the miRNA seed sequence:

TABLE 16
Examples of Anti-miR Truncations

| SEQ ID NO: | Sequence | Length |
|---|---|---|
| SEQ ID NO: 374 | UCAACAUCAGUCUGAUAAGC | 22 nucleotides |
| | UA | |
| SEQ ID NO: 375 | CAACAUCAGUCUGAUAAGCU | 21 nucleotides |
| | A | |
| SEQ ID NO: 376 | AACAUCAGUCUGAUAAGCUA | 20 nucleotides |
| SEQ ID NO: 377 | ACAUCAGUCUGAUAAGCUA | 19 nucleotides |
| SEQ ID NO: 378 | CAUCAGUCUGAUAAGCUA | 18 nucleotides |
| SEQ ID NO: 379 | AUCAGUCUGAUAAGCUA | 17 nucleotides |
| SEQ ID NO: 380 | UCAGUCUGAUAAGCUA | 16 nucleotides |
| SEQ ID NO: 381 | CAGUCUGAUAAGCUA | 15 nucleotides |

For such an exemplary anti-miR strategy, PO or PS backbone linkages are used; optionally 1-3 terminal linkages are PS. Sugars can be a mixture of DNA, 2′-O-Me, 2′-F, and/or LNA. C bases can be 5-methyl-C.

The peptide and the oligonucleotide of the PD-L1-binding peptide oligonucleotide complex of the disclosure are expressed recombinantly or chemically synthesized and the conjugated together via a linker. Optionally the linker is cleavable. Optionally the peptide has reduced affinity for PD-L1 at pH less than 7.4. The nucleic acid portion of the peptide oligonucleotide complex is, targeted against any portion of the miR-21 guide strand RNA (SEQ ID NO: 397), or a functional fragment thereof. The PD-L1-binding peptide oligonucleotide complex is administered to a subject in need thereof. The PD-L1-binding peptide oligonucleotide complex is administered intravenously, subcutaneously, intramuscularly, orally, intrathecally, intravitreally, or intratumorally. The subject is a human or an animal. Mouse models can include any of a number of xenografts of human tumor lines or primary tumor cells or other relevant cancer models. After administration, the PD-L1-binding peptide nucleotide complex accumulates diseased tissue and the miR-21 mRNA is degraded. The PD-L1-binding peptide nucleotide complex causes tumors or cancer cells (e.g., GBM, pancreatic cancer, breast cancer, colon cancer, lung cancer, or head and neck cancer) to grow more slowly, stop growing, or die. Reduced symptoms of cancers (e.g., GBM pancreatic cancer, breast cancer, colon cancer, lung cancer, head and neck cancer) may result. In patients: Reduced symptoms of cancer are exhibited and, reduction of tumor masses and prevention of re-growth (disease control).

This data demonstrates that the PD-L1-binding peptide oligonucleotide complexes of the present disclosure effectively treat Cancers (e.g., GBM, pancreatic cancer, breast cancer, colon cancer, lung cancer, or head and neck cancer).

### Example 40

### PD-L1-Binding Peptide Oligonucleotide Complex Using an Aptamer for Treatment of SARS-CoV-2

This example describes treatment of SARS-CoV-2 using a PD-L1-binding peptide oligonucleotide complex described herein. SARS-CoV-2 uses ACE2 as a major receptor for infection. With the PD-L1-binder's lung tissue accumulation potential based on basal PD-L1 expression in the lung, bringing an ACE2-inhibiting aptamer to the tissue could reduce SARS-CoV-2 proliferation and can therefore have utility in preventing and treating COVID-19. The nucleic acid portion of the peptide oligonucleotide complex comprises an aptamer targeting the ACE2 protein, optionally determined using a SELEX-based screening strategy. ACE2 is membrane-embedded, so soluble ACE2 could be a difficult reagent against which to screen. However, cells or membrane vesicles from cells over-expressing ACE2 could be exposed to a library of 20-40mer sequences of a random nature flanked by a primer-binding site. Cells or vesicles would be rinsed thoroughly and then lysed to release nucleic acids that are bound, which would be amplified by PCR. Negative selection would occur in ACE2-negative material to remove sequences non-specific to ACE2. After several rounds of positive and negative selection and amplification, individual sequences would be synthesized and tested for the ability to bind only to ACE2-expressing cells.

For such a ACE2-targeting aptamer, backbone linkages can be PO or PS; one clinical example of an aptamer, pegaptanib, uses all PO linkages. Sugars could be a mixture of DNA, RNA, 2′-O-Me, 2′-O-MOE, 2′-F, or LNA among others. Optionally, the bases are chemically modified to facilitate tighter binding or even covalent binding.

The peptide and the oligonucleotide of the PD-L1-binding peptide oligonucleotide complex of the disclosure are expressed recombinantly or chemically synthesized and then conjugated together via a linker. The nucleic acid portion of the peptide oligonucleotide complex is, targeted against any portion of the ACE2 protein, or a functional fragment thereof. The PD-L1-binding peptide oligonucleotide complex is administered to a subject in need thereof. The PD-L1-binding peptide oligonucleotide complex is administered intravenously, subcutaneously, intramuscularly, orally, intrathecally, intravitreally, or intratumorally. The subject is a human or an animal. In mouse models, the mice may be humanized for ACE2 expression. After administration, the PD-L1-binding peptide oligonucleotide complex reduces the ability of SARS-CoV-2 or viruses pseudotyped with SARS-CoV-2 Spike protein to infect or reinfect immune cells. The PD-L1-binding peptide oligonucleotide complex offers protection from infection and/or reduction of productive infection upon exposure to SARS-CoV-2 or SARS-CoV-2-Spike-pseudotyped viral particles, preventing a productive infection and the eventual generation of COVID-19 symptoms.

This data demonstrates that the PD-L1-binding peptide oligonucleotide complexes of the present disclosure effectively inhibit SARS-CoV-2 infection and development of COVID19.

Similarly, severe acute respiratory syndrome (SARS) is caused by the SARS-associated coronavirus (SARS-CoV-1), which also uses the angiotensin-converting enzyme 2 (ACE2) as its receptor on human cells. PD-L1-binding peptide oligonucleotide complexes of the present disclosure effectively inhibit SARS-CoV-1 or viruses pseudotyped with SARS-CoV-1 Spike protein to inhibit SARS-CoV-1 infection and development of SARS. Whereas MERS-COV uses the cellular receptor, dipeptidyl peptidase 4 (DPP4), PD-L1-binding peptide oligonucleotide complexes of the present disclosure, for example using a DPP4-targeting aptamer effectively inhibit MERS-COV or viruses pseudotyped with MERS-Cov Spike protein to inhibit SARS-CoV-1 infection and development of MERS.

### Example 41

### PD-L1-Binding Peptide Oligonucleotide Complex Using a U1 Adapter for Treatment of Skin Cancer

This example describes treatment of skin cancer (e.g., melanoma) using a PD-L1-binding peptide oligonucleotide complex described herein. BCL2 is an anti-apoptotic protein implicated in a number of solid tumors. Melanoma, in particular, expresses high levels of BCL2, rendering it resistant to many chemotherapeutics known to induce apoptosis. The BCL2 gene expresses the BCL2 protein, and reduction of BCL2 can have utility in skin cancer (e.g., melanoma). The nucleic acid portion of the peptide oligonucleotide complex which targets the BCL2 gene comprising a complementary nucleotide to BCL2 pre-mRNA linked to a U1 adapter. The 3′ end of the BCL2 pre-mRNA transcript maps to chromosome 18, and polyA mapping software PolyASite identifies the region near Base 63,126,800 (on hg38 genome assembly) as a likely polyA site. Short sequences in the BCL2 gene or pre-mRNA are identified (e.g., overlapping 20 nt sequences in the BCL2 pre-mRNA within 5000 bases on either side of this PolyA region), that are 30%-60% G/C in content and complementary sequence to BCL2 pre-mRNA and placed 5′ or 3′ (this example demonstrates 5′ placement) of a U1-recognition domain used in the complex. For example, any 20 mer complementary to the BCL2 pre-mRNA region that has imperfect complementarity (e.g., no more than 85% complementarity, or having at least 3 to 4 mismatches) or no to low complementarity (e.g., no more than 75%, 65%, 50%, or 30% complementarity) relative to other sequences in transcriptome (to reduce off target effects) may be tested.

An exemplary nucleic acid sequence contains a U1 adapter for modulating BCL2 mRNA that is highly active against BCL2 can include: 5′GCCGUACAGUUCCACAAAGGGCCAGGUAAGUAU-3′ (SEQ ID NO: 382), wherein the underlined portion (GCCGUACAGUUCCACAAAGG (SEQ ID NO: 552)) corresponds to the BCL2 recognition sequence and the italicized portion (GCCAGGUAAGUAU (SEQ ID NO: 370)) corresponds to the U1 recognition sequence. A U1 adapter may bind a target pre-mRNA molecule derived from the BCL2 open reading frame (NCBI Refseq ID: NG_009361.1). Any of the U1 adapters in TABLE 11 can also be linked to the BCL2 recognition sequence. Sugar modifications may include 2′-O-Me, LNA, or standard RNA or DNA among others. Backbone linkages can include PO or PS linkages.

The peptide and the oligonucleotide of the PD-L1-binding peptide oligonucleotide complex of the disclosure are expressed recombinantly or chemically synthesized and then conjugated together via a linker. Optionally, the linker is cleavable. Optionally, the peptide has reduced affinity to PD-L1 at pH less than 7.4. The nucleic acid portion of the peptide oligonucleotide complex is, targeted against BCL2 pre-mRNA derived from the BCL2 open reading frame (NCBI Refseq ID: NG_009361.1), or a functional fragment thereof including mRNA NCBI Refseq IDs NM_000633.3 BCL2 [organism=Homo sapiens] [GeneID=596][transcript=alpha] or NM_000657.3 BCL2 [organism=Homo sapiens] [GeneID=596][transcript=beta], SEQ ID NO: 411, or SEQ ID NO: 412. The PD-L1-binding peptide oligonucleotide complex is administered to a subject in need thereof. The PD-L1-binding peptide oligonucleotide complex is administered intravenously, subcutaneously, intramuscularly, orally, intrathecally, intravitreally, or intratumorally. The subject is a human or an animal. In mouse models, one would test in mouse xenograft models with flank tumors of human melanoma cells, or other relevant model. After administration, the PD-L1-binding peptide nucleotide complex accumulates in diseased tissue and the BCL2 mRNA transcription is reduced and the mRNA degraded, induction of apoptotic markers and reduced tumor growth results in treated animals. The PD-L1-binding peptide oligonucleotide complex ameliorates the skin cancer (e.g., melanoma). Reduced symptoms of skin cancer (e.g., melanoma) are exhibited.

This data demonstrates that the PD-L1-binding peptide oligonucleotide complexes of the present disclosure effectively treat skin cancer (e.g., melanoma).

### Example 42

### Design of an Oligonucleotide Sequence for a Peptide Oligonucleotide Complex

This example describes design of an oligonucleotide sequence for a target binding agent capable of binding a target molecule for use in a peptide oligonucleotide complex. A gene is targeted for modulation by a peptide oligonucleotide complex of this disclosure, optionally by a single stranded (ssDNA, ssRNA) or double stranded (dsDNA, dsRNA) or a combination of single and double stranded (for example with a mismatched sequence, hairpin or other structure), an antisense RNA, complementary RNA, inhibitory RNA, interfering RNA, nuclear RNA, antisense oligonucleotide (ASO), microRNA (miRNA), complementary oligonucleotide to natural antisense transcripts (NATs) sequences, siRNA, snRNA, gapmer, anti-miR, splice blocker ASO, or U1 Adapter. The gene may be targeted for downregulation to improve a disease condition. Short overlapping sequences (e.g., 12, 15, 20, 21, 25, or 30 nt in length) complementary to the gene, walking along up to the entire length of the gene, are generated and tested to determine which provides the most effective regulation. The sequence may be chosen to contain 3 or more mismatches to other sequences in the transcriptome. The sequence may be chosen to avoid any that have 14 or more matches with a nontarget or undesired complementary sequence. The sequence may be chosen to avoid the most common seed regions of 2-8 nts on the 5′ end of siRNA. Chemical modifications to the oligonucleotide are also tested (concurrently or after sequence testing). Chemical modifications may include modifications on the termini of the oligonucleotides to reduce exonuclease cleavage, such as by placing 1-3 phosphorothioate linkages on all ends. Chemical modifications may include 2′F bases such as 2′F pyrimidine bases for increased stabilization and binding. Chemical modifications may also include 2′-OMe or 2′-Omethoxyethyl bases to decrease immune activation, including to offset that which may be increased by the including of 2′F bases. Chemical modifications may also include using BNA or LNA or any other modification of this disclosure. Optionally, the oligonucleotides are tested in pool, such as 5-10 sequences at once, to narrow down to the best sequences. Optionally, the sequences are also tested for immune activation, such as with an IFIT (Interferon-induced proteins with tetratricopeptide repeats) or T cell activation assay or innate immune activation assay such as qRT-PCR, immune cell activation or proliferation or cytokine secretion, and the sequences with lower immune activation are prioritized. Optionally, nontarget AA/TT sequences are added on the ends of the siRNA. Optionally, sequence overhangs are added on the ends of the siRNA. The oligonucleotide sequences may be selected for homology to both human and other species (such as mouse, rat, and non-human primate). Alternatively, a different oligonucleotide sequence to the same target may be used in other species for preclinical development (e.g., mouse or rat) than the oligonucleotide sequence complementary to the human target which is used for clinical development and to treat human patients. Optionally, an siRNA sequence is designed using the methods of. Fakhr et al. Precise and efficient siRNA design: a key point in competent gene silencing Cancer Gene Therapy. 2016; 23, 73-82.

The oligonucleotide or the peptide oligonucleotide complex is tested for its ability to reduce the level of intact functional RNA or to reduce the level of protein which is encoded by the targeted RNA. The oligonucleotide or the peptide oligonucleotide complex is tested for its ability to generate the desired phenotypic response in the cells, tissue, or animals, such as reduced tumor growth rate, reduced cognitive decline, or reduced inflammation. The oligonucleotide or peptide oligonucleotide complex is also tested for safety or undesirable side effects. The testing is performed in vitro, in vivo, or in humans. The oligonucleotide or the peptide oligonucleotide complex with the most desired attributes is selected.

### Example 43

### Design of an Oligonucleotide Sequence for a Peptide Oligonucleotide Complex

A target gene for making target binding agent capable of binding a target molecule is selected based on the association between its expression and disease; this could be direct (e.g. either the transcript itself or a protein encoded by the transcript is associated with or leads to disease phenotype) or indirect (e.g. either the transcript itself or a protein encoded by that transcript modifies a different gene or transcript or protein whose activity is associated with or leads to disease phenotype). The target sequence is derived from the gene's open reading frame. The target sequence may be found in the coding region or in the non-coding region, and it may be found in the mature mRNA (which has been spliced, polyadenylated, capped, and exported to the cytosol for translation) or in the immature pre-mRNA. The target binding agent will be the complement to such open reading frame. If the target sequence is found in the mature mRNA (for example, when planning to use siRNA), then the search for appropriate sequences will begin with identification of the appropriate transcript isoform, taking into consideration such variables as alternative splicing or alternative transcription start sites. If the target sequence is found in the immature pre-mRNA (for example, when planning to use gapmers, splice-blocking oligonucleotides, or U1 adapters), then the search for appropriate sequences will begin with identification of the full open reading frame of the gene in question, taking into consideration such variables as alternative transcription start sites but with less consideration for alternative splice isoforms. If the target is an antisense sequence (e.g., miRNA to be targeted by an anti-miR), the sequence would be based on the mature guide strand sequence. These reference sequences can be found in public genome databases, including but not limited to the National Center for Biotechnology Information (NCBI) or the University of California Santa Cruz (UCSC) Genome Browser. The pre-mRNA sequence is the same as the genomic sequence. Optionally the reference sequences are as given in TABLE 17.

TABLE 17
Examples of Open Reading Frame Reference Sequences

| | Exemplary | NCBI Refseq | |
|---|---|---|---|
| Target | Approach | Gene | NCBI Refseq RNA(s) |
| CBP | siRNA | NG_009873.2 | NM_001079846.1, |
| (gene CREBP) | | | NM_004380.3 |
| p300 | Gapmer | NG_009817.1 | NM_001429.4, |
| (gene EP300) | | | NM_001362843.2 |
| miR-21 | Anti-miR | | NR_029493.1 (NCBI) |
| (gene MIR21) | | | MIMAT0000076 |
| | | | (miRBase) |
| BCL2 | U1 adapter | NG_009361.1 | NM_000633.3, |
| (gene BCL2) | | | NM_000657.3 |

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-214. (canceled)

215. A PD-L1-binding peptide comprising a first PD-L1-binding motif comprising a sequence of:
(a) X¹X²X³X⁴X⁵X⁶CX⁷X⁸X⁹C (SEQ ID NO: 361), wherein X¹ is D, E, H, K, N, Q, S, T, L, V, F, Y, or P; X² is G, E, Q, or F; X³ is D or K; X⁴ is G, V, or P; X⁵ is G, H, R, V, F, W, or P; X⁶ is A, D, or K; X⁷ is E, H, Q, L, or F; X⁸ is D, E, R, S, T, M, L, or F; and X⁹ is G, A, D, E, H, K, R, M, L, or P; or
(b) X¹FX²VFX²CLX³X³C (SEQ ID NO: 363), wherein X¹ is K or P; X² is independently D or K; and X³ is independently any non-cysteine amino acid.

216. The PD-L1-binding peptide of claim 215, comprising at least six cysteine residues.

217. The PD-L1-binding peptide of claim 216, wherein the at least six cysteine residues are located at amino acid positions n, n+4, n+14, n+28, n+38, and n+42, wherein n corresponds to a position of a first cysteine residue of the at least six cysteine residues.

218. The PD-L1-binding peptide of claim 215, wherein amino acid position n corresponds to amino acid position 4, such that at least six cysteine residues are located at amino acid positions 4, 8, 18, 32, 42, and 46.

219. The PD-L1-binding peptide of claim 215, wherein the first PD-L1-binding motif comprising a sequence of X¹X²X³X⁴X⁵X⁶CX⁷X⁸X⁹C (SEQ ID NO: 361), wherein X¹ is D, E, H, K, N, Q, S, T, L, V, F, Y, or P; X² is G, E, Q, or F; X³ is D or K; X⁴ is G, V, or P; X⁵ is G, H, R, V, F, W, or P; X⁶ is A, D, or K; X⁷ is E, H, Q, L, or F; X⁸ is D, E, R, S, T, M, L, or F; and X⁹ is G, A, D, E, H, K, R, M, L, or P.

220. The PD-L1-binding peptide of claim 215, wherein the first PD-L1-binding motif comprises a sequence of KFDVFKCLDHC (SEQ ID NO: 365).

221. The PD-L1-binding peptide of claim 215, further comprising a second PD-L1-binding motif comprising a sequence of CX¹X²X³CX⁴X⁵X⁶X⁷X⁸X⁹X¹⁰X¹¹X¹²C (SEQ ID NO: 360), wherein X¹ is K, R, or V; X² is E, Q, S, M, L, or V; X³ is D, E, H, K, R, N, Q, S, or Y; X⁴ is D, M, or V; X⁵ is A, K, R, Q, S, or T; X⁶ is A, D, E, H, Q, S, T, M, I, L, V, or W; X⁷ is A, E, R, Q, S, T, W, or P; X⁸ is A, E, K, R, N, Q, T, M, I, L, V, or W; X⁹ is G, A, E, K, N, T, or Y; X¹⁰ is G, A, D, E, H, K, R, N, Q, S, T, M, I, L, V, W, Y, or P; X¹¹ is D, K, R, N, L, or V; and X¹² is G, A, D, T, L, W, or P.

222. The PD-L1-binding peptide of claim 221, wherein the second PD-L1-binding motif comprises a sequence of CKVHCVKEWMAGKAC (SEQ ID NO: 364).

223. The PD-L1-binding peptide of claim 215, wherein the PD-L1-binding peptide comprises a sequence of SEQ ID NO: 358.

224. The PD-L1-binding peptide of claim 215, wherein the PD-L1-binding peptide comprises a sequence having at least 80% sequence identity to any one of SEQ ID NO: 1-SEQ ID NO: 4 or SEQ ID NO: 554.

225. The PD-L1-binding peptide of claim 215, wherein the PD-L1-binding peptide comprises a sequence of any one of SEQ ID NO: 1-SEQ ID NO: 4 or SEQ ID NO: 554.
